SEQUENCE LISTING (434 sequences)

SEQ ID NO: 1 - protein, 339 aa, synthetic construct
MLAAVVREFGKPLSIERLPIPDIKPHQILVKVDTCGVCHTDLHAARGDWPSKPNPPFIPGHEGVGHIVAVGSQVGDFVKTGDVVGVPWLYSACGHCEHCLGGWETLCEKQDDTGYTVNGCFAEYVVADPNYVAHLPSTIDPLQASPVLCAGLTVYKGLKMTEARPGQWVAVSGVGGLGQMAVQYAVAMGMNVVAVDIDDEKLATAKKLGASLTVNAKDTDPARFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 2 - protein, 479 aa, synthetic construct
MSVPVQHPMYIDGQFVTWRGDAWIDVVNPATEAVISRIPDGQAEDARKAIDAAERAQPEWEALPAIERASWLRKISAGIRERASEISALIVEEGGKIQQLAEVEVAFTADYIDYMAEWARRYEGEIIQSDRPGENILLFKRALGVTTGILPWNFPFFLIARKMAPALLTGNIIVIKPSEFTPNNAIAFAKIVDEIGLPRGVFNLVLGRGETVGQELAGNPKVAMVSMTGSVSAGEKIMATAAKNITKVCLELGGKAPAIVMDDADLELAVKAIVDSRVINSGQVCNCAERVYVQKGIYDQFVNRLGEAMQAVQFGNPAERNDIAMGPLINAAALERVEQKVARAVEEGARVAFGGKAVEGKGYYYPPTLLLDVRQEMSIMHEETFGPVLPVVAFDTLEDAISMANDSDYGLTSSIYTQNLNVAMKAIKGLKFGETYINRENFEAMQGFHAGWRKSGIGGADGKHGLHEYLQTQVVYLQS

SEQ ID NO: 3 - DNA, 91 bp, synthetic construct
  source 1..91: mol_type = other DNA
tcgtcagtaaaaagttaatcttttcaacacctgtcataaagttgtcacggccgagacttatagtcgctttgtttttattttttaatgtatt

SEQ ID NO: 4 - DNA, 30 bp, synthetic construct
  source 1..30: mol_type = other DNA
ctaccaaccaaccacatttcaggtttacat

SEQ ID NO: 5 - DNA, 19 bp, synthetic construct
  source 1..19: mol_type = other DNA
tgtacatggagaaaataaa

SEQ ID NO: 6 - DNA, 75 bp, synthetic construct
  source 1..75: mol_type = other DNA
aacgcagagttcgaggatcttcccacccagctcttccgcgcgggcaaattcaccggtgtaggctggagctgcttc

SEQ ID NO: 7 - DNA, 75 bp, synthetic construct
  source 1..75: mol_type = other DNA
gtaccggtggtgacccgtggtgcaggcaccgggctttctggtggcgcgctgccgcatgggaattagccatggtcc

SEQ ID NO: 8 - protein, 343 aa, Gluconobacter oxydans
MADTMLAAVVREFGKPLSIERLPIPDIKPHQILVKVDTCGVCHTDLHAARGDWPSKPNPPFIPGHEGVGHIVAVGSQVGDFVKTGDVVGVPWLYSACGHCEHCLGGWETLCEKQDDTGYTVNGCFAEYVVADPNYVAHLPSTIDPLQASPVLCAGLTVYKGLKMTEARPGQWVAVSGVGGLGQMAVQYAVAMGMNVVAVDIDDEKLATAKKLGASLTVNAKDTDPARFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 9 - protein, 343 aa, Gluconobacter sp.
MADTMLAAVVREFGKPLSIERLPIPDIKPHQILVKVDTCGVCHTDLHAARGDWPSKPNPPFIPGHEGVGHIVAVGSQVGDFVKTGDVVGVPWLYSACGHCEHCLGGWETLCEKQDDTGYTVNGCFAEYVVADPNYVAHLPSTIDPLQASPVLCAGLTVYKGLKMTEARPGQWVAVSGVGGLGQMAVQYAVAMGMNVVAVDIDDEKLATAKKLGASLTVNAKDTDPAKFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 10 - protein, 339 aa, Gluconobacter oxydans
  source 1..339: note = strain NBRC 3293
MLAAVVREFGKPLSIERLPIPDIKPHQILVKVDTCGVCHTDLHAARGDWPSKPNPPFIPGHEGVGHIVAVGSQVGDFVKTGDVVGVPWLYSACGHCEHCLGGWETLCEKQDDTGYTVNGCFAEYVVADPNYVAHLPSTIDPLQASPVLCAGLTVYKGLKMTEARPGQWVAVSGVGGLGQMAVQYAVAMGMNVVAVDIDDEKLATAKKLGASLTVNAKDTDPAKFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 11 - protein, 343 aa, Gluconobacter oxydans
MADTMLAAVVREFGKPLSIERLPIPDIKPHQILVKVDTCGVCHTDLHAARGDWPSKPSPPFIPGHEGVGHIVAVGSQVGDFVKTGDVVGVPWLYSACGHCEHCLGGWETLCEKQDDTGYTVNGCFAEYVVADPNYVAHLPSTIDPLQASPVLCAGLTVYKGLKMTEARPGQWVAVSGVGGLGQMAVQYAVAMGMNVVAVDIDDEKLATAKKLGASLTVNAKDTDPAKFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 12 - protein, 343 aa, Gluconobacter oxydans
MADTMLAAVVREFGKPLSIERLPIPDIQPHQILVKVDTCGVCHTDLHAARGDWPSKPNPPFIPGHEGVGHIVAVGSQVGDFVKTGDVVGVPWLYSACGHCEHCLGGWETLCEKQDDTGYTVNGCFAEYVVADPNYVAHLPSTIDPLQASPVLCAGLTVYKGLKMTEARPGQWVAVSGVGGLGQMAVQYAVAMGMNVVAVDIDDEKLATAKKLGASLTVNAKDTDPAEFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 13 - protein, 343 aa, Gluconobacter oxydans
MADTMLAAVVREFGKPLSIERLPIPDIQPNQILVKVDTCGVCHTDLHAARGDWPSKPNPPFIPGHEGVGHIVAVGSQVGDFVKTGDVVGVPWLYSACGHCEHCLGGWETLCEKQDDTGYTVNGCFAEYVVADPNYVAHLPSTIDPLQASPVLCAGLTVYKGLKMTEARPGQWVAVSGVGGLGQMAVQYAVAMGMNVVAVDIDDEKLATAKKLGASLTVNAKDTDPAKFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 14 - protein, 343 aa, Gluconobacter aidae
MADTMLAAVVREFGKPLSIERLPIPDIKPNQILVKVDTCGVCHTDLHAARGDWPSKPNPPFIPGHEGVGHIVAVGSQVGDFVKTGDVVGVPWLYSACGHCEHCLGGWETLCEKQEDTGYTVNGCFAEYVVADPNYVAHLPATIDPLQASPVLCAGLTVYKGLKMTEARPGQWVAVSGVGGLGQMAVQYAVAMGMNVVAVDIDDEKLATAKKLGASLTVNAKDTDPAKFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 15 - protein, 343 aa, Gluconobacter roseus
MAETMLAAVVREFGKPLSIERLPIPDIKPNQILVKVDTCGVCHTDLHAARGDWPTKPNPPFIPGHEGVGHIVAVGSQVGDFVKTSDVVGVPWLYSACGHCEHCLGGWETLCEKQEDTGYTVNGCFAEYVVADPNYVAHLPATIDPLQASPVLCAGLTVYKGLKMTEARPGQWVAVSGVGGLGQMAVQYAVAMGMNVVAVDIDDEKLATAKKLGASLTVNAKDTDPARFIQQQIGGAHGALVTAVGRTAFSQAMGYARRGGTIVLNGLPPGDFPVSIFDMVMNGTTIRGSIVGTRLDMIEAMDFFARGKVKSVVTSGKLENINTIFDDLQNGRLEGRTVLDFRS

SEQ ID NO: 16 - protein, 343 aa, Gluconobacter sp.
  source 1..343: note = strain P1C6_b
MADTMLAAVVREFGKPLSIERLPIPDIQPHQILVKVDTCGVCHTDLHAARGDWPSKPNPPFIPGHEGVGHIVAVGSQVGDFVKTGDVVGVPWLYSACGHCEHCFGGWETLCEKQEDTGYTVNGCFAEYVVADPNYVAHLPSTIDPLQASPVLCAGLTVYKGLKMTEARAGQWVAISGVGGLGQMAVQYAVAMGMNVVAVDIDDEKLATAKTLGAALTVNARDTDPAKFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 17 - protein, 343 aa, Gluconobacter oxydans
MADTMLAAVVREFGKPLSIERLPIPDIQPHQILVKVDTCGVCHTDLHAARGDWPSKPNPPFIPGHEGVGHIVAVGSQVGNFVKTGDVVGVPWLYSACGHCEHCFGGWETLCEKQEDTGYTVNGCFAEYVVADPNYVAHLPSAIDPLQASPVLCAGLTVYKGLKMTEARAGQWVAISGVGGLGQMAVQYAVAMGMNVVAVDIDDEKLATAKTLGASLTVNAKDTDPAKFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 18 - protein, 343 aa, Gluconobacter sp.
MADTMLAAVVREFGKPLTIERLPIPDVRPNQILVKVDTCGVCHTDLHAARGDWPSKPNPPFIPGHEGVGHIVAVGSQVDNFVKVGDVVGVPWLYSACGHCEHCLGGWETLCEKQDDTGYTVNGCFAEYVVADPNYVAHLPSTIDPLQTAPVLCAGLTVYKGLKMTEARPGQWVAISGVGGLGQMAVQYAIAMGMNVVAVDIDDEKLATAKKLGASLTVNAKDTDPAKFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 19 - protein, 343 aa, Gluconobacter sp.
  source 1..343: note = strain Dm-62
MADTMLAAVVREFGKPLSIERLPIPDIQPHQILVKVDTCGVCHTDLHAARGDWPSKPNPPFIPGHEGVGHIVAVGSQVGDFVKTGDVVGVPWLYSACGHCEHCFAGWETLCEKQEDTGYSVNGCFAEYVVADPNYVAHLPSTIDPLQASPVLCAGLTVYKGLKMTEARAGQWVAISGVGGLGQMAVQYAVAMGMNVVAVDIDDEKLTTAKTLGAALTVNAKDTDPAKFIQQQIGGAHGALVTAVGRTAFSQAMGYARRGGTIVLNGLPPGDFPVSIFDMVMNGTTIRGSIVGTRLDMIEAMDFFARGKVKSVVTPGKLESINTIFDDLQNGRLEGRTVLDFRS

SEQ ID NO: 20 - protein, 343 aa, Gluconobacter potus
MADTMLAAVVREFGKPLTIERLPIPDVRPNQILVKVDTCGVCHTDLHAARGDWPSKPNPPFIPGHEGVGHIVAVGSQVDNFVKVGDVVGVPWLYSACGHCEHCLGGWETLCEKQDDTGYTVNGCFAEYVVADPNYIAHLPSTIDPLQTAPVLCAGLTVYKGLKMTEARPGQWVAISGVGGLGQMAVQYAIAMGMNVVAVDIDDEKLATAKKLGASLTVNAKDTDPAKFIQQQIGGAHGALVTAVGRTAFSQAMGYARRGGTIVLNGLPPGDFPVSIFDMVMNGTTIRGSIVGTRLDMIEAMDFFARGKVKSVVTPGKLENINTIFDDLQNGRLEGRTVLDFRS

SEQ ID NO: 21 - protein, 254 aa, Gluconobacter japonicus
VPWLYSACGHCEHCLGGWETLCKDQDDTGYTVNGCFAEYVVADPNYVAHLPSTIDPLQAAPVLCAGLTVYKGLKMTEAHPGQWMAISGVGGLGQMAVQYAVAMGMNVVAVDIDDDKLATAKKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 22 - protein, 342 aa, Gluconobacter sp.
MADTMLAAVVREFGKPLSVERLPIPEIRPNQILVKVDTCGVCHTDLHAAEGDWPAKPNPPFIPGHEGIGHIVAVGSQVNHVKVGDVVGVPWLYSACGHCEHCLGGWETLCKDQDDTGYTVNGCFAEYVVADPNYVAHLPSNIDPLQAAPVLCAGLTVYKGLKMTEARPGQWMAISGVGGLGQMAVQYAVAMGMNVVAVDIDDDKLATAKKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 23 - protein, 342 aa, Gluconobacter thailandicus
MADTMLAAVVREFGKPLSVERLPIPEIRPNQILVKVDTCGVCHTDLHAAEGDWPAKPNPPFIPGHEGIGHIVAVGSQVNHVKVGDVVGVPWLYSACGHCEHCLGGWETLCKDQDDTGYTVNGCFAEYVVADPNYVAHLPSNIDPLQAAPVLCAGLTVYKGLKMTEARPGQWMAISGVGGLGQMAVQYAVAMGMNVVAVDIDEDKLATAKKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 24 - protein, 342 aa, Gluconobacter frateurii
MADTMLAAVVREFGKPLSVERLPIPEIRPNQILVKVDTCGVCHTDLHAAEGDWPAKPNPPFIPGHEGIGHIVAVGSQVNHVKVGDVVGVPWLYSACGHCEHCLGGWETLCKEQDDTGYTVNGCFAEYVVADPNYVAHLPSNIDALQAAPVLCAGLTVYKGLKMTEARPGQWMAISGVGGLGQMAVQYAVAMGMNVVAVDIDDDKLATAKKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 25 - protein, 342 aa, Gluconobacter japonicus
MADTMLAAVVREFGKPLSVERLPIPEIRPNQILVKVDTCGVCHTDLHAAEGDWPAKPNPPFIPGHEGIGHIVAVGSQVNHVKVGDVVGVPWLYSACGHCEHCLGGWETLCKDQDDTGYTVNGCFAEYVVADPNYVAHLPSNIDALQAAPVLCAGLTVYKGLKMTEARPGQWMAISGVGGLGQMAVQYAVAMGMNVVAVDIDDDKLATAKKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 26 - protein, 342 aa, Gluconobacter sp.
MADTMLAAVVREFGKPLSVERLPIPEIRPDQILVKVDTCGVCHTDLHAAEGDWPAKPNPPFIPGHEGVGHIVAVGSQVKHSKVGDMVGVPWLYSACGHCEHCLGGWETLCKDQDDTGYTVNGCFAEYVVADPNYVAHLPSTIDPLQAAPVLCAGLTVYKGLKMTEAHPGQWMAISGVGGLGQMAVQYAVAMGMNVVAVDIDDDKLATAKKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 27 - protein, 342 aa, Gluconobacter cerinus
MADTMLAAVVREFGKPLSVERLPIPEIRPNQILVKVDTCGVCHTDLHAAEGDWPAKPNPPFIPGHEGIGHIVAVGSQVNHVKVGDVVGVPWLYSACGHCEHCLGGWETLCKEQDDTGYTVNGCFAEYVVADPNYVAHLPSNIDALQAAPVLCAGLTVYKGLKMTEARPGQWMAISGVGGLGQMAVQYAVAMGMNVVAVDIDDDKLATATKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 28 - protein, 342 aa, Gluconobacter thailandicus
MADTMLAAVVREFGKPLSVERLPIPEIRPNQILVKVDTCGVCHTDLHAAEGDWPAKPNPPFIPGHEGIGHIVAVGSQVNHVKVGDVVGVPWLYSACGHCEHCLGGWETLCKDQDDTGYTVNGCFAEYVVADPNYVAHLPSNIDALQAAPVLCAGLTVYKGLKMTEARPGQWMAISGVGGLGQMAVQYAVAMGMNVVAVDIDEDKLATAKKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 29 - protein, 342 aa, Gluconobacter sp.
MADTMLAAVVREFGKPLSVERLPIPEIRPNQILVKVDTCGVCHTDLHAAEGDWPAKPNPPFIPGHEGIGHIVAVGSQVSHAKIGDVVGVPWLYSACGHCEHCLGGWETLCKDQDDTGYTVNGCFAEYVVADPNYVAHLPSNIDALQAAPVLCAGLTVYKGLKMTEARPGQWMAISGVGGLGQMAVQYAVAMGMNVVAVDIDDDKLATAKKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 30 - protein, 342 aa, Gluconobacter japonicus
MADTMLAAVVREFGKPLSVERLPIPEIRPDQILVKIDTCGVCHTDLHAAEGDWPAKPNPPFIPGHEGVGHIVAVGSQVKHSKVGDMVGVPWLYSACGHCEHCLGGWETLCKDQDDTGYTVNGCFAEYVVADPNYVAHLPSTIDPLQAAPVLCAGLTVYKGLKMTEAHPGQWMAISGVGGLGQMAVQYAVAMGMNVVAVDIDDDKLATAKKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 31 - protein, 342 aa, Gluconobacter sp.
MADTMLAAVVREFGKPLSVERLPIPEIRPNQILVKVDTCGVCHTDLHAAEGDWPAKPNPPFIPGHEGIGHIVAVGSQVSHAKIGDVVGVPWLYSACGHCEHCLGGWETLCKDQDDTGYTVNGCFAEYVVADPNYVAHLPSNIDALQAAPVLCAGLTVYKGLKMTEARPGQWMAISGVGGLGQMAVQYAVAMGMNVVAVDIDDDKLATATKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 32 - protein, 342 aa, Gluconobacter cerinus
MADTMLAAVVREFGKPLSVERLPIPEIRPNQILVKVDTCGVCHTDLHAAEGDWPAKPNPPFIPGHEGIGHIVAVGSQVSHAKIGDVVGVPWLYSACGHCEHCLGGWETLCKDQDDTGYTVNGCFAEYVVADPNYVAHLPSNIDALQAAPVLCAGLTVYKGLKMTEARPGQWMAISGVGGLGQMAVQYAVAMGMNVVAVDIDDDKLATATKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKVENINTIFDNLKNGRLQGRTVLDFRS

SEQ ID NO: 33 - protein, 342 aa, Gluconobacter cerinus
MADTMLAAVVREFGKPLSVERLPIPEIRPSQILVKVDTCGVCHTDLHAAEGDWPAKPNPPFIPGHEGIGHIVAVGSQVSHAKIGDVVGVPWLYSACGHCEHCLGGWETLCKDQDDTGYTVNGCFAEYVVADPNYVAHLPSNIDALQAAPVLCAGLTVYKGLKMTEARPGQWMAISGVGGLGQMAVQYAVAMGMNVVAVDIDDDKLATATKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 34 - protein, 342 aa, unclassified Gluconobacter
MADTMLAAVVREFGKPLSVERLPIPEIRPNQILVKVDTCGVCHTDLHAAEGDWPAKPNPPFIPGHEGIGHIVAVGSQVSHAKIGDVVGVPWLYSACGHCEHCLGGWETLCKDQDDTGYTVNGCFAEYVVADPNYVAHLPSNIDALQAAPVLCAGLTVYKGLKMTEARPGQWMAISGVGGLGQMAVQYAVAMGMNVVAVDIDDDKLATATKLGATLTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 35 - protein, 342 aa, Gluconobacter japonicus
MADTILAAVVREFGKPLSVERLPIPEIRPDQILVKVDTCGVCHTDLHAAEGDWPAKPNPPFIPGHEGVGHIVAVGSQVKHSRVGDMVGVPWLYSACGHCEHCLGGWETLCKDQDDTGYTVNGCFAEYVVADPNYVAHLPSTIDPLQAAPVLCAGLTVYKGLKMTEAHPGQWMAISGVGGLGQMAVQYAVAMGMNVVAVDIDDDKLATAKKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 36 - protein, 342 aa, Gluconobacter japonicus
MADTMLAAVVREFGKPLSVERLPIPEIRSDQILVKIDTCGVCHTDLHAAEGDWPAKPNPPFIPGHEGVGHIVAVGSQVKHSKIGDMVGVPWLYSACGHCEHCLGGWETLCKDQDDTGYTVNGCFAEYVVADPNYVAHLPSTIDPLQAAPVLCAGLTVYKGLKMTEAHPGQWMAISGVGGLGQMAVQYAVAMGMNVVAVDIDDDKLATAKKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 37 - protein, 342 aa, Gluconobacter cerinus
MADTMLAAVVREFGKPLSVERLPIPEIRPNQILVKVDTCGVCHTDLHAAEGDWPAKPNPPFIPGHEGIGHIVAVGSQVSHAKIGDVVGVPWLYSACGHCEHCLGGWETLCKEQDDTGYTVNGCFAEYVVADPNYVAHLPSNIDALQAAPVLCAGLTVYKGLKMTEARPGQWMAISGVGGLGQMAVQYAVAMGMNVVAVDIDDDKLATATELGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 38 - protein, 342 aa, Gluconobacter cerinus
MADTMLAAVVREFGKPLSVERLPIPEIRPNQILVKVDTCGVCHTDLHAAEGDWPAKPNPPFIPGHEGIGHIVAVGSQVSHAKIGDVVGVPWLYSACGHCEHCLGGWETLCKDQDDTGYTVNGCFAEYVVADPNYVAHLPSNIDALQAAPVLCAGLTVYKGLKMTEARPGQWMAISGIGGLGQMAVQYAVAMGMNVVAVDIDDDKLATATKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 39 - protein, 342 aa, Gluconobacter cerinus
MADTMLAAVVREFGKPLSVERLPIPEIRPNQILVKVDTCGVCHTDLHAAEGDWPAKPNPPFIPGHEGIGHIVAVGSQVSHAKIGDMVGVPWLYSACGHCEHCLGGWETLCKDQDDTGYTVNGCFAEYVVADPNYVAHLPSNIDALQAAPVLCAGLTVYKGLKMTEARPGQWMAISGVGGLGQMAVQYAVAMGMNVVAVDIDDDKLATATKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 40 - protein, 342 aa, Gluconobacter cerinus
MADTMLAAVVREFGKPLSVERLPIPEIRPNQILVKVDTCGVCHTDLHAAEGDWPAKPNPPFIPGHEGIGHIVAVGSQVSHAKIGDVVGVPWLYSACGHCEHCLGGWETLCKDQDDTGYTVNGCFAEYVVADPNYVAHLPSNIDALQAAPVLCAGLTVYKGLKMTEARPGHWMAISGVGGLGQMAVQYAVAMGMNVVAVDIDDDKLATATKLGAALTVNAKDKDPAAFIQQEVGGAHGALVTAVGRTAFSQAMGYARRGGTIVLNGLPPGDFPVSIFDMVMNGTTIRGSIVGTRLDMIEAMDFFARGKVKSVVTADKIENINTIFDNLKNGRLQGRTVLDFRS

SEQ ID NO: 41 - protein, 343 aa, Gluconobacter morbifer
MADTMQAAVVREFGKPLSIERVPVPEIRPNQILVKVETCGVCHTDLHAARGDWPTKPNPPFIPGHEGVGRIVALGSQVGDFVKVGDRVGVPWLYSACGHCEHCLGGWETLCEKQEDTGYSVNGCFAEYVAADPNYVAHLPENIDPLETSPVLCAGLTVYKGLKMTEARPGQWVAVSGIGGLGQMAVQYAVAMGMNVVAVDIDDGKLETAKKLGAVLTVNAKDRDPAKFVQQEIGGAHGALVTAVGRTAFAQAMGYARRGGTIVLNGLPPGDFPISIFDMVMNGTTVRGSIVGTRLDMIEALDFFARGKVKSVVTADKLENINTIFDNLQNGRLAGRTVLDFRS

SEQ ID NO: 42 - protein, 342 aa, Kozakia baliensis
MAEKMKAAVVREFGKPLVIEEVDIPEIRPDQILVKVDACGVCHTDLHAARGDWPAKPNPPFIPGHEGIGHIVAVGSQVKYAKTGDVVGVPWLYSACGHCEHCLGGWETLCEKQDDTGYTVNGCFAEYVVADPNYVAHLPKNIDPVKVAPLLCAGLTVYKGLKMTEARPGQWVAVSGVGGLGQMAIQYGVAMGLNVIAVDIDDEKLATAKTLGAALTVNAKDTDPAAFVHQKVGGAHGALVTAVSRSAFSQAMGYARRGGTIVLNGLPPGDFPISIFDMVMNGTTIRGSIVGTRLDMIEALSFFTDGKVETVTRADKLENINAIFEHLEKGTVDGRIVLDFRH

SEQ ID NO: 43 - protein, 342 aa, Kozakia baliensis
MAEKMKAAVVREFGKPLVIEEVDIPGIRPDQILVKVDACGVCHTDLHAARGDWPAKPNPPFIPGHEGIGHIVAVGSQVKYAKTGDVVGVPWLYSACGHCEHCLGGWETLCEKQDDTGYTVNGCFAEYVVADPNYVAHLPKNIDPVKVAPLLCAGLTVYKGLKMTEARPGQWVAVSGVGGLGQMAIQYGVAMGLNVIAVDIDDEKLATAKTLGAALTVNAKDTDPAAFVHQKVGGAHGALVTAVSRSAFSQAMGYARRGGTIVLNGLPPGDFPISIFDMVMNGTTIRGSIVGTRLDMIEALSFFTDGKVETVTRTDKLENINAIFEHLEKGTVDGRIVLDFRN

SEQ ID NO: 44 - protein, 354 aa, Gluconacetobacter diazotrophicus
  source 1..354: note = strain PA1 5
MRSADTILGGTSMTGKMKAAVVREFGKPLTIEELDIPSIRPDQILVKMDACGVCHTDLHAARGDWPVKPNPPFIPGHEGVGHVVSVGSAVKWVKLGDVVGVPWLYSACGHCEHCLGGWETLCASQDDTGYTVNGCFAEYVVADPSYVAHLPKTIDPLQVAPVLCAGLTVYKGLKMTDTKPGDWVAVSGVGGLGQMAVQYGVAMGMNVIAVDIDDEKLATAKALGAALTVNARATDPAPFIQSQVGGAHGALVTAVSRSAFSQAMGFARRGGTIVLNGLPPGEFPISIFDMVMAGTTVRGSIVGTRLDMIEAVSFFADGKVKTVIEPDRLENINQIFTDLEEGKIQGRKVLDFRQ

SEQ ID NO: 45 - protein, 342 aa, Gluconacetobacter diazotrophicus
MTGKMKAAVVREFGKPLTIEELDIPSIRPDQILVKMDACGVCHTDLHAARGDWPVKPNPPFIPGHEGVGHVVSVGSAVKWVKLGDVVGVPWLYSACGHCEHCLGGWETLCASQDDTGYTVNGCFAEYVVADPSYVAHLPKTIDPLQVAPVLCAGLTVYKGLKMTDTKPGDWVAVSGVGGLGQMAVQYGVAMGMNVIAVDIDDEKLATAKALGAALTVNARATDPAPFIQSQVGGAHGALVTAVSRSAFSQAMGFARRGGTIVLNGLPPGEFPISIFDMVMAGTTVRGSIVGTRLDMIEAVSFFADGKVKTVIEPDRLENINQIFTDLEEGKIQGRKVLDFRQ

SEQ ID NO: 46 - protein, 343 aa, Gluconacetobacter diazotrophicus
MTGKMKAAVVREFGKPLTIEELDIPSIRPDQILVKMDACGVCHTDLHAARGDWPVKPNPPFIPGHEGVGHVVSVGSAVKWVKLGDVVGVPWLYSACGHCEHCLGGWETLCASQDDTGYTVNGCFAEYVVADPSYVAHLPKTIDPLQVAPVLCAGLTVYKGLKMTDTKPGDWVAVSGVGGLGQMAVQYGVAMGMNVIAVDIDDEKLATAKALGAALTVNARATDPAPFIQSQVGGAHGALVTAVSRSAFSQAMGFARRGGTIVLNGLPPGEFPISIFDMVMAGTTVRGSIVGTRLDMIEAVSFFADGKVKTVIEPDRLENINQIFTDLEEGKIQGRKVLDFRKQ

SEQ ID NO: 47 - protein, 342 aa, Acetobacter musti
MAGKMKAAVVREFGKPLSIEELDIPAISTNQILVKVDTCGVCHTDLHAARGDWPTKPNPPFIPGHEGVGHVVQVGSNVNWIREGDVVGVPWLYSACGHCEHCLGGWETLCEKQEDTGYSVNGCFAEYVVADPNYVAHIPAGADPLHIAPVLCAGLTVYKGLKMTDTRAGNWVAVSGVGGLGQMAIQYGVAMGLNMIAVDIDDGKLAEAKQLGATLTVNAKKTDPAAFVQKEVGGAHGALVTAVSRIAFSQAMGYARRGGTIVLNGLPPGEFPVSIFDMVMTGTTVRGSIVGTRLDMIEALSFFSEGKVRSVVKPDKLENINQIFDDLQNGRVEGRVVLDFRT

SEQ ID NO: 48 - protein, 342 aa, Acetobacter oeni
MAGKMKAAVVREFGKPLSIEELDIPSINENQILVKVDTCGVCHTDLHAARGDWPVKPKPPFIPGHEGVGHVVEVGSNVNWIREGDIVGVPYLYSACGHCEHCLGGWETLCEKQEDTGYTVNGCFAEYVVADPNYVAHIPKGVDLLHTAPVLCAGLTVYKGLKMTEARAGNWVAVSGVGGLGQMAIQYGVAMGFNMIAVDIDDGKLAEAKKLGAALTVNAKEADPAAFVQKEVGGAHGALVTAVSRIAFSQAMGYARRGGTIVLNGLPPGEFPVSIFDMVMAGTTVRGSIVGTRLDMIEALSFFSEGKVRSVVKPDKLENINQIFDDLQNGRVEGRVVLDFRT

SEQ ID NO: 49 - protein, 342 aa, Acetobacter fallax
MTGKMKAAVVREFGKPLTIEELDIPTISQNQILVKVDTCGVCHTDLHAARGDWPVKPKPPFIPGHEGVGHVVQVGSNVSWIKEGDVVGVPYLYSACGHCEHCLGGWETLCEKQEDTGYTVNGCFAEYVVADPNYVAHIPPGMDPLHVSPVLCAGLTVYKGLKMTDTRAGNWVAVSGVGGLGQMAIQYGVAMGLNMIAVDIDDAKLAEAKKLGAALTVNAKETDPAAFVQKEVGGAHGALVTAVSRIAFSQAMGYARRGGTIVLNGLPPGEFPVSIFDMVMAGTTVRGSIVGTRLDMIEALSFFSEGKVRSVVKPDKLENINQIFDDLEHGRVEGRVVLDFRT

SEQ ID NO: 50 - protein, 342 aa, Acetobacter farinalis
MCAKMKAAVVREFGKPLTIEELEIPTIDQNHILVKMEACGVCHTDLHAARGDWPTKPTPPFTPGHEGVGRVVQVGSNVKWVKEGDRVGVPWLYSACGHCEHCLGGWETLCEKQDDTGYTVNGCFAEYVVADPNYVARIPDGVDPIQIAPVLCAGLTVYKGLKMTEARPGNWVAISGVGGLGQMAVQYGIAMGLNIVAVDIDDEKLETARKLGAALTVNAKNTDPATFVQKEIGGAHGVLVTAVSRTAFAQAMGYTRRGGTIVLNGLPPGDFPLSIFDMVMAGTTVRGSIVGTRLDMIEALSFFAEGKIHSVVKTDKLENINSIFDDLQNGRIDGRVVLDFRG

SEQ ID NO: 51 - protein, 342 aa, Gluconacetobacter azotocaptans
MAGTMKAAVVRAFGAPLTIEELAVPDIRPDQILVKMDACGVCHTDLHAARGDWPVKPTLPFIPGHEGVGHVVAVGSAVKWVREGDVVGVPWLYSACGHCEHCLGGWETLCKTQDDTGYTVNGCFAEFVVADPNYVAHLPKGVDPIQVAPVLCAGLTVYKGLKMTDTRPGQWVAVSGVGGLGQMAVQYGVAMGLNVIAVDIDDEKLATARSLGAALTVNAAQTDPATFIQDQVGGAHGTLVTAVSRSAFSQAMGYARRGGTIVLNGLPPGEFPISIFDMVMAGTTVRGSIVGTRLDMIEALSFFADGKVRTVVAPERLENINRIFDDLEHGTVQGRMVIDFRN

SEQ ID NO: 52 - protein, 338 aa, Gluconacetobacter azotocaptans
  source 1..338: note = strain DSM 13594
MKAAVVRAFGAPLTIEELAVPDIRPDQILVKMDACGVCHTDLHAARGDWPVKPTLPFIPGHEGVGHVVAVGSAVKWVREGDVVGVPWLYSACGHCEHCLGGWETLCKTQDDTGYTVNGCFAEFVVADPNYVAHLPKGVDPIQVAPVLCAGLTVYKGLKMTDTRPGQWVAVSGVGGLGQMAVQYGVAMGLNVIAVDIDDEKLATARSLGAALTVNAAQTDPATFIQDQVGGAHGTLVTAVSRSAFSQAMGYARRGGTIVLNGLPPGEFPISIFDMVMAGTTVRGSIVGTRLDMIEALSFFADGKVRTVVAPERLENINRIFDDLEHGTVQGRMVIDFRN

SEQ ID NO: 53 - protein, 342 aa, Gluconacetobacter asukensis
MTGKMKAAVVREFGKPLTIEELDIPTIRPDQILVRLDACGVCHTDLHAARGDWPVKPKPPFVPGHEGVGHVVAVGSAVKWVKKGDVVGVPWLYSACGHCEHCLGGWETLCPTQDDTGYTVNGCFAEYVVADPNYVAHLPKNIDPLQVAPVLCAGLTVYKGLKMTDTRPGQWVAVSGVGGLGQMAVQYGVAMGLNVIAIDIDDEKLQTAKVLGAALTVNARSVDPAPYIQSQVGGAHGTLVTAVSRSAFAQAMGFARRGGTIVLNGLPPGEFPISIFDMVMAGTTVRGSIVGTRLDMIEALSFFADGKVKTVIEPDRLENINQIFADLETGNVHGRKVLDLRD

SEQ ID NO: 54 - protein, 342 aa, Gluconacetobacter azotocaptans
MAGTMKAAVVRAFGAPLTIEELAVPDIRPDQILVKMDACGVCHTDLHAARGDWPVKPTLPFIPGHEGVGHVVAVGSAVKWVREGDVVGVPWLYSACGHCEHCLGGWETLCKTQDDTGYTVNGCFAEFVVADPNYAAHLPKGVDPIQVAPVLCAGLTVYKGLKMTDTRPGQWVAVSGVGGLGQMAVQYGVAMGLNVIAVDIDDEKLATARSLGAALTVNAAQTDPATFIQDQVGGAHGTLVTAVSRSAFSQAMGYARRGGTIVLNGLPPGEFPISIFDMVMAGTTVRGSIVGTRLDMIEALSFFADGKVRTVVAPERLENINRIFDDLEHGTVQGRMVIDFRN

SEQ ID NO: 55 - protein, 342 aa, Gluconacetobacter tumulisoli
MAGTMKAAVVRAFGAPLTIEELAVPQIRPDQILVKMDACGVCHTDLHAARGDWPVKPTPPFIPGHEGVGHVVAVGSAVKWVREGDVVGVPWLYSACGHCEHCLGGWETLCKTQDDTGYTVNGCFAEYVVADPNYVAHLPKGVDPVQVAPVLCAGLTVYKGLKMTDARPGQWVAVSGVGGLGQMAVQYGVAMGLNVIAVDIDDGKLATARTLGAALTVNAAQTDPAAFIQDHVGGAHGTLVTAVSRSAFAQAMGYARRGGTIVLNGLPPGEFPISIFDMVMAGTTVRGSIVGTRLDMIEALSFFADGKVRTVVAPERLENINRIFDDLEHGTVQGRMVIDFRN

SEQ ID NO: 56 - protein, 342 aa, Acetobacter aceti
MAGKMKAAVAHEFNKPLTIEELDIPTINQNQILVKMDACGVCHTDLHAVRGDWPVKPTLPFIPGHEGVGHVVQVGSNVNWVKEGDYVGVPYLYSACGHCLHCLGGWETLCEKQEDTGYTVNGCFAEYVVADPNYVAHIPKGADPLQVAPVLCAGLTVYKALKMTDTKPGDWVAVSGVGGLGQMAMQYGVAMGKNMIAVDIDDEKLATAKKLGAALTVNARDTDPAAFIQKEVGGAQGVVVTAVSRIAFSQAMGYARRGGTIVLNGLPPGDFPVSIFDMVMNGTTVRGSIVGTRLDMIEALSFFADGKVHSVVKPDKLENINRIFDDLENGRIDGRVVLDFRN

SEQ ID NO: 57 - protein, 342 aa, Gluconacetobacter tumulicola
MTGKMKAAVVREFGKPLTIEELDIPTIRPDQILVRLDACGVCHTDLHAARGDWPVKPKPPFVPGHEGVGHVVAVGDAVKWVKKGDVVGVPWLYSACGHCEHCLGGWETLCPTQDDTGYTVNGCFAEYVVADPNYVAHLPKTIDPLQVAPVLCAGLTVYKGLKMTDTRAGQWVAVSGVGGLGQMAVQYGVAMGLNVIAIDIDDEKLQTAKVLGAALTVNARSVDPAPYIQSQVGGAHGTLVTAVSRSAFAQAMGFARRGGTIVLNGLPPGEFPISIFDMVMAGTTVRGSIVGTRLDMIEALSFFADGKVKTVIEPDRLENINQIFADLETGNVHGRKVLDLRD

SEQ ID NO: 58 - protein, 342 aa, Acetobacteraceae sp.
MAGKMKAAVAHEFGKPLTIEELDIPTINQNQILVKVDACGVCHTDLHAVSGDWPTKPKPPFIPGHEGVGHVVQVGSNVNWIKEGDVVGVPWLYSSCGHCEHCLGGWETLCEKQQDTGYTVNGCFAEYVVADPNYVAHIPKGVDPIQIAPVLCAGLTVYKGLKMTDTKPGDWVAVSGVGGLGQMAIQYGVAMGKNMIAVDIDDTKLKTAKELGAALTVNAKDTDPAAFVQKEVGGAHGALVTAVSRIAFSQAMGYARRGGTIVLNGLPPGEFPVSIFDMVMTGTTVRGSIVGTRLDMIEALSFFAEGKIHSVVKPDKLENINRIFDDLEHGRIDGRVVLDFRN

SEQ ID NO: 59 - protein, 346 aa, Gluconacetobacter johannae
MEERVAGTMKAAVVRAFGAPLTIEELAIPDIRPDQILVKMDACGVCHTDLHAARGDWPVKPTPPFIPGHEGVGHVVAVGSAVKWVREGDVVGVPWLYSACGHCEHCLGGWETLCKTQDDTGYTVNGCFAEFVVADPNYVAHLPKGVDPVQVAPVLCAGLTVYKGLKMTDTRPGQWVAVSGVGGLGQMAVQYGVAMGLNVIAVDIDDDKLATARSLGAALTVNAAQADPATFIQDHVGGAHGTLVTAVSRSAFAQAMGYARRGGTIVLNGLPPGEFPISIFDMVMAGTTVRGSIVGTRLDMIEALSFFADGKVRTVVAPERLENINRIFDDLEHGTVQGRMVIDFRN

SEQ ID NO: 60 - protein, 342 aa, Gluconacetobacter johannae
MAGTMKAAVVRAFGAPLTIEELAIPDIRPDQILVKMDACGVCHTDLHAARGDWPVKPTPPFIPGHEGVGHVVAVGSAVKWVREGDVVGVPWLYSACGHCEHCLGGWETLCKTQDDTGYTVNGCFAEFVVADPNYVAHLPKGVDPVQVAPVLCAGLTVYKGLKMTDTRPGQWVAVSGVGGLGQMAVQYGVAMGLNVIAVDIDDDKLATARSLGAALTVNAAQADPATFIQDHVGGAHGTLVTAVSRSAFAQAMGYARRGGTIVLNGLPPGEFPISIFDMVMAGTTVRGSIVGTRLDMIEALSFFADGKVRTVVAPERLENINRIFDDLEHGTVQGRMVIDFRN

SEQ ID NO: 61 - protein, 342 aa, Gluconacetobacter liquefaciens
MTGKMKAAVVREFGKPLTIDELDIPTIRADQILVRLDACGVCHTDLHAARGDWPVKPKPPFVPGHEGVGHVVAVGSAVKWVKKGDVVGVPWLYSACGHCEHCLGGWETLCPTQDDTGYTVNGCFAEYVVADPNYVAHLPKNIDPMQVAPVLCAGLTVYKGLKMTDTRPGQWVAISGVGGLGQMAVQYGVAMGLNVIAIDIDDEKLQTAKVLGAALTVNARSVDPAPYIQSQVGGAHGALVTAVSRTAFAQAMGFARRGGTIVLNGLPPGEFPISIFDMVMAGTTVRGSIVGTRLDMIEALSFFADGKVKTVIEPDRLENINEIFADLEAGNVHGRKVLDLRG

SEQ ID NO: 62 - protein, 342 aa, Gluconacetobacter dulcium
MTGKMKAAVVREFGKPLTIEELDIPTIRADQILVRLDACGVCHTDLHAARGDWPVKPKPPFVPGHEGVGHVVAVGSAVKWVKQGDVVGVPWLYSACGHCEHCLGGWETLCPTQDDTGYTVNGCFAEYVVADPNYVAHLPKNIDPMQVAPVLCAGLTVYKGLKMTDTRPGQWVAVSGVGGLGQMAVQYGVAMGLNVIAIDIDDEKLQTAKVLGATLTVNARSVDPAPYIQSQVGGAHGTLVTAVSRTAFAQAMGFARRGGTIVLNGLPPGEFPISIFDMVMAGTTVRGSIVGTRLDMIEALSFFADGKVKTVIEPDRLENINEIFAELEAGNVQGRKVLDLRG

SEQ ID NO: 63 - protein, 342 aa, Gluconacetobacter dulcium
MTGKMKAAVVREFGKPLTIEELDIPTIRADQILVRLDACGVCHTDLHAARGDWPVKPKPPFVPGHEGVGHVVAVGSAVKWVKQGDVVGVPWLYSACGHCEHCLGGWETLCPTQDDTGYTVNGCFAEYVVADPNYVAHLPKNIDPMQVAPVLCAGLTVYKGLKMTDTRPGQWVAVSGVGGLGQMAVQYGVAMGLNVIAIDIDDEKLQTAKVLGAALTVNARSVDPAPYIQSQVGGAHGTLVTAVSRTAFAQAMGFARRGGTIVLNGLPPGEFPISIFDMVMAGTTVRGSIVGTRLDMIEALSFFADGKVKTVIEPDRLENINEIFAELEAGNVQGRKVLDLRG

SEQ ID NO: 64 - protein, 338 aa, Gluconacetobacter liquefaciens
  source 1..338: note = strain NRIC 0522
MKAAVVREFGKPLTIDELDIPTIRADQILVRLDACGVCHTDLHAARGDWPVKPKPPFVPGHEGVGHVVAVGSAVKWVKKGDVVGVPWLYSACGHCEHCLGGWETLCPTQDDTGYTVNGCFAEYVVADPNYVAHLPKNIDPMQVAPVLCAGLTVYKGLKMTDTRPGQWVAISGVGGLGQMAVQYGVAMGLNVIAIDIDDEKLQTAKVLGAALTVNARSVDPAPYIQSQVGGAHGALVTAVSRTAFAQAMGFARRGGTIVLNGLPPGEFPISIFDMVMAGTTVRGSIVGTRLDMIEALSFFADGKVKTVIEPDRLENINEIFADLEAGNVHGRKVLDLRG

SEQ ID NO: 65 - protein, 342 aa, Gluconacetobacter aggeris
MTGKMKAAVVREFGKPLTIEELDIPTIRPDQILVRLDACGVCHTDLHAARGDWPVKPKPPFVPGHEGVGHVVAVGDAVKWVKKGDMVGVPWLYSACGHCEHCLGGWETLCPTQDDTGYTVNGCFAEYVVADPNYVAHLPKTIDPLQVAPVLCAGLTVYKGLKMTDTRAGQWVAVSGVGGLGQMAVQYGVAMGLNVIAIDIDDEKLQTAKVLGAALTVNARSVDPAPYIQSQVGGAHGTLVTAVSRSAFAQAMGFARRGGTIVLNGLPPGEFPISIFDMVMAGTTVRGSIVGTRLDMIEALSFFADGKVKTVIEPDRLENINQIFADLETGNVHGRKVLDLRD

SEQ ID NO: 66 - protein, 342 aa, Gluconacetobacter takamatsuzukensis
MTGKMKAAVVREFGKPLTIEELDIPTIRPDQILVRLDACGVCHTDLHAARGDWPVKPKPPFVPGHEGVGHVVAVGGAVKWVKKGDVVGVPWLYSACGHCEHCLGGWETLCPTQDDTGYTVNGCFAEYVVADPNYVAHLPKNIDPLQVAPVLCAGLTVYKGLKMTDTRPGQWVAVSGVGGLGQMAVQYGVAMGLNVIAIDIDDEKLQTAKVLGAALTVNARSVDPAPYIQAQVGGAHGVLVTAVSRSAFAQAMGFARRGGTIVLNGLPPGEFPISIFDMVMTGVTVRGSIVGTRLDMIEALSFFADGKVKTVIEPDRLENINQIFADLEAGNVHGRKVLDLRG

SEQ ID NO: 67 - protein, 342 aa, Acetobacter estunensis
MAGKMKAAVAHEFGKPLTIEELDIPTINQNQILVKVDACGVCHTDLHAVSGDWPTKPKPPFIPGHEGVGHVVQVGSNVNWIKEGDVVGVPWLYSSCGHCEHCLGGWETLCEKQQDTGYTVNGCFAEYVVADPNYVAHIPKGVDPIQIAPVLCAGLTVYKGLKMTDTKPGDWVAISGVGGLGQMAIQYGVAMGKNMIAVDIDDTKLKTAKELGAALTVNAKDTDPAAFVQKEVGGAHGALVTAVSRIAFSQAMGYARRGGTIVLNGLPPGEFPVSIFDMVMTGTTVRGSIVGTRLDMIEALSFFAEGKIHSVVKPDKLENINRIFDDLEHGRIDGRVVLDFRN

SEQ ID NO: 68 - protein, 344 aa, Komagataeibacter oboediens
MAGKMKAAVVREFGKPLTIEELDIPQINSNQILVKVDACGVCHTDLHAARGDWPTKPNPPFIPGHEGIGHVVEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLAEARKLGARVTVNALKQDPVAYIREQTGGTHGVLVTAVSDKAFSQAVGYARRGGTVVLNGLPPGDFPLSIFDMVMNGTTVRGSIVGTRLDMIEAMSFFTDGKVTTVVTPDRLENINTIFDNLQHGRVEGRIVLDFRNGA

SEQ ID NO: 69 - protein, 342 aa, Acetobacter aceti
MAGKMKAAVAHEFNKPLTIEELDIPTINQNQILVKMDACGVCHTDLHAVRGDWPTKPTLPFIPGHEGVGHVVQVGSNVNWIKEGDYVGVPFLYSACGHCVHCLGGWETLCEKQEDTGYTVNGCFAEYVVADPNYVAHIPKGADPLQIAPVLCAGLTVYKALKMTETKPGDWVAVSGVGGLGQMAMQYGVAMGKNMIAVDIDDEKLATARKLGAALAVNARDTDPAAFIQKEVGGAQGVVVTAVSRIAFSQAMGYARRGGTIVLNGLPPGDFPVSIFDMVMNGTTVRGSIVGTRLDMIEALSFFADGKVHSVVKPDKLENINRIFDDLENGRIDGRVVLDFRN

SEQ ID NO: 70 - protein, 342 aa, Gluconacetobacter entanii
MAGKMKAAVVREFGKPLSIEELDIPSIRPNQILVKVDACGVCHTDLHAARGDWPTKPKPPFIPGHEGIGHVVEVGAAVDWIKTGDVVGVPWLYSACGHCEHCLGGWETLCEKQEDTGYSVNGCFAEYVVADPNYVAHLPKDIDPIRTAPVLCAGLTVYKGLKMTDTRAGEWVAISGAGGLGQMAIQYAVAMGLNVAAVDIDDGKLAEARRLGASVTVNARTTDPVAYIQQKTGGTHGVLVTAVSNTAFTQAIGYARRGGTIVLNGLPPGEFPISIFDMVMNGTTVRGSIVGTRLDMIEAMSFFTEGKVSTVVAADKLENINDIFDRLQHGKVAGRIVLDFRN

SEQ ID NO: 71 - protein, 342 aa, Novacetimonas maltaceti
MAGKMKAAVVREFGKPLSIEELDIPSIRPNQILVKVDACGVCHTDLHAARGDWPTKPKPPFIPGHEGIGHVVEVGASVDWIKTGDVVGVPWLYSACGHCEHCLGGWETLCEKQEDTGYSVNGCFAEYVVADPNYVAHLPKDIDPIRTAPVLCAGLTVYKGLKMTDTRAGEWVAVSGAGGLGQMAIQYAVAMGLNVAAVDIDDAKLAEARRLGASVTVNARTTDPVSYIQQKTGGTHGVLVTAVSNTAFTQAIGYARRGGTIVLNGLPPGEFPLSIFDMVMNGTTVRGSIVGTRLDMIEALSFFTEGKVSTVVATDKLENINDIFDRLQHGKVAGRIVLDFRN

SEQ ID NO: 72 - protein, 342 aa, Acetobacter peroxydans
MAGKMKAAVCHEFGKPLTIEELDIPTINQNQILVKVDTCGVCHTDLHAVRGDWPAKPTPPFIPGHEGVGHVVEVGSNVKWISKGDIVGVPWLYSACGHCEHCYGGWETLCEKQDDTGYTVNGCFAEYVVADPNYVARIPAGVDPLHVAPVLCAGLTVYKGLKMTDTKPGDWVAVSGVGGLGQMAIQYGVAMGMNMIAVDIDDTKLAEAKELGAALTVNAREADPAAFIQKQVGGAHGALVTAVSRSAFSQAMGYARRGGTIVLNGLPPGEFPISIFDMVMAGTTVRGSIVGTRLDMIEALSFYAEGKIRSVVKADKLENINQIFDDLQNGRVAGRVVIDFRN

SEQ ID NO: 73 - protein, 344 aa, Komagataeibacter sp.
MAGKMKAAVVREFGKPLTIEELDIPQINSNQILVKVDACGVCHTDLHAARGDWPTKPNPPFIPGHEGIGHVVEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARKLGARVTVNALKQDPVAYIREQTGGTHGVLVTAVSDKAFSQAVGYARRGGTVVLNGLPPGDFPLSIFDMVMNGTTVRGSIVGTRLDMIEAMSFFTDGKVTTVVTPDRLENINTIFDNLQHGRVEGRIVLDFRNGA

SEQ ID NO: 74 - protein, 344 aa, Komagataeibacter oboediens
MAGKMKAAVVREFGKPLTIEELDIPQINSNQILVKVDACGVCHTDLHAARGDWPTKPNPPFIPGHEGIGHVVEVGSNVSWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARKLGARVTVNALKQDPVAYIREQTGGTHGVLVTAVSDKAFSQAVGYARRGGTVVLNGLPPGDFPLSIFDMVMNGTTVRGSIVGTRLDMIEAMSFFTDGKVTTVVTPDRLENINTIFDNLQHGRVEGRIVLDFRNGA

SEQ ID NO: 75 - protein, 342 aa, Acetobacter sp.
MAGKMKAAVAHAFGQPLTIEELDIPEINSNQILVKMDACGVCHTDLHAVRGDWPSKPTLSFIPGHEGVGHVVQVGSNVSWIREGDYVGVPWLYSACGHCEHCLGAWETLCEKQQDTGYSVNGCFAEYVVADPNYVAHIPKGADPLQIAPVLCAGLTVYKGLKMTDTKPGDWVAISGVGGLGQMAIQYGVAMGLNMIAVDIDDNKLATAKELGAALAVNAKDTDPAAFVQKEVGGAHGALVTAVSRIAFSQAMGYARRGGTIVLNGLPPGDFPVSIFDMVMNGTTIRGSIVGTRLDMIEALSFFADGKVHTVVKPEKLENINRIFDDLENGRIDGRAVLDFRN

SEQ ID NO: 76 - protein, 342 aa, Acetobacter malorum
MAGKMKAAVAHEFGKPLTIEELDIPTINQNQILVKVDACGVCHTDLHAVSGDWPTKPKPPFIPGHEGVGHVVQVGSNVNWIKEGDVVGVPWLYSSCGHCEHCLGGWETLCEKQQDTGYTVNGCFAEYVVADPNYVAHIPKGVDPIQIAPVLCAGLTVYKGLKMTDTKPGDWVVVSGVGGLGQMAIQYGVAMGKNMIAVDIDDTKLKTANELGAALTVNAKDTDPAAFVQKEVGGAHGALVTAVSRIAFSQAMGYARRGGTIVLNGLPPGEFPVSIFDMVMTGTTVRGSIVGTRLDMIEALSFFAEGKIHSVVKPDKLENINRIFDDLEHGRIDGRVVLDFRN

SEQ ID NO: 77 - protein, 342 aa, Novacetimonas pomaceti
MAGKMKAAVVREFGKPLSIEELDIPSIRPNQILVKVDACGVCHTDLHAARGDWPVKPKPPFIPGHEGIGHVVEVGSSVDWIKTGDVVGVPWLYSACGHCEHCLGGWETLCEKQEDTGYSVNGCFAEYVVADPNYVAHLPKDIDPIRTAPVLCAGLTVYKGLKMTDTRAGEWVAISGAGGLGQMAIQYAVAMGLNVAAVDIDDGKLSEARRLGASVTVNARTTDPVSYIQQKTGGTHGVLVTAVSNSAFTQAIGYARRGGTIVLNGLPPGEFPLSIFDMVMNGTTVRGSIVGTRLDMIEALSFFTEGKVSTVVAPDKLENINDIFDRLQHGKVAGRIVLDFRK

SEQ ID NO: 78 - protein, 344 aa, Komagataeibacter oboediens
MAGKMKAAVVREFGKPLTIEELDIPQINSNQILVKVDACGVCHTDLHAARGDWPTKPNPPFIPGHEGIGHVIEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARKLGARVTVNALKQDPVAYIREQTGGTHGVLVTAVSDKAFSQAVGYARRGGTVVLNGLPPGDFPLSIFDMVMNGTTVRGSIVGTRLDMIEAMSFFTDGKVTTVVTPDRLENINTIFDNLQHGRVEGRIVLDFRNGA

SEQ ID NO: 79 - protein, 344 aa, Komagataeibacter medellinensis
MAGKMKAAVVREFGKPLTIEELDIPTIRPDQILVKVDACGVCHTDLHAARGDWPTKPNPPFIPGHEGIGHVVEVGSAVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARKLGARVTVNALKHDPVAYIREQTGGTHGVLVTAVSDKAFSQAVGYARRGGTVVLNGLPPGDFPLSIFDMVMNGTTVRGSIVGTRLDMIEAMSFFTDGKVTTVVSPDRLENINTIFDNLEHGRVEGRIVLDFRNGA

SEQ ID NO: 80 - protein, 342 aa, Novacetimonas sp.
MAGKMKAAVVREFGKPLSIEELDIPSIRPNQILVKVDACGVCHTDLHAARGDWPVKPKPPFIPGHEGIGHVVEVGSSVDWIKTGDVVGVPWLYSACGHCEHCLGGWETLCEKQEDTGYSVNGCFAEYVVADPNYVAHLPKDIDPIRTAPVLCAGLTVYKGLKMTDTRAGEWVAISGAGGLGQMAIQYAVAMGLNVAAVDIDDGKLNEARRLGASVTVNARTTDPVSYIQQKTGGTHGVLVTAVSNSAFTQAIGYARRGGTIVLNGLPPGEFPLSIFDMVMNGTTVRGSIVGTRLDMIEALSFFTEGKVSTVVAPDKLENINDIFDRLQHGKVAGRIVLDFRK

SEQ ID NO: 81 - protein, 342 aa, Novacetimonas hansenii
MAGKMKAAVVREFGKPLSIEELDIPSIRPNQILVKVDACGVCHTDLHAARGDWPTKPKPPFIPGHEGIGHVVEVGAAVDWIKTGDVVGVPWLYSACGHCEHCLGGWETLCEKQEDTGYSVNGCFAEYVVADPNYVAHLPKDIDPIRTAPVLCAGLTVYKGLKMTDTRAGEWVAISGAGGLGQMAIQYAVAMGLNVAAVDIDDGKLAEARKLGASVTVNARTTDPVSYIRQKTGGTHGVLVTAVSNTAFTQAIGYARRGGTIVLNGLPPGEFPISIFDMVMNGTTVRGSIVGTRLDMIEALSFFTEGKVSTVVATDKLENINDIFDRLQHGQVAGRIVLDFRN

SEQ ID NO: 82 - protein, 342 aa, Acetobacter papayae
MAGKMKAAVCHEFGKPLTIEELDIPTINQNQILVKVDTCGVCHTDLHAVRGDWPAKPTPPFIPGHEGVGHVVEVGSNVDWISKGDIVGVPWLYSACGHCEHCYGGWETLCEKQDDTGYTVNGCFAEYVVADPNYVARIPAGVDPLHIAPVLCAGLTVYKGLKMTDTKAGDWVAVSGVGGLGQMAIQYGVAMGLNMIGVDIDDTKLAQAKELGAALTVNAREADPAAFIQKQVGGAHGALVTAVSRTAFSQAMGYARRGGTIVLNGLPPGEFPISIFDMVMAGTTVRGSIVGTRLDMIEALSFYAEGKIRSVVKADKLENINQIFDDLQNGRVAGRIVIDFRN

SEQ ID NO: 83 - protein, 344 aa, Komagataeibacter saccharivorans
MLGKMKAAVVREFGKPLTIEELDIPQIRPHQILVKVDACGVCHTDLHAARGDWPTKPNPPFIPGHEGVGHVVEVGSDVDWIKTGDVVGVPWLYSACGHCEHCLGGWETLCEKQEDTGYSVNGCFAEYVVADPNYVAHLPRDIDPIRTAPVLCAGLTVYKGLKMTDTKAGEWVAISGAGGLGQMAIQYAVAMGLNVAAVDISDEKLDEARKLGARVTVNALKEDPVACIQKHTGGTHGVLVTAVSDKAFTQAIGYARRGGTIVLNGLPPGDFPLSIFDMVMNGTTVRGSIVGTRLDMIEALSFFADGKVSTVVSTDRLENINTIFDNLEHGRVQGRIVLDFRNGA

SEQ ID NO: 84 - protein, 344 aa, Komagataeibacter saccharivorans
MLGKMKAAVVREFGKPLTIEELDIPQIRPHQILVKVDACGVCHTDLHAARGDWPTKPNPPFIPGHEGVGHVVEVGSDVDWIKTGDVVGVPWLYSACGHCEHCLGGWETLCEKQEDTGYSVNGCFAEYVVADPNYVAHLPRDIDPIRTAPVLCAGLTVYKGLKMTDTKAGEWVAISGAGGLGQMAIQYAVAMGLNVAAVDISDEKLDEARKLGARVTVNALKEDPIACIQKHTGGTHGVLVTAVSDKAFTQAIGYARRGGTIVLNGLPPGDFPLSIFDMVMNGTTVRGSIVGTRLDMIEALSFFADGKVSTVVSTDRLENINTIFDNLEHGRVQGRIVLDFRNGA

SEQ ID NO: 85 - protein, 342 aa, Acetobacter conturbans
MAGKMQAAVAREFNKPLTIEELDIPTINQNQILVKMDACGVCHTDLHAVRGDWPTKPTLPFIPGHEGVGHVVQVGSNVNWVKEGDYVGVPFLYSACGHCEHCLGGWETLCEKQEDTGYSVNGCFAEYVVADPNYIAHIPKGADPLQIAPVLCAGLTVYKALKMTNTKPGDWVAVSGVGGLGQMAMQYGVAMGKNMIAVDIDDAKLATAKELGAALTVNARNTDPAAFVQKEVGGAQGVVVTAVSRIAFSQAMGYARRGGTIVLNGLPPGDFPVSIFDMVMNGTTIRGSIVGTRLDMIEALSFFAEGKVRSVVKADKLENINRIFDDLENGRIDGRVVLDFRK

SEQ ID NO: 86 - protein, 342 aa, Acetobacter sicerae
MAGKMKAAVAHEFNKPLTIEELDIPTINENQILVKMDACGVCHTDLHAVRGDWPTKPTLPFIPGHEGVGHVVQVGSNVNWIREGDYVGVPFLYSACGHCVHCLGGWETLCEKQEDTGYTVNGCFAEYVVADPSYVAHIPKGADPLQIAPVLCAGLTVYKALKMTDTKPGDWVAVSGVGGLGQMAMQYGVAMGKNMIAVDIDDEKLATAKKLGAALTVNARDTDPAAFVQKEVGGAQGVVVTAVSRIAFSQAMGYARRGGTIVLNGLPPGDFPVSIFDMVMNGTTVRGSIVGTRLDMIEALSFFADGKVHSVVKPDKLENINRIFDDLENGRIDGRVVLDFRN

SEQ ID NO: 87 - protein, 342 aa, Gluconacetobacter sacchari
MSAKMKAAVVREFGKPLTIEELDIPAIRPDQILVRLDACGVCHTDLHAARGDWPVKPKPPFVPGHEGVGHVVSVGSAVKWVKEGDVVGVPWLYSACGHCEHCLGGWETLCPAQEDTGYSVNGCFAEYVVADPDYVAHLPKSVDPLQVAPVLCAGLTVYKGLKMTDTRPGQWVAVSGVGGLGQMAVQYGVAMGLNVIAIDIDDDKLATARVLGAALTVNARAVDPAPYIQSQVGGAHGVLVTAVSRTAFAQAMGFARRGGTIVLNGLPPGEFPISIFDMVMAGTTVRGSIVGTRLDMIEAVSFFADGKVKTVIEPDRLENINQIFADLEAGNVQGRKVLDLRG

SEQ ID NO: 88 - protein, 342 aa, Acetobacter suratthaniensis
MAGKMKAAVCHEFGKPLTIEELDIPTINQNQILVKVDTCGVCHTDLHAVRGDWPAKPTPPFIPGHEGIGHVVEVGSNVDWIAKGDIVGVPWLYSACGHCEHCYGGWETLCEKQEDTGYTVNGCFAEYVVADPNYVARIPAGVDPLHVAPVLCAGLTVYKGLKMTDTKAGDWVAVSGVGGLGQMAIQYGVAMGLNMIGVDIDDAKLAQATELGAALTVNARDTDPAAFIQKQVGGAHGALVTAVSRTAFSQAMGYARRGGTIVLNGLPPGEFPISIFDMVMAGTTVRGSIVGTRLDMIEALSFYAQGKIRSVVKADKLENINQIFDDLQNGRVAGRIVIDFRN

SEQ ID NO: 89 - protein, 342 aa, Novacetimonas hansenii
MAGKMKAAVVREFGKPLSIEELDIPSIRPNQILVKVDACGVCHTDLHAARGDWPTKPKPPFIPGHEGIGHVVEVGAAVDWIKAGDVVGVPWLYSACGHCEHCLGGWETLCEKQEDTGYSVNGCFAEYVVADPNYVAHLPKDIDPIRTAPVLCAGLTVYKGLKMTDTRAGEWVAISGAGGLGQMAIQYAVAMGLNVAAVDIDDGKLAEARKLGASVTVNARTTDPVSYIRQKTGGTHGVLVTAVSNTAFTQAIGYARRGGTIVLNGLPPGEFPISIFDMVMNGTTVRGSIVGTRLDMIEALSFFTEGKVSTVVATDKLENINDIFDRLQHGQVAGRIVLDFRN

SEQ ID NO: 90 - protein, 344 aa, Komagataeibacter swingsii
MAGKMKAAVVREFGKPLTIEELDIPQINANQILVKVDACGVCHTDLHAARGDWPTKPKPPFIPGHEGIGHVVEVGSNVNWVKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARRLGARVTVNALKQDPVAYIRQQTGGTHGVLVTAVSDKAFSQAVGYARRGGTVVLNGLPPGDFPLSIFDMVMNGTTVRGSIVGTRLDMIEAMSFFTDGKVTTVVSPDRLENINTIFDNLQHGRVQGRVVLDFRNGA

SEQ ID NO: 91 - protein, 342 aa, Acetobacter peroxydans
MAGKMKAAVCHEFGKPLTIEELDIPTINQNQILVKVDTCGVCHTDLHAVRGDWPAKPTPPFIPGHEGVGHVVEVGSNVKWISKGDIVGVPWLYSACGHCEHCYGGWETLCEKQDDTGYTVNGCFAEYVVADPNYVARIPAGVDPLHVAPVLCAGLTVYKGLKMTDTKPGDWVAVSGVGGLGQMAIQYGVAMGLNMIAVDIDDTKLAEAKELGAVLTVNAREADPAAFIQKQVGGAHGTLVTAVSRSAFSQAMGYARRGGTIVLNGLPPGEFPISIFDMVMAGTTVRGSIVGTRLDMIEALSFYAQGKIRSVVKADKLENINQIFDDLQNGRVAGRVVIDFRN

SEQ ID NO: 92 - protein, 344 aa, Komagataeibacter diospyri
MAGKMKAAVVREFGKPLTIEELDIPQINSNQILVKVDACGVCHTDLHAARGDWPTKPRPPFIPGHEGIGHVVEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARKLGARVTVNALKQDPVAYIREQTGGTHGVLVTAVSDKAFSQAVGYARRGGTVVLNGLPPGDFPLSIFDMVMNGTTVRGSIVGTRLDMIEAMSFFTDGKVTTVVSPDKLENINTIFDNLEHGRVEGRVVLDFRNGA

SEQ ID NO: 93 - protein, 344 aa, Komagataeibacter xylinus
MVGKMKAAVVREFGKPLTIEELDIPQINANQILVKVDACGVCHTDLHAARGDWPTKPNPPFIPGHEGIGHVVEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLAEARKLGARVTVNALKEDPVATIRAQTGGTHGVLVTAVSDKAFSQAVGYARRGGTVVLNGLPPGDFPLSIFDMVMNGTTVRGSIVGTRLDMIEAMSFFTDGKVTTVVSPDRLENINTIFDNLEHGRVAGRVVLDFRNGA

SEQ ID NO: 94 - protein, 344 aa, Komagataeibacter melomenusus
MVGKMKAAVVREFGKPLTIEELDIPQINANQILVKVDACGVCHTDLHAARGDWPTKPRPPFIPGHEGIGHVVEVGSNVNWVKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLAEARKLGARVTVNALREDPVATIRAQTGGTHGVLVTAVSDKAFSQAVGYARRGGTVVLNGLPPGDFPLSIFDMVMNGTTVRGSIVGTRLDMIEAMSFFTDGKVTTVVSPDRLENINTIFDNLEHGRVAGRVVLDFRNGA

SEQ ID NO: 95 - protein, 326 aa, Gluconacetobacter entanii
MSIEELDIPSIRPNQILVKVDACGVCHTDLHAARGDWPTKPKPPFIPGHEGIGHVVEVGAAVDWIKTGDVVGVPWLYSACGHCEHCLGGWETLCEKQEDTGYSVNGCFAEYVVADPNYVAHLPKDIDPIRTAPVLCAGLTVYKGLKMTDTRAGEWVAISGAGGLGQMAIQYAVAMGLNVAAVDIDDGKLAEARRLGASVTVNARTTDPVAYIQQKTGGTHGVLVTAVSNTAFTQAIGYARRGGTIVLNGLPPGEFPISIFDMVMNGTTVRGSIVGTRLDMIEAMSFFTEGKVSTVVAADKLENINDIFDRLQHGKVAGRIVLDFRN

SEQ ID NO: 96 - protein, 344 aa, Komagataeibacter sp.
MAGKMKAAVVREFGKPLTIEELDIPQINANQILVKVDACGVCHTDLHAARGDWPTKPKPPFIPGHEGIGHVVEVGSNVNWIRTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARKLGARVTVNALKQDPVAYIRQQTGGTHGVLVTAVSDKAFSQAVGYARRGGTVVLNGLPPGDFPLSIFDMVMNGTTVRGSIVGTRLDMIEAMSFFTDGKVTTVVSPDRLENINTIFDNLQHGRVQGRVVLDFRNGA

SEQ ID NO: 97 - protein, 344 aa, Komagataeibacter intermedius
MAGKMKAAVVREFGRPLTIEELDIPQINSNQILVKVDACGVCHTDLHAARGDWPTKPTPPFIPGHEGIGHVVEVGSNVNWIRTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARKLGARVTVNALKQDPVAYIREQTGGTHGVLVTAVSDKAFSQAVGYARRGGTVVLNGLPPGDFPLSIFDMVMNGTTVRGSIVGTRLDMIEAMSFFTDGKVTTVVTPDRLENINTIFDNLQHGRVEGRIVLDFRNGA

SEQ ID NO: 98 - protein, 344 aa, Komagataeibacter nataicola
MVGKMKAAVVREFGKPLTIEELDIPQINANQILVKVDACGVCHTDLHAARGDWPTKPNPPFIPGHEGIGHVVEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLAEARKLGARVTVNALKEDPVAIIRAQTGGTHGVLVTAVSDKAFSQAVGYARRGGTVVLNGLPPGDFPLSIFDMVMNGTTVRGSIVGTRLDMIEAMSFFTDGKVTTVVSSDRLENINTIFDNLEHGRVQGRVVLDFRNEV

SEQ ID NO: 99 - protein, 344 aa, Komagataeibacter europaeus
MAGKMKAAVVREFGKPLTIEELDIPQINTNQILVKVDACGVCHTDLHAARGDWPTKPKPPFIPGHEGIGHVVEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARKLGARVTVNALKQDPVAYIREQTGGTHGVLVTAVSDKAFSQAVGYARRGGTVVLNGLPPGDFPLSIFDMVMNGTTVRGSIVGTRLDMIEAMSFFTDGKVTTVVSTDKLENINTIFDNLQHGRVKGRVVLDFRNGA

SEQ ID NO: 100 - protein, 344 aa, Komagataeibacter nataicola
MVGKMKAAVVREFGKPLTIEELDIPQINANQILVKVDACGVCHTDLHAARGDWPTKPNPPFIPGHEGIGHVVEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLAEARKLGARVTVNALKEDPVAVIRAQTGGTHGVLVTAVSDKAFSQAVGYARRGGTVVLNGLPPGDFPLSIFDMVMNGTTVRGSIVGTRLDMIEAMSFFTDGKVTTVVSSDRLENINTIFDNLEHGRVQGRVVLDFRNGV

SEQ ID NO: 101 - protein, 344 aa, Komagataeibacter europaeus
MAGKMKAAVVREFGKPLTIEELDIPQINTNQILVKVDACGVCHTDLHAARGDWPTKPRPPFIPGHEGIGHVVEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARKLGARVTVNALKQDPVAYIREQTGGTHGVLVTAVSDKAFSQAVGYARRGGTVVLNGLPPGDFPLSIFDMVMNGTTVRGSIVGTRLDMIEAMSFFTDGKVTTVVSTDKLENINTIFDNLQHGRVKGRVVLDFRNGA

SEQ ID NO: 102 - protein, 344 aa, Komagataeibacter sp.
  source 1..344: note = strain FXV3
MAGKMKAAVVREFGKPLTIEELDIPQINANQILVKVDACGVCHTDLHAARGDWPTKPRPPFIPGHEGIGHVVEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLAEARRLGARVTVNALKQDPVAYIREQTGGTHGVLVTAVSDKAFSQAVGYARRGGTVVLNGLPPGDFPLSIFDMVMNGTTVRGSIVGTRLDMIEAMSFFTDGKVTTVVSPDRLENINTIFDNLEHGRVQGRVVLDFRNGA

SEQ ID NO: 103 - protein, 344 aa, Komagataeibacter sp.
  source 1..344: note = strain FNDCR2
MAGKMKAAVVREFGKPLTIEELDIPQIRPNQILVKVDACGVCHTDLHAARGDWPTKPNPPFIPGHEGIGHVVEVGSNVSWVKTGDVVGVPWLYSACGHCEHCLGGWETLCAEQEDTGYSVNGCFAEYVVADPNYVAHLPKDIDPIRTAPVLCAGLTVYKGLKMTDTRAGEWVAISGAGGLGQMAIQYAVAMGLNVAAVDISEEKLAEARKLGARVTVNALKDDPVAYIQKQTGGTHGVLVTAVSDKAFSQAIGYARRGGTVVLNGLPPGDFPLSIFDMVMNGTTVRGSIVGTRLDMIEALSFFTDGKVSTVVSTDRLENINTIFDNLEHGRVQGRVVLDFRNGA

SEQ ID NO: 104 - protein, 344 aa, Komagataeibacter rhaeticus
MAGKMKAAVVREFGKPLTIEELDIPTIRPDQILVKVDACGVCHTDLHAARGDWPAKPTPPFIPGHEGIGHVVEVGSNVNWIRTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARKLGARVTVNALKQDPVAAIREKTGGTHGVLVTAVSDKAFSQAVGYARRGGTVVLNGLPPGDFPLSIFDMVMNGTTVRGSIVGTRLDMIEAMSFFTEGKVTTVVSPDRLENINTIFDNLEHGRVEGRIVLDFRNGA

SEQ ID NO: 105 - protein, 340 aa, Komagataeibacter rhaeticus
  source 1..340: note = strain DSM 16663
MKAAVVREFGKPLTIEELDIPTIRPDQILVKVDACGVCHTDLHAARGDWPAKPTPPFIPGHEGIGHVVEVGSNVNWIRTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARKLGARVTVNALKQDPVAAIREKTGGTHGVLVTAVSDKAFSQAVGYARRGGTVVLNGLPPGDFPLSIFDMVMNGTTVRGSIVGTRLDMIEAMSFFTEGKVTTVVSPDRLENINTIFDNLEHGRVEGRIVLDFRNGA

SEQ ID NO: 106 - protein, 344 aa, Komagataeibacter sp.
MAGKMKAAVVREFGKPLTIEELDIPTIRPDQILVKVDACGVCHTDLHAARGDWPAKPTPPFIPGHEGIGHVVEVGSNVNWIRTGDVVGVPWLYSACGHCEHCLGGWETLCEKQEDTGYSVNGCFAEYVVADPNYVAHLPKDIDPVRTAPVLCAGLTVYKGLKMTDTRAGEWVAISGAGGLGQMAIQYAVAMGLNVAAVDISDEKLEEARKLGARVTVNALKQDPVAAIREKTGGTHGVLVTAVSDKAFSQAVGYARRGGTVVLNGLPPGDFPLSIFDMVMNGTTVRGSIVGTRLDMIEAMSFFTEGKVTTVVSPDRLENINTIFDNLEHGRVEGRIVLDFRNGA

SEQ ID NO: 107 - protein, 340 aa, Komagataeibacter rhaeticus
MKAAVVREFGKPLTIEELDIPTIRPDQILVKVDACGVCHTDLHAARGDWPAKPTPPFIPGHEGIGHVVEVGSNVNWIRTGDVVGVPWLYSACGHCEHCLGGWETLCEKQEDTGYSVNGCFAEYVVADPNYVAHLPKDIDPVRTAPVLCAGLTVYKGLKMTDTRAGEWVAISGAGGLGQMAIQYAVAMGLNVAAVDISDEKLEEARKLGARVTVNALKQDPVAAIREKTGGTHGVLVTAVSDKAFSQAVGYARRGGTVVLNGLPPGDFPLSIFDMVMNGTTVRGSIVGTRLDMIEAMSFFTEGKVTTVVSPDRLENINTIFDNLEHGRVEGRIVLDFRNGA

SEQ ID NO: 108 - protein, 344 aa, Komagataeibacter xylinus
MVGKMKAAVVREFGKPLTIEELDIPQINANQILVKVDACGVCHTDLHAARGDWPAKPKPPFIPGHEGIGHVVEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLAEARRLGARVTVNALKEDPVATIRAQTGGTHGVLVTAVSDKAFSQAVGYARRGGTVVLNGLPPGDFPLSIFDMVMNGTTVRGSIVGTRLDMIEAMSFFTDGKVTTVVSPDRLENINTIFDNLEHGRVSGRVVLDFRNGA

SEQ ID NO: 109 - protein, 344 aa, Komagataeibacter europaeus
MAGKMKAAVVREFGKPLTIEELDIPQINTNQILVKVDACGVCHTDLHAARGDWPTKPRPPFIPGHEGIGHVVEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTRAGEWVAISGAGGLGQMAIQYAVAMGLNVAAVDISDEKLEEARRLGARVTVNALKQDPVAYIREQTGGTHGVLVTAVSDKAFSQAVGYARRGGTVVLNGLPPGDFPLSIFDMVMNGTTVRGSIVGTRLDMIEAMSFFTDGKVTTVVSTDKLENINTIFDNLQHGRVKGRVVLDFRNGA

SEQ ID NO: 110 - protein, 344 aa, Komagataeibacter europaeus
MAGKMKAAVVREFGKPLTIEELDIPQINTNQILVKVDACGVCHTDLHAARGDWPTKPRPPFIPGHEGIGHVVEVGSNVNWIKTGDVVGVPWLYSACGHCEHCLGGWETLCEKQEDTGYSVNGCFAEYVVADPNYVAHLPKDIDPIKTAPVLCAGLTVYKGLKMTDTQAGEWVAISGAGGLGQMAIQYAVAMGLNVAAVDISDEKLEEARKLGARVTVNALKQDPVAYIREQTGGTHGVLVTAVSDKAFSQAVGYARRGGTVVLNGLPPGDFPLSIFDMVMNGTTVRGSIVGTRLDMIEAMSFFTDGKVTTVVSTDKLENINTIFDNLQHGRVKGRVVLDFRNGA

SEQ ID NO: 111 - protein, 344 aa, Komagataeibacter europaeus
MAGKMKAAVVREFGKPLTIEELDIPQINTNQILVKVDACGVCHTDLHAARGDWPTKPRPPFIPGHEGIGHVVEVGSNVNWIKTGDVVGVPWLYSACGHCEHCLGGWETLCEKQEDTGYSVNGCFAEYVVAAPNYVAHLPKDIDPIKTAPVLCAGLTVYKGLKMTDTRAGEWVAISGAGGLGQMAIQYAVAMGLNVAAVDISDEKLEEARKLGARVTVNALKQDPVAYIREQTGGTHGVLVTAVSDKAFSQAVGYARRGGTVVLNGLPPGDFPLSIFDMVMNGTTVRGSIVGTRLDMIEAMSFFTDGKVTTVVSTDKLENINTIFDNLQHGRVKGRVVLDFRNGA

SEQ ID NO: 112 - protein, 344 aa, Komagataeibacter sucrofermentans
MVGKMKAAVVREFGKPLTIEELDIPQINANQILVKVDACGVCHTDLHAARGDWPTKPKPPFIPGHEGIGHVVEVGSNVNWIKTGDVVGVPWLYSACGHCEHCLGGWETLCEKQEDTGYSVNGCFAEYVVADPNYVAHLPRDIDPIKTAPVLCAGLTVYKGLKMTDTRAGEWVAISGAGGLGQMAIQYAVAMGLNVAAVDISDEKLAEARRLGARVTVNALKEDPVATIRAQTGGTHGVLVTAVSDKAFSQAVGYARRGGTVVLNGLPPGDFPLSIFDMVMNGTTVRGSIVGTRLDMIEAMSFFTDGKVTTVVSPDRLENINTIFDNLEHGRVAGRVVLDFRNGA

SEQ ID NO: 113 - protein, 344 aa, Komagataeibacter xylinus
MAGKMKAAVVREFGKPLTIEELDIPQINANQILVKVDACGVCHTDLHAARGDWPTKPNPPFIPGHEGIGHVVEVGSNVNWIRNGDVVGVPWLYSACGHCEHCLGGWETLCEKQEDTGYSVNGCFAEYVVADPNYVAHLPRDIDPIKTAPVLCAGLTVYKGLKMTDTRAGEWVAISGAGGLGQMAIQYAVAMGLNVGAVDISDEKLAEARKLGARVTVNALKEDPVATIRAQTGGTHGVLVTAVSDKAFSQAVGYARRGGTVVLNGLPPGDFPLSIFDMVMNGTTVRGSIVGTRLDMIEAMSFFTDGKVSTVVSPDRLENINTIFDNLEHGRVAGRVVLDFRNGA

SEQ ID NO: 114 - protein, 341 aa, Acetobacter sacchari
MTTMKAAVCREFGKPLTIEQLAIPTIDENQILVKLDACGVCHTDLHAARGDWPAKPTPPFIPGHEGVGRVARVGSNVKWVKEGDVVGVPWLHSACGHCEHCYGGWETLCEKQEDTGYSVNGCFAEYVVADPNYVAHIPAGVDPLHVAPVLCAGLTVYKGLKMTDTKPGDWVAVSGVGGLGQMAVQYGVAMGLNVVAVDIDDAKLAEAKELGAALTINAKAEDPAAAIQKKVGGAHGVLVTAVSRSAFAQAMGYPRRGGTIVLTGLPPGEFPISIFDMVMAGTTVRGSIVGTRLDMIEALSFFADGKVRSVVKADRLENINQIFDDLENGRVEGRIVIDFRN

SEQ ID NO: 115 - protein, 342 aa, Acetobacter sacchari
MMTTMKAAVCREFGKPLTIEQLAIPTIDENQILVKLDACGVCHTDLHAARGDWPAKPTPPFIPGHEGVGRVARVGSNVKWVKEGDVVGVPWLHSACGHCEHCYGGWETLCEKQEDTGYSVNGCFAEYVVADPNYVAHIPAGVDPLHVAPVLCAGLTVYKGLKMTDTKPGDWVAVSGVGGLGQMAVQYGVAMGLNVVAVDIDDAKLAEAKELGAALTINAKAEDPAAAIQKKVGGAHGVLVTAVSRSAFAQAMGYPRRGGTIVLTGLPPGEFPISIFDMVMAGTTVRGSIVGTRLDMIEALSFFADGKVRSVVKADRLENINQIFDDLENGRVEGRIVIDFRN

SEQ ID NO: 116 - protein, 352 aa, Komagataeibacter kakiaceti
MILELKVQDGWKDEAAVVREFGKPLTIEELDIPQIRPNQILVKVDACGVCHTDLHAARGDWPSKPNPPFIPGHEGIGHVVEVGSNVSWVKTGDVVGVPWLYSACGHCEHCLGGWETLCAKQEDTGYSVNGCFAEYVVADPNYVAHLPKDIDPIRTAPVLCAGLTVYKGLKMTDTRAGEWVAISGAGGLGQMAIQYAVAMGLNVAAVDISEDKLDEARKLGARVTVNALKEDPVAYIQKQTGGTHGVLVTAVSDKAFSQAIGFARRGGTVVLNGLPPGDFPLSIFDMVMNGTTVRGSIVGTRLDMIEALSFFTDGKVSTVVSTDKLENINTIFDNLEHGHVHGRVVLDFRNGA

SEQ ID NO: 117 - protein, 344 aa, Komagataeibacter sp.
  source 1..344: note = strain FNDCF1
MAGKMKAAVVREFGRPLTIEELDIPQINSNQILVKVDACGVCHTDLHAARGDWPAKPNPPFIPGHEGIGHVVEVGSNVNWIRTGDVVGVPWLYSACGHCEHCLGGWETLCEKQEDTGYSVNGCFAEYVVADPNYVAHLPKDIDPIRTAPVLCAGLTVYKGLKMTDTRAGEWVAISGAGGLGQMAIQYAVAMGLNVAAVDISDEKLAEARRLGARVTVNALKQDPVACIREQTGGTHGVLVTAVSDKAFSQAVGYARRGGTVVLNGLPPGDFPLSIFDMVMNGTTVRGSIVGTRLDMIEAMSFFTEGKVTTVVSPDRLENINTIFDNLEHGRVQGRIVLDFRNGA

SEQ ID NO: 118 - protein, 344 aa, Komagataeibacter melaceti
MAGKMKAAVVREFGKPLTIEELDIPQINPNQILVKVDACGVCHTDLHAARGDWPTKPNPPFIPGHEGIGHVVEVGSNVNWIKTGDVVGVPWLYSACGHCEHCLGGWETLCAEQKDTGYSVNGCFAEYVVADPNYVAHLPKDIDPIRTAPVLCAGLTVYKGLKMTDTRAGEWVAISGAGGLGQMAIQYAVAMGLNVAAVDISEDKLEEARRLGARVTVNALKEDPVAYIQQQTGGTHGVLVTAVSDKAFSQAIGYARRGGTVVLNGLPPGDFPLSIFDMVMNGTTVRGSIVGTRLDMIEAMSFFTDGKVSTVVSTDKLENINTIFDNLEHGRVHGRVVLDFRNGA

SEQ ID NO: 119 - protein, 342 aa, Acetobacter pasteurianus
MSGKMKAAVVHEFGKPLTIEELDIPTIKPTQILVKMIACGVCHTDLHAASGDWPKKPHLPFIPGHEGVGTVVQVGSEVDWVKEGDVVGVPWLYSACGHCEHCLAGWETLCAKQEETGYSVNGCFAEYVVADPNYIAHLPKGVDPVKVAPVLCAGLTVYKGLKMTDTRAGNWVAISGVGGLGQMAVQYAVAMGLNVVAVDIDDEKLATAKKLGATYTVNARNTDPAAFMQEKVGGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 120 - protein, 342 aa, Acetobacter sp.
MSGKMKAAVVHEFGKPLTIEELDIPTIKPTQILVKMIACGVCHTDLHAASGDWPKKPHLPFIPGHEGVGTVVQVGSEVDWVKEGDVVGVPWLYSACGHCEHCLAGWETLCPKQEETGYSVNGCFAEYVVADPNYIAHLPKGVDPVKVAPVLCAGLTVYKGLKMTDTRAGNWVAISGVGGLGQMAVQYAVAMGLNVVAVDIDDEKLATAKKLGATYTVNARNTDPAAFMQEKVGGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 121 - protein, 342 aa, Acetobacter pasteurianus
MSGKMKAAVVHEFGKPLTIEELDIPPIKPTQILVKMIACGVCHTDLHAASGDWPKKPHLPFIPGHEGVGTVVQVGSEVDWVKEGDVVGVPWLYSACGHCEHCLAGWETLCAKQEETGYSVNGCFAEYVVADPNYIAHLPKGVDPVKVAPVLCAGLTVYKGLKMTDTRAGNWVAISGVGGLGQMAVQYAVAMGLNVVAVDIDDEKLATAKKLGATYTVNARNTDPAAFMQEKVGGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 122 - protein, 342 aa, Acetobacter sp.
  source 1..342: note = strain DmW_136
MSEKMKAAVVHEFGKPLTIEELDIPTIKPTQILVKMIACGVCHTDLHAASGDWPKKPHLPFIPGHEGVGTVVQVGSEVDWVKEGDVVGVPWLYSACGHCEHCLAGWETLCPKQEETGYSVNGCFAEYVVADPNYIAHLPKGVDPVKVAPVLCAGLTVYKGLKMTDTRAGNWVAISGVGGLGQMAVQYAVAMGLNVVAVDIDDEKLATAKKLGAMYTVNARNTDPATFMQEKVGGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 123 - protein, 342 aa, Acetobacter sp.
MSGKMKAAVVHEFGKPLTIEELDIPTIKPTQILVKMIACGVCHTDLHAASGDWPKKPHLPFIPGHEGVGTVVQVGSEVDWVKEGDVVGVPWLYSACGHCEHCLAGWETLCPKQEETGYSVNGCFAEYVVADPNYIAHLPKGVDPVKVAPVLCAGLTVYKGLKMTDTRAGNWVAISGVGGLGQMAVQYAVAMGLNVVAVDIDDEKLATAKKLGAMYTVNARNTDPATFMQEKVGGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 124 - protein, 342 aa, Acetobacter malorum
MSGKMKAAVVREFGKPLSIEELDIPDIRPNEILVKMIACGVCHTDLHAAEGDWPKKPSLPFIPGHEGVGTVVKAGSAVTHVKVGDEVGVPWLYSACGHCEHCLGGWETLCAKQEDTGYSVNGCFAEYVVADPNYVALLPKGIDPVQVAPVLCAGLTVYKGLKMTDTKAGNWVAISGVGGLGQMAVQYGVAMGLNVVAVDIDDEKLATARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 125 - protein, 342 aa, Acetobacter sp.
MSGKMKAAVVHEFGKPLTIEELDIPTIKPTQILVKMIACGVCHTDLHAASGDWPKKPHLPFIPGHEGVGTVVQVGSEVDWVKEGDVVGVPWLYSACGHCEHCLAGWETLCPKQEETGYSVNGCFAEYVVADPNYIAHLPKGVDPVKVAPVLCAGLTVYKGLKMTDTRAGNWVAISGVGGLGQMAVQYAVAMGLNVVAVDIDDEKLATAKKLGAMYTVNARNTDPATFMQKKVGGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 126 - protein, 342 aa, Acetobacter malorum
MSGKMKAAVVREFGKPLSIEELDIPDIRPNEILVKMIACGVCHTDLHAAEGDWPKKPTLPFIPGHEGVGTVVKAGSAVTHVKVGDEVGVPWLYSACGHCEHCLGGWETLCAKQEDTGYSVNGCFAEYVVADPNYVALLPKGIDPVQVAPVLCAGLTVYKGLKMTDTKAGNWVAISGVGGLGQMAVQYGVAMGLNVVAVDIDDEKLATARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 127 - protein, 341 aa, Acetobacter sp.
  source 1..341: note = strain DsW_063
MTTMKAAVCREFGKPLTIEQLAIPTIDENQILVKLDACGVCHTDLHAARGDWPAKPTLPFIPGHEGVGRVARVGSNVKWVKEGDFVGVPWLHSACGHCEHCYGGWETLCEKQEDTGYSVNGCFAEYVVADPNYVAHIPAGVDPLHVAPVLCAGLTVYKGLKMTDTKPGDWAVVSGVGGLGQMAVQYGVAMGLNVVAVDIDDAKLTEAKVLGAALTINAKTLDPAAEIQKQIGGAHGVLVTAVSRSAFAQAMGYARRGGTIVLTGLPPGEFPISIFDMVMAGTTVRGSIVGTRLDMIEALSFFADGKVRSVVKADRLENINQIFDDLEHGRVEGRIVIDFRS

SEQ ID NO: 128 - protein, 342 aa, Acetobacter pasteurianus
MSGKMKAAVVHEFGKPLTIEELDIPTIKPTQILVKMIACGVCHTDLHAASGDWPKKPHLPFIPGHEGVGTVVQVGSEVDWVKEGDVVGVPWLYSACGHCEHCLAGWETLCAKQEETGYSVNGCFAEYVVADPNYIAHLPKGVDPVKVAPVLCAGLTVYKGLKMTDTRAGNWVAISGVGGLGQMAVQYAVAMGLNVVAVDIDDEKLATAKKLGATYTVNARNTNPAAFMQEKVGGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 129 - protein, 342 aa, Acetobacter ascendens
MSGKMKAAVVHEFGKPLTIEELDIPTIKPTQILVRMIACGVCHTDLHAASGDWPKKPHLPFIPGHEGVGTVVQVGSEVDWVKEGDVVGVPWLYSACGHCEHCLAGWETLCPKQEETGYSVNGCFAEYVVADPNYIAHLPKGVDPVKVAPVLCAGLTVYKGLKMTDTRAGNWVAISGVGGLGQMAVQYAVAMGLNVVAVDIDDEKLATAKKLGATYTVNARNTDPAAFMQEKVGGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 130 - protein, 342 aa, Acetobacter pasteurianus
  source 1..342: note = strain NBRC 101655
MSGKMKAAVVHEFGKPLTIEELDIPTIKPTQILVKMIACGVCHTDLHAASGDWPKKPHLPFIPGHEGVGTVVQVGSEVDWVKEGDVVGVPWLYSACGHCEHCLAGWETLCAKQEETGYSVNGCFAEYVVADPNYIAHLPKGVDPVKVAPVLCAGLTVYKGLKMTDTRAGNWVAISGVGGLGQMAVQYAVAMGLNVVAVDIDDEKLATAKKLGATYTVNARNTDPAAFMQEKVGGVHGGLITAVSTKAFSQAMGYVRAGGTLVLNGLPPGDFPISIFDVVMNAITIRGSIVGTRLDMIEALSFFAEGKVTSVTTTDRIDNINAIFDALKNGRVEGRVVLDFRN

SEQ ID NO: 131 - protein, 342 aa, Acetobacter senegalensis
  source 1..342: note = strain DSM 18889
MSGKMKAAVVHEFGKPLTIEELDIPTIKPTQILVKMIACGVCHTDLHAASGDWPKKPHLPFIPGHEGVGTVVQVGNEVDWVKEGDVVGVPWLYSACGHCEHCLAGWETLCPKQEETGYSVNGCFAEYVVADPNYIAHLPKGVDPVKVAPVLCAGLTVYKGLKMTDTRAGNWVAISGVGGLGQMAVQYAVAMGLNVVAVDIDDEKLATAKKLGATYTVNARNTDPAAFMQEKVGGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 132 - protein, 342 aa, Acetobacter ascendens
MSGKMKAAVVHEFGKPLTIEELDIPTIKPTQILVKMIACGVCHTDLHAASGDWPKKPHLPFIPGHEGVGTVVQVGSEVDWVKEGDVVGVPWLYSACGHCEHCLAGWETLCPKQEETGYSVNGCFAEYVVADPNYIAHLPKGVDPVKIAPVLCAGLTVYKGLKMTDTRAGNWVAISGVGGLGQMAVQYAVAMGLNVVAVDIDDEKLAIAKKLGAAYTVNARNTDPAAFMQEKVGGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 133 - protein, 338 aa, Acetobacter cerevisiae
  source 1..338: note = strain DSM 14362
MKAAVVREFGKPLSIEELDIPDIRPNEILVKMIACGVCHTDLHAADGDWPKKPTLPFIPGHEGVGTVVKTGSAVTHVKVGDEVGVPWLYSACGHCEHCLGGWETLCAKQEDTGYSVNGCFAEYVVADPNYVALLPKSIDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 134 - protein, 342 aa, Acetobacter malorum
MSGKMKAAVVREFGKPLSIEELDIPDIRPNEILVKMIACGVCHTDLHAAEGDWPKKPTLPFIPGHEGVGTVVKAGSAVTHVKVGDEVGVPWLYSACGHCEHCLGGWETLCAKQEDTGYSVNGCFAEYVVADPNYVALLPKGIDPVQVAPVLCAGLTVYKGLKMTDTKAGNWVAISGVGGLGQMAVQYGVAMGLNVVAVDIDDEKLATARQLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 135 - protein, 342 aa, Acetobacter cerevisiae
MSGKMKAAVVREFGKPLSIEELDIPDIRPNEILVKMIACGVCHTDLHAADGDWPKKPTLPFIPGHEGVGTVVKTGSAVTHVKVGDEVGVPWLYSACGHCEHCLGGWETLCAKQEDTGYSVNGCFAEYVVADPNYVALLPKSIDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 136 - protein, 342 aa, Acetobacter pasteurianus
MSGKMKAAVVHEFGKPLTIEELDIPPIKPTQILVKMIAGGVCHTDLHAASGDWPKKPHLPFIPGHEGVGTVVQVGSEVDWVKEGDVVGVPWLYSACGHCEHCLAGWETLCAKQEETGYSVNGCFAEYVVADPNYIAHLPKGVDPVKVAPVLCAGLTVYKGLKMTDTRAGNWVAISGVGGLGQMAVQYAVAMGLNVVAVDIDDEKLATAKKLGATYTVNARNTDPAAFMQEKVGGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 137 - protein, 342 aa, Acetobacter malorum
MSGKMKAAVVREFGKPLSIEELDIPDIRPNEILVKMIACGVCHTDLHAAEGDWPKKPTLPFIPGHEGVGTVVKAGSAVTHVKVGDDVGVPWLYSACGHCEHCLGGWETLCAKQEDTGYSVNGCFAEYVVADPNYVALLPKGIDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 138 - protein, 342 aa, Acetobacter cerevisiae
MSGKMKAAVVREFGKPLSIEELDIPDIRPNEILVKMIACGVCHTDLHAADGDWPKKPTLPFIPGHEGVGTVVKTGSAVTHVKVGDEVGVPWLYSACGHCEHCLGGWETLCAKQEDTGYSVNGCFAEYVVADPNYVALLPKGIDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 139 - protein, 342 aa, Acetobacter malorum
MSGKMKAAVVREFGKPLSIEELDIPDIRPNEILVKMIACGVCHTDLHAAEGDWPKKPSLPFIPGHEGVGTVVKAGSAVTHVKVGDEVGVPWLYSACGHCEHCLGGWETLCAKQEDTGYSVNGCFAEYVVADPNYVALLPKGIDPVQVAPVLCAGLTVYKGLKMTDTKAGNWVAISGVGGLGQMAVQYGVAMGLNVVAVDIDDEKLATARKLGAALTVNARETDPAAFMQKEVGGVHGGLITAVSTKAFSQAMGYVRAGGTLVLNGLPPGDFPISIFDMVMNGTTIRGSIVGTRLDMIEALSFCADGKVKSVTQTDRLENINSIFERLEEGKVEGRIVIDFRA

SEQ ID NO: 140 - protein, 338 aa, Acetobacter malorum
  source 1..338: note = strain DSM 14337
MKAAVVREFGKPLSIEELDIPDIRPNEILVKMIACGVCHTDLHAAEGDWPKKPSLPFIPGHEGVGTVVKAGSAVTHVKVGDEVGVPWLYSACGHCEHCLGGWETLCAKQEDTGYSVNGCFAEYVVADPNYVALLPKGIDPVQVAPVLCAGLTVYKGLKMTDTKAGNWVAISGVGGLGQMAVQYGVAMGLNVVAVDIDDEKLATARKLGAALTVNARETDPAAFMQKEVGGVHGGLITAVSTKAFSQAMGYVRAGGTLVLNGLPPGDFPISIFDMVMNGTTIRGSIVGTRLDMIEALSFCADGKVKSVTQTDRLENINSIFERLEEGKVEGRIVIDFRA

SEQ ID NO: 141 - protein, 342 aa, Acetobacter cerevisiae
MSGKMKAAVVREFGKPLSIEELDIPDIRPNEILVKMIACGVCHTDLHAADGDWPKKPTLPFIPGHEGVGTVVKTGSAVTHVKVGDEVGVPWLYSACGHCEHCLGGWETLCAKQEDTGYSVNGCFAEYVVADPNYVALLPKGIDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLGEGKVEGRIVIDFRA

SEQ ID NO: 142 - protein, 342 aa, Ameyamaea chiangmaiensis
MSLTMKAAVVRAFGKPLTIEELAVPEPGPGQILVKMVACGVCHTDLHAANGDWPAKPNPPFIPGHEGVGRVAAVGRDVKWVKEGDLVGVPWLYSACGHCEHCLGGWETLCHDQQDTGYSVNGCFAEYVIADPGYVAHIPANADPLQIAPVLCAGLTVYKGLKMTDTKPGDWVAISGVGGLGQMAVQYGVAMGLNVVAVDIDDGKLATAKGLGAALTVNALHGDPAATIQKETGGAHGTLVTAVSRGAFSQAMKFGRRGGTMVLNGLPPGDFPVSIFDMVMDGTTIRGSIVGTRLDMIEALSFFGDGKVRTVVKPDRLENINQIFDDLEHGRVDGRIVLDFTH

SEQ ID NO: 143 - protein, 342 aa, Acetobacter persici
MSGKMKAAVVREFGKPLTLEELDIPEIRPDQILVKMIACGVCHTDLHAAAGDWPKKPSLPFIPGHEGVGTIVKAGSAVSHVREGDVVGVPWLYSACGHCEHCLGGWETLCGKQEDTGYSVNGCFAEYVVADPNYVALLPKGVDPVQVAPVLCAGLTVYKGLKMTDTKAGNWVAISGVGGLGQMAVQYGVAMGLNVVAVDIDDEKLAIARKLGAALTVNAKETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 144 - protein, 342 aa, Acetobacter malorum
MSGKMKAAVVREFGKPLSIEELDIPDIRPNEILVKMIACGVCHTDLHAAEGDWPKKPTLPFIPGHEGVGTVVKAGSAVTHVRVGDEVGVPWLYSACGHCEHCLGGWETLCAKQEDTGYSVNGCFAEYVVADPNYVALLPKGIDPVQVAPVLCAGLTVYKGLKMTDTKAGNWVAISGVGGLGQMAVQYGVAMGLNVVAVDIDDEKLTTARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 145 - protein, 342 aa, Acetobacter pasteurianus
MSGKMKAAVVHEFGKPLTIEELDIPTIKPTQILVKMIACGVCHTDLHAASGDWPKKPHLPFIPGHEGVGTVVQVGSEVDWVKEGDVVGVPWLYSACGHCEHCLAGWETLCPKQEETGYSVNGCFAEYVVADPNYIAHLPKGVDPVKIAPVLCAGLTVYKGLKMTDTRAGNWVAISGVGGLGQMAVQYAVAMGLNVVAVDIDDEKLAIAKKLGAAYTVNARNTDPAAFMQEKVGGVHGGLITAVSTKAFSQAMGYVRAGGTLVLNGLPPGDFQISIFDMVMNAITIRGSIVGTRLDMIEALSFFAEGKVTSVTTTDRIDNINAIFDALKNGRVEGRVVLDFRN

SEQ ID NO: 146 - protein, 341 aa, Acetobacter nitrogenifigens
MTTMKAAVCREFGKPLTIEQLAIPTIDENQILVKVDVCGVCHTDLHAVSGDWPAKPTLPFIPGHEGVGRVARVGSNVKWVKEGDVVGVPWLHSACGHCEHCYGGWETLCEKQEDTGYSVNGCFAEYVVADPNYVAHIPAGVDPLHVAPVLCAGLTVYKGLKMTDTKPGDWVAVSGVGGLGQMAVQYGVAMGLNVIAVDIDDAKLAEAKELGAAFVVNAKDEDPAAAIQKKVGGAHGVLVTAVSRSAFAQAMGYPRRGGTIVLTGLPPGEFPISIFDMVMAGTTVRGSIVGTRLDMIEALSFFADGKVRSVVKADRLENINQIFDDLEHGRVEGRIVIDFRN

SEQ ID NO: 147 - protein, 342 aa, Acetobacter cerevisiae
MSGKMKAAVVRKFGKPLSIEELDIPDIRPNEILVKMIACGVCHTDLHAADGDWPKKPTLPFIPGHEGVGTVVKTGSAVTHVKVGDEVGVPWLYSACGHCEHCLGGWETLCAKQEDTGYSVNGCFAEYVVADPNYVALLPKGIDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 148 - protein, 342 aa, Acetobacter persici
MSGKMKAAVVREFGKPLTLEELDIPEIRPEQILVKMIACGVCHTDLHAAAGDWPKKPTLPFIPGHEGVGTIVKAGSAVSHVREGDVVGVPWLYSACGHCEHCLGGWETLCGKQEDTGYSVNGCFAEYVVADPNYVALLPKGVDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 149 - protein, 342 aa, Acetobacter ascendens
MSGKMKAAVVHEFGKPLTIEELDIPTIKPTQILVRMIACGVCHTDLHAASGDWPKKPHLPFIPGHEGVGTVVQVGSEVDWVKEGDVVGVPWLYSACGHCEHCLAGWETLCPKQEETGYSVNGCFAEYVVADPNYIVHLPKGVDPVKIAPVLCAGLTVYKGLKMTDTRAGNWVAISGVGGLGQMAVQYAVAMGLNVVAVDIDDEKLAIAKKLGAVYTVNARNTDPAAFMQEKVGGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 150 - protein, 342 aa, Acetobacter persici
MSGKMKAAVVREFGKPLTLEELDIPEIRPDQILVKMIACGVCHTDLHAAAGDWPKKPSLPFIPGHEGVGTIVKAGSAVSHVREGDVVGVPWLYSACGHCEHCLGGWETLCGKQEDTGYSVNGCFAEYVVADPNYVALLPKGVDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 151 - protein, 342 aa, Acetobacter aceti
  source 1..342: note = strain 1023
MSGKMKAAVVHEFGKPLTIEELDIPSIKPTQILVKMIACGVCHTDLHAASGDWPKKPHLPFIPGHEGVGTVVQVGNEVDWVKQGDVVGVPWLYSACGHCEHCLAGWETLCPKQQETGYSVNGCFAEYVAADPNYIAHLPKGVDPIKVAPVLCAGLTVYKGLKMTDTRAGNWVAISGVGGLGQMAVQYAVAMGLNVVAVDIDDEKLATAKKLGAAYIVNARKTDPAAFIQEKVRGVHGGLITAVSTQAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 152 - protein, 342 aa, Acetobacter orleanensis
MSGKMKAAVVHAFGKPLTIEELDIPAIRPDQILVKMIACGVCHTDLHAAEGDWPKKPTLPFIPGHEGVGTVVKAGSTVTHVKEGDTVGVPWLYSACGHCEHCLGGWETLCAKQEDTGYSVNGCFAEYVVADPNYVALLPKGVDPVQVAPVLCAGLTVYKGLKMTDTKPGNWVAISGVGGLGQMAVQYGVAMGLNVVAVDIDDEKLATARNLGAALTVNAKETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 153 - protein, 342 aa, Acetobacter pomorum
MSGKMKAAVVHEFGKPLTIDELDIPSIKPTQILVKMIACGVCHTDLHAASGDWPKKPHLPFIPGHEGVGTVVQVGNEVDWVKQGDVVGVPWLYSACGHCEHCLAGWETLCPKQQETGYSVNGCFAEYVVADPNYIAHLPKGVDPIKVAPVLCAGLTVYKGLKMTDTRAGNWVAISGVGGLGQMAVQYAVAMGLNVVAVDIDDEKLATAKKLGAAYIVNARKTDPAVFIQEKVRGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 154 - protein, 338 aa, Acetobacter pomorum
  source 1..338: note = strain DSM 11825
MKAAVVHEFGKPLTIDELDIPSIKPTQILVKMIACGVCHTDLHAASGDWPKKPHLPFIPGHEGVGTVVQVGNEVDWVKQGDVVGVPWLYSACGHCEHCLAGWETLCPKQQETGYSVNGCFAEYVVADPNYIAHLPKGVDPIKVAPVLCAGLTVYKGLKMTDTRAGNWVAISGVGGLGQMAVQYAVAMGLNVVAVDIDDEKLATAKKLGAAYIVNARKTDPAVFIQEKVRGVHGGLITAVSTKAFSQAMGYVRAGGTLVLNGLPPGDFPISIFDMVMNAITIRGSIVGTRLDMIEALSFFAEGKVTSVTTTDRIDNINAIFDALKNGRVEGRVVLDFRN

SEQ ID NO: 155 - protein, 342 aa, Acetobacter sp.
  source 1..342: note = strain P1H12_c
MSGKMKAAIVREFGKPLTLEELDIPEIRPDQILVKMIACGVCHTDLHAAAGDWPKKPSLPFIPGHEGVGTIVKAGSAVSHVREGDVVGVPWLYSACGHCEHCLGGWETLCGKQEDTGYSVNGCFAEYVVADPNYVALLPKGVDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 156 - protein, 342 aa, Acetobacter persici
MSGKMKAAVVREFGKPLTLEELDIPEIRPDQILVKMIACGVCHTDLHAAAGDWPKKPSLPFIPGHEGVGTIVKAGSAVSHVREGDIVGVPWLYSACGHCEHCLGGWETLCGKQEDTGYSVNGCFAEYVVADPNYVALLPKGVDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 157 - protein, 342 aa, Acetobacter persici
MSGKMKAAVVREFGKPLTLEELDIPEIRPDQILVKMIACGVCHTDLHAAAGDWPKKPSLPFIPGHEGVGTIVKAGSAVTHVREGDVVGVPWLYSACGHCEHCLGGWETLCGKQEDTGYSVNGCFAEYVVADPNYVALLPKGVDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLKNINSIFERLEEGKVEGRIVIDFRA

SEQ ID NO: 158 - protein, 342 aa, Acetobacter persici
MSGKMKAAVVREFGKPLTLEELDIPEIRPDQILVKMIACGVCHTDLHAAAGDWPKKPSLPFIPGHEGVGTIVKAGSAVSHVREGDVVGVPWLYSACGHCEHCLGGWETLCGKQEDTGYSVNGCFAEYVVADPNYVALLSKGVDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVEGRIVIDFRA

SEQ ID NO: 159 - protein, 342 aa, Acetobacter persici
MLGKMKAAVVREFGKPLILEELDIPEIRPDQILVKMIACGVCHTDLHAAAGDWPKKPSLPFIPGHEGVGTIVKAGSAVSHVREGDVVGVPWLYSACGHCEHCLGGWETLCGKQEDTGYSVNGCFAEYVVADPNYVALLPKGVDPVQVAPVLCAGLTVYKGLKMTDTKAGNWVAISGVGGLGQMAVQYGVAMGLNVVAVDIDDEKLAIARKLGAALTVNARETDPAAFMQKEVGGVHGGLITAVSTKAFSQAMGYVRAGGTLVLNGLPPGDFPISIFDMVMNGTTIRGSIVGTRLDMIEALSFFADGKVKSVTQTDRLENINSIFERLEEGKVDGRIVIDFRA

SEQ ID NO: 160 - protein, 344 aa, Tanticharoenia sakaeratensis
  source 1..344: note = strain NBRC 103193
MHMADTMKAAMVHAFGAPLVIETVNIPAIDRHQILVKIDACGVCHTDLHAAKGDWPVKPRLPFIPGHEGVGHVVAVGADVSWVRQGDVVGVPWLYSACGHCAHCLGGWETLCERQDDTGYSVNGCFAEYVVADPDYVAHLPKDADPLLVAPVLCAGVTVYKGLKMTDTKPGDWVVVSGVGGLGQMAVQYGVAMGLNVIAVDIDEAKLATARTLGAALVVNAQSDDPAAFVKKQVGGAHGALVTAVSRPAFSQAMGMVRRGGTIVLNGLPPGDFPVSIFDMVMNGTTLRGSIVGTRLDMIEALSFFTEGKVHSVIRPDRLENINAIFADLEHGHVEGRVVLDFRN

SEQ ID NO: 161 - protein, 342 aa, Tanticharoenia sakaeratensis
MADTMKAAMVHAFGAPLVIETVNIPAIDRHQILVKIDACGVCHTDLHAAKGDWPVKPRLPFIPGHEGVGHVVAVGADVSWVRQGDVVGVPWLYSACGHCAHCLGGWETLCERQDDTGYSVNGCFAEYVVADPDYVAHLPKDADPLLVAPVLCAGVTVYKGLKMTDTKPGDWVVVSGVGGLGQMAVQYGVAMGLNVIAVDIDEAKLATARTLGAALVVNAQSDDPAAFVKKQVGGAHGALVTAVSRPAFSQAMGMVRRGGTIVLNGLPPGDFPVSIFDMVMNGTTLRGSIVGTRLDMIEALSFFTEGKVHSVIRPDRLENINAIFADLEHGHVEGRVVLDFRN

SEQ ID NO: 162 - protein, 342 aa, Acetobacter sp.
MSGKMKAAVVHEFGKPLTIEELDIPDIRPDQILVKMTACGVCHTDLHAASGDWPKKPTVPFIPGHEGVGVVTKVGSNVTWVKEGDEVGVPWLYSACGHCEHCLGGWETLCAKQQETGYSVNGCFAEYVVADPNYVARLPKGIDPVKVAPVLCAGLTVYKGLKMTDTKAGNWVAISGVGGLGQMAVQYGVAMGLNVIAVDIDDQKLATARKLGAALTVNARTTDPAAFIQKEVGGVHGGLITAVSTKAFSQAMGYVRAGGTLVLNGLPPGDFPISIFDMVMKGTTIRGSIVGTRLDMIEALSFFAEGKVTSVTQTDRLENINNIFTRLEEGKVEGRIVIDFRA

SEQ ID NO: 163 - protein, 342 aa, Acetobacter tropicalis
MSGKMKAAVVHEFGKPLTIEELDIPDIRPDQILVKMTACGVCHTDLHAASGDWPKKPTVPFIPGHEGVGVVTKVGSNVTWVKEGDEVGVPWLYSACGHCEHCLGGWETLCAKQQETGYSVNGCFAEYVVADPNYVARLPKGIDPVKVAPVLCAGLTVYKGLKMTDTKAGNWVAISGVGGLGQMAVQYGVAMGLNVIAVDIDDQKLATARKLGAALTVNARTTDPAAFIQKEVGGVHGGLITAVSTKAFSQAMGYVRAGGTLVLNGLPPGDFPISIFDMVMKGTTIRGSIVGTRLDMIEALSFFAEGKVTSVTQTDRLENINNIFTHLEEGKVEGRIVIDFRA

SEQ ID NO: 164 - protein, 342 aa, Acetobacter sp.
MSGKMKAAVVHEFGKPLTIEELDIPDIQPDQILVKMTACGVCHTDLHAASGDWPKKPTVPFIPGHEGVGVVTKVGSNVTWVKEGDEVGVPWLYSACGHCEHCLGGWETLCAKQQETGYSVNGCFAEYVVADPNYVARLPKGIDPVKVAPVLCAGLTVYKGLKMTDTKAGNWVAISGVGGLGQMAVQYGVAMGLNVIAVDIDDQKLATARKLGAALTVNARTTDPAVFIQKEVGGVHGGLITAVSTKAFSQAMGYVRAGGTLVLNGLPPGDFPISIFDMVMKGTTIRGSIVGTRLDMIEALSFFAEGKVTSVTQTDRLENINNIFTRLEEGKVEGRIVIDFRA

SEQ ID NO: 165 - protein, 342 aa, Acetobacter cibinongensis
MSGKMKAAVVREFGKPLSIEELAIPDINANQILVKMIACGVCHTDLHAAHGDWPAKPTLPFIPGHEGVGTVVKVGANVSWVKEGDEVGVPWLYSACGHCDHCLSGWETLCAKQEDTGYSVNGCFAEYVVADPNYVARLPKGVDPIKVAPVLCAGLTVYKGLKMTDAKAGQWVAVSGVGGLGQMAVQYGVAMGLNIIAVDIDDDKLETARKLGAAHTVNALKTDPAAFVHKEVGGAHGGLITAVSTKAFSQAMGYVRSGGTLVLNGLPPGEFPISIFDMVMKGTTIRGSIVGTRLDMIEALSFFADGKITSVTQTDKLENINTIFEKLEKGQVEGRIVLDFRQ

SEQ ID NO: 166 - protein, 342 aa, Acetobacter senegalensis
MSGKMKAAVVHEFGKPLTIEELDIPDIRPDQILVKMTACGVCHTDLHAASGDWPKKPTVPFIPGHEGVGVVTKVGSNVTWVKEGDEVGVPWLYSACGHCEHCLGGWETLCAKQQETGYSVNGCFAEYVVADPNYVARLPKGIDPVKVAPVLCAGLTVYKGLKMTDTKAGNWVAISGVGGLGQMAVQYGVAMGLNVIAVDIDDQKLATARKLGAALTVNARTTDPAAFIQKEVGGVHGGLITAVSTKAFSQAMGYVRAGGTLVLNGLPPGDFPISIFDMVMKGTTIRGSIVGTRLDMIEALSFFAEGKVTSLTQTDRLENINNIFTRLEEGKVEGRIVIDFRA

SEQ ID NO: 167 - protein, 342 aa, Acetobacter cibinongensis
MSGKMKAAVVREFGKPLSIEELAIPDINANQILVKMIACGVCHTDLHAAHGDWPAKPTLPFIPGHEGVGTVVKVGANVSWVKEGDEVGVPWLYSACGHCDHCLSGWETLCAKQEDTGYSVNGCFAEYVVADPNYVARLPKGVDPIKIAPVLCAGLTVYKGLKMTDAKAGQWVAVSGVGGLGQMAVQYGVAMGLNIIAVDIDDDKLETARKLGAAHTVNALKTDPAAFVHKEVGGAHGGLITAVSTKAFSQAMGYVRSGGTLVLNGLPPGEFPISIFDMVMKGTTIRGSIVGTRLDMIEALSFFADGKITSVTQTDKLENINTIFEKLEKGQVEGRIVLDFRQ

SEQ ID NO: 168 - protein, 342 aa, Acetobacter senegalensis
MSGKMKAAVVHEFGKPLTIEELDIPDIRPDQILVKMTACGVCHTDLHAASGDWPKKPTVPFIPGHEGVGVVTKVGSNVTWVKEGDEVGVPWLYSACGHCEHCLGGWETLCAKQQETGYSVNGCFAEYVVADPNYVARLPKGIDPVKVAPVLCAGLTVYKGLKMTDTKAGNWVAISGVGGLGQMAVQYGVAMGLNVIAVDIDDQKLATARKLGAALTVNARTTDPSAFIQKEVGGVHGGLITAVSTKAFSQAMGYVRAGGTLVLNGLPPGDFPISIFDMVMKGTTIRGSIVGTRLDMIEALSFFAEGKVTSVTQTDRLENINNIFTRLEEGKVEGRIVIDFRA

SEQ ID NO: 169 - protein, 338 aa, Acetobacter cibinongensis
MKAAVVREFGKPLSIEELAIPDINANQILVKMIACGVCHTDLHAAHGDWPAKPTLPFIPGHEGVGTVVKVGANVSWVKEGDEVGVPWLYSACGHCDHCLSGWETLCAKQEDTGYSVNGCFAEYVVADPNYVARLPKGVDPIKIAPVLCAGLTVYKGLKMTDAKAGQWVAVSGVGGLGQMAVQYGVAMGLNIIAVDIDDDKLETARKLGAAHTVNALKTDPAAFVHKEVGGAHGGLITAVSTKAFSQAMGYVRSGGTLVLNGLPPGEFPISIFDMVMKGTTIRGSIVGTRLDMIEALSFFADGKITSVTQTDKLENINTIFEKLEKGQVEGRIVLDFRQ

SEQ ID NO: 170 - protein, 342 aa, Acetobacter senegalensis
MSGKMKAAVVHEFGKPLTIEELDIPDIRPDQILVKMTACGVCHTDLHATSGDWPKKPTVPFIPGHEGVGVVTKVGSNVTWVKEGDEVGVPWLYSACGHCEHCLGGWETLCAKQQETGYSVNGCFAEYVVADPNYVARLPKGIDPVKVAPVLCAGLTVYKGLKMTDTKAGNWVAISGVGGLGQMAVQYGVAMGLNVIAVDIDDQKLATARKLGAALTVNARTTDPAAFIQKEVGGVHGGLITAVSTKAFSQAMGYVRAGGTLVLNGLPPGDFPISIFDMVMKGTTIRGSIVGTRLDMIEALSFFAEGKVTSVTQTDRLENINNIFTRLEEGKVEGRIVIDFRA

SEQ ID NO: 171 - protein, 342 aa, Acetobacter indonesiensis
MSGKMKAAVVHEFGKALTIEELDIPDIRPDQILVKMTACGVCHTDLHAAAGDWPKKPSLPFIPGHEGVGIVAKVGSNVTWVKEGDEVGVPWLYSACGHCEHCLGGWETLCSKQEETGYSVNGCFAEYVVADPNYVARLPKDIDPIKVAPVLCAGLTVYKGLKMTDTKAGNWVAISGVGGLGQMAVQYGVAMGLNVIAVDIDDSKLETARKLGAALTVNARETDPAAFIQKEVGGVHGGLITAVSTKAFSQAMGYVRAGGTLVLNGLPPGEFPISIFDMVMKGTTIRGSIVGTRLDMIEALSFFADGKVTSVTQTDKLENINDIFAKLEEGKVEGRIVIDFRS

SEQ ID NO: 172 - protein, 342 aa, Acetobacter indonesiensis
MSGKMKAAVVHEFGKALTIEELDIPDIRPDQILVKMTACGVCHTDLHAAAGDWPKKPSLPFIPGHEGVGIVAKVGSNVTWVKEGDEVGVPWLYSACGHCEHCLGGWETLCAKQEETGYSVNGCFAEYVVADPNYVARLPKGIDPIKVAPVLCAGLTVYKGLKMTDTKAGNWVAISGVGGLGQMAVQYGVAMGLNVIAVDIDDSKLETARKLGAALTVNARETDPAAFIQKEVGGVHGGLITAVSTKAFSQAMGYVRAGGTLVLNGLPPGEFPISIFDMVMKGTTIRGSIVGTRLDMIEALSFFADGKVTSVTQTDKLENINDIFSKLEEGKVEGRIVIDFRS

SEQ ID NO: 173 - protein, 342 aa, Acetobacter indonesiensis
MSGKMKAAVVHEFGKALTIEELDIPDIRPDQILVKMTACGVCHTDLHAAAGDWPKKPSLPFIPGHEGVGIVAKVGSNVTWVKEGDEVGVPWLYSACGHCEHCLGGWETLCAKQEETGYSVNGCFAEYVVADPNYVARLPKGIDPIKVAPVLCAGLTVYKGLKMTDTKAGNWVAISGVGGLGQMAVQYGVAMGLNVIAVDIDDSKLETARKLGAALTVNARETDPAAFIQKEVGGVHGGLITAVSTKAFSQAMGYVRAGGTLVLNGLPPGEFPISIFDMVMKGTTIRGSIVGTRLDMIEALSFFADGKVTSVTRTDKLENINDIFAKLEEGKVEGRIVIDFRS

SEQ ID NO: 174 - protein, 342 aa, Acetobacter indonesiensis
MSGKMKAAVVHEFGKALTIEELDIPDIRPDQILVKMTACGVCHTDLHAAAGDWPKKPSLPFIPGHEGVGIVAKVGSNVTWVKEGDEVGVPWLYSACGHCEHCLGGWETLCAKQKETGYSVNGCFAEYVVADPNYVARLPKGIDPIKVAPVLCAGLTVYKGLKMTDTKAGNWVAISGVGGLGQMAVQYGVAMGLNVIAVDIDDSKLETARKLGAALTVNARETDPAAFIQKEVGGVHGGLITAVSTKAFSQAMGYVRAGGTLVLNGLPPGEFPISIFDMVMKGTTIRGSIVGTRLDMIEALSFFADGKVTSVTQTDKLENINDIFAKLEEGKVEGRIVIDFRS

SEQ ID NO: 175 - protein, 338 aa, Acetobacter indonesiensis
MKAAVVHEFGKALTIEELDIPDIRPDQILVKMTACGVCHTDLHAAAGDWPKKPSLPFIPGHEGVGIVAKVGSNVTWVKEGDEVGVPWLYSACGHCEHCLGGWETLCAKQEETGYSVNGCFAEYVVADPNYVARLPKGIDPIKVAPVLCAGLTVYKGLKMTDTKAGNWVAISGVGGLGQMAVQYGVAMGLNVIAIDIDDSKLETARKLGAALTVNARETDPAAFIQKEVGGVHGGLITAVSTKAFSQAMGYVRAGGTLVLNGLPPGEFPISIFDMVMKGTTIRGSIVGTRLDMIEALSFFADGKVTSVTQTDKLENINDIFAKLEEGKVEGRIVIDFRS

SEQ ID NO: 176 - protein, 342 aa, Acetobacter indonesiensis
MSGKMKAAVVHEFGKALTIEELDIPDIRPDQILVKMTACGVCHTDLHAAAGDWPKKPSLPFIPGHEGVGIVAKVGSNVTWVKEGDEVGVPWLYSACGHCEHCLGGWETLCAKQEETGYSVNGCFAEYVVADPNYVARLPKGIDPIKVAPVLCAGLTVYKGLKMTDTKAGNWVAISGVGGLGQMAVQYGVAMGLNVIAIDIDDSKLETARKLGAALTVNARETDPAAFIQKEVGGVHGGLITAVSTKAFSQAMGYVRAGGTLVLNGLPPGEFPISIFDMVMKGTTIRGSIVGTRLDMIEALSFFADGKVTSVTQTDKLENINDIFAKLEEGKVEGRIVIDFRS

SEQ ID NO: 177 - protein, 342 aa, Acetobacter indonesiensis
MSGKMKAAVVHEFGKALTIEELDIPDIRPDQILVKMTACGVCHTDLHAEAGDWPKKPSLPFIPGHEGVGIVAKVGSNVTWVKEGDEVGVPWLYSACGHCEHCLGGWETLCAKQKETGYSVNGCFAEYVVADPNYVARLPKGIDPIKVAPVLCAGLTVYKGLKMTDTKAGNWVAISGVGGLGQMAVQYGVAMGLNVIAVDIDDSKLETARKLGAALTVNARETDPAAFIQKEVGGVHGGLITAVSTKAFSQAMGYVRAGGTLVLNGLPPGEFPISIFDMVMKGTTIRGSIVGTRLDMIEALSFFADGKVTSVTQTDKLENINDIFSKLEKGKVEGRIVIDFRS

SEQ ID NO: 178 - protein, 342 aa, Acetobacter syzygii
MSGKMKAAVVHEFGKPLTLEELDIPSINTNQILVKMIACGVCHTDLHAASGDWPKKPTLPFIPGHEGVGTVVAVGSNVDWIKEGDVVGVPFLYSACGHCEHCFAGWETLCQKQEETGYSVNGCFAEYVVADPNYVAHIPAGVDPIKVAPVLCAGLTVYKGLKMTDTRAGDWVAVSGAGGLGQMAIQYGVAMGLNMIAVDIDDAKLETARKLGAVHTVNAKTTDPAAFMQKEVGGVHGGLITAVSTKAFSQAMGYVRAGGTLVLNGLPPGDFPISIFDMVMNAITIRGSIVGTRLDMIEALSFFAEGKVTSVTKTDRLENINNIFDDLKNGRVEGRVVLDFRN

SEQ ID NO: 179 - protein, 342 aa, Acetobacter syzygii
MSGKMKAAVVHEFGKPLTLEELDIPSINTNQILVKMIACGVCHTDLHAASGDWPKKPTLPFIPGHEGVGTVVAVGSNVDWIKEGDVVGVPFLYSACGHCEHCFAGWETLCQKQEETGYSVNGCFAEYVVADPNYVAHIPAGVDPIKIAPVLCAGLTVYKGLKMTDTRAGDWVAVSGAGGLGQMAIQYGVAMGLNMIAVDIDDAKLETARKLGAVHTVNAKTTDPAAFMQKEVGGVHGGLITAVSTKAFSQAMGYVRAGGTLVLNGLPPGDFPISIFDMVMNAITIRGSIVGTRLDMIEALSFFAEGKVTSVTKTDRLENINNIFDDLKNGRVEGRVVLDFRN

SEQ ID NO: 180 - protein, 342 aa, Acetobacter okinawensis
MSGKMKAAVVHEFGKPLTLEELDIPSINSNQILVKMIACGVCHTDLHAASGDWPKKPTLPFIPGHEGVGTVVAVGSNVDWVKEGDVVGVPFLYSACGHCEHCYAGWETLCQKQEETGYSVNGCFAEYVVADPGYVAHIPQGVDPIKVAPVLCAGLTVYKGLKMTDTRAGNWVAVSGAGGLGQMAIQYGVAMGLNMIAVDIDDAKLDTARKLGAAYTVNARTTDPAAFIQKEVGGAHGGLITAVSTKAFSQAMGYVRPGGTLVLNGLPPGDFPISIFDMVMNAITIRGSIVGTRLDMIEALSFFAEGKVTSVTKTDRLENINQIFDDLEHGRVEGRIVLDFRN

SEQ ID NO: 181 - protein, 342 aa, Acetobacter okinawensis
MSGKMKAAVVHEFGKPLTLEELDIPSINSNQILVKMIACGVCHTDLHAASGDWPKKPTLPFIPGHEGVGTVVAVGSNVDWVKEGDVVGVPFLYSACGHCEHCYAGWETLCQKQEETGYSVNGCFAEYVVADPGYVAHIPQGVDPIKVAPVLCAGLTVYKGLKMTDTRAGNWVAISGAGGLGQMAIQYGVAMGLNMIAVDIDDAKLETARKLGAAYTVNARTTDPAAFIQKEVGGAHGGLITAVSTKAFSQAMGYVRPGGTLVLNGLPPGDFPISIFDMVMNAITIRGSIVGTRLDMIEALSFFAEGKVTSVTKTDRLENINQIFDDLEHGRVEGRIVLDFRN

SEQ ID NO: 182 - protein, 342 aa, Acetobacter lambici
MSGKMKAAVVHEFSKPLTLEELDIPSINPNQILVKMIACGVCHTDLHAASGDWPKKPTLPFIPGHEGVGTVVEVGSNVDWIKEGDVVGVPFLYSACGHCEHCYAGWETLCQKQEETGYSVNGCFAEYVVADPGYVAHIPAGADPIKIAPVLCAGLTVYKGLKMTDTRAGDWVAVSGAGGLGQMAIQYGVAMGLNMIAVDIDDAKLETARKLGAAHTVNAKTTDPAAFIQKEVGGAHGGLITAVSTKAFSQAMGYVRAGGTLVLNGLPPGDFPISIFDMVMNAITIRGSIVGTRLDMIEALSFFAEGKVTSVTKTDRLENINQIFDDLEHGRVEGRVVLDFRN

SEQ ID NO: 183 - protein, 342 aa, Acetobacter ghanensis
MSGKMKAAVVHEFGKPLTLEELDIPSINTNQILVKMIACGVCHTDLHAASGDWPKKPTLPFIPGHEGVGTVVAVGSNVDWIKEGDVVGVPFLYSACGHCEHCFAGWETLCQKQEETGYSVNGCFAEYVVADPNYVAHIPAGADPIKIAPVLCAGLTVYKGLKMTDTHAGDWVAVSGAGGLGQMAIQYGVAMGLNMIAVDIDDAKLETARKLGAVHTVNAKTTDPAAFMQKEVGGVHGGLITAVSTKAFSQAMGYVRAGGTLVLNGLPPGDFPISIFDMVMNAITIRGSIVGTRLDMIEALSFFAEGKVTSVTKTDRLENINQIFDDLKNGRVEGRVVLDFRN

SEQ ID NO: 184 - protein, 342 aa, Acetobacter lovaniensis
MSGKMKAAVVHEFGKPLTLEELDIPSINSNQILVKMIACGVCHTDLHAANGDWPKKPTLPFIPGHEGVGTVVEIGSNVDWIKKGDIVGVPFLYSACGHCEHCYAGWETLCQKQEDTGYSVNGCFAEYVVADPGYVAHIPAGVDPIKVAPVLCAGLTVYKGLKMTDTRAGDWVAVSGAGGLGQMAIQYGVAMGLNMIAVDIDDAKLETARKLGAAHTVNARTTDPAAFIQKEVGGAHGGLITAVSTKAFSQAMGYVRAGGTLVLNGLPPGDFPISIFDMVMNAITIRGSIVGTRLDMIEALSFFAEGKVTSVTKTDRLENINQIFDDLEHGRVEGRVVLDFRN

SEQ ID NO: 185 - protein, 342 aa, Acetobacter fabarum
MSGKMKAAVVHEFGKPLTLEELDIPSINSNQILVKMIACGVCHTDLHAASGDWPKKPTLPFIPGHEGVGTVVEVGSNVDWIKKGDIVGVPFLYSACGHCEHCYAGWETLCQKQEETGYSVNGCFAEYVVADPGYVAHIPPGVDPIKIAPVLCAGLTVYKGLKMTDTRAGDWVAVSGAGGLGQMAIQYGVAMGLNMIAVDIDDAKLETARKLGAAHTVNAKTTDPAAFIQKEVGGAHGGLITAVSTKAFSQAMGYVRAGGTLVLNGLPPGDFPISIFDMVMNAITVRGSIVGTRLDMIEALSFFAEGKVTSVTKTDRLENINQIFDDLEHGRVEGRIVLDFRN

SEQ ID NO: 186 - protein, 342 aa, Acetobacter sp.
MSGKMKAAVVHEFGKPLTLEELDIPSINSNQILVKMIACGVCHTDLHAASGDWPKKPTLPFIPGHEGVGTVVEVGSNVDWIKKGDIVGVPFLYSACGHCEHCYAGWETLCQKQEETGYSVNGCFAEYVVADPGYVAHIPPGVDPIKIAPVLCAGLTVYKGLKMTDTRAGDWVAVSGAGGLGQMAIQYGVAMGLNMIAVDIDDAKLETARKLGAAHTVNAKTTDPAAFIQKEVGGAHGGLITAVSTKAFSQAMGYVRAGGTLVLNGLPPGDFPISIFDMVMNAITVRGSIVGTRLDMIEALSFFAEGKVTSVTKTDRLENINQIFDDLEHGRVEGRVVLDFRN

SEQ ID NO: 187 - protein, 342 aa, Acetobacter vaccinii
MSGTMKAAVVHEFGKPLSLEELAIPTINDTQVLVRMIACGVCHTDLHAARGDWPQKPTLPFIPGHEGVGTVVQVGREVSWVKVGDVVGIPWLHSACGHCPHCLGGWETLCPQQETSGYSVNGCFAEYTVADPNYVAHIPAGVDPIAVAPVLCAGLTVYKGLKMTDARPGDWVAISGVGGLGQMAVQYAIAMGMNVIAVDVSDEKLATAKRLGATHTVNALTTDPAAYIQKEVGGAHAGLITAVSTSAFEQAMGYIRAGGTLVLNGLPPGDFPISIFDMVMNAITIRGSIVGTRLDMIEALSFFAEGKVTSVTQTDTLENINHIFEKLEKGTVEGRIVLDFRN

SEQ ID NO: 188 - protein, 340 aa, Acetobacter sp.
MTDTMLAAVVREFGKPLVIERVPVPDYGPDHILVKMEACGVCHTDLHAAHGDWPVKPTLPFIPGHEGVGRVVAVGSNVKWVSEGERVGVPWLYSACGHCEHCLGGWETLCEKQDDTGYTVNGCFAEYVVADPKYVAHVPEDADPLQVAPVLCAGLTVYKGLKMTEAKAGDWVAVSGVGGLGQMALQYGKAMGFRMIAIDIDDDKLKVATQLGAAVTINALKDDPAAVVRKEAGGAHGVLVTAVSPSAFSQAVGCVRRGGTIVLNGLPPGEFPVSIFGMVLDGLTIRGSIVGTRLDMIEALSFFSDGKIHIPTKTENLENINSIFDDLEKGKVEGRIVLGF

SEQ ID NO: 189 - protein, 342 aa, Beijerinckia indica
MATKMKAALVHAFGKPLSIEEIDIPSIGPDQILVKLTACGVCHTDLHAASGDWPVKPKLPFIPGHEGVGQIAAVGQSVTWVKEGDIVGVPWLYSACGHCDHCLAGWETLCEHQQETGYSVNGCFAEYVVADPNYVAHLPQGLDPIKVAPVLCAGLTVYKGLKMTDTKPGNWVAISGVGGLGQMAVQYGIAMGLNVIAIDIDEEKLATAKKLGAALTINALHQDPGEEIHKAVGGVHGALITAVSPKAFSQAMKVTRRGGTIVLNGLPPGEFPISIFDMVMDGTTVRGSIVGTRLDMVEALDFFKQGKISTVVATDRLDNINAIFDRLQHGKVNGRIVIDYRS

SEQ ID NO: 190 - protein, 345 aa, Sphingomonas echinoides
MNKTMKAAVVREFGQPLVIEEVPVTMPGPGQILVKIAATGVCHTDLHAAEGDWPVKPNPPFIPGHEGVGHVVAVGSGVTYVKEGDRVGVPWLYTACGHCVHCLGGWETLCEAQQNTGYSVNGSFAEYVLADPNYVGHLPANIGFIDIAPILCAGVTVYKGLKVTETRPGNWVAISGIGGLGHMAVQYAKAMGLNVVAVDIDDAKLALAERLGADLTVNARNVDPAAFIKKEIGGAHGALVTAVSPIAFQQALGMVRRGGTVALNGLPPGDFPLSIFDTVLNGITVRGSIVGTRLDLQEALDFAAEGKVRATVTTDTLENINDIFARMHHGEIEGRIVIDFEGGAH

SEQ ID NO: 191 - protein, 345 aa, Sphingobium xenophagum
MNKTMKAAVVREFGQPLVIEEVPVTMPGPGQILVKIAATGVCHTDLHAAEGDWPVKPNPPFIPGHEGVGHVVAVGSGVTYVKEGDRVGVPWLYTACGHCVHCLGGWETLCEAQQNTGYSVNGSFAEYVLADPNYVGHLPANIGFIDIAPILCAGVTVYKGLKVTETRPGNWVAISGIGGLGHMAVQYAKAMGLNVVAVDIDDAKLALAERLGADLTVNARNVDPAAFIKKEIGGAHGALVTAVSPIAFKQALGMVRRGGTVALNGLPPGDFPLSIFDTVLNGITVRGSIVGTRLDLQEALDFAAEGKVHATVTTDTLENINDIFARMHHGEIEGRIVIDFEGGAQ

SEQ ID NO: 192 - protein, 348 aa, Altererythrobacter sp.
  source 1..348: note = strain B11
MLKTMKAAVVREFGQPLVMEEVPIPTVGPGQILVKIAATGVCHTDLHAANGDWPVKPSPPFIPGHEGVGHVVAVGSGVSHVKEGDRVGVPWLYTACGHCVHCLGGWETLCEQQQNTGYSVNGSFAEYVLADPNYVGHLPANVSFVEIAPVLCAGVTVYKGLKTTDTKPGDWVAISGIGGLGHMAVQYAKAMGLNIVAVDIDDEKLALAKRLGADLTINARDADPAAVVKKEIGGAQGVLVTAVSTKAFQQAMGMARRGGTISLNGLPPGDFPLSIFDTVLNGTTIRGSIVGTRLDLQEALDFAAAGKVHATVSTDRLENINDVFARMHRGEIEGRIVLDFESTPAAAA

SEQ ID NO: 193 - protein, 345 aa, Sphingomonas sp.
  source 1..345: note = strain TF3
MNKTMKAAVVREFGQPLVIEEVPVTMPGPGQILVKIAATGVCHTDLHAAEGDWPVKPNPPFIPGHEGVGHVVAVGSGVTYVKEGDRVGVPWLYTACGHCVHCLGGWETLCEAQQNTGYSVNGSFAEYVLADPNYVGHLPANIGFIDIAPILCAGVTVYKGLKVTETRPGNWVAISGIGGLGHMAVQYAKAMGLNVVAVDIDDAKLALAERLGADLTVNARNFDPAAFIKKEIGGAHGALVTAVSPIAFQQALGMVRRGGTVALNGLPPGDFPLSIFDTVLNGITVRGSIVGTRLDLQEALDFASEGKVRATVTTDTLENINDIFARMHHGEIEGRIVIDFEGGAH

SEQ ID NO: 194 - protein, 345 aa, Sphingomonas sp.
  source 1..345: note = unclassified
MNKTMKAAVVREFGQPLVIEEVPVTMPGPGQILVKIAATGVCHTDLHAAEGDWPVKPNPPFIPGHEGVGHVVAVGSGVTYVKEGDRVGVPWLYTACGHCVHCLGGWETLCEAQQNTGYSVNGSFAEYVLADPNYVGHLPANIGFIDIAPILCAGVTVYKGLKVTETRPGNWVAISGIGGLGHMAVQYAKAMGLNVVAVDIDDAKLVLAERLGADLTVNARNVDPAAFIKKEIGGAHGALVTAVSPIAFQQALGMVRRGGTVALNGLPPGDFPLSIFDTVLNGITVRGSIVGTRLDLQEALDFASEGKVRATVTTDTLENINDIFARMHHGEIEGRIVIDFEGGAH

SEQ ID NO: 195 - protein, 345 aa, Sphingobium sp.
MNKTMKAAVVREFGQPLVIEEVPVTMPGPGQILVKIAATGVCHTDLHAAEGDWPVKPNPPFIPGHEGVGHVVAVGSGVTYVKEGDRVGVPWLYTACGHCVHCLGGWETLCEAQQNTGYSVNGSFAEYVLADPNYVGHLPANIGFIDIAPILCAGVTVYKGLKVTDTRPGNWVAISGIGGLGHMAVQYAKAMGLNVVAVDIDDAKLALAERLGADLSVNARNVDPAAFIKKEIGGAHGALVTAVSPIAFKQALGMVRRGGTVALNGLPPGDFPLSIFDTVLNGITVRGSIVGTRLDLQEALDFAAEGKVHATVTTDTLENINDIFARMHHGEIEGRIVIDFEGGAQ

SEQ ID NO: 196 - protein, 344 aa, Sphingomonas sp.
  source 1..344: note = strain AR_OL41
MEKTMKAAVVRAFGQPLVIEEVAVPMPGPGQILVRIAATGVCHTDLHAAQGDWPVKPNPPFIPGHEGVGHVVAVGAGVGHVKEGDRVGVPWLYTACGHCGHCLGGWETLCETQQNTGYSVNGSFAEYVLADPNYVGHLPDNVGFVEIAPVLCAGVTVYKGLKVTDTKPGDWVAISGIGGLGHMAVQYAKAMGLNVVAVDIDDAKLALATELGAALTVNARDTDPAAFIKKEIGGAQGVLVTAVSPVAFQQAMGMVRRGGTVSLNGLPPGDFPLSIFDTVLNGITVRGSIVGTRLDLQEALDFAAAGKVHATVTTDRLENINDIFGRMHRGEIEGRIVIDFDKAA

SEQ ID NO: 197 - protein, 359 aa, Sphingomonas sp.
  source 1..359: note = strain 3F27F9
MQKTMKAAVVREFGKPLVIEEVTVPMPGPGQILVKIAATGVCHTDLHAAEGDWPVKPNPPFIPGHEGVGHVVAVGSGVTHVKEGDRVGVPWLYSACGHCVHCLGGWETLCETQQNTGYSVNGSFAEYVLADPNYVGHLPDNIGFVDISPVLCAGVTVYKGLKVTDTKPGDWVAISGIGGLGHMAVQYAKAMGLNVVAVDVDDAKLKLAEELGASLTVNARDADPAAFIKKEIGGAQGVLVTAVSPIAFKQAMGMVRRGGTVSLNGLPPGEFPMSIFDTVLNGITVRGSIVGTRLDLQESLDFAAAGKVRATVTTDTLENINDVFGRMHRGEIEGRVVIDFENPPVAANATAAAALVSAA

SEQ ID NO: 198 - protein, 359 aa, Sphingomonas sp.
  source 1..359: note = strain TF3
MQKTMKAAVVREFGKPLVIEEVTVPMPGPGQILVKIAATGVCHTDLHAAEGDWPVKPNPPFIPGHEGVGHVVAVGSGVTHVKEGDRVGVPWLYSACGHCVHCLGGWETLCETQQNTGYSVNGSFAEYVLADPNYVGHLPDNIGFVDISPVLCAGVTVYKGLKVTDTKPGDWVAISGIGGLGHMAVQYAKAMGLNVVAVDIDDAKLKLAEELGASLTVNARNVDPAAFIKKEIGGAQGVLVTAVSPIAFKQAMGMVRRGGTVSLNGLPPGEFPMSIFDTVLNGITVRGSIVGTRLDLQESLDFAAAGKVRATVTTDTLENINDVFGRMHRGEIEGRVVIDFENPPVAANATAAAALVSAA

SEQ ID NO: 199 - protein, 345 aa, Sphingobium sp.
  source 1..345: note = unclassified
MNKTMKAAVVREFGQPLVIEEVPVTMPGPGQILVKIAATGVCHTDLHAAEGDWPVKPNPPFIPGHEGVGHVVAVGSGVTYVKEGDRVGVPWLYTACGHCVHCLGGWETLCEAQQNTGYSVNGSFAEYVLADPNYVGHLPANIGFIDIAPILCAGVTVYKGLKVTDTRPGNWVAISGIGGLGHMAVQYAKAMGLNVVAVDIDDAKLALAERLGADLSVNARNVDPAAFIKKEIGGAHGALVTAVSPIAFKQALGMVRRGGTVALNGLPPGDFPLSIFDTVLNGITVRGSIVGTRLDLQEALDFASEGKVRATVTTDTLENINDIFARMHHGEIEGRIVIDFEGGAH

SEQ ID NO: 200 - protein, 342 aa, Pseudomonas sp.
  source 1..342: note = unclassified
MNKTMKAAVVHAFGEPLAIREVPVPMPGAGQVLVKVAASGVCHTDLHAAEGDWPVKPNPPFIPGHEGVGHVVAVGAGVTHVKEGDRVGVPWLYSACGHCEHCLGGWETLCESQQNTGYSVNGGFAEYTLADAGYVGRLPDNVGFLEIAPILCAGVTVYKGLKMTETRPGQWVVVSGIGGLGHMAVQYAKAMGMNVAAVDIDDSKLALAKRLGAEVTVNARNQDPAKYLKQQIGGAHGALVTAVSPKAFEQAMGMTRRGGTIALNGLPPGDFPLSIFDMVLNGTTVRGSIVGTRQDLQEALDFAGEGKVKATVAADKLENINEVFDKMRAGKIDGRIVIDMEH

SEQ ID NO: 201 - protein, 345 aa, Sphingobium psychrophilum
MNKTMKAAVVREFGQPLVIEEVPVTMPGPGQILVKIAATGVCHTDLHAAEGDWPVKPNPPFIPGHEGVGHVVAVGSGVTYVKEGDRVGVPWLYTACGHCVHCLGGWETLCEAQQNTGYSVNGSFAEYVLADPNYVGHLPANIGFIDIAPILCAGVTVYKGLKVTDTRPGNWVAISGIGGLGHMAVQYAKAMGLNVVAVDIDDAKLALAERQGADLSVNARNVDPAAFIKKEIGGAHGALVTAVSPIAFKQALGMVRRGGTVALNGLPPGDFPLSIFDTVLNGITVRGSIVGTRLDLQEALDFAAEGKVHATVTTDTLENINDIFARMHHGEIEGRIVIDFEGGAQ

SEQ ID NO: 202 - protein, 347 aa, Sphingobium sp.
  source 1..347: note = strain SYK-6
MQKTMKAAVVRAFGKPLVIEEVKVPEVGPGQILVKVAAAGVCHTDLHAAEGDWPVKPTPPFIPGHEGVGHVVAVGAGVAHVREGDRVGVPWLYSACGHCVHCLGGWETLCEVQQNTGYSVNGSFAEYVLADPNYVGHLPANVGFVEIAPVLCAGVTVYKGLKVTDTKPGDWVAISGIGGLGHMAVQYAKAMGLNVVAVDIDDEKLALAERLGADLSVNALHADPAAFVRKEIGGAQGVLVTAVSPKAFQQAMGMARRGGTVSLNGLPPGDFPVSIFDTVLNGTTIRGSIVGTRLDLQEALDFAQAGKVHATVKTDALENINDVFARMHRGEIEGRIVLDFEGAGTPA

SEQ ID NO: 203 - protein, 342 aa, Pseudomonas typographi
MTKTMKAAVVHAFGQPLAIREVPVPTPGPGQLLVKVAASGVCHTDLHAAEGDWPAKPNPPFIPGHEGVGHVVAVGAGVTHVKEGDRVGVPWLYSACGHCEHCLGGWETLCETQENTGYSVNGGFAEYTLADANYVGKLPDTIGFVEIAPILCAGVTVYKGLKMTETKPGQWVVISGIGGLGHMAVQYAKAMGMNVAAVDIDDAKLELARRLGATVTVNARELDPAKYLKEQIGGAHGALVTAVSPKAFEQAMGMTRRGGTITLNGLPPGDFPMSIFDMVLNGTTVRGSIVGTRQDLQEALDFAAEGKVKATVATEKLENINGVFDQMRAGTIEGRIVIDMEQ

SEQ ID NO: 204 - protein, 342 aa, Pseudomonas typographi
MTKTMKAAVVHAFGQPLAIREVPVPTPGPGQLLVKVAASGVCHTDLHAAEGDWPAKPNPPFIPGHEGVGHVVAVGAGVTHVKEGDRVGVPWLYSACGHCEHCLGGWETLCETQENTGYSVNGGFAEYTLADANYVGKLPDTIGFVEIAPILCAGVTVYKGLKMTETKPGQWVVISGIGGLGHMAVQYAKAMGMNVAAVDIDDAKLDLARRLGATVTVNARELDPAKYLKEQIGGAHGALVTAVSPKAFEQAMGMTRRGGTITLNGLPPGDFPMSIFDMVLNGTTVRGSIVGTRQDLQEALDFAAEGKVKATVATEKLENINGVFDQMRAGTIEGRIVIDMEQ

SEQ ID NO: 205 - protein, 342 aa, Pseudomonas typographi
MTKTMKAAVVHAFGQPLAIREVPVPTPGPGQLLVKVAASGVCHTDLHAAEGDWPAKPNPPFIPGHEGVGHVVAVGAGVTHVKEGDRVGVPWLYSACGHCEHCLGGWETLCETQENTGYSVNGGFAEYTLADANYVGKLPDTIGFVEIAPILCAGVTVYKGLKMTETKPGQWVVISGIGGLGHMAVQYAKAMGMNVAAVDIDDAKLDLARRLGATVTVNAGELDPAKYLKEQIGGAHGALVTAVSPKAFEQAMGMTRRGGTITLNGLPPGDFPMSIFDMVLNGTTVRGSIVGTRQDLQEALDFAAEGKVKATVATEKLENINGVFDQMRAGTIEGRIVIDMEQ

SEQ ID NO: 206 - protein, 343 aa, Gluconobacter oxydans; identical to SEQ ID NO: 8

SEQ ID NO: 207 - protein, 343 aa, Gluconobacter morbifer; identical to SEQ ID NO: 41

SEQ ID NO: 208 - protein, 354 aa, Gluconobacter diazotrophicus; identical to SEQ ID NO: 44

SEQ ID NO: 209 - protein, 338 aa, Moraxella sp.
  source 1..338: note = strain TAE123
MKAAVLHEFGQSLQIEEVDIPTPGAGEIVVKMQASGVCHTDLHAVEGDWPVKPSPPFIPGHEGVGLITAVGEGVTHVKEGDRVGVAWLYSACGHCTHCLGGWETLCESQQNSGYSVNGSFAEYVLANANYVGIIPESVDSIEIAPVLCAGVTVYKGLKMTDTKPGDWVVISGIGGLGHMAVQYAIAMGLNVAAVDIDDDKLAFAKKLGAKVTVNAKNTDPAEYLQKEIGGAHGALVTAVSAKAFDQALSMLRRGGTLVCNGLPPGDFPVSIFDTVLNGITIRGSIVGTRLDLQESLDMAAAGKVKATVTAEPLENINDIFERMRQGKIEGRIVIDYTM

SEQ ID NO: 210 - protein, 336 aa, Escherichia coli
MKAAVVTKDHHVDVTYKTLRSLKHGEALLKMECCGVCHTDLHVKNGDFGDKTGVILGHEGIGVVAEVGPGVTSLKPGDRASVAWFYEGCGHCEYCNSGNETLCRSVKNAGYSVDGGMAEECIVVADYAVKVPDGLDSAAASSITCAGVTTYKAVKLSKIRPGQWIAIYGLGGLGNLALQYAKNVFNAKVIAIDVNDEQLKLATEMGADLAINSHTEDAAKIVQEKTGGAHAAVVTAVAKAAFNSAVDAVRAGGRVVAVGLPPESMSLDIPRLVLDGIEVVGSLVGTRQDLTEAFQFAAEGKVVPKVALRPLADINTIFTEMEEGKIRGRMVIDFRH

SEQ ID NO: 211 - protein, 339 aa, Geobacillus stearothermophilus
MKAAVVEQFKEPLKIKEVEKPTISYGEVLVRIKACGVCHTDLHAAHGDWPVKPKLPLIPGHEGVGIVEEVGPGVTHLKVGDRVGIPWLYSACGHCDYCLSGQETLCEHQKNAGYSVDGGYAEYCRAAADYVVKIPDNLSFEEAAPIFCAGVTTYKALKVTGAKPGEWVAIYGIGGLGHVAVQYAKAMGLNVVAVDIGDEKLELAKELGADLVVNPLKEDAAKFMKEKVGGVHAAVVTAVSKPAFQSAYNSIRRGGACVLVGLPPEEMPIPIFDTVLNGIKIIGSIVGTRKDLQEALQFAAEGKVKTIIEVQPLEKINEVFDRMLKGQINGRVVLTLEDK

SEQ ID NO: 212 - protein, 340 aa, Streptomyces coelicolor
MKAAVVREFGKPLVIEDRPDPEPGPGQVLVAVEASGLCHTDIHAAHGDWPVKPTPPFVPGHEGVGLVEKLGDGVTHLAVGQRVAVPWLGKACGRCEHCRSGWETLCEDQVNTGYGCDGGYAEKMLAWADFAQPVPEGVSALDAAPLTCAGVTTYKALKVAEVGPAQLVAVSGVGGLGHLAVQYAKIAGARVVAIDVTDDKLQLARELGADLVIDARTQDVGAELKRHGGAHAALALAVNPAAFQAVNSGLRRGGKLVMVALPAHGTLQVPIFDTVLNGTSVIGSIVGTRQDLAEVFQLHAEGRTKVIRETRPLAAVNDCIDEVLGGQVKARIVFDLNAGG

SEQ ID NO: 213 - protein, 336 aa, Colwellia psychrerythraea
MKAAVNHQFKGKLEIEQLDKPTISSHEVLVKIHACGVCHTDLHACHGDWPVKPKMPLVPGHEGVGEIVEVGDQVSHCKVGDRVGIPWLYSACGYCDYCLTGDENLCLSQQNAGYSVDGSYAEYCKADGNYVVKIPDGIDYVDAAPLFCAGVTTYKALKVSTAKPGEWVAIFGIGGLGHLAVQYAVAMGLNVIAVDTGEAKLELAKKLGASLCLDFKHDDVVAKVLAETGGVHASICTAVSKSGFEQSYKVIRRGGKCVLVGLPPEDMPLPIFDTVLNGVSVVGSIVGTRKDLVECLDFAARGKVKAITIEKSLEDINDIFDEMLNGEITGRVVMKF

SEQ ID NO: 214 - protein, 343 aa, Rhodococcus jostii
MAAAQGGTGTMAAWRVVAPGPVAGHPVELGRETVPEPGTGELLVKVLACGVCRTDLHVTEGDLPVHRPRVIPGHEVVGEVAALGDSVSGSFAVGDRVGIAWLRHTCGHCAACLRGAENLCRQSQYTGWDADGGYAEFATVPADYALHLPEGYSDAELAPLLCAGIIGYRALERATVPDGGALGVYGFGGSAHLTAQVASIRGARVHVMTRGAEARALAQRLGATSVQGVRDVPPEPLDSAILFAPVGDLVPVAMEALADGGTLAVAGIHLTDIPALEYQKHLFRERQIRSVTANTRSDSVEFLRIAAEHRLSIDIHPYPLDRADRALRDLAGGKFSGAAVLEP

SEQ ID NO: 215 - protein, 353 aa, Mycobacterium marinum
MTQTLANPGATQATETLCAAVVTGFGEPLQIKELALPTPGYGEALVKLETSGVCHTDLHAAHGDWPVKPAPPFVPGHEGCGTVVALGDGVTDLKVGDKVGNAWLWSACGSCEQCRTGWETLCENQRNGGYTVNGSFGTYMLVNADYAARIPDGADPLEVAPILCAGVTVYKGLKVTDTRPGQWVAISGIGGLGHVAVQYARAMGLRVVAIDIDDDKLALAARLGAELVVNARTSDVVAQVQEATGGVHGVLVTAVHPQAFGQAIGLARRGGTIVFNGLPPGDFPAPIFDIVLKGLTIRGSIVGTRQDMAEALDFYARGLIHPTVEGAALEDINHVFERMERGQIDGRVVIDYR

SEQ ID NO: 216 - protein, 343 aa, Gluconobacter aidae; identical to SEQ ID NO: 14

SEQ ID NO: 217 - protein, 343 aa, Gluconobacter oxydans; identical to SEQ ID NO: 13

SEQ ID NO: 218 - protein, 343 aa, Gluconobacter roseus; identical to SEQ ID NO: 15

SEQ ID NO: 219 - protein, 343 aa, Gluconobacter sp.; identical to SEQ ID NO: 9

SEQ ID NO: 220 - protein, 339 aa, Gluconobacter oxydans; identical to SEQ ID NO: 10
  source 1..339: note = strain NBRC 3293

SEQ ID NO: 221 - protein, 343 aa, Gluconobacter oxydans; identical to SEQ ID NO: 8

SEQ ID NO: 222 - protein, 343 aa, Gluconobacter oxydans; identical to SEQ ID NO: 11

SEQ ID NO: 223 - protein, 343 aa, Gluconobacter oxydans; identical to SEQ ID NO: 12

SEQ ID NO: 224 - protein, 343 aa, Gluconobacter sp.; identical to SEQ ID NO: 18

SEQ ID NO: 225 - protein, 343 aa, Gluconobacter potus; identical to SEQ ID NO: 20

SEQ ID NO: 226 - protein, 343 aa, Gluconobacter sp.; identical to SEQ ID NO: 16
  source 1..343: note = strain P1C6_b

SEQ ID NO: 227 - protein, 343 aa, Gluconobacter sp.; identical to SEQ ID NO: 19
  source 1..343: note = strain Dm-62

SEQ ID NO: 228 - protein, 343 aa, Gluconobacter oxydans; identical to SEQ ID NO: 17

SEQ ID NO: 229 - protein, 342 aa, Gluconobacter thailandicus; identical to SEQ ID NO: 22

SEQ ID NO: 230 - protein, 342 aa, Gluconobacter thailandicus; identical to SEQ ID NO: 23

SEQ ID NO: 231 - protein, 342 aa, Gluconobacter frateurii; identical to SEQ ID NO: 24

SEQ ID NO: 232 - protein, 342 aa, Gluconobacter japonicus; identical to SEQ ID NO: 25

SEQ ID NO: 233 - protein, 342 aa, Gluconobacter cerinus; identical to SEQ ID NO: 27

SEQ ID NO: 234 - protein, 342 aa, Gluconobacter thailandicus; identical to SEQ ID NO: 28

SEQ ID NO: 235 - protein, 342 aa, Gluconobacter sp.; identical to SEQ ID NO: 29

SEQ ID NO: 236 - protein, 342 aa, Gluconobacter cerinus; identical to SEQ ID NO: 38

SEQ ID NO: 237 - protein, 342 aa, Gluconobacter japonicus; identical to SEQ ID NO: 26

SEQ ID NO: 238 - protein, 342 aa, Gluconobacter cerinus; identical to SEQ ID NO: 39

SEQ ID NO: 239 - protein, 342 aa, Gluconobacter sp.; identical to SEQ ID NO: 34

SEQ ID NO: 240 - protein, 342 aa, Gluconobacter sp.; identical to SEQ ID NO: 31

SEQ ID NO: 241 - protein, 342 aa, Gluconobacter cerinus; identical to SEQ ID NO: 32

SEQ ID NO: 242 - protein, 342 aa, Gluconobacter japonicus; identical to SEQ ID NO: 30

SEQ ID NO: 243 - protein, 342 aa, Gluconobacter cerinus; identical to SEQ ID NO: 37

SEQ ID NO: 244 - protein, 342 aa, Gluconobacter cerinus; identical to SEQ ID NO: 33

SEQ ID NO: 245 - protein, 342 aa, Gluconobacter cerinus; identical to SEQ ID NO: 40

SEQ ID NO: 246 - protein, 342 aa, Gluconobacter japonicus; identical to SEQ ID NO: 35

SEQ ID NO: 247 - protein, 342 aa, Gluconobacter japonicus; identical to SEQ ID NO: 36

SEQ ID NO: 248 - protein, 342 aa, Gluconacetobacter diazotrophicus; identical to SEQ ID NO: 45

SEQ ID NO: 249 - protein, 343 aa, Gluconacetobacter diazotrophicus; identical to SEQ ID NO: 46

SEQ ID NO: 250 - protein, 342 aa, Gluconacetobacter asukensis; identical to SEQ ID NO: 53

SEQ ID NO: 251 - protein, 342 aa, Gluconacetobacter tumulicola; identical to SEQ ID NO: 57

SEQ ID NO: 252 - protein, 342 aa, Gluconacetobacter aggeris; identical to SEQ ID NO: 65

SEQ ID NO: 253 - protein, 342 aa, Gluconacetobacter sacchari; identical to SEQ ID NO: 87

SEQ ID NO: 254 - protein, 342 aa, Gluconacetobacter dulcium; identical to SEQ ID NO: 63

SEQ ID NO: 255 - protein, 342 aa, Gluconacetobacter takamatsuzukensis; identical to SEQ ID NO: 66

SEQ ID NO: 256 - protein, 342 aa, Gluconacetobacter liquefaciens; identical to SEQ ID NO: 61

SEQ ID NO: 257 - protein, 342 aa, Gluconacetobacter dulcium; identical to SEQ ID NO: 62

SEQ ID NO: 258 - protein, 338 aa, Gluconacetobacter liquefaciens; identical to SEQ ID NO: 64
  source 1..338: note = strain NRIC 0522

SEQ ID NO: 259 - protein, 338 aa, Gluconacetobacter azotocaptans; identical to SEQ ID NO: 52
  source 1..338: note = strain DSM 13594

SEQ ID NO: 260 - protein, 342 aa, Gluconacetobacter azotocaptans; identical to SEQ ID NO: 51

SEQ ID NO: 261 - protein, 342 aa, Gluconacetobacter azotocaptans; identical to SEQ ID NO: 54

SEQ ID NO: 262 - protein, 342 aa, Gluconacetobacter johannae; identical to SEQ ID NO: 60

SEQ ID NO: 263 - protein, 342 aa, Gluconacetobacter tumulisoli; identical to SEQ ID NO: 55

SEQ ID NO: 264 - protein, 346 aa, Gluconacetobacter johannae; identical to SEQ ID NO: 59

SEQ ID NO: 265 - protein, 479 aa, Escherichia coli
MSVPVQHPMYIDGQFVTWRGDAWIDVVNPATEAVISRIPDGQAEDARKAIDAAERAQPEWEALPAIERASWLRKISAGIRERASEISALIVEEGGKIQQLAEVEVAFTADYIDYMAEWARRYEGEIIQSDRPGENILLFKRALGVTTGILPWNFPFFLIARKMAPALLTGNTIVIKPSEFTPNNAIAFAKIVDEIGLPRGVFNLVLGRGETVGQELAGNPKVAMVSMTGSVSAGEKIMATAAKNITKVCLELGGKAPAIVMDDADLELAVKAIVDSRVINSGQVCNCAERVYVQKGIYDQFVNRLGEAMQAVQFGNPAERNDIAMGPLINAAALERVEQKVARAVEEGARVAFGGKAVEGKGYYYPPTLLLDVRQEMSIMHEETFGPVLPVVAFDTLEDAISMANDSDYGLTSSIYTQNLNVAMKAIKGLKFGETYINRENFEAMQGFHAGWRKSGIGGADGKHGLHEYLQTQVVYLQS

SEQ ID NO: 266 - protein, 477 aa, A. vinelandii
MSSSVPVHRNYIDGAFVESAAHLEVFNPANGALLSRVPAASAEEVERALAAARAAQKDWARKPAIERAGHLRRIAAKIRADAGRIARTITLEQGKIASLAEVEVNFTADYLDYMAEWARRLEGEIIASDRPGENIFLFRKPLGVVAGILPWNFPFFLIARKMAPALLTGNTIVVKPSEETPNNCFEFARLVAETDLPRGVFNVVCGAGQVGGALSSHPGVDLISFTGSVETGARIMAAAAPNLTKLNLELGGKAPAIVLADADLELAVKAIRDSRIINSGQVCNCAERVYVQRQVAEPFIERIAAAMAATRYGDPLAEPEVEMGPLINRLGLEKIDAKVRTALAQGATLVTGGAIAERPGHHYQPTVLTGCRADTRIMREEIFGPVLPIQIVDDLDEAIALANDCEYGLTSSVFTRDLNKAMHALRELDFGETYINREHFEAMQGFHAGVRKSGIGGADGKHGLYEYTHTHVVYLQS

SEQ ID NO: 267 - protein, 503 aa, P. putida
MRYPMKDSSDTHPNHLPDSCYGLFIDNQWVTAEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKEEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 268 - protein, 477 aa, Pseudomonas sp.
  source 1..477: note = strain M47T1
MSSVPVYQNYINGQFADSAEHLEVFNPATGALLSRVPAASGEEVDRALAAARTAQKDWARKPAIERAGYLRRIAAKLREHAPALARTITSEQGKISALAEVEVNFTADYLDYMAEWARRIEGEIISSDRAGENIFLFRKPLGVVAGILPWNFPFFLIARKMAPALITGNTIVVKPSEETPNNCFEFARLVAETDLPAGVFNVVCGDGRVGAALSAHAKVDMISFTGSVDTGARIMAAAAPNLTKLNLELGGKAPAIVLADADLDLAVKAIRDSRIINTGQVCNCAERVYVERKVADQFIERIGAAMAATRYGDPLADSSVEMGPLINRQGLDSVDAKVRRALADGAQLITGGAVADRPAGFHYQPTVLAGCRADMDIMRKEIFGPVLPVQIVDDLDEAIALANDCEYGLTSSLYTRDLNKAMQAMREIDFGETYINRENFEAMQGFHAGVRKSGVGGADGKHGLYEYTHTHVVYLQA

SEQ ID NO: 269 - protein, 478 aa, Stutzerimonas azotifigens
MSESVPVHQNYIDGVFHDSEAHLDVFNPATGARLSRVPAASPEQIERAIGAARAAQKPWSRKPAIERAGYLRKIAAKIRENVPRLARTITLEQGKIAALAEVEVNFTADYLDYVAEWARRIEGEIITSDRPGENIFLFRKPLGVVAGILPWNFPFFLIARKMAPAMLTGNTIVIKPSEETPNNCFEFARLVAETDLPPGVFNVVGGDGRVGGALSAHAGVDMVSFTGSVATGQRIMAAAAPNLTKLNLELGGKAPAIVLADADLELAVKAIRDSRIINTGQVCNCAERVYVERKVADAFIDRIAGAMAATRFGDPSAQPDVEMGPLINRAGLDKVDAKVRAAIAQGASVVTGGGPANLPAGFHYQPTVLAGCRADMPIMREEIFGPVLPIQIIDDLDEAIALANDCEYGLTSSVYTRDLSKAMHAMQELDFGETYVNRENFEAMQGFHAGVRKSGIGGADGKHGLYEYTHTHVVYLQC

SEQ ID NO: 270 - protein, 477 aa, Pseudomonas sp.
MPSVPVYQNFINGCFTASSAHIDVLNPATGALLSKVPAATAEDVDQALAAARAAQKDWSRKPAIERAGHLRRIAAKLRENVAHLARTVTLEQGKISSLAEIEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGQVGGALTAHKGVDMISFTGSVATGSRIMTAAAPNITKLNLELGGKAPAIVLADADLELAVKAIRDSRIINSGQVCNCAERVYVQRSVADQFIERISAAMAATRYGDPLAQADVEMGPLINRQGLESVNRKVRTALAQGASLVTGGQIADLGAGFHFQPTVLAGGRADMDIMREEIFGPVLPIQIIDDLDEAIALANDCDYGLTSSIYTRDLGKAMHAVRELDFGETYVNRENFEAMQGFHAGVRKSGIGGADGKHGLYEYTHTHVVYLQS

SEQ ID NO: 271 - protein, 477 aa, Pseudomonas proteolytica
MPSVPVYQNFINGCFTASSAHIDVLNPATGALLSKVPAATAEDVDQALAAARAAQKDWSRKPAIERAGHLRRIAAKLRENVAHLARTVTLEQGKISSLAEIEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGQVGGALTAHKGVDMISFTGSVATGSRIMTAAAPNITKLNLELGGKAPAIVLADADLELAVKAIRDSRIINSGQVCNCAERVYVQRSVADQFIERISAAMAATRYGDPLAQADVEMGPLINRQGLENVNRKVRTALAQGASLVTGGQIADLGAGFHFQPTVLVGGRADMDIMREEIFGPVLPIQIIDDLDEAIALANDCDYGLTSSIYTRDLGKAMHAVRELDFGETYVNRENFEAMQGFHAGVRKSGIGGADGKHGLYEYTHTHVVYLQS

SEQ ID NO: 272 - protein, 477 aa, Pseudomonas proteolytica
MPSVPVYQNFINGRFTASSAHIDVLNPATGALLSKVPAATAEDVDQALAAARAAQKDWSRKPAIERAGHLRRIAAKLRENVAHLARTVTLEQGKISSLAEIEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGQVGGALTAHKGVDMISFTGSVATGSRIMTAAAPNITKLNLELGGKAPAIVLADADLELAVKAIRDSRIINSGQVCNCAERVYVQRSVADQFIERISAAMAATRYGDPLAQADVEMGPLINRQGLESVNRKVRTALAQGASLVTGGQIADLGAGFHFQPTVLAGGRADMDIMREEIFGPVLPIQIIDDLDEAIALANDCDYGLTSSIYTRDLGKAMHAVRELDFGETYVNRENFEAMQGFHAGVRKSGIGGADGKHGLYEYTHTHVVYLQS

SEQ ID NO: 273 - protein, 477 aa, Pseudomonas proteolytica
MSSVPVYQNFINGCFTASSAHIDVLNPATGALLSKVPAATAEDVDQALAAARAAQKDWSRKPAIERAGHLRRIAAKLRENVAHLARTVTLEQGKISSLAEIEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGQVGGALTAHKGVDMISFTGSVATGSRIMTAAAPNITKLNLELGGKAPAIVLADANLELAVKAIRDSRIINSGQVCNCAERVYVQRSVADQFIERISAAMAATRYGDPLAQADVEMGPLINRQGLESVNRKVRTALAQGASLVTGGQIADLGAGFHFQPTVLAGGRADMDIMREEIFGPVLPIQIIDDLDEAIALANDCDYGLTSSIYTRDLGKAMHAVRELDFGETYVNRENFEAMQGFHAGVRKSGIGGADGKHGLYEYTHTHVVYLQS

SEQ ID NO: 274 - protein, 477 aa, Pseudomonas fluorescens
MSSVPVYQNFINGCFTASSAHIDVLNPATGALLSKVPAATAEDVDQALAAARAAQKDWSRKPAIERAGHLRRIAAKLRENVAHLARTVTLEQGKISSLAEIEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGQVGGALTAHKGVDMISFTGSVATGSRIMTAAAPNITKLNLELGGKAPAIVLADANLELAVKAIRDSRIINSGQVCNCAERVYVQRSVADQFIERISAAMAATRYGDPLAQADVEMGPLINRHGLESVNRKVRTALAQGASLVTGGQIADLGAGFHFQPTVLAGGRADMDIMREEIFGPVLPIQIIDDLDEAIALANDCDYGLTSSIYTRDLGKAMHAVRELDFGETYVNRENFEAMQGFHAGVRKSGIGGADGKHGLYEYTHTHVVYLQS

SEQ ID NO: 275 - protein, 477 aa, Pseudomonas fluorescens
MPSVPVYQNFINGRFTASSAHLDVFNPATGALLSKVPAATAEDVDQALAAARTAQPGWARKPAIERAGHLRRIAAKLRENVAHLARTITLEQGKISSLAEVEVNFTADYLDYMAEWARRIEGEIITSDRPDENIFLFRKPLGVVAGILPWNFPFFLIARKMAPALLTGNTLVIKPSEETPNNCFEFARLVAETDLPAGVFNVVCGDGQVGGALTAHKGVDMISFTGSVATGARIMTAAAPNITKLNLELGGKAPAIVLADADLDLAVKAIRDSRIINSGQVCNCVERVYVQRSIADPFIERISAAMAATRYGDPLAQADVEMGPLINRQGLDSVNRKVRTALAQGATLVTGGQIADLGAGFHFQPTVLAGGRADMAIMREEIFGPVLPIQIVDDLDEAIALANDCDYGLTSSIYTRDLGKAMQAVRALDFGETYVNRENFEAMQGFHAGVRKSGIGGADGKHGLYEYTHTHVVYLQS

SEQ ID NO: 276 - protein, 477 aa, Pseudomonas proteolytica
MPSVPVYQNFINGCFTASSAHIDVLNPATGALLSKVPAATAEDVDQALAAARAAQKDWSRKPAIERAGHLRRIAAKLRENVAHLARTVTLEQGKISSLAEIEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGQVGGALTAHKGVDMISFTGSVATGSRIMTAAAPNITKLNLELGGKAPAIVLADADLELAVKAIRDSRIINSGQVCNCAERVYVQRSVADQFIERISAAMATTRYGDPLAQADVEMGPLINRQGLESVNRKVRTALAQGASLVTGGQIADLGAGFHFQPTVLVGGRADMDIMREEIFGPVLPIQIIDDLDEAIALANDCDYGLTSSIYTRDLGKAMHAVRELDFGETYVNRENFEAMQGFHAGVRKSGIGGADGKHGLYEYTHTHVVYLQS

SEQ ID NO: 277 - protein, 477 aa, Pseudomonas sp.
MPSVPVYQNFINGCFTASSAHIDVLNPATGALLSKVPAATAEDVDQALAAARAAQKDWSRKPAIERAGHLRRIAAKLRENVAHLARTVTLEQGKISSLAEIEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGQVGGALTAHKGVDMISFTGSVATGSRIMTAAAPNITKLNLELGGKAPAIVLADANLELAVKAIRDSRIINSGQVCNCAERVYVQRSVADQFIERISAAMAATRYGDPLAQADVEMGPLINRQGLESVNRKVRTALAQGASLVTGGQIADLGAGFHFQPTVLAGGRADMDIMREEIFGPVLPIQIIDDLDEAIALANDCDYGLTSSIYTRDLGKAMHAVRELDFGETYVNRENFEAMQGFHAGVRKSGIGGADGKHGLYEYTHTHVVYLQS

SEQ ID NO: 278 - protein, 477 aa, Pseudomonas proteolytica
MPSVPVYQNFINGCFTASSAHIDVLNPATGALLSKVPAATAEDVDQALAAARAAQKDWSRKPAIERAGHLRRIAAKLRENVAHLARTVTLEQGKISSLAEIEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGQVGSALTAHKGVDMISFTGSVATGSRIMTAAAPNITKLNLELGGKAPAIVLADADLELAVKAIRDSRIINSGQVCNCAERVYVQRSVADQFIERISAAMAATRYGDPLAQADVEMGPLINRQGLENVNRKVRTALAQGASLVTGGQIADLGAGFHFQPTVLAGGRADMDIMREEIFGPVLPIQIIDDLDEAIALANDCDYGLTSSIYTRDLGKAMHAVRELDFGETYVNRENFEAMQGFHAGVRKSGIGGADGKHGLYEYTHTHVVYLQS

SEQ ID NO: 279 - protein, 477 aa, Pseudomonas sp.
MSSVPVYQNYIDGQFTASAEHLEVFNPATGALLSRVPAASPDEVDRALAAARTAQTEWARKPAIERAGYLRRIAAKLRENAVDLARTITLEQGKITALAEVEVNFTADYLDYMAEWARRIEGEIITSDRPGENIFLFRKPLGVVAGILPWNFPFFLIARKLAPALITGNTIVIKPSEETPNNCFEFTRLVAQTDLPAGVFNVVCGDGRVGAALSAHTKVDMISFTGSVDTGARIMAAAAPNLTKLNLELGGKAPAIVLADADLDLAVKAIRDSRIINTGQVCNCTERVYVERKVADQFIERIGAAMAATRYGDPLADSTVEMGPLTNRQGLDSVSAKVRRALADGAELITGGAVADRSAGFHYQPTVLTHCRADMEIMRKEIFGPVLPIQIVDSLDEAIALANDCEYGLTSSLFTRDLGKAMQAVREVDFGETYINRENFEAMQGFHAGVRKSGIGGADGKHGLYEYTHTHVVYLQA

SEQ ID NO: 280 - protein, 477 aa, Pseudomonas orientalis
MSSVPVYQNFINGRFAASEAHIDVLNPATGALLSKVPASTVADVDQALAAARSAQKDWARKPAIERAGHLRRIAAKLRENVAHLARTITLEQGKITSLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGTRIMAAAAPNITKLNLELGGKAPAIVLADADLELAVKAIRDSRIINTGQVCNCAERVYVQRKVADQFIERISAAMSATRYGDPIAQTDVEMGPLINRQGLESVDRKVRTALSQGATLISGGQIADLGAGFHFQPTVLAGCRADMEIMREEIFGPVLPIQIVDDLDEAIALANDCDYGLTSSIYTRDLGKAMHAIREIDFGETYVNRENFEAMQGFHAGVRKSGIGGADGKHGLYEYTHTHVVYLQS

SEQ ID NO: 281 - protein, 477 aa, Pseudomonas sp.
  source 1..477: note = strain KBW05
MSSVPVYQNFINGRFVPSEAHLDVLNPATGALLSKVPASTAADVDQALAAARHAQKDWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGQVGGALTSHTGVDMISFTGSVATGSRIMAAAAPNITKLNLELGGKAPAIVLADADLELAVKAIRDSRIINTGQVCNCAERVYVERKVADQFIERISAAMATTRYGDPLAQADVEMGPLINRQGLDNVNRKVRTALSQGANLITGGQIADLGAGFHFQPTVLAGCRADMEIMREEIFGPVLPIQIVDDLDEAIAMANDCDYGLTSSIYTRDLGKAMHAVRALDFGETYVNRENFEAMQGFHAGVRKSGIGGADGKHGLYEYTHTHVVYLQS

SEQ ID NO: 282 - protein, 477 aa, Pseudomonas fluorescens
MSSVPVFQNFINGQFTHSEAHLEVFNPATGALLSRVPASSAADVDQALAAARAAQKDWSAKPAIERAGHLRRIAAKLRENVAHLARTITLEQGKISALAEVEVNFTADYLDYMAEWARRIEGEIITSDRPGENIFLFRKPLGVVAGILPWNFPFFLIARKMAPALLTGNTIVIKPSEETPNNCFEFARLVAETDLPPGVFNVVCGDGQVGAALSGHKGVDMISFTGSVETGSRIMTAAAANITKLNLELGGKAPAIVLADADLELAVKAIRDSRIINTGQVCNCAERVYVQREVADQFIERISAAMSATRYGDPLAEPDVEMGPLINRQGLDSVERKVRTALSQGASLISGGSVADRTSGFHFQPTVLAGCNASMDIMREEIFGPVLPIQIIDDLDEAIALANDCDYGLTSSVYTRDLSRAMHAIRGLDFGETYVNRENFEAMQGFHAGVRKSGVGGADGKHGLYEYTHTHAVYLQS

SEQ ID NO: 283 - protein, 477 aa, Pseudomonas sp.
  source 1..477: note = strain Marseille-Q1929
MSSVPVYQNFINGRFVPSEAHLDVLNPATGALLSKVPASTAADVDQALAAARQAQKDWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVADQFIERISAAMAATRYGDPLAQADVEMGPLINRQGLDNVNRKVRTALSQGASLITGGQIADLGAGFHFQPTVLAGCRADMEIMREEIFGPVLPIQIVDDLDEAIAMANDCDYGLTSSIYTRDLGKAMHAVRELDFGETYVNRENFEAMQGFHAGVRKSGIGGADGKHGLYEYTHTHVVYLQS

SEQ ID NO: 284 - protein, 477 aa, Pseudomonas brenneri
MSSVPVYQNFINGRFTPSSAHIDVLNPATGALLSKVPAATAEDVDQALTAARAAQQGWARKPAIERAGHLRRIAAKLRENVAHLARTITLEQGKISSLAEVEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGQVGAALTAHKGVDMISFTGSVATGSRIMTAAAPNITKLNLELGGKAPAIVLADADLALAVKAIRDSRIINSGQVCNCAERVYVQRSVADQFIERISAVMAATRYGDPLAQSDVEMGPLINRQGLDNVNRKVRTALAQGASLVTGGQIADLGAGFHFQPTVLAGGRADMDIMREEIFGPVLPIQIIDDLDEAIALANDCDYGLTSSIYTRDLSKAMHAVRELDFGETYVNRENFEAMQGFHAGVRKSGIGGADGKHGLYEYTHTHVVYLQG

SEQ ID NO: 285 - protein, 477 aa, Pseudomonas sp.
  source 1..477: note = strain TNT2022 ID1044
MSSVPVYENFINGQFVASAAHLDVINPATGAVLSKVPASTAEDVERALAAARAAQKDWSRKPANDRAGYLRRIAAKLRENVSHLARTITLEQGKISGLAEVEVNFTADYLDYMAEWARRIEGEIITSDRQSENIFLFRKPLGVVAGILPWNFPFFLIARKMAPALLTGNTIVIKPSEETPNNCFEFARLVAETDLPPGVFNVVCGDGQVGAALSAHKGVDMISFTGSVATGSRIMAAAAPNITKLNLELGGKAPAIVLADADLDLAVKAIRDSRIINSGQVCNCAERVYVERKVADQFIERIAAAMSATRYGDPIAQPDVEMGPLINRQGLDSVNRKVRTALSQGANLISGGQIADRPDGFHFQPTVLAGCRADMDIMREEIFGPVLPIQIIDDLDEAVALANDCEYGLTSSIYTRDLNKTLHAMRELDFGETYVNRENFEAMQGFHAGVRKSGIGGADGKHGLYEYTHTHAVYLQG

SEQ ID NO: 286 - protein, 477 aa, Pseudomonas fluorescens
MSSVPVYENFINGQFVASAAHLDVINPATGAVLSKVPASTAEDVERALAAARAAQKDWSRKPANDRAGYLRRIAAKLRDNVAHLARTITLEQGKISSLAEVEVNFTADYLDYMAEWARRIEGEIITSDRPNENIFLFRKPLGVVAGILPWNFPFFLIARKMAPALLTGNTIVIKPSEETPNNCFEFARLVAETDLPPGVFNVVCGDGRVGAALSAHKGVDMISFTGSVDTGSRIMAAAAPNITKLNLELGGKAPAIVLADADLELAVKAIRDSRIINSGQVCNCAERVYVERKVADQFIERIAAAMNATRYGDPIARPDVEMGPLINRQGLDSVNSKVRTALSQGASLISGGQIADLPNGFHFQPTVLAGCRADMDIMRQEIFGPVLPIQIIDDLDEAIALANDCEYGLTSSIYTRDLGKTLHAMRELDFGETYVNRENFEAMQGFHAGVRKSGIGGADGKHGLYEYTHTHAVYLQG

SEQ ID NO: 287 - protein, 477 aa, Pseudomonas sp.
  source 1..477: note = strain 1912-s
MSSVPVYENFIDGQFVASAAHLDVINPATGAVLSKVPASTAEDVERALAAARAAQKGWARKPANDRAGHLRRIAAKLRENVAHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPPGVFNVVCGDGQVGAALSSHKGVDMISFTGSVDTGSRIMAAAAPNITKLNLELGGKAPAIVLADADLDLAVRAIRDSRIINSGQVCNCAERVYVERKVADQFIERIAAAMSATRYGDPIAQPDVEMGPLINRQGLESVNRKVRTALSQGASLISGGQMADLPNGFHFQPTVLAGCRADMEIMREEIFGPVLPIQIIDDLDEAIALANDCDYGLTSSIYTRDLNKTLQAMRELDFGETYVNRENFEAMQGFHAGVRKSGIGGADGKHGLYEYTHTHAVYLQG

SEQ ID NO: 288 - protein, 477 aa, Pseudomonas sp.
  source 1..477: note = strain ANT_H12B
MSSVPVYQNFINGRFTPSDAHLEVLNPATGALLSKVPASTAEDVDQALAAARHAQKDWARKPAIERAGHLRRIAAKLRENVAHLARTITLEQGKVTGLAEVEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGHVGGALTAHKGVDMISFTGSVATGSRIMAAAAPNITKLNLELGGKAPAIVLADADLDLAVNAIRDSRIINSGQVCNCAERVYVERKVADQFIERISAAMAATRYGDPIAQADVEMGPLINRQGLESVNRKVRTALGQGASLVTGGQIADLGQGFHFQPTVLAGCRADMEIMREEIFGPVLPIQIVDDLDEAIALANDCDYGLTSSIYTRDLGKAMQAVRELSFGETYVNRENFEAMQGFHAGVRKSGIGGADGKHGLYEYTHTHAVYLQG

SEQ ID NO: 289 - protein, 477 aa, Pseudomonas azerbaijanoccidentalis
MSSVPVYENFINGQFVASAAHLEVINPATGAVLSKVPASTADDVDRALAAARAAQKDWSRKPAIERAGHLRRIAAKLRENVTHLARTITLEQGKVSGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPAGVFNVVCGDGRVGAALSAHKGVDMISFTGSVDTGSRIMAAAAPNITKLNLELGGKAPAIVLADADLELAVKAIRDSRIINSGQVCNCAERVYVERKVADPFIERIAAAMSATRYGDPIAQPDIEMGPLINRQGLDSVNRKVRTALSQGASLISGGQIADLPNGFHFQPTVLAGCHAEMEIMREEIFGPVLPIQIVDDLDEAIALANDCDYGLTSSIYTRDLSKTMHAMRELDFGETYVNRENFEAMQGFHAGVRKSGIGGADGKHGLYEYTHTHAVYLQG

SEQ ID NO: 290 - protein, 477 aa, Pseudomonas sp.
MPSVPVYENFINGQFVASAAHLDVINPATGAVLSKVPASTADDVERALSAARAAQKDWARKPANDRAGYLRRIAAKLRENAPHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPPGVFNVVCGDGQVGAALSAHKGVDMISFTGSVATGSRIMAAAAPNITKLNLELGGKAPAIVLADADLDLAVKAIRDSRIINSGQVCNCAERVYVERKVADQFIERIAAAMSATRYGDPIAQPDVEMGPLINRQGLDSVNRKVRTALSQGASLISGGQIADLPNGFHFQPTVLAGCRADMEIMREEIFGPVLPIQIIDDLDEAIALANDCDYGLTSSIYTRDLNKTLHAMRELDFGETYVNRENFEAMQGFHAGVRKSGIGGADGKHGLYEYTHTHAVYLQG

SEQ ID NO: 291 - protein, 477 aa, Pseudomonas sp.
  source 1..477: note = strain NFACC45
MPSVPVYENFINGQFVASAAHLDVINPATGAVLSKVPASTADDVERALSAARAAQKDWARKPANDRAGYLRRIAAKLRENAPHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPPGVFNVVCGDGQVGAALSAHQGVDMISFTGSVATGSRIMAAAAPNITKLNLELGGKAPAIVLADADLDLAVKAIRDSRIINSGQVCNCAERVYVERKVADQFIERIAAAMSATRYGDPIAQPDVEMGPLINRQGLDSVNRKVRTALSQGASLISGGQIADLPNGFHFQPTVLAGCRADMEIMREEIFGPVLPIQIIDDLDEAIALANDCDYGLTSSIYTRDLNKTLHAMRELDFGETYVNRENFEAMQGFHAGVRKSGIGGADGKHGLYEYTHTHAVYLQG

SEQ ID NO: 292 - protein, 477 aa, Pseudomonas sp.
MSSVPVFQNFINGQFTHSEAHLEVFNPATGALLSRVPASSAANVDQALAAARATQKDWAAKPAIERAGHLRRIAAKLRENVAHLARTITLEQGKISALAEVEVNFTADYLDYMAEWARRIEGEIITSDRRGENIFLFRKPLGVVAGILPWNFPFFLIARKMAPALLTGNTIVIKPSEETPNNCFEFARLVAETDLPPGVFNVVCGDGQVGAALSAHQGVDMISFTGSVDTGSRIMMAAAPNITKLNLELGGKAPAIVLADADLALAVKAIRDSRIINTGQVCNCAERVYVERKVADQFIERISAAMSATRYGDPIAEADVEMGPLINRQGLDSVERKVRTALSQGASLVSGGRVAERPQGFHFQPTVLAGCNASMDIMREEIFGPVLPIQIIDDLDEAIALANDCDYGLTSSVYTRDLSRAMHAIRGLDFGETYVNRENFEAMQGFHAGVRKSGVGGADGKHGLYEYTHTHAVYLQS

SEQ ID NO: 293 - protein, 477 aa, Pseudomonas sp.
  source 1..477: note = strain YuFO8
MSSVPVYQNFINGRFVSSEAHIDVLNPATGALLSKVPASSTSDVDQALAAARSAQKNWARKPAIERAGHLRRIAAKLRENVAHLARTITLEQGKITGLAEVEVNFTADYLDYMAEWARRIEGEIITSDRPNENIFLFRKPLGVVAGILPWNFPFFLIARKLAPALLTGNTIVIKPSEETPNNCFEFAKLVAETDLPAGVFNVVCGDGQVGGALTAHKGVDMISFTGSVATGTRIMAAAAPNITKLNLELGGKAPAIVLADADLELAVKAIRDSRIINTGQVCNCAERVYVERKVADQFIERISAAMAATRYGDPIAQADVEMGPLINRQGLDSVDRKVRTALSQGATLISGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQG

SEQ ID NO: 294 - protein, 477 aa, Pseudomonas putida
MSSVPVYENFINGQFVASAAHIDVINPATGALLSKVPASNAEDVDRALAAARSAQKDWARKPANERAGYLRRIAAKLRENVAHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPPGVFNVVCGDGQVGAALSGHKGVDMISFTGSVATGSRIMAAAAPNITKLNLELGGKAPAIVLADADLDLAVKAIRDSRIINSGQVCNCAERVYVERKVADQFIERIAAAMSATRYGDPIAQPDVEMGPLINRQGLDSVNRKVRTALSQGASLISGGQVADLPNGFHFQPTVLAGCRADMEIMREEIFGPVLPIQIVDDLDEAIALANDCDYGLTSSIYTRDLSKTLHAMRELDFGETYVNRENFEAMQGFHAGVRKSGIGGADGKHGLYEYTHTHAVYLQG

SEQ ID NO: 295 - protein, 477 aa, Pseudomonas sp.
  source 1..477: note = strain YuFO20
MSSVPVYENFINGQFVASAAHIDVINPATGALLSKVPASNAEDVDRALAAARSAQKDWARKPANERAGYLRRIAAKLRENVAHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPPGVFNVVCGDGQVGAALSGHKGVDMISFTGSVATGSRIMAAAAPNITKLNLELGGKAPAIVLADADLDLAVKAIRDSRIINSGQVCNCAERVYVERKVADQFIERIAAAMSATRYGDPIAQPDVEMGPLINRQGLDSVNRKVRTALSQGASLISGGQVADLPNGFHFQPTVLAGCRADMEIMREEIFGPVLPIQIVDDLDEAIALANDCDYGLTSSIYTRDLNKTLYAMRELDFGETYVNRENFEAMQGFHAGVRKSGIGGADGKHGLYEYTHTHAVYLQG

SEQ ID NO: 296 - protein, 477 aa, Pseudomonas fluorescens
MSSVPVYENFINGQFVASAAHIDVINPATGALLSKVPASNAEDVDRALAAARSAQKDWARKPANERAGYLRRIAAKLRENVAHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPPGVFNVVCGDGQVGAALSGHKGVDMISFTGSVTTGSRIMAAAAPNITKLNLELGGKAPAIVLADADLDLAVKAIRDSRIINSGQVCNCAERVYVERKVADQFIERIAAAMSATRYGDPIAQPDVEMGPLINRQGLDSVNRKVRTALSQGASLISGGQVADLPNGFHFQPTVLAGCRADMEIMREEIFGPVLPIQIVDDLDEAIALANDCDYGLTSSIYTRDLSMTMHAMRELDFGETYVNRENFEAMQGFHAGVRKSGIGGADGKHGLYEYTHTHAVYLQG

SEQ ID NO: 297 - protein, 477 aa, Pseudomonas sp.
  source 1..477: note = strain GM78
MSSVPVYQNFINGRFTASDSHIDVLNPATGALLSKVPASIAADVDRALAAARSAQKDWARKPAIERAGHLRRIAAKLRENVAHLARTITLEQGKITGLAEVEVNFTADYLDYMAEWARRLEGEIITSDRPNENIFLFRKPLGVVAGILPWNFPFFLIARKMAPALLTGNTIVIKPSEETPNNCFEFAKLVAETDLPAGVFNVVCGDGNVGGALTAHKGVDMISFTGSVATGTRIMAAAAPNITKLNLELGGKAPAIVLADADLELAVKAIRDSRIINTGQVCNCAERVYVERKVADQFIERISAAMSATRYGDPIAQADVEMGPLINRQGLDSVDRKVRTALSQGANLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQG

SEQ ID NO: 298 - protein, 477 aa, Pseudomonas sp.
  source 1..477: note = strain TH32
MSSVPVYENYIDGQFVASAAHLDVINPATGALLSKVPASNAEDVDRALAAARTAQKDWARKPANDRAGYLRRIAAKLRENVAHLARTITLEQGKINGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPPGVLNVVCGDGRVGAALSAHKGVDMISFTGSVATGSRIMAAAAPNITKLNLELGGKAPAIVLADADLDLAVKAIRDSRIINSGQVCNCAERVYVERKVADQFIERIAAAMNATRYGDPIAQPDVEMGPLINRQGLDSVNRKVRTALNQGASLISGGQIADLPNGFHFQPTVLAGCHADMEIMREEIFGPVLPIQIIDDLDEAIALANDCDYGLTSSIYTRDLSKTMHAMRELDFGETYVNRENFEAMQGFHAGVRKSGIGGADGKHGLYEYTHTHAVYLQG

SEQ ID NO: 299 - protein, 477 aa, Pseudomonas sp.
MSSVPVYQNFINGRFTPSDAHLEVLNPATGALLSKVPASTAEDVDQALAAARHAQKDWARKPAIERAGHLRRIAAKLRENVAHLARTITLEQGKVTGLAEVEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGHVGGALTAHKGVDMISFTGSVATGSRIMAAAAPNITKLNLELGGKAPAIVLANADLDLAVNAIRDSRIINSGQVCNCAERVYVERKVADQFIERISAAMAATRYGDPIAQADVEMGPLINRQGLESVNRKVRTALGQGASLVTGGQIADLGQGFHFQPTVLAGCRADMEIMREEIFGPVLPIQIVDDLDEAIALANDCDYGLTSSIYTRDLGKAMQAVRELSFGETYVNRENFEAMQGFHAGVRKSGIGGADGKHGLYEYTHTHAVYLQG

SEQ ID NO: 300 - protein, 477 aa, Pseudomonas sp.
MSSVPVYQNFINGRFTPSDAHLDVLNPATGALLSKVPASTTEDVDQALAAARHAQKDWARKPAIERAGHLRRIAAKLRENVAHLARTITLEQGKVTGLAEVEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGRVGGALTAHKGVDMISFTGSVATGSRIMAAAAPNITKLNLELGGKAPAIVLADADLDLAVKAIRDSRIINSGQVCNCAERVYVERKVADQFIERISAAMAATRYGDPIAQADVEMGPLINRQGLDSVNRKVRTALGQGASLVTGGQIADLGQGFHFQPTVLAGCRADMEIMREEIFGPVLPIQIVDDLDEAIALANDCDYGLTSSIYTRDLGKAMQAVRELSFGETYINRENFEAMQGFHAGVRKSGIGGADGKHGLYEYTHTHAVYLQG

SEQ ID NO: 301 - protein, 477 aa, Pseudomonas fluorescens
  source 1..477: note = strain ABAC62
MSSVPVYQNFINGQFTASAAHLDVLNPATGALLSKVPASTAEDVDRALAAARNAQKDWARKPAIERAGHLRRIAAKLRENVAHLARTITLEQGKVTGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGSVGGALTAHKGVDMISFTGSVATGSRIMVAAAPNITKLNLELGGKAPAIVLADANLDLAVKAIRDSRIINSGQVCNCAERVYVERKVADQFIERISAAMSATRYGDPIAQSDVEMGPLINRQGLDSVNRKVRTALSQGATLVSGGQIADLGQGFHFQPTVLAGCRADMEIMREEIFGPVLPIQIVDDLDEAIAMANDCDYGLTSSIYTRDLGQAMHAVRELDFGETYVNRENFEAMQGFHAGVRKSGIGGADGKHGLYEYTHTHVVYLQS

SEQ ID NO: 302 - protein, 477 aa, Pseudomonas sp.
  source 1..477: note = strain 2822-17
MSSVPVYQNFINGRFVSSEAHLDVLNPATGALLSKVPASTAAEVDQALAAARGAQKEWARKPAIERAGHLRRIAARLRENVAHLARTITLEQGKVSGLAEVEVNFTADYLDYMAEWARRIEGEIITSDRPNENIFLFRKPLGVVAGILPWNFPFFLIARKLAPALLTGNTIVIKPSEETPNNCFEFAKLVAETDLPAGVFNVVCGDGQVGGALTSHAGVDMISFTGSVATGSRIMAAAAPNITKLNLELGGKAPAIVLADADLELAVKAIRDSRIINTGQVCNCAERVYVERKVADQFIERISAAMSATRYGDPLAMADAQMGPLINRQGLDNVNRKVRTALSQGATLVSGGQIADLGAGFHFQPTVLAGCRADMQIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQS

SEQ ID NO: 303 - protein, 477 aa, Pseudomonas marginalis
  source 1..477: note = pv. marginalis
MSSVPVYQNFINGRFVSSEAHLDVLNPATGALLSKVPASTAADVDQALAAARGAQKEWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLDLAVKAIRDSRIINTGQVCNCAERVYVERKVADQFIERISAAMSATRYGDPLAMADAEMGPLINRQGLDNVNRKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQS

SEQ ID NO: 304 - protein, 477 aa, Pseudomonas cyclaminis
MSSVPVYQNFINGRFVPSEAHLDVLNPATGALLSKVPASTAADVDQALAAARGAQKAWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVADLFIERISAAMSATRYGDPMAMAGAEMGPLINRQGLDNVNRKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQS

SEQ ID NO: 305 - protein, 477 aa, Pseudomonas mandelii
MSSVPVYQNFINGCFVPSEAHLDVLNPATGALLSKVPASTAADVDQALAAARGAQKAWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVADLFIERISAAMSATRYGDPMAMAGAEMGPLINRQGLDNVNRKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQS

SEQ ID NO: 306 - protein, 477 aa, Pseudomonas sp.
  source 1..477: note = strain GL-RE-29
MSSVPVYENFINGQFVASTAHLDVINPATGVVLSKVPASTAEDVDRALTAARAAQKDWSRKPAIERAGHLRRIAAKLRENVAHLARTITLEQGKVSGLAEVEVNFTADYLDYMAEWARRIEGEIIASDRQNENIFLFRKPLGVVAGILPWNFPFFLIARKMAPALLTGNTIVIKPSEETPNNCFEFARLVAETDLPAGVFNVVCGDGRVGAALSAHKGVDMISFTGSVDTGSRIMAAAAPNITKLNLELGGKAPAIVLADADLDLAVKAIRDSRIINSGQVCNCAERVYVERKVADSFIERIAAAMSATRYGDPIARPEVEMGPLINRQGLDSVNRKVRTALSQGASLISGGQIADLPNGFHFQPTVLAGCSADMEIMREEIFGPVLPIQIIDDLDEAIALANDCDYGLTSSIYTRDLSKTMHAMRELDFGETYVNRENFEAMQGFHAGVRKSGIGGADGKHGLYEYTHTHAVYLQG

SEQ ID NO: 307 - protein, 477 aa, Pseudomonas sp.
  source 1..477: note = strain CFBP13528
MSSVPVYENFINGQFVASAAHLDVINPATGAVLSKVPASTAEDVERALTAARTAQKDWARKPANDRAGYLRRIAAKLRENVARLARTITLEQGKISGLAEVEANFTADYLDYMAEWARRIEGEIITSDRQNENIFLFRKPLGVVAGILPWNFPFFLIARKMAPALLTGNTIVIKPSEETPNNCFEFARLVAETDLPPGVFNVVCGDGRVGAALSAHKGVDMISFTGSVDTGSRIMAAAAPNITKLNLELGGKAPAIVLADADLDLAVKAIRDSRIINSGQVCNCAERVYVERKVADQFIERIAAAMSATRYGDPIAQPDVEMGPLINRQGLDSVNRKVRTALSQGASLISGGHIADLPNGFHFQPTVLAGCRADMEIMREEIFGPVLPIQIIDDLDEAIALANDCEYGLTSSIYTRDLSKTLHAMRELDFGETYVNRENFEAMQGFHAGVRKSGIGGADGKHGLYEYTHTHAVYLQG

SEQ ID NO: 308 - protein, 477 aa, Pseudomonas yamanorum
MSSVPVYQNFINGRFTASDSHIDVLNPATGALLSKVPASTAADVDRALAAARSAQKDWARKPAIERAGHLRRIAAKLRENVAQLARTITLEQGKITGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGNVGGALTAHKGVDMISFTGSVATGTRIMAAAAPNITKLNLELGGKAPAIVLADADLELAVKAIRDSRIINTGQVCNCAERVYVERKVADQFIERISAAMSATRYGDPIAQADVEMGPLINRQGLDSVDRKVRTALSQGANLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQG

SEQ ID NO: 309 - protein, 477 aa, Pseudomonas sp.
MSSIPVYQNFINGRFTPSDAHLDVVNPATGTLLSKVPASTTEDVDQALAAARHAQKDWARKPAIERAGHLRRIAAKLRENVAHLARTITLEQGKVTGLAEVEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGRVGGALTAHKGVDMISFTGSVATGSRIMAAAAPNITKLNLELGGKAPAIVLADADLDLAVKAIRDSRIINSGQVCNCAERVYVERKVADQFIERISAAMAATRYGDPIAQPDVEMGPLINRQGLDSVNRKVRTALDQGASLVTGGQIADLGQGFHFQPTVLAGCRADMEIMREEIFGPVLPIQIVDDLDEAIALANDCDYGLTSSIYTRDLGKAMQAVRELSFGETYINRENFEAMQGFHAGVRKSGIGGADGKHGLYEYTHTHAVYLQG

SEQ ID NO: 310 - protein, 477 aa, Pseudomonas sp.
  source 1..477: note = strain Z18(2022)
MSSIPVYQNFINGRFTPSDAHLDVLNPATGALLSKVPASTTEDVDQALAAARHAQKDWARKPAIERAGHLRRIAAKLRENVAHLARTITLEQGKVTGLAEVEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGRVGGALTAHKGVDMISFTGSVATGSRIMAAAAPNITKLNLELGGKAPAIVLADADLDLAVKAIRDSRIINSGQVCNCAERVYVERKVADQFIERISAAMAATRYGDPVAQADVEMGPLINRQGLDSVNRKVRTALGQGASLVTGGQIADLGQGFHFQPTVLAGCRADMEIMREEIFGPVLPIQIVDDLDEAIALANDCDYGLTSSIYTRDLGKAMQAVRELSFGETYINRENFEAMQGFHAGVRKSGIGGADGKHGLYEYTHTHAVYLQG

SEQ ID NO: 311 - protein, 476 aa, Pseudomonas umsongensis
MSSVPVYQNFINGQFVASAAHLDVINPATGALLSKVPASNAEDVDRALAAARTAQKDWARKPANDRAGYLRRIAAKLRENVAHLARTITLEQGKISALAEVEVNFTADYLDYMAEWARRIEGEIITSDRQDENIFLFRKPLGVVAGILPWNFPFFLIARKMAPALLTGNTIVIKPSEETPNNCFEFARLVAQTDLPPGVFNVVCGDGQVGAALSAHKRVDMISFTGSVATGSRIMAAAAPNITKLNLELGGKAPAIVLADADLELAVKAIRDSRIINSGQVCNCAERVYVERKVADQFIERIATAMNATRYGDPVAQPDVEMGPLINRQGLDSVNRKVRTALSQGASLISGGQIADLPGFHFQPTVLAGCHADMEIMREEIFGPVLPIQIVDDLDEAIALANDCDYGLTSSIYTRDLSKTLHAMRELDFGETYVNRENFEAMQGFHAGVRKSGIGGADGKHGLYEYTHTHAVYLQG

SEQ ID NO: 312 - protein, 477 aa, Pseudomonas kairouanensis
MSSVPVYQNFINGRFVPSEAHIDVLNPATGALLSKVPAATAADVDQALAAARHAQKDWGRKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTSHKGVDMISFTGSVATGSRIMTAAAPNITKLNLELGGKAPAIVLADADLDLAVKAIRDSRIINTGQVCNCAERVYVERKVADQFIERISAAMSATRYGDPIAQADVEMGPLINRQGLDNVNRKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGVGGADGKHGLYEYTHTHVVYLQS

SEQ ID NO: 313 - protein, 477 aa, Pseudomonas cedrina
MSSVPVYQNFINGRFTASDNHIDVLNPATGALLSKVPASAAADVDQALAAARSAQKDWARKPAIERAGHLRRIAAKLRENVAHLARTITLEQGKITGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGTRIMAAAAPNITKLNLELGGKAPAIVLADADLELAVKAIRDSRIINTGQVCNCAERVYVERKVADQFIERISAAMSATRYGDPIAQADVEMGPLINRQGLDSVDRKVRTALSQGAALISGGQIADLGAGFHFQPTVLAGCRADMEIMREEIFGPVLPIQIVDDLDEAIALANDCDYGLTSSIYTRDLGKAMHAIREIDFGETYVNRENFEAMQGFHAGVRKSGIGGADGKHGLYEYTHTHVVYLQS

SEQ ID NO: 314 - protein, 477 aa, Pseudomonas sp.
MSSVPVYQNFINGRFVPSEAHLDVLNPASGALLSKVPASTAADVDQALAAARGAQKAWARKPAIERAGHLRRIAAKLRENVAHLARTITLEQGKVTGLAEVEVNFTADYLDYMAEWARRIEGEIITSDRPNENIFLFRKPLGVVAGILPWNFPFFLIARKLAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVADPFIERISAAMSATRYGDPMAMADAEMGPLINRQGLENVNRKVRTALSQGATLISGGQIADLGAGFHFQPTVLAGCRADMEIMREEIFGPVLPIQIVDDLDEAIAMANDCDYGLTSSIYTRDLGKAMHAVREIDFGETYVNRENFEAMQGFHAGVRKSGIGGADGKHGLYEYTHTHVVYLQS

SEQ ID NO: 315 - protein, 477 aa, Pseudomonas lurida
MSSVPVYQNFINGRFTPSEAHIDVLNPATGALLSKVPASSAAEVDQALAAARQAHKDWARKPAIERAGHLRRIAAKLRENVAHLARTITLEQGKVSGLAEVEVNFTADYLDYMAEWARRIEGEIITSDRANENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVADSFIERISAAMSATRYGDPIAQADVEMGPLINRQGLESVNRKVRTALSQGASLISGGQIADLGAGFHFQPTVLAGCRADMEIMREEIFGPVLPIQIVDDLDEAIALANDCEYGLTSSIYTRDLGKAMHAVREIDFGETYVNRENFEAMQGFHAGVRKSGIGGADGKHGLYEYTHTHVVYMQS

SEQ ID NO: 316 - protein, 477 aa, Pseudomonas sp.
  source 1..477: note = 18.1.10
MSSVPVYQNFINGRFTASDAHIDVLNPATGALLSKVPASTAADVDQALAAARNAQKDWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKLAPALLTGNTIVIKPSEETPNNCFEFARLVAETDLPAGVFNVVCGDGQVGGALTAHKGVDMVSFTGSVATGSRIMAAAAPNITKLNLELGGKAPAIVLADADLDLAVKAIRESRIINTGQVCNCAERVYVERKVADQFIERISAAMAATRYGDPLAQANVEMGPLINRQGLDNVNRKVRTALSQGATLVTGGQIADLGAGFHFQPTVLAGCRADMEIMREEIFGPVLPIQIVDDLDEAIAMANDCDYGLTSSIYTRDLGKAMHAVREIDFGETYVNRENFEAMQGFHAGVRKSGIGGADGKHGLYEYTHTHVVYLQS

SEQ ID NO: 317 - protein, 477 aa, Pseudomonas sp.
MSSVPVYQNFINGRFVSSEAHLDVLNPATGALLSKVPASTGADVDQALAAARGAQKEWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLDLAVKAIRDSRIINTGQVCNCAERVYVERKVADQFIERISAAMSATRYGDPLAMADAEMGPLINRQGLDNVNRKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQS

SEQ ID NO: 318 - protein, 477 aa, Pseudomonas sp.
  source 1..477: note = strain S35
MSSVPVYQNFINGRFVPSEAHLDVLNPATGALLSKVPASTAADVDQALAAARGAQKEWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKLAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAIKAIRDSRIINTGQVCNCAERVYVERKVADQFIERISAAMSATRYGDPLAMAGAEMGPLINRQGLDNVNRKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQS

SEQ ID NO: 319 - protein, 477 aa, Pseudomonas fluorescens
MSYVPVYQNFINGRFVPSEAHLDVLNPATGALLSKVPASTAADVDQALAAARGAQKAWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVADLFIERISAAMSATRYGDPMAMAGAEMGPLINRQGLDNVNRKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQS

SEQ ID NO: 320 - protein, 477 aa, Pseudomonas sp.
MSSVPVYENFINGQFVASTAHLDVINPATGVVLSKVPASTAEDVDRALTAARAAQKDWSRKPAIERAGHLRRIAAKLRENVAHLARTITLEQGKVSGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPAGVFNVVCGDGRVGAALSAHKGVDMISFTGSVDTGSRIMAAAAPNITKLNLELGGKAPAIVLADADLDLAVKAIRDSRIINSGQVCNCAERVYVERKVADSFIERIAAAMSATRYGDPIARPEVEMGPLINRQGLDSVNRKVRTALSQGASLISGGQIADLPNGFHFQPTVLAGCSADMEIMREEIFGPVLPIQIIDDLDEAIALANDCDYGLTSSIYTRDLSKTMHAMRELDFGETYVNRENFEAMQGFHAGVRKSGIGGADGKHGLYEYTHTHAVYLQG

SEQ ID NO: 321 - protein, 477 aa, Pseudomonas sp.
  source 1..477: note = strain PD9R
MSSVPVYENFINGQFVASTAHLDVINPATGAVLSKVPASNAQDVERALAAARSAQKDWARKPANDRAGYLRRIAAKLRENVAHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPPGVFNVVCGDGQVGAALSVHKGVDMISFTGSVDTGSRIMAAAAPNITKLNLELGGKAPAIVLADADLDLAVKAIRNSRIINSGQVCNCAERVYVERKVADQFIERIAAAMTATRYGDPIAQPDVEMGPLINRQGLDSVNRKVRTALSQGASLISGGQIADLPNGFHFQPTVLAGCRADMEIMREEIFGPVLPIQIVDDLDEAIALANDCDYGLTSSIYTRDLSKTLHAMRELDFGETYVNRENFEAMQGFHAGVRKSGIGGADGKHGLYEYTHTHAVYLQG

SEQ ID NO: 322 - protein, 477 aa, Pseudomonas putida
MSSVPVFDNYINGQFVASAAHLDVINPATGAALSKVPASTAEDVDRALVAARVAQKDWSRKPANERAGYLRRIAAKLRENVAHLARTITLEQGKVSSLAEVEVNFTADYLDYMAEWARRIEGEIITSDRQNENIFLFRKPLGVVAGILPWNFPFFLIARKMAPALLTGNTIVIKPSEETPNNCFEFARLVAETDLPPGVFNVVCGDGQVGAALSGHKGVDMVSFTGSVDTGSRIMAAAAPNITKLNLELGGKAPAIVLADADLELAVKAIRDSRIINTGQVCNCAERVYVERKVADQFIERIAAAMSATRYGDPIAQPDVEMGPLINRQGLDSVNRKVRTALNQGASLISGGQIADVPNGFHFQPTVLADCRADMEIMRQEIFGPVLPIQIIDDLDEAIALANDCEYGLTSSIYTRDLSKTMHAMRELDFGETYVNRENFEAMQGFHAGVRKSGIGGADGKHGLYEYTHTHAVYLQG

SEQ ID NO: 323 - protein, 477 aa, Pseudomonas fluorescens
MSSVPVYENFINGQFVASAAHLDVINPATGAVLSKVPASTAEDVERALTAARTAQKDWARKPANDRAGYLRRIAAKLRENVAHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPPGVFNVVCGDGRVGAALSAHKGVDMISFTGSVDTGSRIMAAAAPNITKLNLELGGKAPAIVLADADLDLAVKAIRDSRIINSGQVCNCAERVYVERKVADQFIERIAAAMSATRYGDPIAQPDVEMGPLINRQGLDSVNRKVRTALSQGANLISGGQIADLPNGFHFQPTVLAGCSADMEIMREEIFGPVLPIQIIDDLDEAIALANDCEYGLTSSIYTRDLSKTLHAMRELDFGETYVNRENFEAMQGFHAGVRKSGIGGADGKHGLYEYTHTHAVYLQG

SEQ ID NO: 324 - protein, 477 aa, Pseudomonas sp.
MSSVPVYQNFINGQFVASAAHLDVINPATGALLSKVPASNAEDVDRALAAARTAQKDWARKPANDRAGYLRRIAAKLRENVAHLARTITLEQGKISALAEVEVNFTADYLDYMAEWARRIEGEIITSDRQDENIFLFRKPLGVVAGILPWNFPFFLIARKMAPALLTGNTIVIKPSEETPNNCFEFARLVAQTDLPPGVFNVVCGDGQVGAALSAHKRVDMISFTGSVATGSRIMAAAAPNITKLNLELGGKAPAIVLADADLELAVKAIRDSRIINSGQVCNCAERVYVERKVADQFIERIATAMNATRYGDPVAQPDVEMGPLINRQGLDSVNRKVRTALSQGASLISGGQIADLPHGFHFQPTVLAGCHADMEIMREEIFGPVLPIQIVDDLDEAIALANDCDYGLTSSIYTRDLSKTLHAMRELDFGETYVNRENFEAMQGFHAGVRKSGIGGADGKHGLYEYTHTHAVYLQG

SEQ ID NO: 325 - protein, 477 aa, Pseudomonas yamanorum
MSSIPVYQNFINGRFTPSDAHLDVLNPATGALLSKVPASTTEDVDQALAAARHAQKDWARKPAIERAGHLRRIAAKLRENVAHLARTITLEQGKVTGLAEVEVNFTADYLDYMAEWARRIEGEIITSDRPNENIFLFRKPLGVVAGILPWNFPFFLIARKMAPALLTGNTIVIKPSEETPNNCFEFARLVAETDLPAGVFNVVCGDGRVGGALTAHKGVDMISFTGSVATGSRIMAAAAPNITKLNLELGGKAPAIVLADADLDLAVKAIRDSRIINSGQVCNCAERVYVERKVADQFIERISAAMAATRYGDPVAQADVEMGPLINRQGLDSVNRKVRTALGQGASLVTGGQIADLGQGFHFQPTVLAGCRADMEIMREEIFGPVLPIQIVDDLDEAIALSNDCDYGLTSSIYTRDLSKAMQAVRELSFGETYINRENFEAMQGFHAGVRKSGIGGADGKHGLYEYTHTHAVYLQG

SEQ ID NO: 326 - protein, 477 aa, Pseudomonas yamanorum
MSSVPVYQNFINGQFVASAAHLDVINPATGAVLSKVPASTADDVDRALAAARAAQKDWSRKPAIERAGHLRRIATKLRENVAHLARTITLEQGKVSGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPAGVFNVVCGDGRVGAALSAHKGVDMISFTGSVDTGSRIMAAAAPNITKLNLELGGKAPAIVLADADLDLAVKAIRDSRIINSGQVCNCAERVYVERKVADQFIERIAAAMSATRYGDPIAQPDVEMGPLINRQGLDSVNQKVRTALSQGASLISGGQVADLPNGYHFQPTVLAGCRADMEIMREEIFGPVLPIQIVDDLDEAIAMANDCDYGLTSSIYTRDLSKTMHAMRELDFGETYVNRENFEAMQGFHAGVRKSGIGGADGKHGLYEYTHTHAVYLQG

SEQ ID NO: 327 - protein, 477 aa, Pseudomonas sp.
  source 1..477: note = strain GM79
MSSVPVYQNFINGQFVASAAHLDVINPATGAVLSKVPASTADDVDRALAAARAAQKDWSRKPAIERAGHLRRIATKLRENVAHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPAGVFNVVCGDGRVGAALSAHKDVDMISFTGSVDTGSRIMAAAAPNITKLNLELGGKAPAIVLADADLDLAVKAIRDSRIINSGQVCNCAERVYVERKVADQFIERIAAAMSATRYGDPIAQPDVEMGPLINRQGLDSVNQKVRTALSQGASLISGGQVADLPNGYHFQPTVLAGCRADMEIMREEIFGPVLPIQIVDDLDEAIAMANDCDYGLTSSIYTRDLSKTMHAMRELDFGETYVNRENFEAMQGFHAGVRKSGIGGADGKHGLYEYTHTHAVYLQG

SEQ ID NO: 328 - protein, 477 aa, Pseudomonas fluorescens
MSSVPVYENFINGQFVASTAHLDVINPATGVVLSKVPASTAEDVDHALAAARAAQKDWSRKPAIERAGHLRRIAAKLRENVAHLARTITLEQGKVSGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPAGVFNVVCGDGRVGAALSAHKGVDMISFTGSVDTGSRIMAAAAPNITKLNLELGGKAPAIVLADADLDLAVKAIRDSRIINSGQVCNCAERVYVERKVADSFIERIAAAMSATRYGDPIAQPEVEMGPLINRQGLDSVNRKVRTALSQGASLISGGQIADLPNGFHFQPTVLAGCSADMEIMREEIFGPVLPIQIIDDLDEAIALANDCDYGLTSSIYTRDLSKTMHAMRELDFGETYVNRENFEAMQGFHAGVRKSGIGGADGKHGLYEYTHTHAVYLQG

SEQ ID NO: 329 - protein, 477 aa, Pseudomonas reinekei
MPSVPVYENFINGQFVASAAHLDVINPATGAVLSKVPASTAEDVDRALASARAAQKDWSRKPANDRAGYLRRIAAKLRENVSHLARTITLEQGKISGLAEVEVNFTADYLDYMAEWARRIEGEIITSDRQNENIFLFRKPLGVVAGILPWNFPFFLIARKMAPALLTGNSIVIKPSEETPNNCFEFARLVAETDLPPGVFNVVCGDGQVGAALSAHKGVDMISFTGSVVTGSRIMAAAAPNITKLNLELGGKAPAIVLADADLGLAVKAIRDSRIINSGQVCNCAERVYVERKVADQFIERIAAAMSATRYGDPIAQPDVEMGPLINRQGLDSVNRKVRTALSQGANLISGGQIADRPNGFHFQPTVLAGCRADMDIMREEIFGPVLPIQIIDDLDEAITLANDCDYGLTSSIYTRDLNKTLHAMRELDFGETYVNRENFEAMQGFHAGVRKSGIGGADGKHGLYEYTHTHAVYLQG

SEQ ID NO: 330 - protein, 476 aa, Pseudomonas umsongensis
MSSVPVYQNFINGQFVASAAHLDVINPATGALLSKVPASNAEDVDRALAAARTAQKDWAHKPANDRAGYLRRIAAKLRENVAHLARTITLEQGKISALAEVEVNFTADYLDYMAEWARRIEGEIITSDRQDENIFLFRKPLGVVAGILPWNFPFFLIARKMAPALLTGNTIVIKPSEETPNNCFEFARLVAQTDLPPGVFNVVCGDGQVGAALSAHKRVDMISFTGSVATGSRIMAAAAPNITKLNLELGGKAPAIVLADADLELAVKAIRDSRIINSGQVCNCAERVYVERKVADQFIERIATAMNATRYGDPVAQPDVEMGPLINRQGLDSVNRKVRTALSQGASLISGGQIADLPGFHFQPTVLAGCHADMEIMREEIFGPVLPIQIVDDLDEAIALANDCDYGLTSSIYTRDLSKTLHAMRELDFGETYVNRENFEAMQGFHAGVRKSGIGGADGKHGLYEYTHTHAVYLQG

SEQ ID NO: 331 - protein, 477 aa, Pseudomonas sp.
  source 1..477: note = strain B14
MSSVPVYQNFINGRFTPSQAHIDVFNPATGVLLSKVPASTADDVDQALAAARSAQKDWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKLAPALLTGNTIVIKPSEETPNNCFEFAKLVAQTDLPAGVFNVVCGDGQVGAALTAHKGVDMISFTGSVATGSRIMAAAAPNITKLNLELGGKAPAIVLADADLDLAVKAIRDSRIINTGQVCNCAERVYVERKVAEPFIERISAAMSATRYGDPLAMADAEMGPLINRQGLDSVNRKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQS

SEQ ID NO: 332 - protein, 477 aa, Pseudomonas cedrina
MSSVPVYQNFINGRFTASDSHIDVLNPATGALLSKVPASTAADVDQALAAARSAQKDWARKPAIERAGHLRRIAAKLRENVAHLARTITLEQGKVTGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGTRIMAAAAPNITKLNLELGGKAPAIVLADADLELAVKAIRDSRIINTGQVCNCAERVYVERKVADQFIERISAAMSATRYGDPIAQADVEMGPLINRQGLDSVDRKVRTALSQGAALISGGQIADLGAGFHFQPTVLAGCRADMEIMREEIFGPVLPIQIVDDLDEAIALANDCDYGLTSSIYTRDLGKAMHAIREIDFGETYVNRENFEAMQGFHAGVRKSGIGGADGKHGLYEYTHTHVVYLQS

SEQ ID NO: 333 - protein, 477 aa, Pseudomonas karstica
MSSVPVYQNFINGHFTASQAHLDVLNPATGALLSKVPASSAADVDRALSAARGAQKDWGRKPAIERAGHLRRIAAKLRENVAHLARTITLEQGKVTGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPPGVFNVVCGDGRVGGALTAHPGVDMISFTGSVATGSRIMAAAAPNITKLNLELGGKAPAIVLADADLDLAVKAIRDSRIINSGQVCNCAERVYVERKVADQFIERISAAMAATRYGDPIAQPDVDMGPLINRQGLDSVERKVRTALSQGATLISGGQVADLGQGFHFQPTVLAGCRADMEIMREEIFGPVLPIQIVDDLDEAIALANDCDYGLTSSLYTRDLGKAMHGVRELSFGETYVNRENFEAMQGFHAGVRKSGIGGADGKHGLYEYTHTHVMYLQS

SEQ ID NO: 334 - protein, 477 aa, Pseudomonas sp.
  source 1..477: note = strain 2995-1
MSSVPVYQNFINGRFVPSEAHLDVLNPASGALLSKVPASTAADVDQALAAARGAQKAWARKPAIERAGHLRRIAAKLRENVAHLARTITLEQGKVTGLAEVEVNFTADYLDYMAEWARRIEGEIITSDRPNENIFLFRKPLGVVAGILPWNFPFFLIARKLAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVADPFIERISAAMSATRYGDPMAMADAEMGPLINRQGLENVNRKVRTALSQGATLISGGQTADLGAGFHFQPTVLAGCRADMEIMREEIFGPVLPIQIVDDLDEAIAMANDCDYGLTSSIYTRDLGKAMHAVREIDFGETYVNRENFEAMQGFHAGVRKSGIGGADGKHGLYEYTHTHVVYLQS

SEQ ID NO: 335 - protein, 477 aa, Pseudomonas fluorescens
MSSVPVYQNFINGRFTPSEAHIDVLNPATGALLSKVPASSAAEVDQALAAARQAHKAWARKPAIERAGHLRRIAAKLRENVAHLARTITLEQGKVSGLAEVEVNFTADYLDYMAEWARRIEGEIITSDRANENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVADSFIERISAAMAATRYGDPIAQADVEMGPLINRQGLDSVNRKVRTALSQGASLISGGQIADLGAGFHFQPTVLAGCRADMEIMREEIFGPVLPIQIVDDLDEAIALANDCEYGLTSSIYTRDLGKAMHAVREIDFGETYVNRENFEAMQGFHAGVRKSGIGGADGKHGLYEYTHTHVVYMQS

SEQ ID NO: 336 - protein, 477 aa, Pseudomonas sp.
MSSVPVYQNFINGRFVSSEAHLDVLNPATGALLSKVPASTGADVDQALAAARGAQKEWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLDLAVKAIRDSRIINTGQVCNCAERVYVERKVADQFIERISAAMSATRYGDPLAMADAEMGPLINRQGLDNVNRKVRTALSQGATLVSGGQIADLGAGFHFQPTVLAGCSADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQS

SEQ ID NO: 337 - protein, 477 aa, Pseudomonas sivasensis
MSSVPVYQNFINGRFVPSEAHLDVLNPASGALLSKVPASTAADVDHALAAARGAQKAWARKPAIERAGHLRRIAAKLRENVAHLARTITLEQGKVTGLAEVEVNFTADYLDYMAEWARRIEGEIITSDRRDENIFLFRKPLGVVAGILPWNFPFFLIARKLAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVAAPFIERISAAMSATRYGDPMAMADAEMGPLINRQGLENVNRKVRTALSQGATLISGGQIADLGAGFHFQPTVLAGCRADMEIMREEIFGPVLPIQIVDDLDEAIAMANDCDYGLTSSIYTRDLGKAMHAVREIDFGETYVNRENFEAMQGFHAGVRKSGIGGADGKHGLYEYTHTHVVYLQS

SEQ ID NO: 338 - protein, 477 aa, Pseudomonas brassicacearum
MSSVPVYQNFINGRFTASDSHIDVLNPATGALLSKVPASTADDVDQALAAARSAQKDWARKPAIERAGHLRRIAAKLRENVAHLARTITLEQGKITGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGTRIMAAAAPNITKLNLELGGKAPAIVLADADLELAVKAIRDSRIINTGQVCNCAERVYVERKVADQFIERISAAMSATRYGDPIAQADVEMGPLINRQGLDSVDRKVRTALSQGANLISGGQIADLGTGFHFQPTILAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQG

SEQ ID NO: 339 - protein, 477 aa, Pseudomonas sp.
  source 1..477: note = strain BIGb0381
MPSVPVYQNFINGRFTPSQAHIDVFNPATGVLLSKVPASTADDVDQALAAARSAQKDWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKLAPALLTGNTIVIKPSEETPNNCFEFAKLVAQTDLPAGVFNVVCGDGQVGAALTTHKGVDMISFTGSVATGSRIMAAAAPNITKLNLELGGKAPAIVLADADLDLAVKAIRDSRIINTGQVCNCAERVYVERKVAEPFIERISAAMSATRYGDPLAMADAEMGPLINRQGLDSVNRKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQS

SEQ ID NO: 340 - protein, 477 aa, Pseudomonas sp.
MPSVPVYQNFINGRFTPSQAHIDVFNPATGVLLSKVPASTADDVDQALAAARSAQKDWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKLAPALLTGNTIVIKPSEETPNNCFEFAKLVAQTDLPAGVFNVVCGDGQVGAALTAHKGVDMISFTGSVATGSRIMAAAAPNITKLNLELGGKAPAIVLADADLDLAVKAIRDSRIINTGQVCNCAERVYVERKVAEPFIERISAAMSATRYGDPLAMADAEMGPLINRQGLDSVNRKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHVVYLQS

SEQ ID NO: 341 - protein, 477 aa, Pseudomonas sp.
  source 1..477: note = strain Xaverov 83
MSSVPVYQNFINGRFTPSDAHLDVLNPATGVLLSKVPAATAEDVDQALAAARHAQKDWARKPAIERAGHLRRIAAKLRENVGHLARTITLEQGKVSSLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGRVGGALTAHKGVDMISFTGSVATGSRIMAAAAPNITKLNLELGGKAPAIVLADADLDLAVKAIRDSRIINSGQVCNCAERVYVERKVADQFIERISATMAATRYGDPIAQADVEMGPLINRQGLDSVNRKVRTALSQGASLVTGGQIDDLGQGFHFQPTVLAGCHADMEIMREEIFGPVLPIQIVDDLDEAIAMANDCDYGLTSSIYTRDLGKAMQAVRELSFGETYVNRENFEAMQGFHAGVRKSGIGGADGKHGLYEYTHTHAVYLQG

SEQ ID NO: 342 - protein, 477 aa, Pseudomonas yamanorum
MSSVPVYQNFINGRFTPSDAHLDVLNPATGVLLSKVPAATAEDVDQALAAARHAQKDWARKPAIERAGHLRRIAAKLRENVGHLARTITLEQGKVSSLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGRVGGALTAHKGVDMISFTGSVATGSRIMAAAAPNITKLNLELGGKAPAIVLADADLDLAVNAIRDSRIINSGQVCNCAERVYVERKVADQFIERISATMAATRYGDPIAQADVEMGPLINRQGLDSVNRKVRTALSQGASLVTGGQIADLGQGFHFQPTVLAGCHADMEIMREEIFGPVLPIQIVDDLDEAIAMANDCDYGLTSSIYTRDLGKAMQAVRELSFGETYVNRENFEAMQGFHAGVRKSGIGGADGKHGLYEYTHTHAVYLQG

SEQ ID NO: 343 - protein, 477 aa, Pseudomonas sp.
  source 1..477: note = strain MWU16-30323
MSSVPVYQNFINGRFTPSDAHLDVLNPATGVLLSKVPAATAEDVDQSLAAARHAQKDWARKPAIERAGHLRRIAAKLRENVGHLARTITLEQGKVSGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGRVGGALTAHKGVDMISFTGSVATGSRIMAAAAPNITKLNLELGGKAPAIVLADADLDLAVKAIRDSRIINSGQVCNCAERVYVERKVADQFIERISAAMAATRYGDPIAQADVEMGPLINRQGLDSVNRKVRTALSQGASLVTGGQIADLGQGFHFQPTVLAGCHADMEIMREEIFGPVLPIQIVDDLDEAIAMANDCDYGLTSSIYTRDLGKAMQAVRELSFGETYVNRENFEAMQGFHAGVRKSGIGGADGKHGLYEYTHTHAVYLQG

SEQ ID NO: 344 - protein, 474 aa, Pseudomonas migulae
MPVYENFINGQFVASAAHIDVINPATGAVLSKVPASTAEDLDRALAAARGAQKDWSRKPAIERAGHLRRIAAKLRENVAHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPPGVFNVVCGDGRVGAALSAHTGVDMISFTGSVDTGSRIMAAAAPNITKLNLELGGKAPAIVLADADLDLAVKAIRDSRIINSGQVCNCAERVYVERKVADQFIERIAAAMSATRFGDPIAQPDIEMGPLINRQGLDSVNRKVRTALSQGANLISGGQIADLPNGFHFQPTVLAGCRADMEIMREEIFGPVLPIQIIDDLDEAIALANDCEYGLTSSIYTRDLSKTMHAMRELDFGETYVNRENFEAMQGFHAGVRKSGIGGADGKHGLYEYTHTHAVYLQG

SEQ ID NO: 345 - protein, 474 aa, Pseudomonas sp.
  source 1..474: note = strain SJZ080
MPVYENFINGQFVASAAHIDVINPATGAVLSKVPASTAEDVDRALAAARGAQKDWSRKPAIERAGHLRRIAAKLRENVAHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPPGVFNVVCGDGRVGAALSAHKGVDMISFTGSVDTGSRIMAAAAPNITKLNLELGGKAPAIVLADADLDLAVKAIRNSRIINSGQVCNCAERVYVERKVADQFIERIAAAMSATRFGDPIAQPDIEMGPLINRQGLDSVNRKVRTALSQGANLISGGQIADLPSGFHFQPTVLAGCRADMEIMREEIFGPVLPIQIIDDLDEAIALANDCEYGLTSSIYTRDLSKTMHAMRELDFGETYVNRENFEAMQGFHAGVRKSGIGGADGKHGLYEYTHTHAVYLQG

SEQ ID NO: 346 - protein, 474 aa, Pseudomonas fluorescens
MPVYENFINGQFVASAAHLDVINPATGAVLSKVPASTAEDVDRALAAARTAQKDWSRKPAIERAGHLRRIAAKLRENVAHLARTITLEQGKISALAEVEVNFTADYLDYMAEWARRIEGEIITSDRQNENIFLFRKPLGVVAGILPWNFPFFLIARKMAPALLTGNTIVIKPSEETPNNCFEFARLVAKTDLPPGVFNVVCGDGRVGAALSAHKGVDMISFTGSVDTGSRIMAAAAPNITKLNLELGGKAPAIVLADADLDLAVKAIRDSRIINSGQVCNCAERVYVERRVADQFIERIAAAMSATRFGDPIAQPDVEMGPLINRQGLDSVNRKVRTALSQGASLISGGQIADLPNGFHFQPTVLAGCNATMDIMREEIFGPVLPIQIIDDLDEAIALANDCEYGLTSSIYTRDLSKTMHAMRELDFGETYVNRENFEAMQGFHAGVRKSGIGGADGKHGLYEYTHTHAVYLQG

SEQ ID NO: 347 - protein, 477 aa, Pseudomonas sp.
MSSVPVYQNFINGRFTPSQAHIDVFNPATGVLLSKVPASTADDVDQALAAARSAQKDWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKLAPALLTGNTIVIKPSEETPNNCFEFAKLVAQTDLPAGVFNVVCGDGQVGAALTAHKGVDMISFTGSVATGSRIMAAAAPNITKLNLELGGKAPAIVLADADLDLAVKAIRDSRIINTGQVCNCAERVYVERKVAEPFIERISAAMSATRYGDPLAMADAEMGPLINRQGLDSVNRKVRTALSQGATLVSGGQIADLGAGFHFQPTVLAGCRADMEIMREEIFGPVLPIQIVDDLDEAIVLANDCDYGLTSSIYTRDLGKAMHAVREIDFGETYVNRENFEAMQGFHAGVRKSGIGGADGKHGLYEYTHTHVVYLQS

SEQ ID NO: 348 - protein, 477 aa, Pseudomonas lurida
MSSVPVYQNFINGRFTPSEAHIDVLNPATGALLSKVPASSAAEVDQALAAARQAHKAWARKPAIERAGHLRRIAAKLRENVAHLARTITLEQGKVSGLAEVEVNFTADYLDYMAEWARRIEGEIITSDRANENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVADSFIERISAAMSATRYGDPIAQADVEMGPLINRQGLDSVNRKVRTALSQGASLISGGQIADLGAGFHFQPTVLAGCRADMEIMREEIFGPVLPIQIVDDLDEAIALANDCEYGLTSSIYTRDLGKAMHAVREIDFGETYVNRENFEAMQGFHAGVRKSGIGGADGKHGLYEYTHTHVVYMQS

SEQ ID NO: 349 - protein, 477 aa, Pseudomonas lurida
MSSVPVYQNFINGRFTPSEAHIDVLNPATGALLSKVPASSAAEVDQALAAARQAHKAWARKPAIERAGHLRRIAAKLRENVAHLARTITLEQGKVRGLAEVEVNFTADYLDYMAEWARRIEGEIITSDRANENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVADSFIERISAAMSATRYGDPIAQADVEMGPLINRQGLDSVNRKVRTALSQGASLISGGQIADLGAGFHFQPTVLAGCRADMEIMREEIFGPVLPIQIVDDLDEAIALANDCEYGLTSSIYTRDLGKAMHAVREIDFGETYVNRENFEAMQGFHAGVRKSGIGGADGKHGLYEYTHTHVVYMQS

SEQ ID NO: 350 - protein, 477 aa, Pseudomonas fluorescens
MSSVPVFQNFINGQFTHSEAHLDVFNPATGALLSRVPASNAADVDQALAAARVAQKGWAAKPAIERAGHLRRIAAKLRENVAHLARTITLEQGKISALAEVEVNFTADYLDYMAEWARRIEGEIITSDRPGENIFLFRKPLGVVAGILPWNFPFFLIARKMAPALLTGNTIVIKPSEETPNNCFEFARLVAETDLPPGVFNVVCGDGQVGAALSAHQGVDMISFTGSVDTGSRIMTAAAPNITKLNLELGGKAPAIVLADADLALAVKAIRDSRIINTGQVCNCAERVYVERKVADQFIERISAAMSATRYGDPIAEADVEMGPLINRQGLDSVERKVRTALSQGASLVSGGHVADRPQGFHFQPTVLAGCNTSMDIMREEIFGPVLPIQIIDDLDEAIALANDCDYGLTSSVYTRDLSRAMHAIRGLDFGETYVNRENFEAMQGFHAGVRKSGVGGADGKHGLYEYTHTHAVYLQS

SEQ ID NO: 351 - protein, 477 aa, Pseudomonas sp.
MSSVPVYENFINGQFVASADHLDVINPATGALLSKVPASNAEDVDRALAVARTAQKDWARKPANDRAGYLRRIAAKLRENVAHLARTITLEQGKVSSLAAVEVNFTADYLDYMAEWARRIEGEIITSDRQDENIFLFRKPLGVVAGILPWNFPFFLIARKMAPALLTGNTIVIKPSEETPNNCFEFARLVAETDLPPGVFNVVCGDGRVGAALSAHKGVDMISFTGSVATGSRIMAAAAPNITKLNLELGGKAPAIVLADADLDLAVKAIRDSRIINSGQVCNCAERVYVERKVADQFIERIAAAMTATRYGDPIAQPDVEMGPLINRQGLDSVNQKVRTALSQGASLISGGQIADLPNGFHFQPTVLAGCRADMEIMREEIFGPVLPIQIIDDLDEAIALANDCDYGLTSSIYTRDLSKTLHAMRELDFGETYVNRENFEAMQGFHAGVRKSGIGGADGKHGLYEYTHTHAVYLQG

SEQ ID NO: 352 - protein, 477 aa, Pseudomonas putida
MSSVPVYENFINGQFVASADHLDVINPATGALLSKVPASNPEDVDRALAAARTAQKDWARKPANDRAGYLRRIAAKLRENVAHLARTITLEQGKVSSLAAVEVNFTADYLDYMAEWARRIEGEIITSDRQDENIFLFRKPLGVVAGILPWNFPFFLIARKMAPALLTGNTIVIKPSEETPNNCFEFARLVAETDLPPGVFNVVCGDGRVGAALSAHKGVDMISFTGSVATGSRIMAAAAPNITKLNLELGGKAPAIVLADADLDLAVKAIRDSRIINSGQVCNCAERVYVERKVADQFIERIAAAMTATRYGDPIAQPDVEMGPLINRQGLDSVNQKVRTALSQGASLISGGQIADLPNGFHFQPTVLAGCRADMEIMREEIFGPVLPIQIIDDLDEAIALANDCDYGLTSSIYTRDLSKTLHAMRELDFGETYVNRENFEAMQGFHAGVRKSGIGGADGKHGLYEYTHTHAVYLQG

SEQ ID NO: 353 - protein, 477 aa, Pseudomonas sp.
  source 1..477: note = strain A-R-19
MPSVPVYENFINGQFVASTAHLDVINPATGAVLSKVPASTAEDVDRALAAARSAQKDWARKPANERAGYLRRIAAKLRENVAHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPAGVFNVVCGDGRVGSALSAHKGVDMISFTGSVDTGSRIMAAAAPNITKLNLELGGKAPAIVLADADLDLAVRAIRDSRIINSGQVCNCAERVYVERKVADQFIERIAAALSATRYGDPIAQPDVEMGPLINRQGLDSVNRKVRNALSQGASLISGGQIADLPYGFHFQPTVLAGCRADMEIMREEIFGPVLPIQIVDDLDEAIAMANDCEYGLTSSIYTRDLSKTMQAMRELDFGETYVNRENFEAMQGFHAGVRKSGIGGADGKHGLYEYTHTHAVYLQG

SEQ ID NO: 354 - protein, 477 aa, Pseudomonas sp.
  source 1..477: note = strain PB101
MPSVPVYENFINGQFVASAAHLDVINPATGAVLSKVPASTAEDVDRALAAARSAQKDWARKPANERAGYLRRIAAKLRENVAHLARTITLEQGKISGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPAGVFNVVCGDGRVGSALSAHKGVDMISFTGSVDTGSRIMAAAAPNITKLNLELGGKAPAIVLADADLDLAVRAIRDSRIINSGQVCNCAERVYVERKVADQFIERIAAAMSATRYGDPIAQPDVEMGPLINRQGLDSVNRKVRTALSQGANLISGGQIADLPNGFHFQPTVLAGCRADMEIMREEIFGPVLPIQIIDDLDEAIAMANDCDYGLTSSIYTRDLSKTMQAMRELDFGETYVNRENFEAMQGFHAGVRKSWIGGADGKHGLYEYTHTHAVYLQG

SEQ ID NO: 355 - protein, 477 aa, Pseudomonas fluorescens
MSSVPVYQNFINGRFIASEAHIDVLNPATGALLSKVPASTAADVDQALAAARHAQKDWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMTAAAPNITKLNLELGGKAPAIVLADADLDLAVKAIRDSRIINTGQVCNCAERVYVERKVADQFIERISAAMSATRYGDPIAQADVEMGPLINRQGLDSVNRKVRTALSQGATLVSGGQIADLGAGFHFQPTVLAGCRADMEIMREEIFGPVLPIQIVDDLDEAIAMANDCDYGLTSSIYTRDLGKAMHAVREIDFGETYVNRENFEAMQGFHAGVRKSGVGGADGKHGLYEYTHTHVVYLQS

SEQ ID NO: 356 - protein, 477 aa, Pseudomonas sp.
MSSVPVYQNFINGRFTPSQAHIDVLNPATGVLLSKVPASTADDVDQALAAARSAQKDWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKLAPALLTGNTIVIKPSEETPNNCFEFAKLVAQTDLPAGVFNVVCGDGQVGGALTAHKGVDMISFTGSVATGSRIMAAAAPNITKLNLELGGKAPAIVLADADLDLAVKAIRDSRIINTGQVCNCAERVYVERKVADPFIERISAAMSATRYGDPLAMADAEMGPLINRQGLDSVNRKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHAVYLQS

SEQ ID NO: 357 - protein, 477 aa, Pseudomonas azotoformans
MSSVPVYQNFINGRFTPSQAHIDVLNPATGVLLSKVPASTADDVDQALAAARSAQKDWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKLAPALLTGNTIVIKPSEETPNNCFEFAKLVAQTDLPAGVFNVVCGDGQVGGALTAHKGVDMISFTGSVATGSRIMAAAAPNITKLNLELGGKAPAIVLADADLDLAVKAIRDSRIINTGQVCNCAERVYVERKVADPFIERISAAMSATRYGDPLAMADAEMGPLINRQGLDSVNHKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHAVYLQS

SEQ ID NO: 358 - protein, 477 aa, Pseudomonas lurida
MSSVPIYQNFINGRFTPSEAHIDVLNPATGALLSKVPASSAAEVDQALAAARQAHKAWARKPAIERAGHLRRIAAKLRENVAHLARTITLEQGKVSGLAEVEVNFTADYLDYMAEWARRIEGEIITSDRANENIFLFRKPLGVVAGILPWNFPFFLIARKMAPALLTGNTIVIKPSEETPNNCFEFAKLVAETDLPAGVFNVVCGNGQVGGALTAHKGVDMISFTGSVATGSRIMAAAAPNITKLNLELGGKAPAIVLADADLELAVKAIRDSRIINTGQVCNCAERVYVERKVADSFIERISAAMSATRYGDPIAQADVEMGPLINRQGLDSVNRKVRTALSQGASLISGGQIADLGAGFHFQPTVLAGCRADMEIMREEIFGPVLPIQIVDDLDEAIALANDCEYGLTSSIYTRDLGKAMHAVREIDFGETYVNRENFEAMQGFHAGVRKSGIGGADGKHGLYEYTHTHVVYMQS

SEQ ID NO: 359 - protein, 477 aa, Pseudomonas lurida
MSSVPIYQNFINGRFTPSEAHIDVLNPATGALLSKVPASSAAEVDQALAAARQAHKAWARKPAIERAGHLRRIAAKLRENVAHLARTITLEQGKVSGLAEVEVNFTADYLDYMAEWARRIEGEIITSDRANENIFLFRKPLGVVAGILPWNFPFFLIARKMAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVADSFIERISAAMSATRYGDPIAQADVEMGPLINRQGLDSVNRKVRTALSQGASLISGGQIADLGAGFHFQPTVLAGCRADMEIMREEIFGPVLPIQIVDDLDEAIALANDCEYGLTSSIYTRDLGKAMHAVREIDFGETYVNRENFEAMQGFHAGVRKSGIGGADGKHGLYEYTHTHVVYMQS

SEQ ID NO: 360 - protein, 477 aa, Pseudomonas lurida
MSSVPVYQNFINGRFTPSEAHIDVLNPATGALLSKVPASSAAEVDQALAAARQAHKAWARKPAIERAGHLRRIAAKLRENVAHLARTITLEQGKVSGLAEVEVNFTADYLDYMAEWARRIEGEIITSDRANENIFLFRKPLGVVAGILPWNFPFFLIARKLAPALLTGNTIVIKPSEETPNNCFEFAKLVAETDLPAGVFNVVCGDGQVGGALTAHKGVDMISFTGSVATGSRIMAAAAPNITKLNLELGGKAPAIVLADADLELAVKAIRDSRIINTGQVCNCAERVYVERKVADSFIERISAAMSATRYGDPIAQADVEMGPLINRQGLDSVNRKVRTALSQGASLISGGQIADLGAGFHFQPTVLAGCRADMEIMREEIFGPVLPIQIVDDLDEAIALANDCEYGLTSSIYTRDLGKAMHAVREIDFGETYVNRENFEAMQGFHAGVRKSGIGGADGKHGLYEYTHTHVVYMQS

SEQ ID NO: 361 - protein, 477 aa, Pseudomonas fluorescens
MSSVPVYENFINGQFVASAAHLDVINPATGALLSKVPASTTEEVDRALAAARTAQKDWSRKPAIERAGHLRRIAARLRENVTHLARTITLEQGKVSGLAEVEVNFTADYLDYMAEWARRIEGEIITSDRQNENIFLLRKPLGVVAGILPWNFPFFLIARKMAPALLTGNTIVIKPSEETPNNCFEFARLVAETDLPAGVFNVVCGDGRVGAALSAHKGVDMISFTGSVATGSRIMAAAAPNITKLNLELGGKAPAIVLADADLDLAVKAIRDSRIINSGQVCNCAERVYVERKVADQFIERIAAVMSATRYGDPIAQPDIEMGPLINRQGLDSVNRKVRTALSQGASLISGGQIADLPNGFHFQPTVLAGCRADMEIMREEIFGPVLPIQIIDDLDEAIALANDCDYGLTSSIYTRDLSKTLQAMRELDFGETYVNRENFEAMQGFHAGVRKSGIGGADGKHGLYEYTHTHAVYLQG

SEQ ID NO: 362 - protein, 477 aa, Pseudomonas sp.
MSSVPVYENFINGQFVASTDHIDVINPATGVTLSKVPASTAEDVDHALAAARSAQKDWSRKPAIERAGHLRRIAAKLRENVQHLARTITLEQGKVSGLAEVEVNFTADYLDYMAEWARRIEGEIITSDRQNENIFLFRKPLGVVAGILPWNFPFFLIARKMAPALLTGNTIVIKPSEETPNNCFEFARLVAETDLPAGVFNVVCGDGRVGAALSAHKGVDMISFTGSVDTGSRIMAAAAPNITKLNLELGGKAPAIVLADADLDLAVKAIRDSRIINSGQVCNCAERVYVERKVADSFIERIAAAMSATRYGDPIARPEVEMGPLINRQGLDSVNRKVRTALSQGASLISGGQIADLPNGFHFQPTVLAGCSADMEIMREEIFGPVLPIQIIDDLDEAIALANDCDYGLTSSIYTRDLSKTMHAMRELDFGETYVNRENFEAMQGFHAGVRKSGIGGADGKHGLYEYTHTHAVYLQG

SEQ ID NO: 363 - protein, 477 aa, Pseudomonas pisciculturae
MSSVPVYQNFINGRFVSSEAHIDVLNPATGALLSKVPSSTAADVDQALAAARHAQKDWGRKPAIERAGHLRRIAAKLRENVAHLARTITLEQGKVSGLAEVEVNFTADYLDYMAEWARRIEGEIITSDRQNENIFLFRKPLGVVAGILPWNFPFFLIARKMAPALLTGNTIVIKPSEETPNNCFEFAKLVAETDLPAGVFNVVCGDGQVGGALTSHKGVDMISFTGSVATGSRIMTAAAPNITKLNLELGGKAPAIVLADADLELAVKAIRDSRIINTGQVCNCAERVYVERKVADQFIQRISAAMSATRYGDPIAQADVEMGPLINRQGLDNVNRKVRTALSQGATLVSGGQIADLGAGFHFQPTVLAGCRADMEIMREEIFGPVLPIQIVDDLDEAIAMANDCDYGLTSSIYTRDLGKAMHAVREIDFGETYVNRENFEAMQGFHAGVRKSGVGGADGKHGLYEYTHTHVVYLQS

SEQ ID NO: 364 - protein, 477 aa, Pseudomonas sp.
  source 1..477: note = strain Tri1
MSSVPVFQNFINGQFTRSEAHLDVFNPATGALLSRVPASTAADIDQALAAARVAQKDWSAKPAIERAGHLRRIAAKLRENVAHLARTITLEQGKISALAEVEVNFTADYLDYMAEWARRIEGEIITSDRPGENIFLFRKPLGVVAGILPWNFPFFLIARKMAPALLTGNTIVIKPSEETPNNCFEFARLVAETDLPPGVFNVVCGDGQVGAALSGHKGVDMISFTGSVDTGSRIMTSAAANITKLNLELGGKAPAIVLADADLELAVKAIRDSRIINTGQVCNCAERVYVERKVADQFIERISAAMSATRYGDPLAEPDVEMGPLINRQGLDSVERKVKKALSQGASLISGGSVAGRPSGFHFQPTVLAGCNASMEIMREEIFGPVLPIQIIDDLDEAIALANDCDYGLTSSVYTRDLSRAMHAIRGLDFGETYVNRENFEAMQGFHAGVRKSGVGGADGKHGLYEYTHTHAVYLQS

SEQ ID NO: 365 - protein, 477 aa, Pseudomonas marginalis
MSSVPVYQNFINGRFTPSQAHIDVLNPATGVLLSKVPASTADDVDQALAAARSAQKDWARKPAIERAGHLRRIAAKLRENVAHLARTITLEQGKVSGLAEVEVNFTADYLDYMAEWARRIEGEIITSDRPNENIFLFRKPLGVVAGILPWNFPLFLIARKLAPALLTGNTIVIKPSEETPNNCFEFAKLVAQTDLPAGVFNVVCGDGQVGGALTAHKGVDMISFTGSVATGSRIMAAAAPNITKLNLELGGKAPAIVLADADLDLAVKAIRDSRIINTGQVCNCAERVYVERKVADPFIERISAAMSATRYGDPLAMADAEMGPLINRQGLDSVNHKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHAVYLQS

SEQ ID NO: 366 - protein, 477 aa, Pseudomonas sp.
  source 1..477: note = strain NFACC42-2
MSSVPVYQNFINGRFTPSQAHIDVLNPATGVLLSKVPASTADDVDQALAAARSVQKDWARKPAIERAGHLRRIAAKLRENVAHLARTITLEQGKVSGLAEVEVNFTADYLDYMAEWARRIEGEIITSDRPNENIFLFRKPLGVVAGILPWNFPFFLIARKLAPALLTGNTIVIKPSEETPNNCFEFAKLVAQTDLPAGVFNVVCGDGQVGGALTAHKGVDMISFTGSVATGSRIMAAAAPNITKLNLELGGKAPAIVLADADLDLAVKAIRDSRIINTGQVCNCAERVYVERKVADPFIERISAAMSATRYGDPLAMADAEMGPLINRQGLDSVNHKVRTALSQGATLVSGGQIADLGAGFHFQPTVLAGCRADMEIMREEIFGPVLPIQIVDDLDEAIALANDCDYGLTSSIYTRDLGKAMHAVREIDFGETYVNRENFEAMQGFHAGVRKSGIGGADGKHGLYEYTHTHAVYLQS

SEQ ID NO: 367 - protein, 499 aa, Pseudomonadaceae sp.
MKDSSDTHPNHLPDSCYGLFIDNQWVTAEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKEEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 368 - protein, 499 aa, Pseudomonas aeruginosa
MKDSSDTHPNHLPDSCYGLFIDNQWVTAEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKEEGAEVLIGGGRLTGADYDAGFFIQPTILVGVRNDMRIAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 369 - protein, 499 aa, Pseudomonas aeruginosa
MKDSSDTHPNHLPDSCYGLFIDNQWVTAEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVNIAKEEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 370 - protein, 499 aa, Pseudomonas aeruginosa
MKDSSDTHPNHLPDSCYGLFIDNQWVTAEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQAILDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKEEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 371 - protein, 499 aa, Pseudomonas sp.
  source 1..499: note = strain KBS0802
MKDSSDTHPNHLPDSCYGLFIDNQWVTAEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVIAILWNQGQVCESGARLFVHESIYERFLAELKHKFEAVRVGDPLNPDTMMGAQVSKTQMERILGYVDIAKEEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 372 - protein, 499 aa, Pseudomonas aeruginosa
MKDSSDTHPNHLPDSCYGLFIDNQWVTAEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSFALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKEEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 373 - protein, 499 aa, Pseudomonas aeruginosa
MKDSSDTHPNHLPDSCYGLFIDNQWVTAEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPVGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKEEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 374 - protein, 499 aa, Stutzerimonas balearica
MKDSSDTHPNHLPDSCYGLFIDNQWVTAEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAALAILWNQGQVCESGARLFVHESIYERFLAELKHKFEAVRVGDPLNPDTMMGAQVSKTQMERILGYVDIAKEEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 375 - protein, 488 aa, Pseudomonas aeruginosa
MKDSSDTHPNHLPDSCYGLFIDNQWVTAEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKEEGAEVLIGGGRLTGADYDAGFFIQPTILVGVRNDMRIAYEEIFGPVLCVIPFKDEAEVIAMANDSEYGLAGAVWTQDINRALRVARAVETGRMWVNTYHEIPAHAPFGGYKKSGLGRETHKSMLEPVLFMRALK

SEQ ID NO: 376 - protein, 499 aa, Pseudomonas aeruginosa
MKDSSDTHPNHLPDSCYGLFIDNQWVTAEYGETLDIINPANGKILTNISNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKEEGAEVLIGGGRLTGADYDAGFFIQPTILVGVRNDMRIAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 377 - protein, 499 aa, Gammaproteobacteria bacterium
MKDSSDTHPNHLPDSCYGLFIDNQWVTAEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTSVGQALLDHPDLRKLAFTGSTRVGELVANAAAKKIIPATLELGGKSANIVFPDANWDKAVEGAVLAILWNQGQVCESGARLFVHESIYEQFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 378 - protein, 499 aa, Pseudomonas sp.
  source 1..499: note = strain GOM6
MKDSSDTHPNHLPDSCYGLFIDNQWVTAEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKSQMERILGYVDIAKQEGAEVLIGGGRLTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 379 - protein, 499 aa, Pseudomonas sp.
  source 1..499: note = strain S9
MKDSSDTHPNHLPDSCYGLFIDNQWVTAEHGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLPLLWKQGQVCESGARLVVHESIYERFLAELKHKFEAVRVGDPLNPDTMMGAQVSKTQMERILGYVDIAKEEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 380 - protein, 499 aa, Pseudomonas sp.
MKDSSDTHPNHLPDSSYGLFIDNQWVSDEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKQEGAEVLIGGGRLTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 381 - protein, 443 aa, Pseudomonas sp.
DRAVQAAQRAFMTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSALTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKSQMERILGYVDIAKQEGAEVLIGGGRLTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 382 - protein, 499 aa, Pseudomonas sp.
  source 1..499: note = uncultured
MKNSSDTHPNHLPDSSYGLFIDNQWVSGEYGETLDIINPANGKILTNIPNATAADVDRAVQAAQRAFVTWRTTSPAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 383 - protein, 499 aa, Pseudomonas aeruginosa
MKDSSDTHPNHLPDSSYGLFIDNQWVSDEYGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAALAILWNQGQVCESGARLFVHESIYERFLAELKHKFEAVRVGDPLNPDTMMGAQVSKTQMERILGYVDIAKQEGAEVLIGGGRLTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 384 - protein, 499 aa, Pseudomonadota sp.
MKDSSDTHPNHLPDSSYGLFIDNQWVSDEYGETLDIINPANGKILTNIPNATAADVDRAVQAAQRAFMTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKSQMERILGYVDIAKQEGAEVLIGGGRLTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 385 - protein, 499 aa, Pseudomonas juntendi
MKDSSDTHPNHLPDSRYGLFIDNQWVSDEYGETLDIINPANGEILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKQEGAEVLIGGGRFTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 386 - protein, 425 aa, Pseudomonas sp.
  source 1..425: note = strain ES3-33
PAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTSVGQAQLDHPDLRKLAFTGSTRVGELVANAAAKKIIPATLELGGKSANIVFPDANWDKAVEGAVLAILWNQGQVCESGARLFVHESIYEQFLAELKHKFEAVRVGDPLNPDTMMGAQVSKTQMERILGYVDIAKQEGAEVLIGGGRLTGADYDAGFFIQPTILVDVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 387 - protein, 499 aa, Pseudomonas aeruginosa
MKDSSDTHPNHLPDSSYGLFIDNQWVSDEYGETLDIINPATGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPALAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAALAILWNQGQVCESGARLFVHESIYERFLAELKHKFEAVRVGDPLNPDTMMGAQVSKTQMERILGYVDIAKQEGAEVLIGGGRLTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 388 - protein, 499 aa, Stutzerimonas zhaodongensis
MKDSSDTHPNHLPDSSYGLFIDNQWVSDEYGETLDIINPANGKILTNIPNATAADVDRAVQAAQRAFMTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKSQMERILGYVDIAKQEGAEVLIGGGRLTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEVYSQKKNIYVSLNEAPLGLF

SEQ ID NO: 389 - protein, 499 aa, Pseudomonadaceae sp.
MKDSSDTHPNHLPDSSYGLFIDNQWVSDEYGETLDIINPANGKILTNIPNATAADVDRAVQAAQRAFMTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKYKFEAVRVGDPLNPDTMMGAQVSKSQMERILGYVDIAKQEGAEVLIGGGRLTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 390 - protein, 499 aa, Pseudomonas putida
MKDSSDTHPNHLPDSRYGLFIDNQWVSDEYGETLDIINPANGEILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALNEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKQEGAEVLIGGGRFTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 391 - protein, 499 aa, Pseudomonas fluorescens
MQNSSDTHPSYLPDNSYGLFIDNQWVAAENGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKEEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 392 - protein, 517 aa, Stutzerimonas frequens
MNTWVSFQSAEQPLLGQFSVSGNNTHPNHLPDSSYGLFIDNQWVSDEYGETLDIINPANGKILTNIPNATAADVDRAVQAAQRAFMTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKSQMERILGYVDIAKQEGAEVLIGGGRLTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 393 - protein, 503 aa, Stutzerimonas frequens
MGQFSVSGNNTHPNHLPDSSYGLFIDNQWVSDEYGETLDIINPANGKILTNIPNATAADVDRAVQAAQRAFMTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKSQMERILGYVDIAKQEGAEVLIGGGRLTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 394 - protein, 499 aa, Pseudomonas monteilii
MQNSSDTHPSYLPDNSYGLFIDNQWVAAENGETLDIINPANGKILTNIPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALNEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 395 - protein, 499 aa, Pseudomonas sp.
MQNSSDTHPSYLPDNSYGLFIDNQWVAAENGETLDIINPANGKILTNIPNATAADVDRAVQAAKRAFVTWHTTSPAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 396 - protein, 499 aa, Pseudomonas sp.
MQNSSDTHPSYLPDNSYGLFIDNQWVAAENGETLDIINPANGKILTNIPNATAADVDRAVQAAKRAFVTWRTTSPAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTSVGQALLDHPDLRKLAFTGSTRVGELVANAAAKKIIPATLELGGKSANIVFPDANWDKAVEGAVLAILWNQGQVCESGARLFVHESIYEQFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 397 - protein, 499 aa, Pseudomonas sp.
MKDSSDTHPNHLPDSRYGLFIDNQWVSDEYGETLDIINPANGEILTNTPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGEVLAILWNQGQVCESGARLFVHESIYERFLAELKHKFEAVRVGDPLNPDTMMGAQVSKSQMERILGYVDIAKQEGAEVLIGGSRLTGANYDAGFFIQPTILVGVRNDMRVAYQEIFGPVLCVIAFKDEAEVIAMANDSEYGLAGAVWTQDINRALRVARAVETGRMWVNTYHEIPAHAPFGGYKKSGLGRETHKSMLEAYSQKKNIYVNLNEAPLGLF

SEQ ID NO: 398 - protein, 499 aa, Pseudomonas sp.
  source 1..499: note = strain NIBR-H-19
MQNSSDTHPSYLPDNSYGLFIDNQWVAAENGETLDIINPANGKVLTNIPNATAADVDRAVQAAKRAFVTWRTTSPAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTSVGQALLDHPDLRKLAFTGSTRVGELVANAAAKKIIPATLELGGKSANIVFPDANWDKAVEGAVLAILWNQGQVCESGARLFVHESIYEQFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 399 - protein, 499 aa, Pseudomonas veronii
MQNSSDTHPSYLPDNSYGLFIDNQWVAAENGETLDIINPSNGKILTNIPNATAADVDRAVQAAKRAFVTWRTTSPAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTSVGQALLDHPDLRKLAFTGSTRVGELVANAAAKKIIPATLELGGKSANIVFPDANWDKAVEGAVLAILWNQGQVCESGARLFVHESIYEQFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 400 - protein, 499 aa, Pseudomonas putida
MQNSSDTHPSYLPDNSYGLFIDNQWVAAENGETLDIINPANGKILTNIPNATAADVDRAVQAAKRAFVTWHTTSPAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTSVGQALLDHPDLRKLAFTGSTRVGELVANAAAKKIIPATLELGGKSANIVFPDANWDKAVEGAALAILWNQGQVCESGARLFVHESIYERFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 401 - protein, 499 aa, Pseudomonas putida
MKDSSDTHPNHLPDSRYGLFIDNQWVSDEYGETLDIINPANGEILTNTPNATAADVDRAVQAAQRAFVTWRTTSPAERANALLKIADLLEADADRFAVLETLDVGKPIRESRSVDIPLAIDHFRYFAGVIRSQSDEAVMLDEQTLSIALSEPLGVVGQVIPWNFPLLMAAWKIAPAIAAGNTVVIKPSELTPVTILELAKIFAKVLPAGVVNIVTGLGTTVGQALLDHPDLRKLAFTGSTRVGELVANAAAKKIIPATLELGGKSANIVFPDANWDKAVEGEVLAILWNQGQVCESGARLFVHESIYERFLAELKHKFEAVRVGDPLNPDTMMGAQVSKSQMERILGYVDIAKQEGAEVLIGGSRLTGANYDAGFFIQPTILVGVRNDMRVAYQEIFGPVLCVIAFKDDAEVIAMANDSEYGLAGAVWTQDINRALRVARAVETGRMWVNTYHEIPAHAPFGGYKKSGLGRETHKSMLEAYSQKKNIYVNLNEAPLGLF

SEQ ID NO: 402 - protein, 499 aa, Pseudomonas carnis
MQNSSDTHPSYLPDNSYGLFIDNQWVAAENGETLDIINPANGKNLTNIPNATAADVDRAVQAAKRAFVTWRTTSPAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTSVGQALLDHPDLRKLAFTGSTRVGELVANAAAKKIIPATLELGGKSANIVFPDANWDKAVEGAVLAILWNQGQVCESGARLFVHESIYEQFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 403 - protein, 499 aa, Pseudomonas shahriarae
MQNSSDTHPSYLPDNSYGLFIDNQWVAAENGETLDIINPSNGKILTNIPNATAADVDRAVQAAKRAFVTWRTTSPAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSEITPVTILELAKIFAKVLPAGVVNIVTGLGTSVGQALLDHPDLRKLAFTGSTRVGELVANAAAKKIIPATLELGGKSANIVFPDANWDKAVEGAVLAILWNQGQVCESGARLFVHESIYEQFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 404 - protein, 499 aa, Pseudomonas sp.
MQNSSDTHPSYLPDNSYGLFIDNQWVAAENSETLDIINPANGKILTNIPDATAADVDRAVQAAKRAFVTWRTTSPAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTSVGQALLDHPDLRKLAFTGSTRVGELVANAAAKKIIPATLELGGKSANIVFPDANWDKAVEGAVLAILWNQGQVCESGARLFVHESIYEQFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 405 - protein, 499 aa, Pseudomonas putida
MQNSSDTHPSYLPDNSYGLFIDNQWVAAENGETLDIINPANGKILTNIPNATAADVDRAVQAAKRAFVTWHTTSPAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTSVGQALLDHPDLRKLAFTGSTRVGELVANAAAKKIIPATLELGGKSANIVFPDANWDKAVEGAVLAILWNQGQVCESGARLFVHESIYERFLAELKHKFEAVRVGDPLNPDTMMGAQVSKTQMERILGYVDIAKQEGAEVLIGGGRFTGANYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 406 - protein, 499 aa, Pseudomonas sp.
MQNSSDTHPSYLPDHSYGLFIDNQWVAAENGETLNIINPANGKILTNIPNATAADVDRAVQAAKRAFVTWHTTSPAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTSVGQALLDHPDLRKLAFTGSTRVGELVANAAAKKIIPATLELGGKSANIVFPDANWDKAVEGAALAILWNQGQVCESGARLFVHESIYERFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 407 - protein, 499 aa, Pseudomonas shahriarae
MQNSSDTHPSYLPDNSYGLFIDNQWVAAENGETLDIINPANGKNLTNIPNATAADVDRAVQAAKRAFVTWRTTSPAERANALLKIADLLEADADRFAGLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTSVGQALLDHPDLRKLAFTGSTRVGELVANAAAKKIIPATLELGGKSANIVFPDANWDKAVEGAVLAILWNQGQVCESGARLFVHESIYEQFLAELKHKFEAVRVGDPLNPDTMMGAQVSKTQMERILGYVDIAKQEGAEVLIGGGRLTGADYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYVSLNEAPLGLF

SEQ ID NO: 408 - protein, 499 aa, Pseudomonas putida
MQNSSDTHPSYLPDHSYGLFIDNQWVAAENGETLDIINPANGKILTNIPNATAADVDRAVQAAKRAFVTWHTTSPAERANALLKIADLLEADADRFAVLETLDVGKPIRESRSVDIPLAIDHFRYFAGAIRSQSDEAVMLDEQTLSIALSEPLGVVGQVIPWNFPLLMAAWKIAPAIAAGNTVVIKPSELTPVTILELAKIFAKVLPAGVVNIVTGLGTSVGQALLDHPDLRKLAFTGSTRVGELVANAAAKKIIPATLELGGKSANIVFPDANWDKAVEGAALAILWNQGQVCESGARLFVHESIYERFLAELKHKFEAVRVGDPLNPDTMMGAQVSKTQMERILGYVDIAKQEGAEVLIGGGRLTGVDYDAGFFIQPTILVGVRNDMRVAYEEIFGPVLCVIPFKDEAEVIAMANDSEYGLAGAVWTQDINRALRVARAVETGRMWVNTYHEIPAHAPFGGYKKSGLGRETHKSMLEAYSQKKNIYFSLNEAPLGLF

SEQ ID NO: 409 - protein, 499 aa, Azotobacter vinelandii
MQASNDTRSNYAPDSSYGLFIDNQWVAGENGETITILNPANGKTLTGIPNATAVDVDRAVQAAQRAFEAWRSTTPIERANALLKIADLLEADAERFAALESLDVGKPIRESSSVDIPLAIDHFRYFAGVIRSHSDEAVMLDEQTLSIVLSEPLGVVGQVIPWNFPLLMAAWKIAPAIAIGNTVVIKPSELTPVSILELAKIFAQVLPAGVVNIVTGTGASAGQALLDHPDVRKLAFTGSTSVGHRVADAAAKKLIPATLELGGKSANIVFPDANWDKAVEGAALAILWNQGQVCESGARLFVHESIYERFLDEVKQKFEAVRVGDPLHPDTMMGAQVSKTQMERILGYVDIAKQEGAKVLLGGGRLTGADYDAGFFIQPTILVDVRNDMRVAYEEIFGPVLCVIPFKDEADVIAMANDSEYGLAGAVWTQDINRALRVARAVETGRMWVNTYHEIPAHAPFGGYKKSGLGRETHKSILEAYSQKKNIYVSLNEAPLGLF

SEQ ID NO: 410 - protein, 499 aa, Stutzerimonas azotifigens
MQASSDIRPNHAPDSSYGLFIDNQWVAGESAETLAILNPANGKTLTSIPNATAFDVDRAVRAAQRAFETWRSTTPIERANALLKIADLLEADAERFSYLESLDVGKPIRESSSVDIPLAIDHFRYFAGVIRSHSDEAVMLDEQTLSIVLSEPLGVVGQVIPWNFPLLMAAWKIAPAIAAGNTVVIKPSELTPVSILELAKIFAQVLPAGVVNIVTGTGASAGQALLDHPGVRKLAFTGSTGVGHRVADAAAKKLIPATLELGGKSANIVFPDANWDKAVEGAALAILWNQGQVCESGARLFLHESIYERFLGELKQKFEAVRVGDPQHPETMMGAQVSKTQMERILGYIDIARQEGATVLLGGGRLTGPGYDDGFFIQPTILVDVRNDMRVAYEEIFGPVLCVIPFKDEADVIAMANDSEYGLAGAVWTQDINRALRVSRAVETGRMWVNTYHEIPAHAPFGGYKKSGLGRETHKSILEAYTQKKNVYVSLNEAPLGLF

SEQ ID NO: 411 - protein, 499 aa, Stutzerimonas stutzeri
MQKLNVTLLTYAPDSSYGLFIDNQWVAGEGGETLSIINPANGQTLTSIPNATAADVDRAVQAAQRAFETWRFTTPIERANALLKIADLLEADAERFSVLETLDVGKPIRESSSIDIPLAIDHFRYFAGVIRSHSDEAVMLDEQTLSIVLSEPLGVVGQVIPWNFPLLMAAWKIAPAIAAGNTVVIKPSELTPVTILELAKIFAQTLPAGVVNIVTGTGASAGQALLDHPDVRKLAFTGSTGVGMRVADAAAKKLIPATLELGGKSANIVFPDANWDKAVEGAALAILWNQGQVCESGARLFVHESIYDRFLAEVKQKFEAVRVGDPLHRDTMMGAQVSKTQMERILGYIDIAKQEGARVLIGGGRLTGLAYDNGFFIQPTILVDVRNDMRVAYEEIFGPVLCVIPFKDEAEVIAMANDSDYGLAGAVWTQDINRALRVARAVETGRMWVNTYHDIPAHAPFGGYKKSGLGRETHKSILDAYSQKKNILVSLKEAAHGLF

SEQ ID NO: 412 - protein, 499 aa, Stutzerimonas nitrititolerans
MQASSDTRANYRPDTSYGLFIDNQWVAGDSGETLDILNPANGDIITRIPNATAVDVDHAVQAAQRAFATWRTTTPTERANALLKIADLMEADAERFSVLETLDVGKPIRESSTVDIPLAIDHFRYFAGVIRSHSDEAVMLDEQTLSIVLSEPLGVVGQVIPWNFPLLMAAWKIAPAIAAGNTVVIKPSELTPISILELAKIFAKVLPAGVVNIITGTGASTGQALLDHPHVRKLAFTGSTGVGHRVADAAAKKLIPATLELGGKSANIVFPDANWDKAVEGAALAILWNQGQVCESGARLFVHESIYERFLAEVKQKFEAVRVGNPLHPDTMMGAQVSKTQMERILGYIDIAKQEGATVLLGGGRLTGPGYDDGFFIQPTILVDVRNDMRVAYEEIFGPVLCVIPFKDEADVIAMANDSEYGLAGAVWTQDINRALRVSRAVETGRIWVNTYHEIPAHAPFGGYKKSGLGRETHKSILEAYTQKKNVYISLNEAPLGLF

SEQ ID NO: 413 - protein, 499 aa, Pseudomonas sp.
  source 1..499: note = strain Q1-7
MQKLDVTLPTYVPDSSYGLFIDNQWVAGESGETLSIINPANGQTLTSIPNGTAADIDRAVQAAQRAFETWRLTSPIERANALLKIADLMEADAERFSVLETLDVGKPIRESSSIDVPLAIDHFRYFAGVIRSHSDEAVMLDEQTLSIVLSEPLGVVGQVIPWNFPLLMAAWKIAPAIAAGNTVVIKPSELTPVTILELAKILAKVLPAGVVNIVTGTGASAGQALLDHPDVRKLAFTGSTGVGMRVADAAAKKLIPATLELGGKSANIVFPDANWDKAVEGAALAILWNQGQVCESGARLFVHESIYERFLAEVQQKFEAVRVGDPLHRDTMMGAQVSKTQMERILGYIDIAKQEGARVLIGGGRLTGPAYDNGFFIQPTILVDVRNDMRVAYEEIFGPVLCVIPFKDEAEVIAMANDSDYGLAGAVWTQDINRAVRVARAVETGRMWVNTYHDIPAHAPFGGYKKSGLGRETHKSILEAYSQKKNILVSLKEAAHGLF

SEQ ID NO: 414 - protein, 499 aa, Pseudomonas sp.
MQKLDVTLPTYVPDSSYGLFIDNQWVAGESGETLSIINPANGQTLTSIPNGTAADIDRAVQAAQRAFETWRFTSPIERANALLKIADLMEADAERFSVLETLDVGKPIRESSSIDVPLAIDHFRYFAGVIRSHSDEAVMLDEQTLSIVLSEPLGVVGQVIPWNFPLLMAAWKIAPAIAAGNTVVIKPSELTPVTILELAKIIAKVLPAGVVNIVTGTGASAGQALLDHPDVRKLAFTGSTGVGMRVADAAAKKLIPATLELGGKSANIVFPDANWDKAVEGAALAILWNQGQVCESGARLFVHESIYERFLAEVQQKFEAVRVGDPLHRDTMMGAQVSKTQMERILGYIDIAKQEGARVLIGGGRLTGPAYDNGFFIQPTILVDVRNDMRVAYEEIFGPVLCVIPFKDEAEVIAMANDSDYGLAGAVWTQDINRAVRVARAVETGRMWVNTYHDIPAHAPFGGYKKSGLGRETHKSILEAYSQKKNILVSLKEAAHGLF

SEQ ID NO: 415 - protein, 499 aa, Stutzerimonas stutzeri
MQASSDTRANYRPDTSYGLFIDNQWVAGDSGETLDILNPANGDIITRIPNATAVDVDHAVQAAQRAFATWRTTTPTERANALLKIADLMEADAERFSVLETLDVGKPIRESSTVDIPLAIDHFRYFAGVIRSHSDEAVMLDEQTLSIVLSEPLGVVGQVIPWNFPLLMAAWKIAPAIAAGNTVVIKPSELTPISILELAKIFAKVLPAGVVNIITGTGASTGQALLDHPHVRKLAFTGSTGVGHRVADAAAKKLIPATLELGGKSANIVFPDANWDKAVEGAALAILWNQGQVCESGARLFVHESIYERFLAEVKQKFEAVRVGNPLHPDTMMGAQVSKTQMERILGYIDIAKQEGATVLLGGGRLTGPGYDDGFFIQPTILVDVRNDMRVAYEEIFGPVLCVIPFKDEADVIAMANDSEYGLAGAVWTQDINRALRVSRAVETGRIWVNTYHEIPAHAPFGGFKKSGLGRETHKSILEAYTQKKNVYISLNEAPLGLF

SEQ ID NO: 416 - protein, 499 aa, Stutzerimonas nitrititolerans
MQASSDTRANYRPDTSYGLFIDNQWVAGDSGETLDILNPANGDIITRIPNATAVDVDHAVQAAQRAFATWRTTTPTERANALLKIADLMEADAERFSVLETLDVGKPIRESSTVDIPLAIDHFRYFAGVIRSHSDEAVMLDEQTLSIVLSEPLGVVGQVIPWNFPLLMAAWKIAPAIAAGNTVVIKPSELTPISILELAKILAKVLPAGVVNIITGTGASTGQALLDHPHVRKLAFTGSTGVGHRVADAAAKKLIPATLELGGKSANIVFPDANWDKAVEGAALAILWNQGQVCESGARLFVHESIYERFLAEVKQKFEAVRVGNPLHPDTMMGAQVSKTQMERILGYIDIAKQEGATVLLGGGRLTGPGYDDGFFIQPTILVDVRNDMRVAYEEIFGPVLCVIPFKDEADVIAMANDSEYGLAGAVWTQDINRALRVSRAVETGRIWVNTYHEIPAHAPFGGYKKSGLGRETHKSILEAYTQKKNVYISLNEAPLGLF

SEQ ID NO: 417 - protein, 499 aa, Pseudomonas aeruginosa
MQASSDTRANYRPDTSYGLFIDNQWIAGDSGETLDILNPANGDIITRIPNATAVDVDHAVQAAQRAFATWRTTTPIERANALLKIADLMEADAERFSVLETLDVGKPIRESSTVDIPLAIDHFRYFASVIRSHSDEAVMLDEQTLSIVLSEPLGVVGQVIPWNFPLLMAAWKIAPAIAAGNTVVIKPSELTPVSILELAKIFAQVLPAGVVNIITGTGTSAGQALLDHPHVRKLAFTGSTGVGHRVADAAAKKLIPATLELGGKSANIVFPDANWDKAVEGAALAILWNQGQVCESGARLFVHESIYERFLAEVKQKFEAVRVGNPLHPDTMMGAQVSKTQMERILGYIDIAKQEGAMVLLGGGRLTGPGYDDGFFIQPTILVDVRNDMRVAYEEIFGPVLCVIPFKDEADVIAMANDSEYGLAGAVWTQDINRALRVSRAVETGRIWVNTYHEIPAHAPFGGYKKSGLGRETHKSILEAYTQKKNVYISLNEAPLGLF

SEQ ID NO: 418 - protein, 499 aa, Stutzerimonas nitrititolerans
MQASSDTRANYRPDTSYGLFIDNQWVAGDSGETLDILNPANGDIITRIPNATAVDVDHAVQAAQRAFATWRTTTPIERANALLKIADLMEADAERFSVLETLDVGKPIRESSTVDIPLAIDHFRYFAGVIRSHSDEAVMLDEQTLSIVLSEPLGVVGQVIPWNFPLLMAAWKIAPAIAAGNTVVIKPSELTPISILELAKIFAKVLPAGVVNIITGTGASTGQALLDHPHVRKLAFTGSTGVGHRVADAAAKKLIPATLELGGKSANIVFPDANWDKAVEAAALAILWNQGQVCESGARLFVHESIYERFLAEVKQKFEAVRVGNPLHPDTMMGAQVSKTQMERILGYIDIAKQEGATVLLGGGRLTGPGYDDGFFIQPTILVDVRNDMRVAYEEIFGPVLCVIPFKDEADVIAMANDSEYGLAGAVWTQDINRALRVSRAVETGRIWVNTYHEIPAHAPFGGYKKSGLGRETHKSILEAYTQKKNVYISLNEAPLGLF

SEQ ID NO: 419 - protein, 499 aa, Pseudomonadaceae sp.
  source 1..499: note = strain T75
MQASSDTRANYRPDTSYGLFIDNQWVAGDSGETLDILNPANGDIITRIPNATAVDVDHAVQAAQRAFATWRTTTPIERANALLKIADLMEADAERFSVLETLDVGKPIRESSTVDIPLAIDHFRYFAGVIRSHSDEAVMLDEQTLSIVLSEPLGVVGQVIPWNFPLLMAAWKIAPAIAAGNTVVIKPSELTPISILELAKILAKVLPAGVVNIITGTGASTGQALLDHPHVRKLAFTGSTGVGHRVADAAAKKLIPATLELGGKSANIVFPDANWDKAVEGAALAILWNQGQVCESGARLFVHESIYERFLAEVKQKFEAVRVGNPLHPDTMMGAQVSKTQMERILGYIDIAKQEGATVLLGGGRLTGPGYDDGFFIQPTILVDVRNDMRVAYEEIFGPVLCVIPFKDEADVIAMANDSEYGLAGAVWTQDINRALRVSRAVETGRIWVNTYHEIPAHAPFGGYKKSGLGRETHKSILEAYTQKKNVYISLNEAPLGLF

SEQ ID NO: 420 - protein, 499 aa, Stutzerimonas nitrititolerans
MQASSDTRANYRPDTSYGLFIDNQWVAGDSGETLDILNPANGDIITRIPNATAVDVDHAVQAAQRAFATWRTTTPIERANALLKIADLMEADAERFSVLETLDVGKPIRESSTVDIPLAIDHFRYFAGVIRSHSDEAVMLDEQTLSIVLSEPLGVVGQVIPWNFPLLMAAWKIAPAIAAGNTVVIKPSELTPISILELAKIFAKVLPAGVVNIITGTGASTGQALLDHPHVRKLAFTGSTGVGHRVADAAAKKLIPAILELGGKSANIVFPDANWDKAVEGAALAILWNQGQVCESGARLFVHESIYERFLAEVKQKFEAVRVGNPLHPDTMMGAQVSKTQMERILGYIDIAKQEGATVLLGGGRLTGPGYDDGFFIQPTILVDVRNDMRVAYEEIFGPVLCVIPFKDEADVIAMANDSEYGLAGAVWTQDINRALRVSRAVETGRIWVNTYHEIPAHAPFGGYKKSGLGRETHKSILEAYTQKKNVYISLNEAPLGLF

SEQ ID NO: 421 - protein, 499 aa, Pseudomonas urethralis
MQASNDIRKNYRPDSSYGLFIDNQWVGSDSGETLDILNPANGEVITQIPNANAIDINRAVQAAQRAFETWRATTPIERANALLKIADLMEADAERFSVLETLDVGKPIRESTTVDIPLAIDHFRYFAGVIRSHSDEAVMLDEQTLSIVLSEPLGVVGQVIPWNFPLLMAAWKIAPAIAAGNTVVIKPSELTPVSILELAKIFAQVLPAGVVNIVTGTGASAGQALLDHPNVSKLAFTGSTGVGQRVADAAAKKLIPATLELGGKSANIVFPDANWDKAVEGAALAILWNQGQVCESGARLFVHESIYERFLAEVKQKFEAVRVGNPLHPDTMMGAQVSKTQMERILGYIDIARQEGATVLLGGGRLTGPDYDNGFFIQPTILVDVRNDMRVAYEEIFGPVLCVIPFKDEAEVIAMANDSEYGLAGAVWTQDINRALRVSRAVETGRMWVNTYHEIPAHAPFGGYKKSGLGRETHKSILEAYTQKKNVYISLNEAPLGLF

SEQ ID NO: 422 - protein, 499 aa, Pseudomonas sp.
MQASNDIRKNYRPDSSYGLFIDNQWVGSDSGETLDILNPANGEVITQIPNANAIDINRAVQAAQRAFETWRATTPIERANALLKIADLMEADAERFSILETLDVGKPIRESTTVDIPLAIDHFRYFAGVIRSHSDEAVMLDEQTLSIVLSEPLGVVGQVIPWNFPLLMAAWKIAPAIAAGNTVVIKPSELTPVSILELAKIFAQVLPAGVVNIVTGTGASAGQALLDHPNVSKLAFTGSTGVGQRVADAAAKKLIPATLELGGKSANIVFPDANWDKAVEGAALAILWNQGQVCESGARLFVHESIYERFLAEVKQKFEAVRVGNPLHPDTMMGAQVSKTQMERILGYIDIARQEGATVLLGGGRLTGPDYDNGFFIQPTILVDVRNDMRVAYEEIFGPVLCVIPFKDEAEVIAMANDSEYGLAGAVWTQDINRALRVSRAVETGRMWVNTYHEIPAHAPFGGYKKSGLGRETHKSILEAYTQKKNVYISLNEAPLGLF

SEQ ID NO: 423 - protein, 499 aa, Pseudomonas sp.
MQASNDIRKNYRPDTSYGLFIDNQWVGSDSGETLDILNPANGEVITQIPNANAIDINRAVQAAQRAFETWRATTPIERANALLKIADLMEADAERFSVLETLDVGKPIRESTTVDIPLAIDHFRYFAGVIRSHSDEAVMLDEQTLSIVLSEPLGVVGQVIPWNFPLLMAAWKIAPAIAAGNTVVIKPSELTPVSILELAKIFAQVLPAGVVNIVTGTGASAGQALLDHPNVSKLAFTGSTGVGQRVADAAAKKLIPATLELGGKSANIVFPDANWDKAVEGAALAILWNQGQVCESGARLFVHESIYERFLAEVKQKFEAVRVGNPLHPDTMMGAQVSKTQMERILGYIDIARQEGATVLLGGGRLTGPDYDNGFFIQPTILVDVRNDMRVAYEEIFGPVLCVIPFKDEAEVIAMANDSEYGLAGAVWTQDINRALRVSRAVETGRMWVNTYHEIPAHAPFGGYKKSGLGRETHKSILEAYTQKKNVYISLNEAPLGLF

SEQ ID NO: 424 - protein, 499 aa, Stutzerimonas nitrititolerans
MQASSDTRANYRPDTSYGPFIDNQWVAGDSGETLDILNPANGDIITRIPNATAVDVDHAVQAAQRAFATWRTTTPIERANALLKIADLMEADAERFSVLETLDVGKPIRESSTVDIPLAIDHFRYFAGVIRSHSDEAEMLDEQTLSIVLSEPLGVVGQVIPWNFPLLMAAWKIAPAIAAGNTVVIKPSELTPISILELAKIFAKVLPAGVVNIITGTGASTGQALLDHPHVRKLAFTGSTGVGHRVADAAAKKLIPATLELGGKSANIVFPDANWDKAVEAAALAILWNQGQVCESGARLFVHESIYKRFLAEVKQKFEAVRVGNPLHPDTMMGAQVSKTQMERILGYIDIAKQEGATVLLGGGRLTGPGYDDGFFIQPTILVDVRNDMRVAYEEIFGPVLCVIPFKDEADVIAMANDSEYGLAGAVWTQDINRALRVSRAVETGRIWVNTYHEIPAHAPFGGYKKSGLGRETHKSILEAYTQKKNVYISLNEAPLGLF

SEQ ID NO: 425 - protein, 499 aa, Pseudomonas asiatica
MQATNDIRKNYRPDTSYGLFIDNQWVGSDSGETLDILNPANGEVITQIPNANAIDINRAVQAAQRAFETWRATTPIERANALLKIADLMEADAERFSVLETLDVGKPIRESTTVDIPLAIDHFRYFAGVIRSHSDEAVMLDEQTLSIVLSEPLGVVGQVIPWNFPLLMAAWKIAPAIAAGNTVVIKPSELTPVSILELAKIFAQVLPAGVVNIVTGTGASAGQALLDHPNVSKLAFTGSTGVGQRVADAAAKKLIPATLELGGKSANIVFPDANWDKAVEGAALAILWNQGQVCESGARLFVHESIYERFLAEVKQKFEAVRVGNPLHPDTMMGAQVSKTQMERILGYIDIARQEGATVLLGGGRLTGPDYDNGFFIQPTILVDVRNDMRVAYEEIFGPVLCVIPFKDEAEVIAMANDSEYGLAGAVWTQDINRALRVSRAVETGRMWVNTYHEIPAHAPFGGYKKSGLGRETHKSILEAYTQKKNVYISLNEAPLGLF

SEQ ID NO: 426 - protein, 499 aa, Pseudomonas sp.
  source 1..499: note = strain ACN8
MQILHDVPPSYVPDQSYGLFIDNQWVEGESGETIDSRNPANGKFLTRIPNANAADVDRAVQAANRAFQTWRTTTPIERSNLLLKIADLLERDFERFAILESLDVGKPLRESKAIDVPLAIDHFRYFAGVIRSHSDEAVMLDEQTLSITLSEPLGVVGQVIPWNFPLLMAAWKIAPAIAMGNTVVIKPSEMTPVTILELAKIFAKVLPPGVVNIVTGTGVTTGQPLLDHPLIRKLAFTGSTGIGGVVAAAAAKKIIPATLELGGKSANIVFPDANWEKAVEGAALAILWNQGQVCESGARLFVHESIYERFLGELKQKFEQVRVGDPLSPETTMGAQVSETQMNRILGYIDIALTEGANVLVGGERLTGGDYDAGFFIQPTILVDVRNDMRVAYEEIFGPVLCVIPFKDEAEVIALANDSEYGLAGAVWTQDINRALRVSRAVETGRMWVNTYHEIPAHAPFGGYKKSGIGRETHKSMLEAYSQKKNIYVSLNEAPLGLF

SEQ ID NO: 427 - protein, 499 aa, Cupriavidus basilensis
MQTLKDIPPSYVPDRSYGLFIDNQWVDGATGETLDSINPANGEFLTRIPNASAEDVDRAVKAATRAFRTWRTTTAIERSTALLKIADLLEADAERFATLETLDVGKPIRESRAIDVPLAIDHFRYFAGVIRSHSDEAVMLDEKTLSIVLSEPLGVVGQVIPWNFPLLMAAWKIAPAIAMGNTVVIKPSEMTPVTILELAKIFAKVLPAGVVNIVTGTGVSAGQPLLDHPDIRKLAFTGSTRIGEAVAVAAAKKIIPATLELGGKSANIVFPDANWDKAVEGAALAILWNQGQVCESGARLFVHASIYERFLEELKEKFRAVRVGDPMSPETQMGAQVSKTQMERILGYIDIAQAEGARVLVGGGRLVGGDHDAGYFIQPTILVGVRNDMRVAYEEIFGPVLCVIPFKDEAEVIAMANDSEYGLAGGVWTQDLNRALRVARAVETGRMWVNTYHDIPAHAPFGGYKKSGLGRETHKSMLEAYSQKKNILVSLGETPHGLF

SEQ ID NO: 428 - protein, 499 aa, Cupriavidus sp.
  source 1..499: note = strain WS
MQTLKDIPPSYVPDRSYGLFIDNQWVDGATGETLDSINPANGEFLTRIPNASAEDVDRAVKAATRAFRTWRTTTAIERSTALLKIADLLEADAERFATLETLDVGKPIRESRAIDVPLAIDHFRYFAGVIRSHSDEAVMLDEKTLSIVLSEPLGVVGQVIPWNFPLLMAAWKIAPAIAMGNTVVIKPSEMTPVTILELAKIFAKVLPAGVVNIVTGTGVSAGQPLLDHPDIGKLAFTGSTRIGEAVAVAAAKKIIPATLELGGKSANIVFPDANWDKAVEGAALAILWNQGQVCESGARLFVHASIYERFLEELKEKFRAVRVGDPMSPETQMGAQVSKTQMERILGYIDIAQAEGARVLVGGGRLVGGDHDAGYFIQPTILVDVRNDMRVAYEEIFGPVLCVIPFKDEAEVIAMANDSEYGLAGGVWTQDLNRALRVARAVETGRMWVNTYHDIPAHAPFGGYKKSGLGRETHKSMLEAYSQKKNILVSLSETPHGLF

SEQ ID NO: 429 - protein, 499 aa, Cupriavidus sp.
  source 1..499: note = strain BIC8F
MQSLKDISSSYVPDSSYGLFIDNQWVEGGSGETIESINPANGEFLTHIPNASAEDVDRAVKAATRAFRTWRTTTAIERSNALLKIADLLEADAERFATLETLDVGKPIRETRAIDVPLAIDHFRYFAGVIRSHSDEAVMLDEKTLSIVLSEPLGVVGQVIPWNFPLLMAAWKIAPAIAMGNTVVIKPSEMTPVTILELAKIFAQVLPPGVVNIVTGTGVSAGQPLLDHPGIRKLAFTGSTRIGEVVAAAAAKKIIPATLELGGKSANIVFPDANWDKAVEGAALAILWNQGQVCESGARLFVHESIYERFLGELKEKFQAVRVGDPLSSETQMGAQVSKTQMERILGYIDIAQTEGARVLTGGGRLVGGDYDAGYFIEPTILVDVRNDMRVAYEEIFGPVLCVIPFKDEAEVIAMANDSEYGLAGGIWTQDINRALRVARAVETGRMWVNTYHDIPAHAPFGGYKKSGLGRETHKSMLEAYSQKKNILVSLNEATLGLF

SEQ ID NO: 430 - protein, 494 aa, Rhodococcus qingshengii
MESLEFGPDRSYGLFIDNQWVDGESGDTIESVNPATGEFLTRIPNATAADVDRAVQAANRAFGTWRTTTASERANALLKIADLLEADAERFAVLETLDVGKPIREITAIDVPLAIDHFRYFAGVIRSHSDEAVMLDDQTLSIVLSEPIGVVGQVIPWNFPLLMAAWKIAPAIAAGNTLVIKPSEMTPITILELAKIFAQVLPPGVVNIVTGTGVSAGQPLLEHPLVRKLAFTGSTRVGEVVASVAASKIIPATLELGGKSANIVFPDANWDKAVEGAALAILWNQGQVCESGARLFVHESIYERFLGELKEKFEAARVGDPLSPETQIGSQISKTQVDRVLGYIDLAKEEGARILTGGGRLNGENYDGGFFVEPTIIVDVRNDMRVASEEIFGPVLCVIPFKDEAEVIALANDSEYGLAGAVWTQDINRALRVSRAVETGRMWVNTYHDIPAHAPFGGYKKSGLGRETHKSMLDAYTQKKNIYVSLDEAPLGVF

SEQ ID NO: 431 - protein, 494 aa, Rhodococcus ruber
MESLEYSPDRSYGLFIDNQWVDGESGDTIESVNPATGEFLTRIPNATAADVDRAVQSAHRAFGAWRATTAAERAHALLKIADLLEANAERFAVLETLDVGKPIREIHAIDVPIAIDHFRYFAGVIRSHSDEAVMLDDRTLSIVLSEPLGVVGQVIPWNFPLLMAAWKIAPAIASGNTLVIKPSEMTPITILELAKILAQVLPPGVVNIVTGTGASAGQPLLEHPLVRKLAFTGSTRVGEVVASVAANKIIPATLELGGKSANIVFPDANWDKAVEGAALAILWNQGQVCESGARLFVHESIYERFLGELKEKFETARVGDPLSPETQIGSQISKTQLDRVLGYIDIAKEEGARVLTGGGRLNGEKYDGGFFIEPTILVDVRNDMRVASEEIFGPVLCVIPFKDEAEVIALANDSEYGLAGAVWTQDINRALRVSRAVETGRMWVNTYHEIPAHAPFGGYKKSGLGRETHKSMLDAYTQKKNIYVSLDEAPLGLF

SEQ ID NO: 432 - protein, 831 aa, Stutzerimonas nitrititolerans
MQASSDTRANYRPDTSYGLFIDNQWVAGDSGETLDILNPANGDIITRIPNATAVDVDHAVQAAQRAFATWRTTTPIERANALLKIADLMEADAERFSVLETLDVGKPIRESSTVDIPLAIDHFRYFAGVIRSHSDEAVMLDEQTLSIVLSEPLGVVGQVIPWNFPLLMAAWKIAPAIAAGNTVVIKPSELTPISILELAKIFAKVLPAGVVNIITGTGASTGQALLDHPHVRKLAFTGSTGVGHRVADAAAKKLIPATLELGGKSANIVFPDANWDKAVEAAALAILWNQGQVCESGARLFVHESIYERFLAEVKQKFEAVRVGNPLHPDTMMGAQVSKPQMERILGYINIAKQEGATVLLGGGRLTGPGYDDGFFIQPTILVDVRNDMRVAYEEIFGPVLCVIPFKDEADVIAMANDSEYGLAGAVWTQDINRALRVSRAVETGRIWVNIDMQVNEMNQPALFKTTQLGPYTLKNRIVLPPLTRSRSSQPGNIPNDLMATYYRQRTGAGFMVTEGTQIEPRGQGYAWTPGIHSAEQVEGWRKVTNAVHAEGGVIFAQLWHVGRVSHTTLQPNGEAPVAPSAIRADSVKVFIETGPGTGALAEPSTPRALSTAEVKELVQLYAKAARNALDAGFDGVEIHCANGYLVNQFISAHTNLRDDEYGGSLHNRLRFLREVTEAVAGVVGKERLGVRFAPLFATTDEDRVYLGLVEDDPHQTYIEAVKVLERVGIAYLSLAEADWENAPELPETFREDVRKTFSGKILYAGKYTAERGNRVIQAGWGDLIAFGRPFIANPDLPERIANGWPLNPVDPASMYGGTDKGYTDYPTYRL

SEQ ID NO: 433 - protein, 526 aa, Marinobacterium profundum
MNIWILTNFNIANSITSLLAQPTVEAHMKHLEDIPSAFRPDTSYGLFIDNQWVEGQSGETIDNLNPATGEFLTRIPNATAADVDRAVQAASRAFNTWRTSTPTERSNALLKIADLLEAEAERFAVLETLDVGKPIRESRAIDVPMAIDHFRYFAGAIRSHSDEAVMLDELTLSIALSEPLGVIGQVIPWNFPLLMAAWKLAPAIATGNTVVIKPSEMTPITIFELAKLIAKVLPPGVVNIITGVGAVAGQALLDHPHVRKLAFTGSTRVGEAVAAAAAKKIIPATLELGGKSANIVFADANWEKAVEGAALAILWNQGQVCESGARLFVHESIFERFVGQLKKKFENVRIGDPLDSETMMGAQISKVQMDRILGYIDIAKKEGARVLTGGGRIAGGQYDAGFYLEPTILVDVRNDMRVAYEEIFGPVLCVIPFKNEAEVIAMANDSEYGLAGAVWTQDINRALRVARAVETGRIWVNTYHEIPAHAPFGGYKKSGLGRETHKSMLEAYSQKKNVFISLGEAPLGLF

SEQ ID NO: 434 - protein, 481 aa, Mycobacteriaceae sp.
MFIDNQWVDAESGETIESINPATGEVLTRIPSGTAADVDRAVQAAQRAFAGWRATTVTERANALNRIADLLEADAERFAVLESLDVGKPIRESRVLDVPIAIDHFRYFAGVIRSHSDEAAVLDEQTLSLVLSEPLGVVGQVIPWNFPLLMAAWKIAPAIATGNTVVIKPSEMTPVSISELAKIFAQVLPPGVVNIVTGTGPVAGQAVLDHPTVQKLAFTGSTRVGYTVAEAAAKRLVPATLELGGKSANIVFPDANWDKAVEGAALAILWNQGQACESGARLFIHESIYDRFIDELKAKFTTARVGDPLSPDTQVGSLISKAQLERVLGYVETGRAEGATVLTGGRRLTGGDYDSGFFVEPTILVDVRNDMRVASEEIFGPVLVVIPFKDEDEVIALANDSEYGLAGTVWTQDINRALRVARAVETGRMWVNTAHEIPAHAPFGGYKKSGLGREAYKSMLDAYTHKKNIYVSMNEAPRGLF

(19) United States
(12) Patent Application Publication
(10) Pub. No.: US 2026/0357306 A1
(43) Pub. Date: Nov. 12, 2026

(54) ONE-POT FERMENTATIVE PRODUCTION OF GLYCOLATE OR OTHER HYDROXYCARBOXYLIC ACIDS

(71) Applicant: PHYCUS BIOTECHNOLOGIES INC., Markham (CA)

(72) Inventors: Christian EULER, Markham (CA); Aditya Vikram PANDIT, Markham (CA); Jonathan ALEXIOU, Markham (CA)

(21) Appl. No.: 19/489,094

(22) PCT Filed: Jun. 1, 2024
(86) PCT No.: PCT/CA2024/050738

§ 371 Date: Dec. 1, 2025

Related U.S. Application Data
(60) Provisional application No. 63/505,864, filed on Jun. 2, 2023.

(51) Int. Cl. C12P 7/42

(52) U.S. Cl. CPC C12P 7/42

(57) ABSTRACT

A one-pot fermentation process for producing a glycolic acid (or other hydroxycarboxylic acids) from ethylene glycol (or other suitable aliphatic polyhydric alcohol having a terminal hydroxyl group) is described herein. The process general comprises providing a microorganism expressing one or more biosynthetic pathway enzymes under control of an inducible expression cassette such that expression of the one or more of the biosynthetic pathway enzymes is “off” at the start of the growth phase and is turned “on” upon the microorganism reaching a target biomass concentration suitable for production. In some embodiments, the one or more biosynthetic pathway enzymes are placed under control of a nutrient-sensing promoter and the starting fermentation broth contains a limiting concentration of the nutrient such that deletion of the nutrient both induces expression of the biosynthetic pathway enzymes and governs the transition from the growth phase to the production phase.

The present description relates to a one-pot fermentative process for the production of glycolic acid or other hydroxycarboxylic acids, from a non-native feedstock such as ethylene glycol or other aliphatic polyhydric alcohol having a terminal hydroxyl group, as well as microorganisms relating thereto. All documents referenced herein are incorporated by reference in their entirety.

### BACKGROUND

Hydroxycarboxylic acids represent commercially valuable molecules useful as raw materials for ingredients in a variety of industrial and/or household products. For example, glycolic acid is used in the manufacture of biodegradable polymers such as polyglycolic acid, polylactic-glycolic acid, and other degradable polymers, as well as an ingredient in a number of industrial and household products such as solvents, paints, and particularly cosmetics. Currently, commercial production of glycolic acid involves petrochemical feedstocks and/or employs toxic starting materials such as formaldehyde. Less toxic, bio-based approaches to produce glycolic acid from renewable feedstocks have thus been explored, including fermentation approaches. However, processes for the fermentative production of glycolic acid or other hydroxycarboxylic acids that have been reported generally employ multi-pot fermentation strategies in which biomass is first accumulated to a high density, then separated from the growth medium, washed, and subsequently used as a catalyst in a biotransformation step to produce the product of interest. Examples of such multi-pot fermentation strategies for the production of glycolic acid from ethylene glycol include those described in Kataoka et al., 2001, Wada et al., 2005, Morishige et al., 2007, and Hua et al., 2018. Although some eventually reached reasonable glycolic acid titers, these multi-pot processes are highly intensive, require significant operator intervention, and/or employ long fermentation times, which all greatly hamper their commercial viability at industrial scales. Thus, there is a demand for a one-pot fermentation approach to produce glycolic acid or other hydroxycarboxylic acids from a renewable feedstock at industrial scales.

### SUMMARY

In a first aspect, described herein is a one-pot fermentation process for producing a hydroxycarboxylic acid (e.g., glycolic acid), as a product or intermediate, from a substrate that is an aliphatic polyhydric alcohol having a terminal hydroxyl group (e.g., ethylene glycol). The process generally comprises providing a microorganism that expresses biosynthetic pathway enzymes enabling or facilitating the production of the hydroxycarboxylic acid from the substrate. In some embodiments, the microorganism comprises: a first polynucleotide encoding a first enzyme which is a zinc-dependent alcohol dehydrogenase or other oxygen-insensitive oxidoreductase, that catalyzes the conversion of the aliphatic polyhydric alcohol substrate to its corresponding aldehyde; and a second polynucleotide encoding a second enzyme which is an aldehyde dehydrogenase that catalyzes the conversion of the corresponding aldehyde to a corresponding alpha-hydroxy acid. The one-pot fermentation process described herein comprises a growth phase and a production phase. The growth phase comprises culturing the microorganism for biomass accumulation in a fermentation broth comprising a native carbon source and under conditions of excess oxygen. The production phase comprises culturing the microorganism for hydroxycarboxylic acid production in the fermentation broth in the presence of the aliphatic polyhydric alcohol substrate and under conditions of excess oxygen. In some embodiments, at least the first polynucleotide encoding the first enzyme may be comprised in an inducible expression cassette that is uninduced at the start of the growth phase and that becomes induced to initiate transition from the growth phase to the production phase upon the microorganism reaching a target biomass concentration suitable for hydroxycarboxylic acid production. In some embodiments, the inducible expression cassette may comprise a nutrient-sensing control element (e.g., a nutrient-sensing promoter) that turns “on” transcription of the expression cassette upon depletion of the nutrient in the fermentation broth below an expression threshold, and wherein the initial concentration of said nutrient in the fermentation broth prior to the start of the growth phase is tuned to achieve the target biomass concentration at which transcription from the expression cassette is turned “on,” thereby initiating transition from the growth phase to the production phase. In some embodiments, the microorganism's endogenous glycolate oxidase activity is impaired or inactivated.

In a further aspect, described herein is a process for reducing the production of by-products of overflow metabolism in a one-pot fermentation process. The process may comprise providing a microorganism engineered to express a biosynthetic pathway comprising multiple exogenous enzymes to produce a product of interest from a non-native feedstock, wherein expression of each of the biosynthetic pathway is controlled by a nutrient-sensing control element that turns on transcription of the multiple enzymes upon depletion of the nutrient below an expression threshold. The process further comprises a one-pot fermentation process comprising a growth phase and a production phase. The growth phase comprises culturing the microorganism for biomass accumulation in a fermentation broth comprising a native carbon source and under conditions of excess oxygen. The production phase comprises culturing the microorganism for production of the product of interest in the fermentation broth in the presence of the non-native feedstock and under conditions of excess oxygen, wherein the initial concentration of said nutrient in the fermentation broth prior to the start of the growth phase is set to achieve a target biomass concentration at which nutrient depletion below the expression threshold turns on transcription of the biosynthetic pathway, thereby producing a delay in the expression of the biosynthetic pathway that reduces metabolic burden of the microorganism during the growth phase and a reduction in the production of by-products of overflow metabolism in the production phase.

In a further aspect, microorganisms engineered for implementing the above processes are also described herein.

### GENERAL DEFINITIONS

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one,” but it is also consistent with the meaning of “one or more”, “at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

FIG. 1 is schematic diagram of the orthogonality of the ethylene glycol (EG) to glycolic acid (GA) pathway. EG is not a native carbon source for E. coli, so its consumption pathway (the ‘Production Module’) does not overlap significantly with native metabolism. GA enters native metabolism (the ‘Growth “Module”’) via the product of the glcD gene (dashed line) and only via this route. Deletion or impairment of the glcD gene, or otherwise reducing activity of its encoded enzyme, is therefore sufficient to ensure accumulation of GA in cells capable of transforming EG. If the glcD gene is not deleted, impaired or its encoded enzyme's activity is not reduced, GA may be used to generate biomass.

FIG. 2 shows a plasmid map schematic in which the phoA promoter (PphoA) drives expression of the pathway enzymes, Gox0313 and AldA, in a synthetic operon. The translation initiation rate (TIR) of each of these genes is controlled by a custom ribosome binding sequence (RBS). The plasmid also bears a kanamycin resistance cassette (KanR) to ensure that it is propagated.

FIG. 3 shows flask characterization of the phoA system for glycolic acid production. Cells harbouring either the constitutively expressed (‘const.’) or phoA promoter-driven (‘phoA’) pathway were grown in phosphate-limited media on glucose supplemented with ethylene glycol. Biomass (solid lines), glycolic acid (double lines), and acetate (dashed lines) were measured over the course of the 24-h fermentation period. Biomass is reported as optical density at 600 nm (‘OD’; arbitrary units) on the left axis and concentrations in g/L on the right axis. Error represents±SD of biological replicates (n=3).

FIG. 4 shows acetate yield (gram acetate per gram cell dry weight; g/gCDW) for flask-scale glycolic acid production fermentation. Cells harbouring the phoA system (‘phoA Promoter’) produced significantly less acetate than those with the constitutive expression system (‘Constitutive’). In the negative control fermentation, cells did not carry any non-native production pathways, and therefore produced negligible amounts of acetate.

FIG. 5 exemplifies the non-trivial nature of selecting initial phosphate concentrations at fermentation scales of 100 L or greater. The bar graph the variability of biomass yield on phosphate concentration (Y_{OD/PO4}) over different scales and growth rates. Fermentation “F229” was grown at a growth rate lower than fermentations “F233” and “F233a,” while fermentations “F233” and “F233a” had approximately the same growth rate, but were at different reactor scales (100 L vs. 1000 L).

FIG. 6 shows the relationship between glycolic acid titer and production phase duration for the two different fermentation modes over many tested scales (50 mL-1000 L). In the constitutive system (‘Const.’), glycolic acid titer was not significantly correlated with the duration of production phase (R2=0.02), whereas in the phoA promoter-driven system (‘phoA promoter’), titer was strongly coupled to the duration of production phase (R²=0.86). In the phoA system, extending the production phase duration resulted in increased titer for all production phase durations tested. The dashed line at 60 g/L represents an arbitrarily set threshold for commercial viability.

FIG. 7 shows a schematic representation of the oxidation of ethylene glycol to glycolic acid in a two-step enzymatic process.

FIG. 8A illustrates a multiple sequence alignment between the enzymes goxADH, gmoADH, and gdiADH (SEQ ID NOs: 206-208), with bolded residues indicating positions of potential engineering. FIG. 8B illustrates a multiple sequence alignment between the enzymes goxADH, gmoADH, gdiADH, mtaADH, and mmaADH (SEQ ID NOs: 206-209 and 215).

FIG. 9 illustrates a multiple sequence alignment between the enzyme gmoADH (SEQ ID NO: 207) and orthologs thereof sharing at least 85% amino acid sequence identity (SEQ ID NOs: 216-247).

FIG. 10 shows a phylogenetic tree constructed with respect to the multiple sequence alignment of FIG. 9.

FIG. 11 illustrates a multiple sequence alignment between the enzyme gdiADH (SEQ ID NO: 208) and orthologs thereof sharing at least 85% amino acid sequence identity (SEQ ID NOs: 248-264).

FIG. 12 shows a phylogenetic tree constructed with respect to the multiple sequence alignment of FIG. 11.

FIG. 13 illustrates a multiple sequence alignment between the enzyme aviALDH (SEQ ID NO: 266) and orthologs thereof sharing at least 85% amino acid sequence identity (SEQ ID NOs: 268-366).

FIG. 14 illustrates a multiple sequence alignment between the enzyme ppuALDH (SEQ ID NO: 267) and orthologs thereof sharing at least 85% amino acid sequence identity (SEQ ID NOs: 367-434).

### SEQUENCE LISTING

This application contains a Sequence Listing in computer readable form created May 31, 2024. The computer readable form is incorporated herein by reference.

| SEQ ID NO: | Description |
|---|---|
| 1 | Gox0313 polypeptide sequence |
| 2 | AldA polypeptide sequence |
| 3 | Phosphate promoter polynucleotide sequence |
| 4 | RBS polynucleotide sequence upstream of the |
| | aldA gene |
| 5 | RBS polynucleotide sequence upstream of the |
| | gox gene |
| 6 | GlcD inactivation forward primer |
| 7 | GlcD inactivation reverse primer |
| 8-205 | Alcohol dehydrogenases sharing at least 70% |
| | overall identity at the amino acid level with the |
| | Gox0313 polypeptide sequence of SEQ ID NO: 1. |
| 206 | goxADH polypeptide sequence |
| 207 | gmoADH polypeptide sequence |
| 208 | gdiADH polypeptide sequence |
| 209 | mtaADH polypeptide sequence |
| 210 | ecoADHP polypeptide sequence |
| 211 | gstADH polypeptide sequence |
| 212 | scoADH polypeptide sequence |
| 213 | cpsADH polypeptide sequence |
| 214 | rjoADH polypeptide sequence |
| 215 | mmaADH polypeptide sequence |
| 216-247 | Orthologs of gmoADH polypeptide sequences |
| 248-264 | Orthologs of gdiADH polypeptide sequences |
| 265 | ecoALDH polypeptide sequence |
| 266 | aviALDH polypeptide sequence |
| 267 | ppuALDH polypeptide sequence |
| 268-366 | Orthologs of aviALDH polypeptide sequences |
| 367-434 | Orthologs of ppuALDH polypeptide sequences |

### DETAILED DESCRIPTION

Described herein is a one-pot fermentation process for producing a fermentative product of interest, as well as microorganisms engineered for same. As used herein, the expression “one-pot fermentation” refers to the production of a product of interest from the culture of an engineered microbial host in a single fermentation vessel, not requiring separation of accumulated biomass from fermentation broth. In general, the process comprises providing a microorganism engineered to express or overexpress one or more enzymes of a biosynthetic pathway to consume and convert a non-native feedstock to a product of interest, wherein one or more of the enzymes of the biosynthetic pathway is expressed under the control of an inducible expression cassette that is uninduced at the start of fermentation. In a growth phase, the microorganism is cultured in a fermentation broth comprising a native carbon source and under conditions favoring biomass accumulation (e.g., excess oxygen) until a target biomass concentration suitable for production of the product of interest is reached. Towards the conclusion of the growth phase, expression of one or more of the enzymes of the biosynthetic pathway from the inducible expression cassette is initiated, thereby governing the transition from the growth phase to a production phase. In the production phase, the microorganism is further cultured in the fermentation broth in the presence of a non-native feedstock, which is converted by the microorganism to the product of interest by the one or more enzymes of the biosynthetic pathway. In some embodiments, the resulting temporal delay in expression of the one or more enzymes of the biosynthetic pathway (e.g., because nutrient sensing promoter does not induce expression) during the growth phase, results in an increase in productivity of the product of interest, reduced metabolic burden for the microorganism, and a decrease in the production of undesirable by-products of overflow metabolism, in the production phase.

As used herein, the expression “native carbon source” or “native feedstock” refers to a biological material that can be readily utilized for growth and/or energy by a corresponding wild-type microorganism. Conversely, as used herein, the expression “non-native carbon source” or “non-native feedstock” refers to a biological material that cannot be readily utilized for growth and/or energy by a corresponding wild-type microorganism.

In a first aspect, described herein is a one-pot fermentation process for the production of a hydroxycarboxylic acid, as a product or an intermediate, from a substrate that is an aliphatic polyhydric alcohol having a terminal hydroxyl group. In some embodiments, the process comprises providing a microorganism comprising a first polynucleotide encoding a first enzyme which is a zinc-dependent alcohol dehydrogenase or other oxygen-insensitive oxidoreductase, that catalyzes the conversion of the aliphatic polyhydric alcohol substrate to its corresponding aldehyde; and a second polynucleotide encoding a second enzyme which is an aldehyde dehydrogenase that catalyzes the conversion of the corresponding aldehyde to a corresponding alpha-hydroxy acid. In some embodiments, the microorganism employed in the one-pot fermentation process described herein is one in which endogenous glycolate oxidase activity is impaired or inactivated. In some embodiments, the microorganism's endogenous glycolate oxidase (or a subunit thereof required for catalytic activity) is deleted, truncated, or mutated to impair or abrogate glycolate oxidase activity. In some embodiments, expression of the microorganism's endogenous glycolate oxidase (or a subunit thereof required for catalytic activity) may be inhibited, silenced, knocked-down, or repressed. In some embodiments, the microorganism described herein comprises an inactivated or impaired glcD gene encoding a glycolate oxidase subunit GlcD. In some embodiments, the activity of the microorganism's endogenous glycolate oxidase may be reduced, for example, by decreasing oxygen in the production phase as described in Pandit et al., 2019 and Pandit et al., 2021.

The one-pot fermentation process described herein further comprises a growth phase and a production phase. The growth phase comprises culturing the microorganism for biomass accumulation in a fermentation broth comprising a native carbon source (e.g., glucose) and under conditions of excess oxygen. The production phase comprises further culturing the microorganism for hydroxycarboxylic acid production in the fermentation broth in the presence of the aliphatic polyhydric alcohol substrate and under conditions of excess oxygen. In some embodiments, at least the first polynucleotide encoding the first enzyme is comprised in an inducible expression cassette that is uninduced (e.g., transcription is ‘off’) at the start of the growth phase and that becomes induced (e.g., transcription is turned ‘on’) to initiate transition from the growth phase to the production phase upon the microorganism reaching a target biomass concentration suitable for hydroxycarboxylic acid production.

In some embodiments, the inducible expression cassette may comprise a nutrient-sensing control element that turns on transcription of the expression cassette upon depletion of the nutrient in the fermentation broth below an expression threshold, and wherein the initial concentration of said nutrient in the fermentation broth prior to the start of the growth phase is set or tuned to achieve the target biomass concentration at which transcription from the expression cassette is turned on, thereby governing, controlling, or initiating transition from the growth phase to the production phase in the one-pot fermentation process described herein. In some embodiments, the nutrient-sensing control element may comprise or consist of a nutrient-sensing promoter, such as a phosphorous-sensing (e.g., phosphate-sensing), sulfur-sensing (e.g. sulfate-sensing), nitrogen-sensing, oxygen-sensing, or magnesium-sensing promoter. In some embodiments, the nutrient-sensing promoter is a promoter that is repressed at high (or replete) nutrient concentrations and is activated when nutrient levels decrease to below a threshold nutrient level. In some embodiments, the nutrient-sensing promoter is the phoA promoter of the E. coli alkaline phosphatase gene (phoA), which is repressed by high or replete phosphate concentrations. In some embodiments, the nutrient-sensing promoter is a phosphorous-sensing promoter (e.g., phosphate-sensing promoter) and the concentration of phosphorous or phosphate at the start of the growth phase (or at the start of fermentation) is less than 50, 45, 40, 35, 30, 25, 20, 15, or 10 mM. In some embodiments, the one-pot fermentation process described herein is carried out at an industrial scale, such as in at least a 50-, 100-, 150-, 200-, 250-, 500-, 750-, or 1000-L fermenter. In some embodiments, the concentration of phosphorous or phosphate at the start of the growth phase (or at the start of fermentation) is set based on set based on

In some embodiments, the expression cassette may be a polycistronic expression cassette comprising at least the first and second polynucleotides (e.g., to be expressed from a single mRNA transcript). In some embodiments, at least one of the first and second polynucleotides is heterologous or exogenous with respect to the microorganism. In some embodiments, the expression cassette may be comprised in an expression vector (e.g., plasmid, cosmid, phage, or virus), or may be comprised in the genome of the microorganism.

In some embodiments, the first enzyme may be a medium-chain zinc-dependent alcohol dehydrogenase (e.g., Gox0313; Zhang et al., 2015; SEQ ID NO: 1) or a functional fragment thereof. In some embodiments, the first enzyme may be a polypeptide having zinc-dependent alcohol dehydrogenase activity comprising an amino acid sequence at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ ID NO: 1. Examples of polypeptides sharing at least 70% overall identity to SEQ ID NO: 1 are shown in SEQ ID NOs: 6-203. In some embodiments, the first enzyme may be a polypeptide having zinc-dependent alcohol dehydrogenase activity comprising an amino acid sequence at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequences of any one of SEQ ID NOs: 8 to 205. In some embodiments, the first enzyme may a variant of a Gluconobacter oxydans alcohol dehydrogenase, wherein the Gluconobacter oxydans alcohol dehydrogenase is encoded by the Gluconobacter oxydans Gox0313 gene. In some embodiments, the first enzyme may be a medium-chain zinc-dependent alcohol dehydrogenase comprising: (a) a catalytic zinc binding comprising residues corresponding to C38, H61, and C149 of SEQ ID NO: 1; and/or (b) a structural zinc binding site comprising residues corresponding to C93, C96, C99, and C107 of SEQ ID NO: 1. As used herein, the expression “comprising residues corresponding to” refers to the presence of the same amino acid residue at the same amino acid position in two or more structurally related polypeptides, when the two or more polypeptides are subjected to a multiple sequence alignment.

In some embodiments, the first enzyme may be an aldehyde dehydrogenase such as a lactaldehyde reductase (e.g., encoded by the gene fucO) or a functional variant thereof having reduced sensitivity to oxygen (e.g., reduced sensitivity to metal catalyzed oxidation). In some embodiments, the lactaldehyde reductase may include an amino acid substitution I7L and/or L8V or L8M, based on the amino acid numbering of the native lactaldehyde reductase encoded by fucO from E. coli MG1655.

In some embodiments, the second enzyme may be an aldehyde dehydrogenase (e.g., encoded by the gene aldA of E. coli). In some embodiments, the second enzyme may be a polypeptide having aldehyde dehydrogenase activity comprising an amino acid sequence at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ ID NO: 2. In some embodiments, the second enzyme may a variant of a of E. coli aldehyde dehydrogenase, wherein the E. coli aldehyde dehydrogenase is encoded by the aldA gene.

In some embodiments, the microorganism is one in which an endogenous hydroxycarboxylic acid-consuming activity is inactivated or decreased. In some embodiments, the microorganism is one in which an endogenous glycolate oxidase and/or endogenous pyruvate formate-lyase activity is inactivated or decreased.

In some embodiments, the microorganism may be bacteria (e.g., Escherichia coli). In some embodiments, the microorganism may be a yeast or fungus. In some embodiments, the yeast or fungus may be from the species Candida boidinii, Candida etchellsii, Candida geochares, Candida lambica, Candida sorbophila, Candida sorbosivorans, Candida sorboxylosa, Candida vanderwaltii, Candida zemplinina, Debaryomyces castellii, Issatchenkia orientalis (also known as Pichia kudriavzevii, Candida krusei, or Saccharomyces krusei), Kluyveromyces lactis, Kluyveromyces marxianus, Pichia anomala, Pichia jadinii, Pichia jadinii, Pichia membranifaciens, Saccharomyces bayanus, Saccharomyces bulderi, Saccharomycopsis crataegensis, Zygosaccharomyces bisporus, Zygosaccharomyces kombuchaensis, or Zygosaccharomyces lentus. In some embodiments, the microorganism may be from Pseudomonas species, Clostridium species, Chlorella species or other algae, Gluconobacter oxydans, Pichia naganishii, Corynebacterium species, or Corynebacterium glutamicum. In some embodiments, the microorganism may be from the species Haloferax mediterranei, Halobactreium salinarum, Nicotiana tabacum, or Thermus thermophilus. In some embodiments, microorganism described herein may be further genetically modified for improved tolerance to acidic pH, as compared to a corresponding wild-type microorganism.

In some embodiments, the non-native substrate described herein (e.g., aliphatic polyhydric alcohol substrate) may be present in the fermentation broth at the start of the growth phase for convenience, even though the non-native substrate is not utilized as a carbon source for biomass accumulation (due to the enzymes required for doing so not yet being expressed in the process described herein). In some embodiments, the non-native substrate described herein (e.g., aliphatic polyhydric alcohol substrate) may be added to the fermentation broth after the start of the growth phase and prior to the start of the production phase.

In some embodiments, the process described herein achieves a titer of glycolic acid of at least about 25, 30, 35, 40, 45, 50, 55, or 60 g/L (e.g., within 48, 36, or 24 hours of production phase). In some embodiments, the process described herein achieve a peak productivity of at least about 4, 4.5, 5, 5.5, 6, 6.5, 7, or 7.5 g/L/h. In some embodiments, the process described herein occurs in a fermentation volume of at least about 5, 10, 25, 50, 100, 200, 300, 400, 500, 600, 700, 800, 900, or 1000 L.

In some embodiments, the aliphatic polyhydric alcohol substrate described herein may be is a substrate of the zinc-dependent alcohol dehydrogenase expressed by the microorganism described herein. In some embodiments, the aliphatic polyhydric alcohol substrate described herein may be an aliphatic diol whose terminal hydroxyl group is selectively oxidized for example by Gox0313 to the corresponding hydroxyl aldehydes (Zhang et al., 2015). In some embodiments, the aliphatic polyhydric alcohol substrate describe herein may be ethylene glycol, diethylene glycol, triethylene glycol, tetraethylene glycol, propylene glycol, glycerol, 1,3-propanediol, 1,2-butanediol, 1,3-butanediol, 1,4-butanediol, or 1,2,4-butanetriol. In some embodiments, the aliphatic polyhydric alcohol substrate describe herein may be ethylene glycol and the hydroxycarboxylic acid may be glycolic acid.

In some aspects, described herein is a process for reducing the production of by-products of overflow metabolism in a one-pot fermentation process. In some embodiments, the process may comprise providing a microorganism engineered to express a biosynthetic pathway comprising multiple exogenous enzymes to produce a product of interest from a non-native feedstock, wherein expression of each of the biosynthetic pathway is controlled by a nutrient-sensing control element that turns on transcription of the multiple enzymes upon depletion of the nutrient below an expression threshold. The process further comprises, in a growth phase, culturing the microorganism for biomass accumulation in a fermentation broth comprising a native carbon source and under conditions of excess oxygen; and in a production phase, further culturing the microorganism for production of the product of interest in the fermentation broth in the presence of the non-native feedstock and under conditions of excess oxygen. In some embodiments, the initial concentration of the nutrient in the fermentation broth prior to the start of the growth phase is set to achieve a target biomass concentration at which nutrient depletion below the expression threshold turns on transcription of the biosynthetic pathway, thereby producing a delay in the expression of the biosynthetic pathway that reduces metabolic burden of the microorganism during the growth phase and a reduction in the production of by-products of overflow metabolism in the production phase.

In some embodiments, the multiple exogenous enzymes are encoded by a polycistronic polynucleotide whose expression is controlled by the nutrient-sensing control element. In some embodiments, the nutrient-sensing control element comprises or consists of a nutrient-sensing promoter, such as a phosphorous-sensing (e.g., phosphate-sensing), sulfur-sensing (e.g. sulfate-sensing), nitrogen-sensing, oxygen-sensing, or magnesium-sensing promoter. In some embodiments the nutrient-sensing control element may be wild-type or engineered. In some embodiments, at least one of the multiple exogenous enzymes is heterologous with respect to the microorganism. In some embodiments, at least one of the multiple exogenous enzymes encoded by a polynucleotide is comprised in an expression vector (e.g., plasmid, cosmid, phage, or virus). In some embodiments, at least one of the multiple exogenous enzymes encoded by a polynucleotide is integrated into the genome of the microorganism in one or more copies.

In some embodiments, the microorganism is a bacteria, yeast, or fungus as described herein. In some embodiments, the by-products of overflow metabolism comprise one or more organic acids (e.g., acetic acid, formic acid, lactic acid, and/or ethanol).

In some aspects, the present description is a microorganism comprising the inducible expression cassette as described herein. In some embodiments, the microorganism is for use in a process as described herein.

In some embodiments, described herein is an alcohol dehydrogenase that catalyzes the conversion of ethylene glycol to glycolaldehyde comprising an amino acid sequence at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to any one of SEQ ID NOs: 207 to 209 and 215 to 264; or a catalytically active fragment thereof. As used herein, the expression “alcohol dehydrogenase” is intended to refer to the desired reaction to be catalyzed as described herein and does not exclude enzymes assigned other names in the scientific literature, as long as the enzymes otherwise meet the structural and catalytic characteristics of the enzymes described herein.

In some embodiments, described herein is an alcohol dehydrogenase from a Gluconobacter species, such as G. morbifer, G. diazotrophicus, G. aidae, G. oxydans, G. roseus, G. potus, G. thailandicus, G. frateurii, G. japonicus, G. cerinus, or G. cerinus. In some embodiments, described herein is an alcohol dehydrogenase from a Gluconacetobacter species, such as G. diazotrophicus, G. asukensis, G. tumulicola, G. aggeris, G. sacchari, G. dulcium, G. takamatsuzukensis, G. liquefaciens, G. azotocaptans, G. johannae, or G. tumulisoli. In some embodiments, described herein is an alcohol dehydrogenase from an organism listed in Table 2, 6 or 7.

In some embodiments, the first enzyme may be an alcohol dehydrogenase that is oxygen-insensitive, such as an alcohol dehydrogenase that employs an oxygen-insensitive cofactor such zinc (instead of iron). Enzymes that employ iron as cofactor may be prone to degradation via metal-catalyzed oxidation, which becomes exacerbated upon exposure to high aeration fermentation conditions.

In some embodiments, described herein is an alcohol dehydrogenase that is not Gox0313 from Gluconobacter oxydans, and/or does not comprise the amino acid sequence of SEQ ID NO: 206. While Zhang et al., 2015 reported the production of glycolaldehyde from oxidation of ethylene glycol using the zinc-dependent medium-chain alcohol dehydrogenase (Gox0313) from Gluconobacter oxydans, the authors also reported that the reverse reaction was more favored for this enzyme. Specifically, the authors at section 3.3 reported that, compared to the oxidation of ethylene glycol, Gox0313 not only had a higher affinity and catalytic rate for glycolaldehyde reduction to ethylene glycol, but also higher turnover and catalytic efficiency for glycolaldehyde as a substrate compared to ethylene glycol. Thus, in some embodiments, described herein are alcohol dehydrogenases having catalytic properties more advantageous for industrial applications. In some embodiments, described herein is an alcohol dehydrogenase that catalyzes the conversion of ethylene glycol to glycolaldehyde at a higher maximum velocity (Vₘₐₓ) and/or at a higher turnover number (k_{cat}) than the zinc-dependent medium-chain alcohol dehydrogenase Gox0313 from Gluconobacter oxydans set forth in SEQ ID NO: 206. Such enzyme characteristics may be assessed in vitro on purified enzymes under the same reaction conditions by, for example, titrating substrate concentrations and measuring product formation of time.

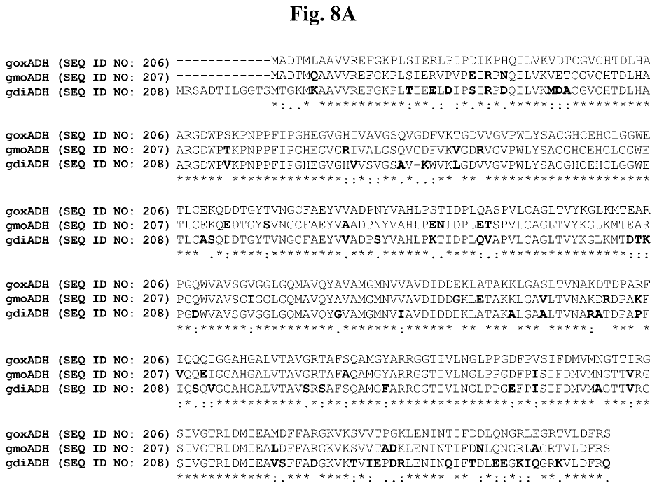

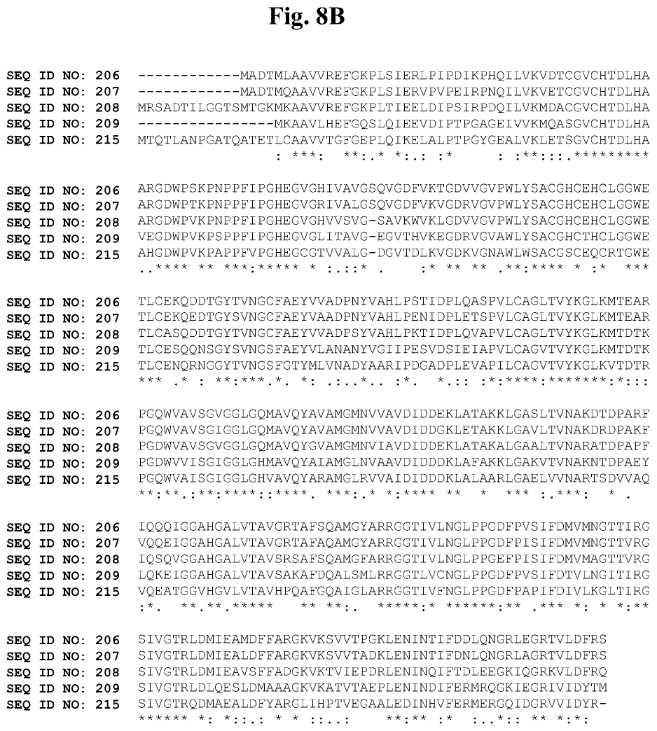

In some embodiments, described herein is an alcohol dehydrogenase that is a Gox0313 variant that differs or differs only from SEQ ID NO: 206 by having at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions at corresponding non-conserved amino acid positions with respect to any one of SEQ ID NOs: 207 to 209 or 215. In some embodiments, amino acid substitutions may comprise replacing the amino acid of SEQ ID NO: 206 with the corresponding amino acid of any one of SEQ ID NOs: 207 to 209 or 215, for example as shown in the multiple sequence alignment of FIGS. 8A and 8B.

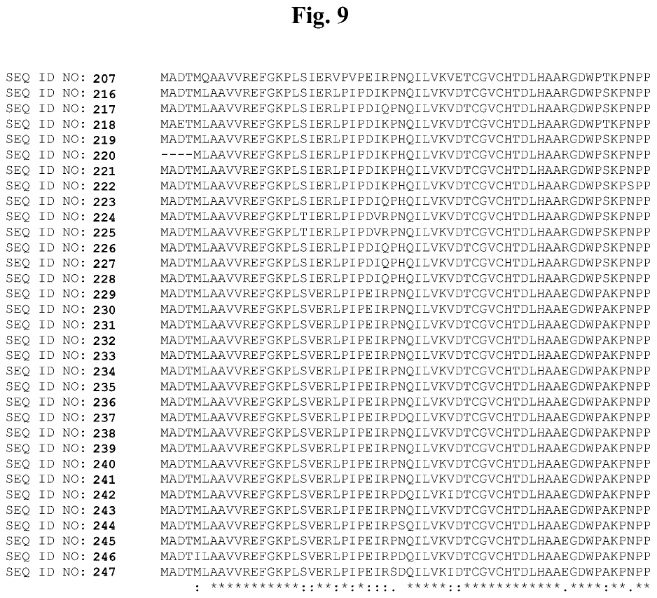

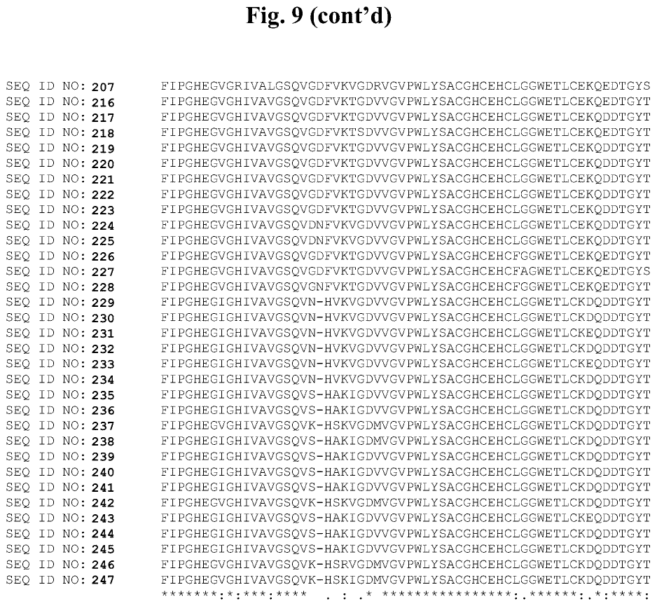

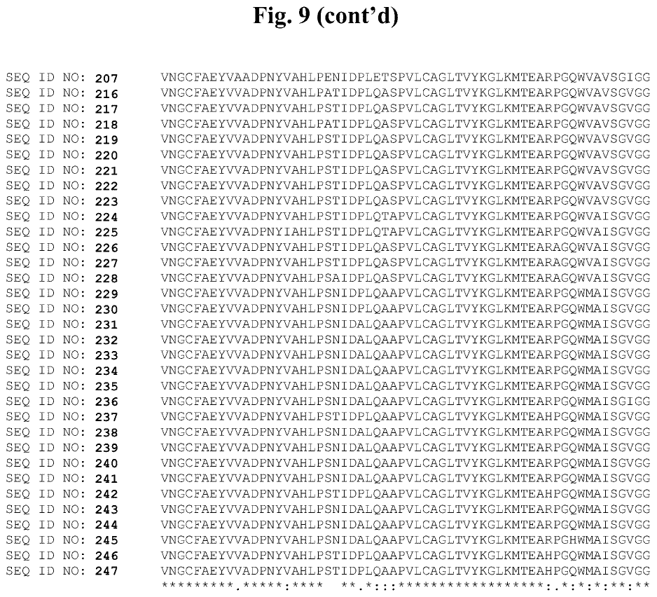

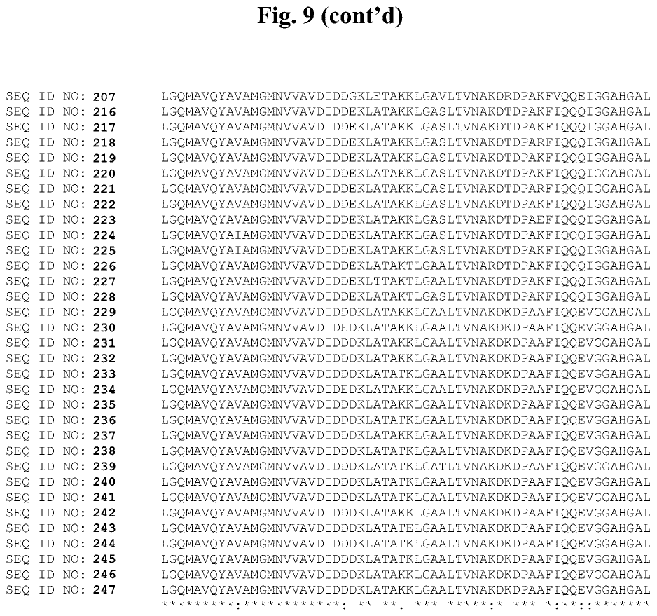

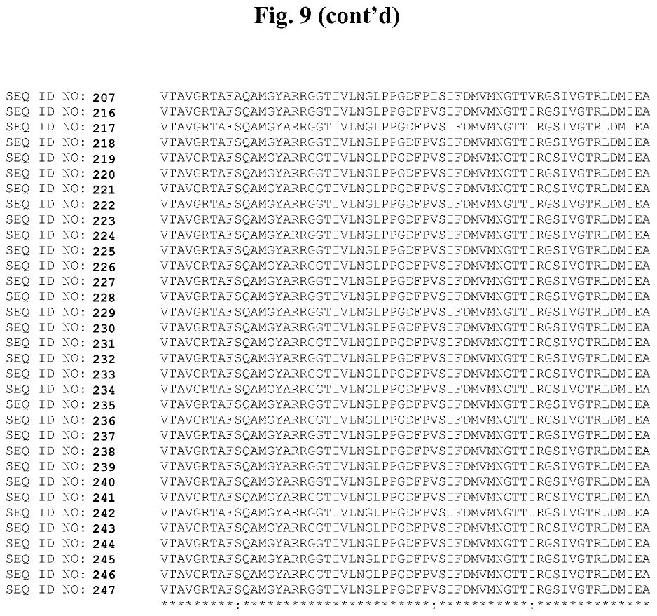

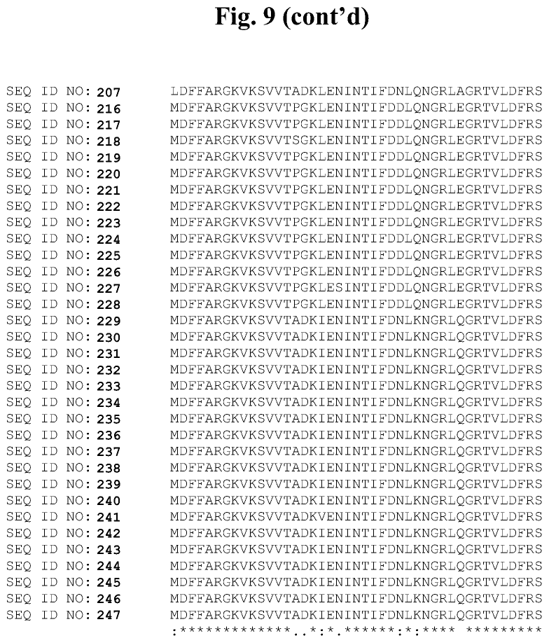

In some embodiments, described herein is a variant of the G. morbifer enzyme (gmoADH) of SEQ ID NO: 207, wherein the variant differs or differs only from SEQ ID NO: 207 by having at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions at corresponding non-conserved amino acid positions with respect to any one of SEQ ID NOs: 216 to 247. In some embodiments, these amino acid substitutions may comprise replacing the amino acid of SEQ ID NO: 207 with the corresponding amino acid of any one of SEQ ID NOs: 216 to 247, for example as shown in the multiple sequence alignment of FIG. 9.

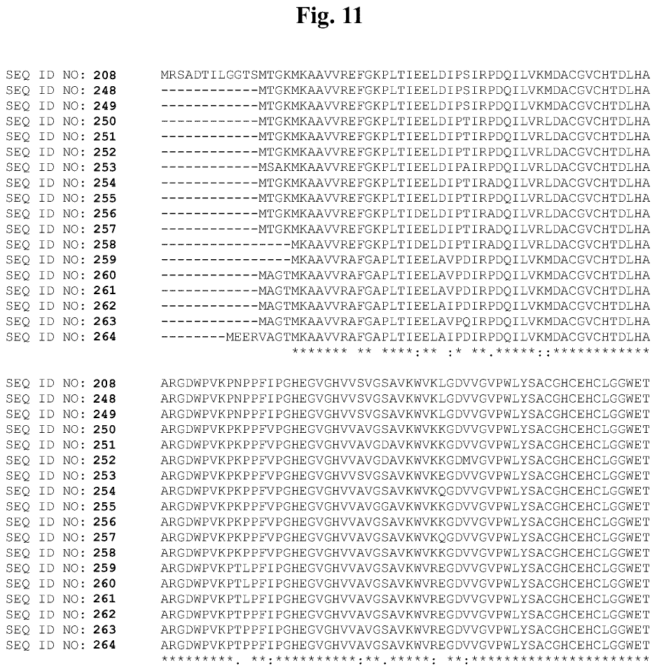

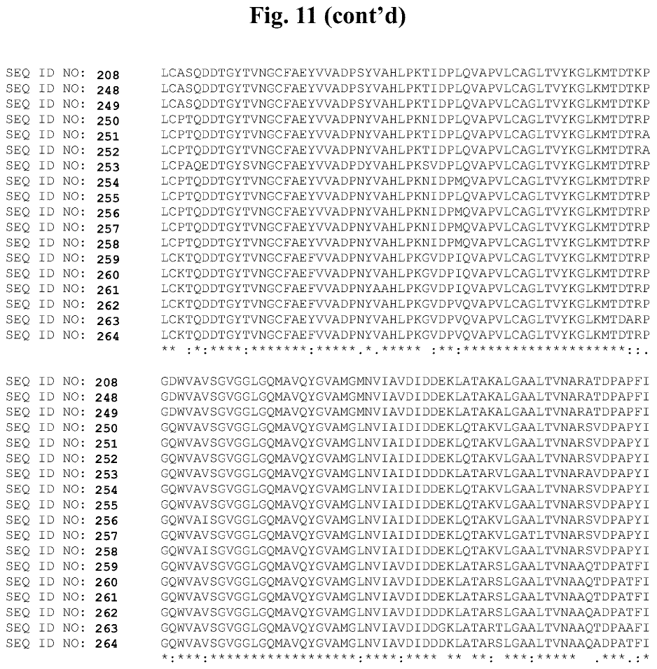

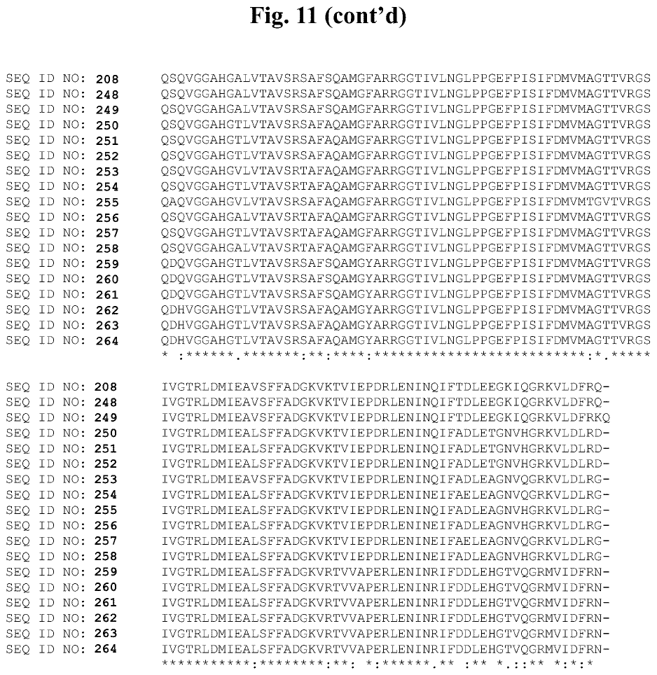

In some embodiments, described herein is a variant of the G. diazotrophicus enzyme (gdiADH) of SEQ ID NO: 208, wherein the variant differs or differs only from SEQ ID NO: 208 by having at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions at corresponding non-conserved amino acid positions with respect to any one of SEQ ID NOs: 248 to 264. In some embodiments, these amino acid substitutions may comprise replacing the amino acid of SEQ ID NO: 208 with the corresponding amino acid of any one of SEQ ID NOs: 248 to 264, for example as shown in the multiple sequence alignment of FIG. 11.

In some embodiments, described herein is an aldehyde dehydrogenase that catalyzes the conversion of glycolaldehyde to glycolic acid comprising an amino acid sequence at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to any one of SEQ ID NOs: 266 to 434; or a catalytically active fragment thereof. As used herein, the expression “aldehyde dehydrogenase” is intended to refer to the desired reaction to be catalyzed as described herein and does not exclude enzymes assigned other names in the scientific literature, as long as the enzymes otherwise meet the structural and catalytic characteristics of the enzymes described herein.

In some embodiments, described herein is an aldehyde dehydrogenase from an Azotobacter species, such as A. vinelandii. In some embodiments, the second enzyme may be an aldehyde dehydrogenase from a Cupriavidus species, such as C. basilensis, C. sp. BIC8F, or C. sp. WS. In some embodiments, the second enzyme may be an aldehyde dehydrogenase from a Pseudomonas species, such as P. aeruginosa, P. asiatica, P. azerbaijanoccidentalis, P. azotoformans, P. brassicacearum, P. brenneri, P. carnis, P. cedrina, P. cyclaminis, P. fluorescens, P. fluorescens, P. fluorescens ABAC62, P. juntendi, P. kairouanensis, P. karstica, P. lurida, P. mandelii, P. marginalis, P. marginalis pv. marginalis, P. migulae, P. monteilii, P. orientalis, P. pisciculturae, P. proteolytica, P. putida, P. putida, P. reinekei, P. shahriarae, P. sivasensis, P. sp. 18.1.10, P. sp. 1912-s, P. sp. 2822-17, P. sp. 2995-1, P. sp. ACN8, P. sp. ANT_H12B, P. sp. A-R-19, P. sp. B14 (2022), P. sp. BIGb0381, P. sp. CFBP13528, P. sp. ES3-33, P. sp. GL-RE-29, P. sp. GM78, P. sp. GM79, P. sp. GOM6, P. sp. KBS0802, P. sp. KBW05, P. sp. M47T1, P. sp. Marseille-Q1929, P. sp. MWU16-30323, P. sp. NFACC42-2, P. sp. NFACC45, P. sp. NIBR-H-19, P. sp. PB101, P. sp. PD9R, P. sp. Q1-7, P. sp. S35, P. sp. S9, P. sp. SJZ080, P. sp. TH32, P. sp. TNT2022 ID1044, P. sp. Tril, P. sp. Xaverov 83, P. sp. YuFO20, P. sp. YuFO8, P. sp. Z18 (2022), P. umsongensis, P. urethralis, P. veronii, or P. yamanorum. In some embodiments, described herein is an aldehyde dehydrogenase from a Stutzerimonas species, such as S. azotifigens, S. azotifigens, S. balearica, S. frequens, S. nitrititolerans, S. stutzeri, or S. zhaodongensis. In some embodiments, described herein is an aldehyde dehydrogenase from a Gammaproteobacteria bacterium, a Marinobacterium species (e.g., M. profundum), a Pseudomonadaceae bacterium T75, or a Rhodococcus species (e.g., R. qingshengii, or R. ruber). In some embodiments, described herein is an aldehyde dehydrogenase from an organism listed in Table 11 or 12.

In some embodiments, described herein is aldehyde dehydrogenases having catalytic properties more advantageous for industrial applications than ecoALDH. In some embodiments, the aldehyde dehydrogenase catalyzes the conversion of glycolaldehyde to glycolic acid at a higher maximum velocity (Vₘₐₓ) and/or at a higher turnover number (k_{cat}) than the aldehyde dehydrogenase aldA from Escherichia coli set forth in SEQ ID NO: 265. In some embodiments, the second enzyme is not aldA from Escherichia coli and/or does not comprise the amino acid sequence of SEQ ID NO: 265.

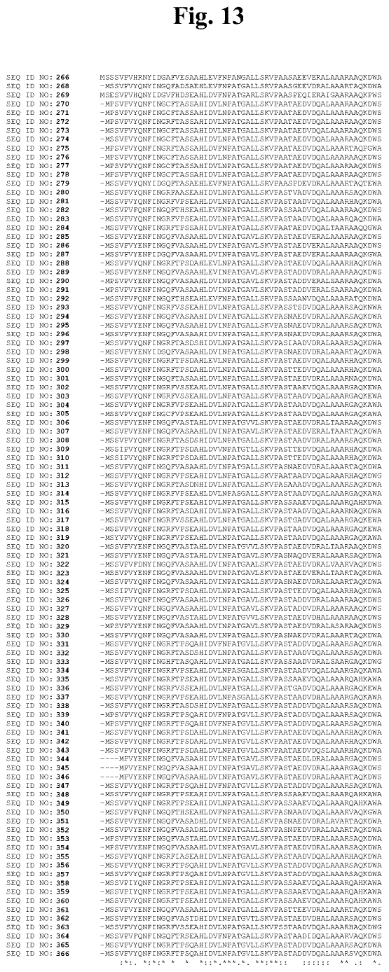

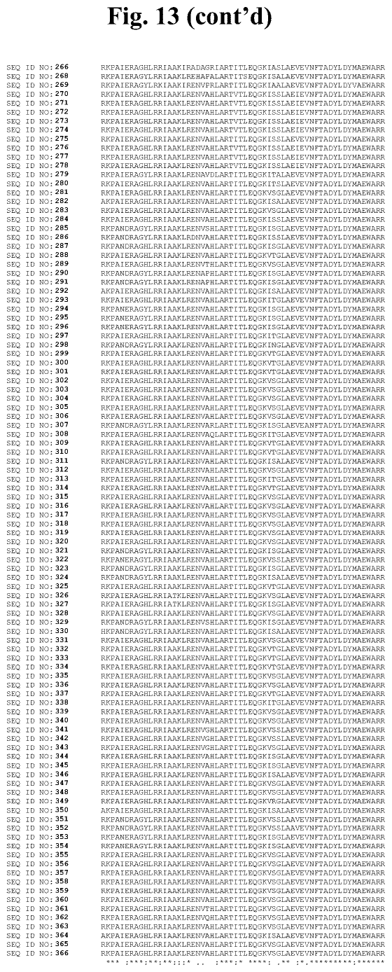

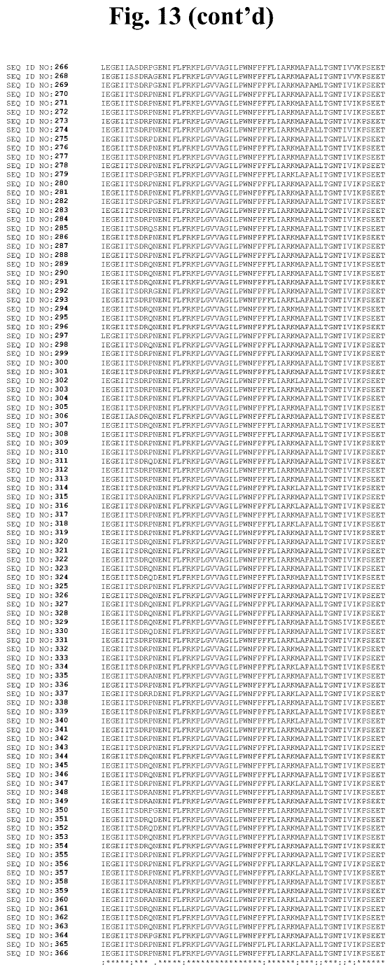

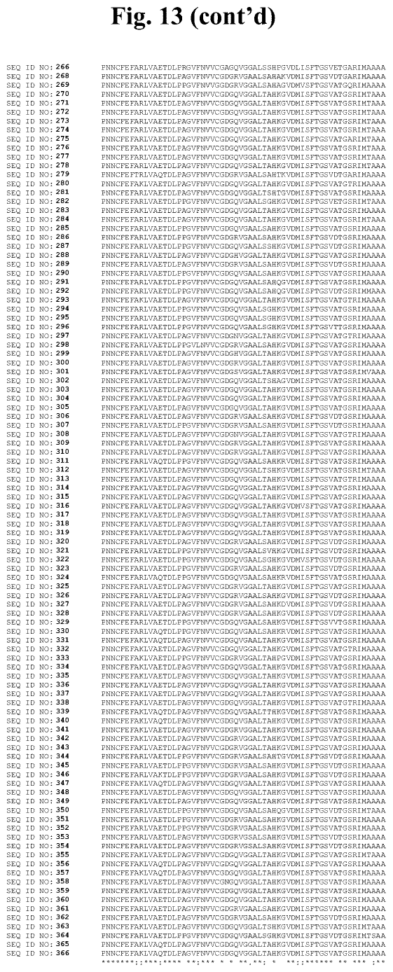

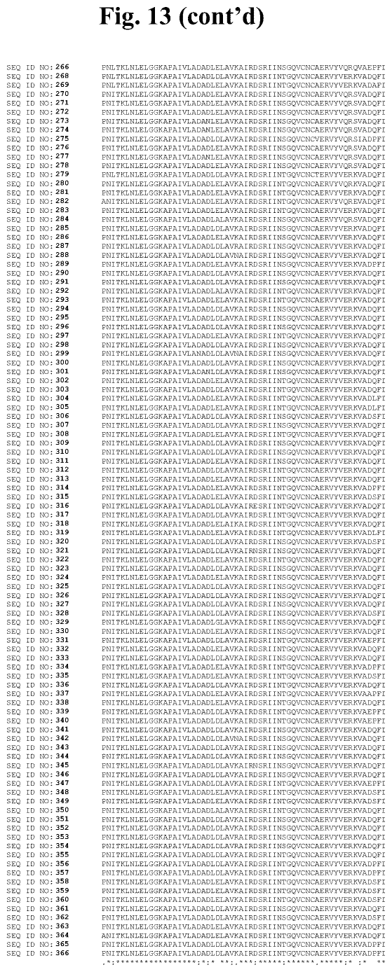

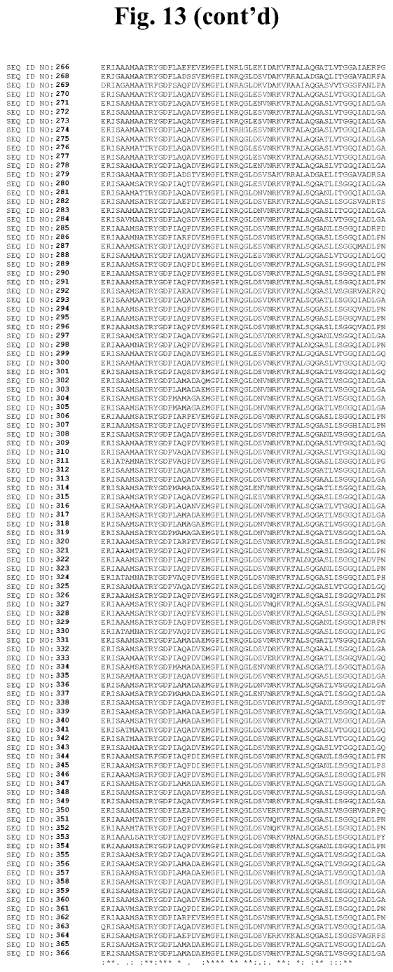

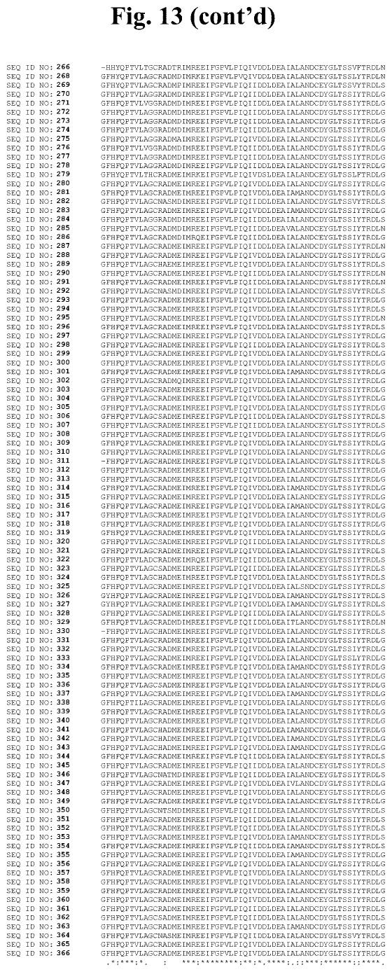

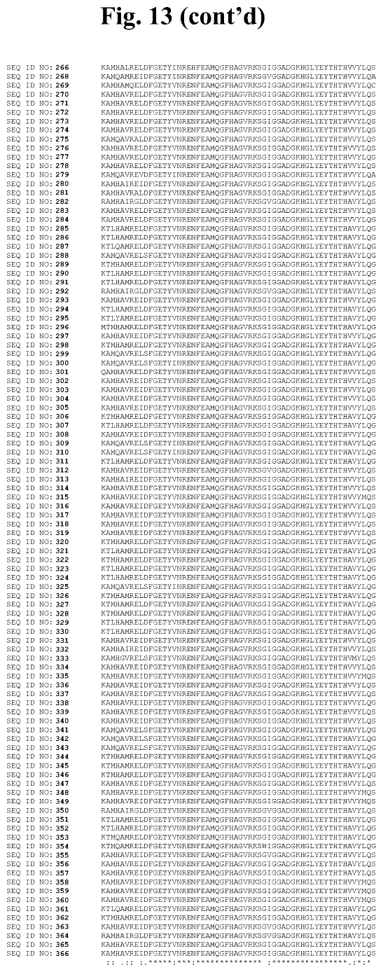

In some embodiments, described herein is a variant of the A. vinelandii enzyme (aviALDH) of SEQ ID NO: 266, wherein the variant differs or differs only from SEQ ID NO: 266 by having at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions at corresponding non-conserved amino acid positions with respect to any one of SEQ ID NOs: 63 to 161. In some embodiments, these amino acid substitutions may comprise replacing the amino acid of SEQ ID NO: 266 with the corresponding amino acid of any one of SEQ ID NOs: 268 to 366, for example as shown in the multiple sequence alignment of FIG. 13.

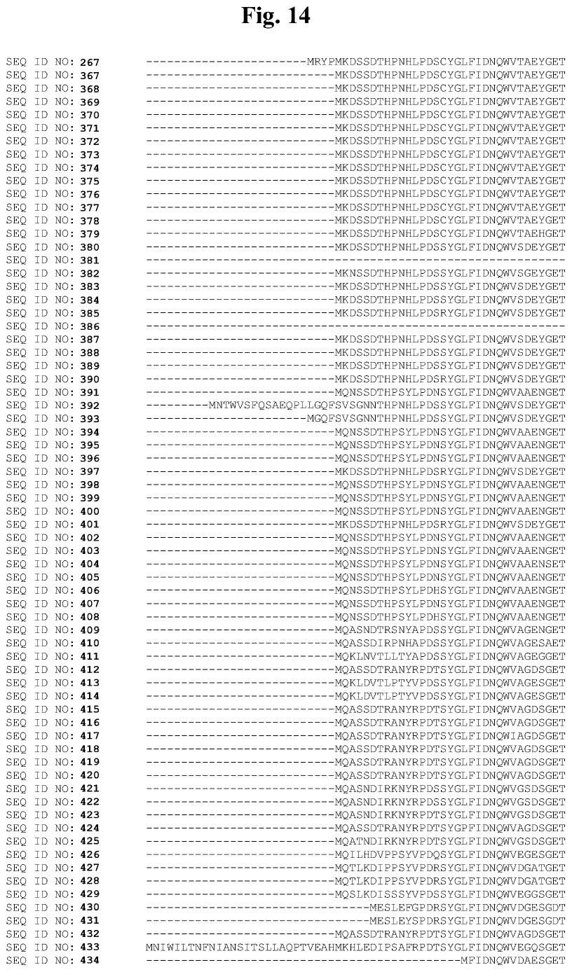

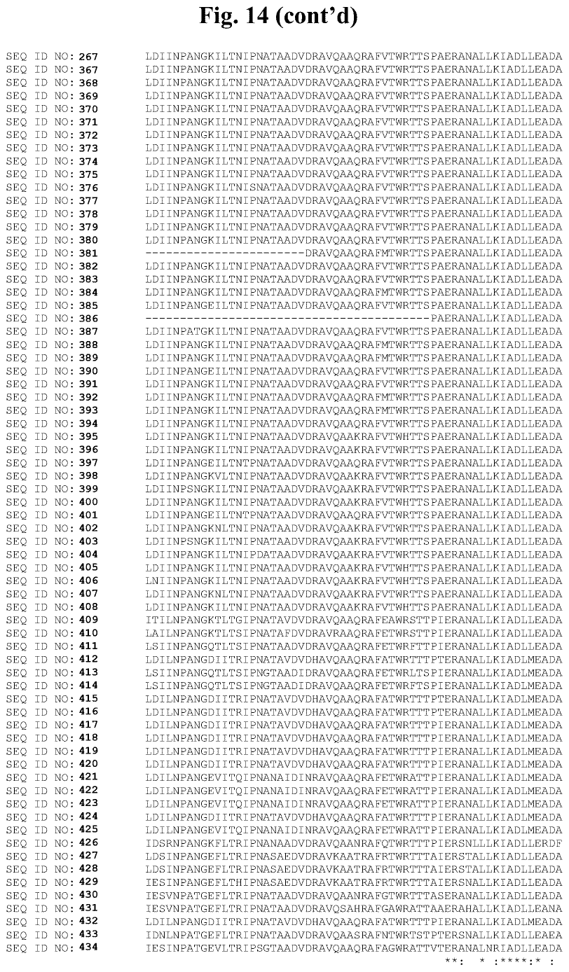

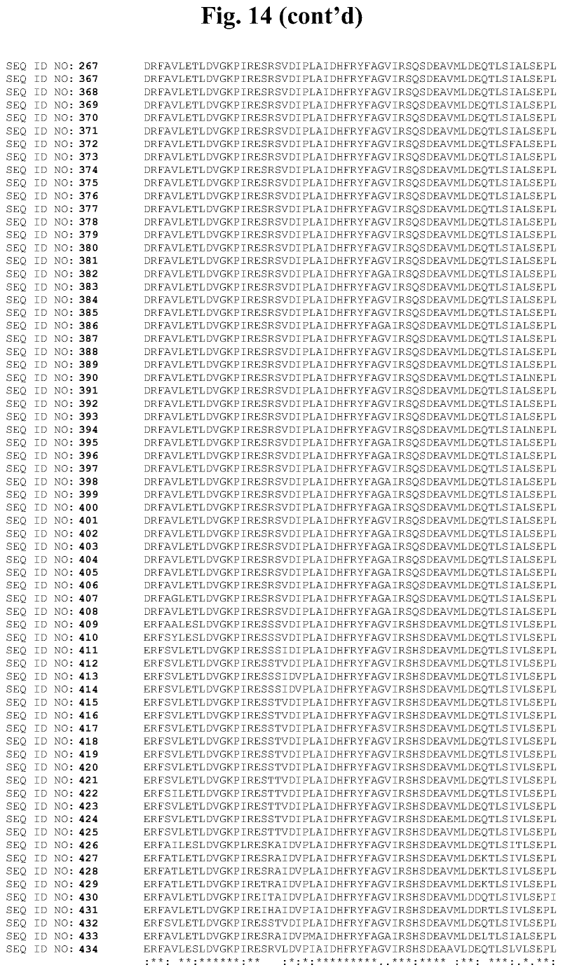

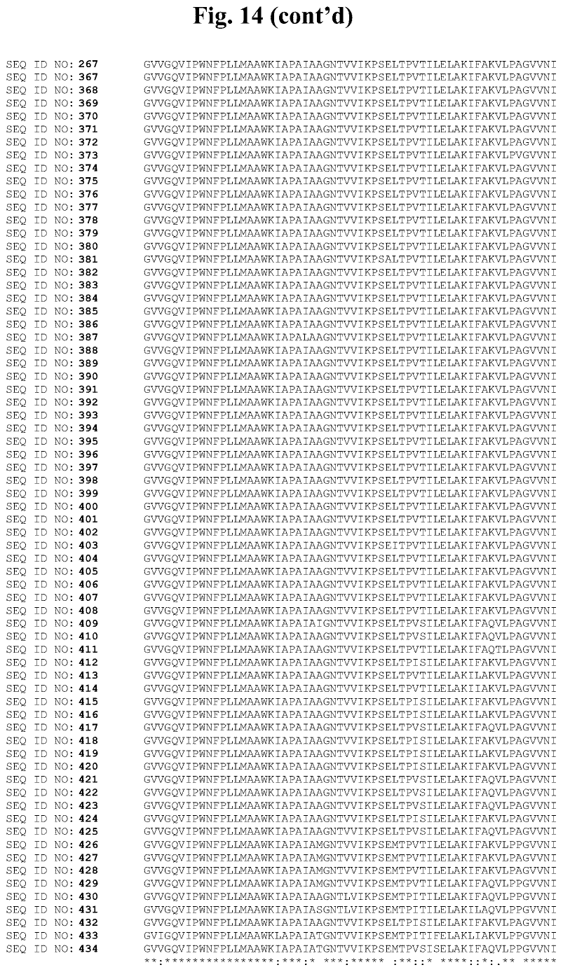

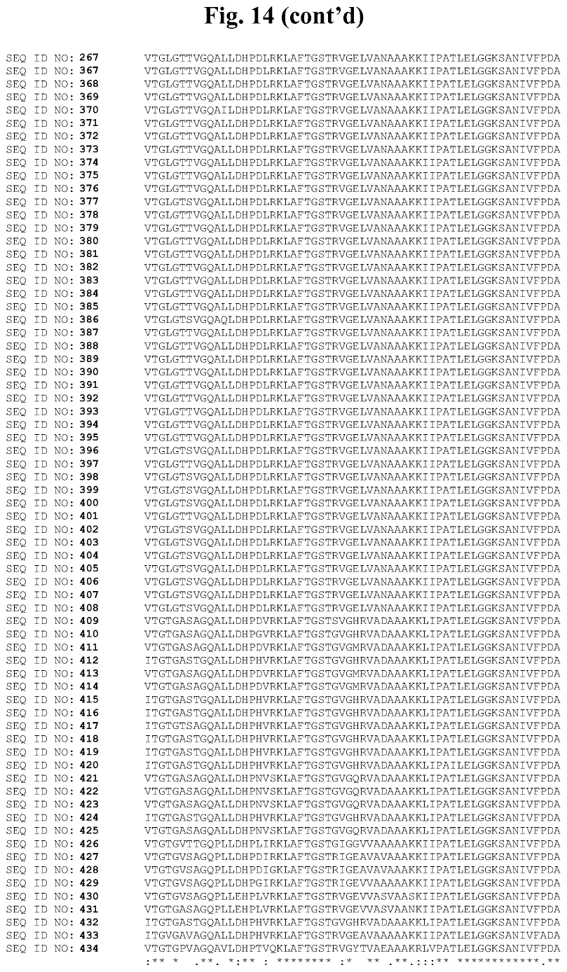

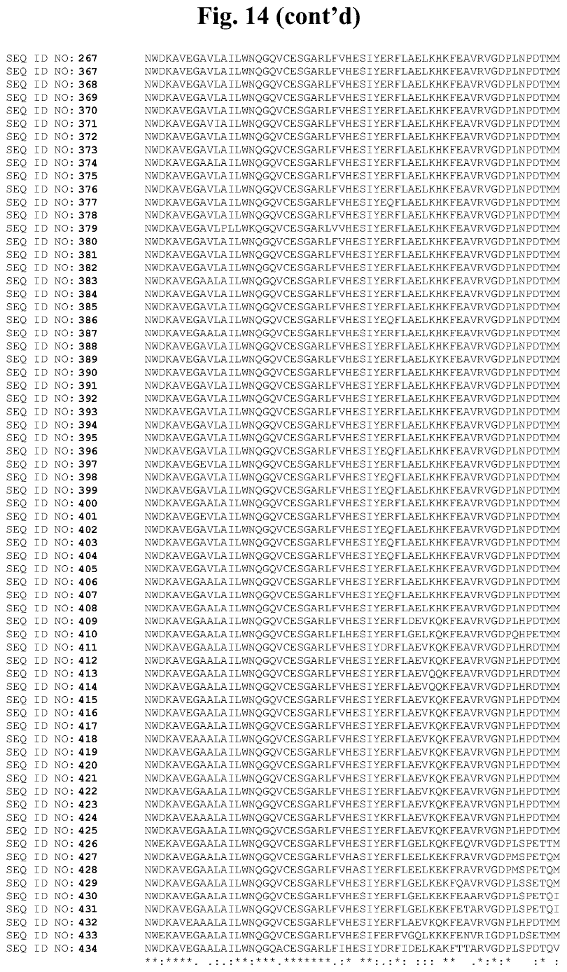

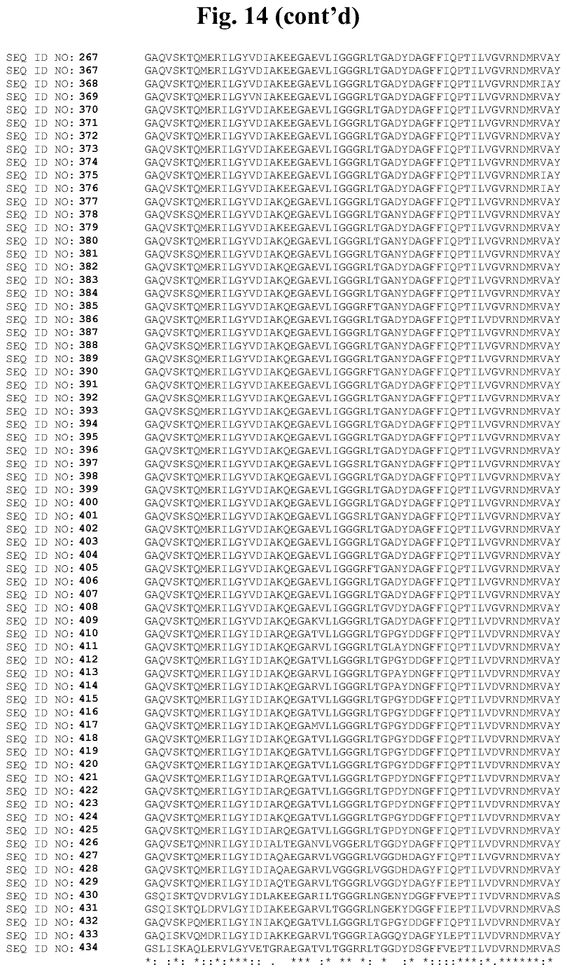

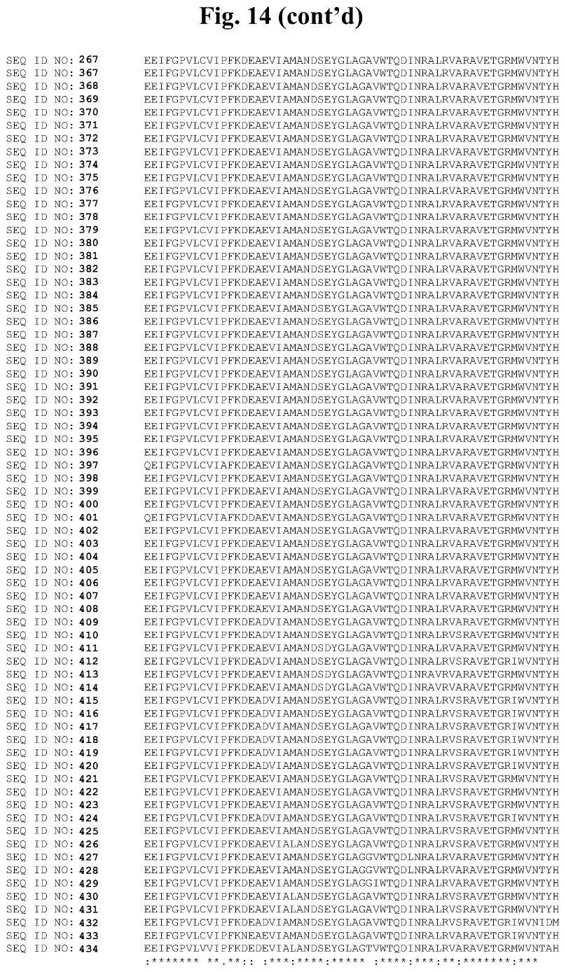

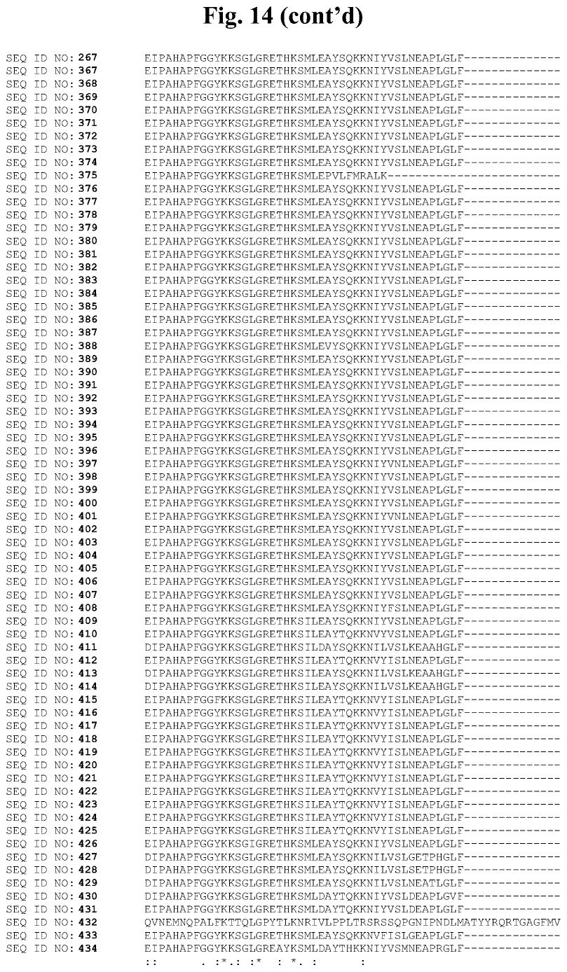

In some embodiments, described herein is a variant of the P. putida enzyme (ppuALDH) of SEQ ID NO: 267, wherein the variant differs or differs only from SEQ ID NO: 267 by having at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions at corresponding non-conserved amino acid positions with respect to any one of SEQ ID NOs: 367 to 434. In some embodiments, these amino acid substitutions may comprise replacing the amino acid of SEQ ID NO: 266 with the corresponding amino acid of any one of SEQ ID NOs: 367 to 434, for example as shown in the multiple sequence alignment of FIG. 14.

In some embodiments, at least one of the alcohol dehydrogenase and the aldehyde dehydrogenase enzymes described herein may be heterologous or exogenous with respect to the microorganism from which they are expressed. In some embodiments, the alcohol dehydrogenase and the aldehyde dehydrogenase enzymes described herein may be encoded by polynucleotides comprised in a single polycistronic expression cassette (e.g., integrated into the genome of the microorganism in one or more copies, such as at least 1, 2, 3, 4, 5, 6, 7, 8, 9, 10, 11, 12, 13, 14, 15, 16, 17, 18, 19, or 20 copies). In some embodiments, the alcohol dehydrogenase and the aldehyde dehydrogenase enzymes described herein may be encoded by polynucleotides comprised in an expression vector (e.g., plasmid). In some embodiments, the alcohol dehydrogenase and/or the aldehyde dehydrogenase enzymes described herein may be encoded by polynucleotide(s) operably linked to a heterologous promoter (i.e., the promoter being heterologous with respect to the enzyme under control thereof).

In some embodiments, the microorganism described herein may be engineered or further engineered to comprise genetic modifications to increase product titres. In some embodiments, the microorganism described herein may be engineered or further engineered to comprise genetic modifications to inactive or disrupt genes (e.g., endogenous genes) that encode for glycolate oxidase, lactate dehydrogenase, pyruvate formate lyase, and/or ethanol dehydrogenase. In some embodiments, the microorganism may be one in which an endogenous glycolate oxidase, endogenous pyruvate formate-lyase, or ethanol dehydrogenase activity is/are inactivated or decreased. In some embodiments, the microorganism's endogenous glycolate oxidase (or a subunit thereof required for catalytic activity) may deleted, truncated, or mutated to impair or abrogate glycolate oxidase activity. In some embodiments, expression of the microorganism's endogenous glycolate oxidase, lactate dehydrogenase, pyruvate formate lyase, and/or ethanol dehydrogenase (or any subunit thereof required for catalytic activity), may be inhibited, silenced, knocked-down, or repressed. In some embodiments, the microorganism described herein comprises an inactivated or impaired glcD gene encoding a glycolate oxidase subunit GlcD.

In some embodiments, the microorganism described herein may be a bacteria (e.g., Escherichia coli), yeast, or fungus. In some embodiments, the microorganism may be from a Pseudomonas species, Clostridium species, Chlorella species or other algae, Gluconobacter oxydans, Pichia naganishii, Corynebacterium species, or Corynebacterium glutamicum. In some embodiments, the microorganism may be from the species Candida boidinii, Candida etchellsii, Candida geochares, Candida lambica, Candida sorbophila, Candida sorbosivorans, Candida sorboxylosa, Candida vanderwaltii, Candida zemplinina, Debaryomyces castellii, Issatchenkia orientalis (also known as Pichia kudriavzevii, Candida krusei, or Saccharomyces krusei), Kluyveromyces lactis, Kluyveromyces marxianus, Pichia anomala, Pichia jadinii, Pichia jadinii, Pichia membranifaciens, Saccharomyces bayanus, Saccharomyces bulderi, Saccharomycopsis crataegensis, Zygosaccharomyces bisporus, Zygosaccharomyces kombuchaensis, or Zygosaccharomyces lentus. In some embodiments, the microorganism may be from Haloferax mediterranei, Halobactreium salinarum, Nicotiana tabacum, or Thermus thermophilus.

In some aspects, described herein are compositions comprising one or more enzymes that catalyze the conversion of an aliphatic polyhydric alcohol having a terminal hydroxyl group to its corresponding aldehyde, and/or that catalyze its subsequent conversion to a corresponding alpha-hydroxy acid. In some embodiments, described herein are compositions comprising one or more enzymes that catalyze the conversion of ethylene glycol to glycolaldehyde, and/or the conversion of glycolaldehyde to glycolic acid. In some embodiments, the compositions may comprise an isolated alcohol dehydrogenase as described herein. In some embodiments, the compositions may comprise an isolated aldehyde dehydrogenase as described herein.

In some embodiments, the microorganisms and/or compositions described herein are for use in (i) the conversion of an aliphatic polyhydric alcohol having a terminal hydroxyl group to its corresponding aldehyde, as a product or intermediate; (ii) the conversion of the corresponding aldehyde to a corresponding alpha-hydroxy acid, as a product or intermediate; or (iii) the conversion of an aliphatic polyhydric alcohol having a terminal hydroxyl group to a corresponding alpha-hydroxy acid, as a product or intermediate. In some embodiments, the aliphatic polyhydric alcohol having a terminal hydroxyl group described herein is a substrate of an alcohol dehydrogenase described herein, and the corresponding aldehyde is a substate of an aldehyde dehydrogenase described herein.

In some aspects, described here is a polynucleotide encoding an alcohol dehydrogenase as described herein. In some aspects, described here is a polynucleotide encoding an aldehyde dehydrogenase as described herein. In some aspects, described here is a polynucleotide encoding both an alcohol dehydrogenase and aldehyde dehydrogenase as described herein. In some aspects, described herein is an expression cassette comprising a polynucleotide described herein operably linked to a heterologous promoter. In some aspects, described herein is a vector comprising a polynucleotide or expression cassette described herein. In some embodiments, the polynucleotides described herein (including the full complements thereof) may be useful as tools for screening and/or hybridization (e.g., to identify novel polypeptides potentially encoding alcohol dehydrogenases or aldehyde dehydrogenases of interest), independent the activity of the polypeptides them encode.

In some embodiments, described herein is a process for producing glycolaldehyde, as a product or intermediate, from ethylene glycol, the process comprising contacting ethylene glycol with the isolated alcohol dehydrogenase as described herein, or with a microorganism overexpressing same, under reaction or fermentation conditions enabling the conversion of ethylene glycol to glycolaldehyde. In some embodiments, described herein is a process for producing glycolic acid, as a product or intermediate, from glycolaldehyde, the process comprising contacting glycolaldehyde with the isolated aldehyde dehydrogenase as described herein, or with a microorganism overexpressing same, under reaction or fermentation conditions enabling the conversion of glycolaldehyde to glycolic acid. In some embodiments, described herein is a process for producing glycolic acid, as a product or intermediate, from ethylene glycol, the process comprising contacting ethylene glycol with the isolated aldehyde dehydrogenase as described herein, or with a microorganism overexpressing same, under reaction or fermentation conditions enabling the conversion of ethylene glycol to glycolaldehyde; and contacting the glycolaldehyde with the isolated aldehyde dehydrogenase as described herein, or with a microorganism overexpressing same, under reaction or fermentation conditions enabling the conversion of glycolaldehyde to glycolic acid.

In some embodiments, the process described herein may comprise a growth phase during which the microorganism is cultured under conditions promoting biomass accumulation, and a production phase during which the microorganism is cultured under conditions promoting the production of the product or intermediate. In some embodiments, transition from the growth phase to the production phase is not triggered by a modification in aeration conditions from the growth phase to the production phase (e.g., a decrease in oxygen uptake rate from the growth phase to the production phase).

In some aspects, described herein is a fermentation broth comprising: (i) ethylene glycol, glycolaldehyde, and/or glycolic acid; and (ii) a microorganism as described herein, and/or a composition as described herein.

### EXAMPLES

### Example 1: Materials and Methods

### Media and Cultivation Conditions

Cells were grown using lysogeny broth (LB) as per manufacturer's instructions (Bioshop, Burlington, ON) for all strain construction and to generate pre-culture inoculum for fermentations. Pre-cultures for bioreactor fermentation were grown in M9 media. Cells were grown at 37 °C in culture tubes with shaking at 200+ RPM.

For bioreactor characterization, cells were grown in minimal defined media providing sufficient carbon, nitrogen, and phosphate to reach the desired biomass concentration. Ammonium sulfate and ammonium chloride were used as sources of nitrogen. Glucose monohydrate was used as the carbon source for biomass accumulation. Phosphate was provided as a mixture of monobasic and dibasic salts adjusted to pH 7.2. A trace metal solution was prepared according to the following composition in 0.1 M HCl and added to the media at a concentration of 1/1000:1.6 g FeCl₃, 0.2 g CoCl₂·6 H₂O, 0.1 g CuCl₂, 0.2 g ZnCl₂·4H₂O, 0.2 g NaMoO₄, 0.05 g H₃BO₃. Magnesium sulfate, calcium chloride, and thiamine were supplemented to the fermentation broth. Kanamycin was added as an antibiotic, when appropriate.

### Culturing Techniques in Reactors

Fermentation vessels were equipped with pH and oxygen control. pH was maintained at about 7 using either 25% or 50% NaOH solution. Dissolved oxygen and pH probes were used in accordance with the manufacturers' operating guidelines. Aeration was provided up 1 vvm for cultivation in bioreactors. All bioreactor cultivations were carried out in fed-batch to maintain a growth rate between 0.32-0.35 h⁻¹. Systematic changes in aeration rate and impeller speed were applied between cultivations to maintain a dissolved oxygen of at least 20% relative to the saturated condition, where possible.

### Plasmids and Strains

Gox, FucO, and AldA genes were either cloned or synthesized into a vector with a pBR332 origin of replication and a low copy number. The phosphate promoter sequence of SEQ ID NO: 3 was used for phosphate dependent expression. A ribosome binding site (RBS) with the sequence of SEQ ID NO: 4 was placed upstream of the aldA gene. An RBS with the sequence of SEQ ID NO: 5 was placed upstream of the gox gene. GlcD was inactivated using conventional lambda-red recombinant engineering. A number of primers may be used to delete the gene. One example is to use the forward primer of SEQ ID NO: 6 and the reverse primer of SEQ ID NO: 7 to PCR a cassette from pkD3 for the purpose of introducing at the glcD locus of the E. coli strain and inactive it by introducing an antibiotic marker instead.

### Analytical Methods

Fermentation product composition was measured via high performance liquid chromatography (HPLC). A Bio-Rad™ HPX-87H organic acids column was used with 10 mM H₂SO₄ as the eluent and a flowrate of 0.5 mL/min at 50 °C. Organic acids were detected at 210 nm. Cell densities of the cultures were determined by measuring optical density at 600 nm. Cell density samples were diluted, as necessary, to fall within the linear range of less than 0.4, diluted.

### Example 2: Construction of Phosphate-Sensing Expression System for GA-Producing Enzymes

Previously described strategies for the production of glycolic acid (GA) from ethylene glycol (EG), such as those described in Kataoka et al., 2001, Wada et al., 2005, Morishige et al., 2007, and Hua et al., 2018, all employ multi-pot fermentation approaches in which GA-production pathway enzymes are expressed using strong, constitutive promoters so that the enzymes accumulate in the biomass, which is then collected, washed, and used for the biotransformation of EG to GA. These approaches are highly intensive, require significant operator intervention, and/or employ long fermentation times, which all greatly hamper their commercial viability at industrial scales. Our group previously described a “one-pot” process in which the transition between growth and production is controlled by oxygen availability instead of through physical separation via centrifugation and washing (WO/2019/046946 and Pandit et al., 2021). More particularly, cells constitutively-expressing enzymes for converting EG to GA are first provided excess oxygen during the growth phase, leading to biomass accumulation, and then oxygen is reduced in the production phase to stop growth by blocking the GA assimilation pathway via glycolate oxidase. While this process reduced the need for operator intervention and physical separation of biomass, the resulting process productivity was throttled because oxygen is required for the EG to GA transformation and, therefore, reduced oxygen during the production phase limited the rate at which this conversion could take place. An alternate strategy in which both the growth and production phases could be performed under excess oxygen was thus explored herein.

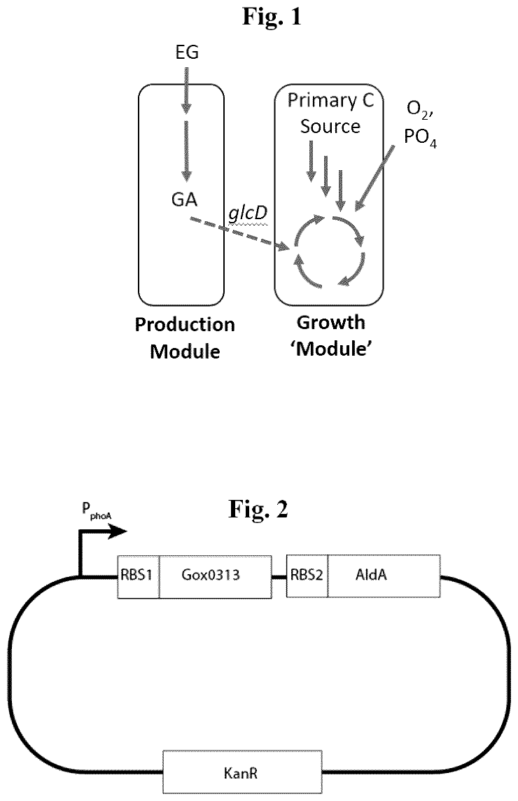

Because EG is not a native carbon source for most organisms, including E. coli, the conversion of EG to GA can be made completely orthogonal to (i.e., non-overlapping with) native metabolism. As shown in FIG. 1, only a single gene (glcD; Glycolate oxidase subunit GlcD) may be deleted, impaired, or inactivated to prevent consumption of the accumulated GA via native metabolic pathways, and deletion/impairment/inactivation of this gene does not affect growth relative to wild type cells with using a native carbon source. This means that switching to the productive state only requires the turning ON of gene expression, and does not rely on turning genes OFF, or on the slow degradation of proteins associated with the growth state. We thus investigated nutrient limitation as the signal to switch from growth to production because the same signal that causes the expression of the production pathway enzymes to turn ON is that which stops growth. We selected E. coli as a model host organism and phosphate as the limiting nutrient by utilizing the E. coli phosphate-sensing phoA promoter. However, it is expected that the same production pathway enzymes can be expressed in other microorganisms, for example using that organism's own nutrient-sensing machinery (e.g., the organism's native phosphate-sensing machinery).

We initially built a plasmid-borne genetic construct in which the phoA promoter drives expression of red fluorescent protein (RFP) as a reporter to characterize the phosphate concentration threshold required to turn gene expression ON. We grew cells carrying this plasmid in various vessels, from 50 μL to 300 mL, with varied initial phosphate concentration in the growth medium and measured the presence of RFP over the duration of growth. Interestingly, we found that the initial phosphate concentration in the growth medium can be tightly tuned and set to achieve a target biomass concentration at which expression from the phoA promoter turned ON.

From here, we swapped out the RFP reporter gene in our plasmid construct for the genes involved in our oxygen-resistant GA-producing pathway, along with ribosome binding sites (RBS) to control their expression levels, as described in Example 1. The plasmid construct is shown in FIG. 2.

### Example 3: Constitutive and phoA-Driven Expression Cultures Exhibit Similar Biomass Accumulation Via Glucose Consumption

In parallel, we grew cultures of cells carrying the constitutively-expressed pathway enzymes and cultures of those carrying the phoA-driven expression system, both in phosphate-limited media containing EG, in flasks (50 mL fermentation volume).

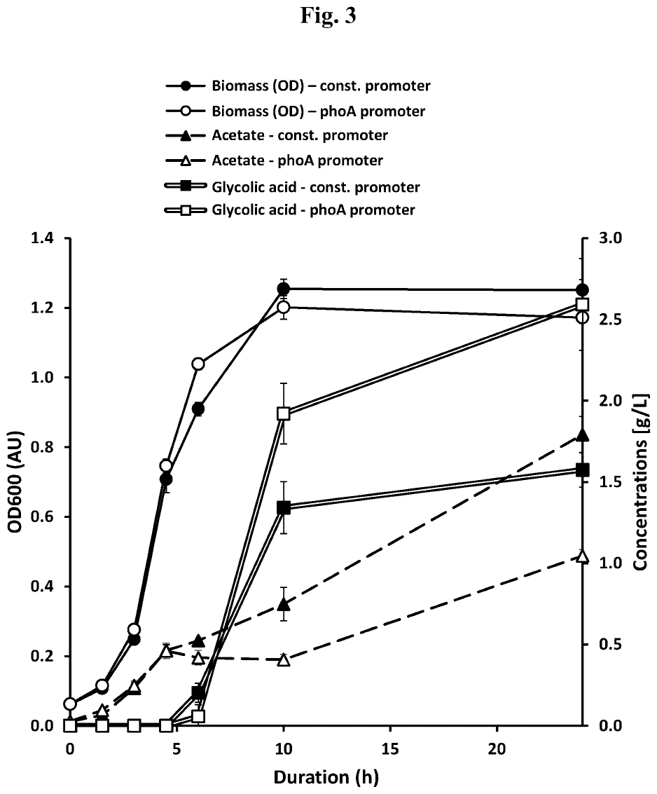

Constitutive expression of non-native proteins and/or overexpression of native proteins is conventionally known to create metabolic stress on a host organism, for example, by disrupting the cell's overall protein budget (Basan et al., 2015). This metabolic stress, which is manifested as acetate production in E. coli, diverts both carbon and energy away from biomass accumulation. However, this was surprisingly not what was observed, as both the constitutive and phoA-driven expression cultures accumulated similar biomass over the same time frame via glucose consumption (FIG. 3, compare filled and open circles), despite initial phosphate levels being sufficiently high to inhibit expression of the GA-producing enzymes under control of the phoA promoter. Furthermore, acetate production levels were very similar between the constitutive and phoA-driven expression cultures from the start of fermentation until late exponential phase (FIG. 3, compare filled and open triangles, particularly from 0 to 5 h). Collectively, these results suggest that constitutive expression of the GA-producing pathway enzymes had no detrimental effect on host microorganism growth.

### Example 4: PhoA-Driven Expression Cultures Yielded Superior GA Production Over Constitutive Expression Cultures

Despite continuously expressing the GA-producing pathway enzymes, and despite EG being present in the broth since the start of fermentation, the constitutive expression cultures began producing GA in late exponential phase, similar to the phoA-driven expression cultures (FIG. 3, compare filled and open squares from 0 to 5 h). However, in cells expressing the GA-producing pathway enzymes constitutively, GA production began slightly earlier (FIG. 3, compare filled and open squares at 6 h). Despite this “head start,” the GA titer achieved in the constitutive expression culture was strikingly lower than that of the phoA-driven expression culture (FIG. 3, compare filled and open squares from >6 to 24 h). At the end of the 24 h fermentation, the constitutive system had produced ~1.6 g/L of GA, whereas the phoA system had produced ~2.6 g/L of GA, representing a 62.5% increase. Given that the biomass accumulation in both cultures was similar, this difference in titer (and therefore productivity) was striking.

### Example 5: Coupling Expression of GA-Production Pathway Enzymes to Nutrient Depletion Reduces Host Metabolic Burden During the Production Phase

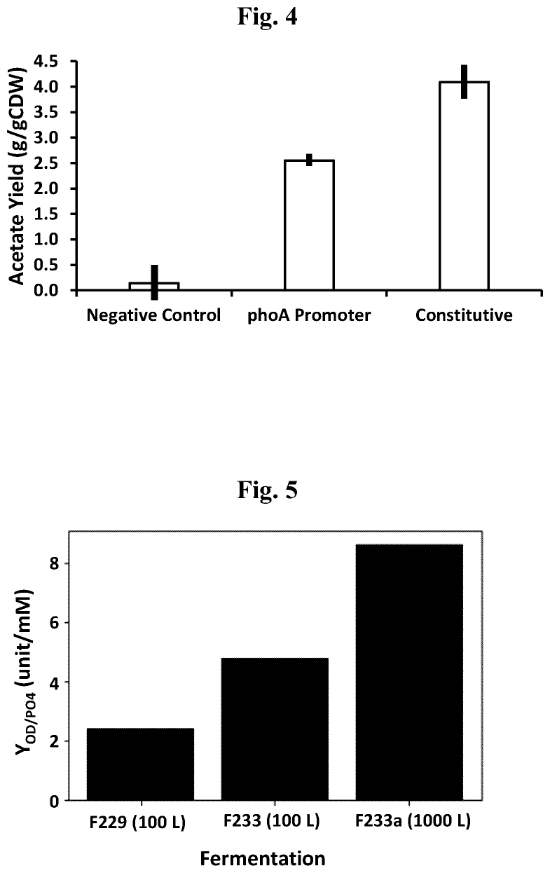

We measured the acetate production in both cultures to find that, in the constitutive expression culture, cells produced nearly 2 g/L of acetate, whereas cells of the phoA-driven expression culture only produced ~1 g/L of acetate (FIG. 3, compare filled and open triangles at 24 h). Further, we observed that the difference in acetate production occurred primarily during the production phase, not the growth phase, in both cultures, as indicated by a divergence in acetate production curves after ~7 h (FIG. 3, compare filled and open triangles). Overall, the acetate yield (acetate produced per gram of biomass produced) was 67% higher in the constitutive system than in the phoA system (FIG. 4). This neatly accounts for the increase in GA titer/productivity in the latter and demonstrates that delaying expression of the GA-producing pathway enzymes until the end of growth phase by coupling expression to a sensor of nutrient depletion (e.g., a phosphate-sensing system) reduced the metabolic burden of the host in the production phase, thereby allowing more resources (i.e., energy) to go to maintaining the production pathway following growth. This was unexpected given that there were no observable signs of metabolic burden of the host by the constitutive system during the growth phase. Since EG is not a carbon source for E. coli, energy savings—not carbon savings—is believed to explain the decreased metabolic burden of the phoA-driven system relative to the constitutive-expressed system.

### Example 6: Scale-Up of phoA System for GA Production

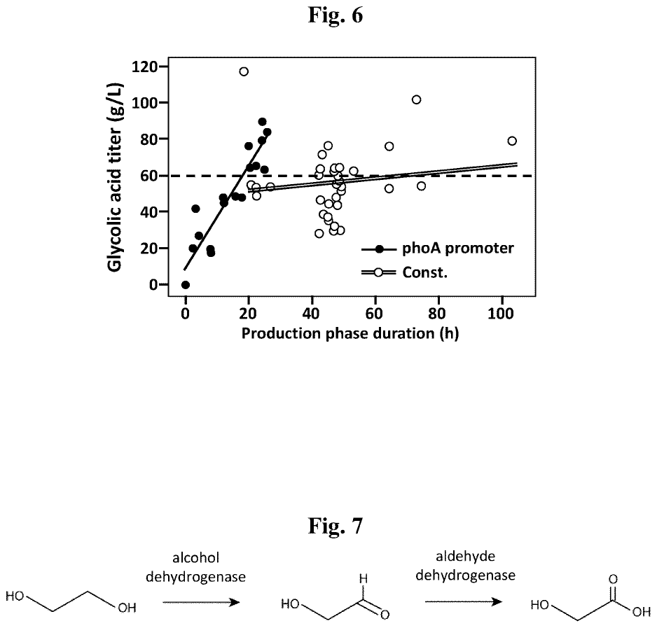

The promising benchtop experimental results in Example 2 motivated the scale-up of the two-phase phosphate-based system to 5 L, then 100 L, and on to 1000 L. At the 50-mL benchtop scale, we had found that the initial phosphate concentration in the growth medium could be tightly tuned to achieve a target biomass concentration at which expression from the phoA promoter turned ON. However, we observed that the relationship between the initial phosphate concentration and the biomass concentration at which expression turned ON was very inconsistent across scales, particularly at scales of 100 L or greater. These inconsistencies negatively impacted overall glycolic acid productivity at scales of 100 L or greater. The results shown in FIG. 5 exemplify the inconsistencies observed across scales, in which three different fermentations operating at different scales (100 L or 1000 L) and at different growth rates were carried out. Fermentation “F229” was grown at a growth rate lower than fermentations “F233” and “F233a.” Fermentations “F233” and “F233a” had approximately the same growth rate, but were at different reactor scales (i.e., 100 L and 1000 L, respectively). The bar graph in FIG. 5 shows that the biomass yield on phosphate concentration (YOD/PO4) was variable over scales and growth rates. Hence, empirical testing was undertaken to determine initial phosphate concentrations that would result in commercially relevant glycolic acid productivities at fermentation scales of 100 L or greater. Interestingly, this empirical testing revealed that, while higher initial phosphate concentrations (e.g., greater than 50 mM) may be advantageous in terms of biomass accumulation and glycolic acid productivity at smaller scales (e.g., 5 L and under), such was not the case for scales of 100 L or greater. More specially, empirical testing revealed that initial phosphate concentrations of less than 50 mM were advantageous for glycolic acid productivity at larger scales (e.g., 50 or 100 L), with initial phosphate concentrations of less than 25 mM being further advantageous at even larger scales (e.g., 1000+L).

Across all scales, once optimal initial phosphate concentrations were determined, moving to the phosphate limitation system with glucose as a main carbon source in the growth phase, resulted in striking peak productivity increases as compared to the constitutive system. Our best constitutive systems had peak productivities of ~2 g/L/h, which matched other state-of-the-art in vivo GA fermentation processes. While these batches therefore operated at or above the threshold for commercial viability, implementation of the phoA system increased peak productivity by up to four times. At all scales, we typically observed peak productivities of ~7.5 g/L/h and sometimes achieved productivities even higher. This level of productivity is on par with and may exceed the productivities reported in “two-pot” processes, such as the processes described in Wada et al., 2005, and in Morishige et al., 2007, but with significantly less operator intervention since the process can be performed in a single pot. The high productivities we achieved with the phoA system significantly reduced the fermentation batch time required to achieve commercially relevant titers, which has implications for commercial viability. For example, production phase duration was reduced from 50-100+hours using the production process of Pandit et al., 2019, down to ~24 hours using the production process described herein (FIG. 6, compare solid and open circles), to achieve the same final titer of 60+g/L (FIG. 6, dashed line). Further, in the phosphate limited batches, production phase duration and final titer were tightly correlated (FIG. 6, solid circles), whereas in the constitutive system, titer was uncorrelated with production phase duration (FIG. 6, open circles). In practice, this means that extending the production phase enables conversion of more EG to GA in our process, which is paramount to scaling the process.

### Example 7: Fermentation Profiles of Genome-Integrated Vs. Plasmid Strains

A strain corresponding to the plasmid-containing strain described and characterized in preceding Examples was engineered to integrate two copies of each of the Gox and AldA genes into its genome using standard techniques (“integrated strain”). The fermentation profile of the integrated strain was compared to a corresponding plasmid-containing strain expected to have about 20 copies of each of the Gox and AldA genes, in a small scale 500 mL Applikon bioreactor. The results are shown in Table 1. Interestingly, these results show that the integrated strain reached about 37% of the glycolic acid productivity of the plasmid strain, despite the plasmid strain probably having ten times the number of copies of each of the Gox and AldA genes than the integrated strain.

TABLE 1
GA fermentation profiles of genome-integrated vs. plasmid strains

| | OD600 | | Glycolic acid (g/L) | |
|---|---|---|---|---|
| Fermentation time (h) | Integrated | Plasmid | Integrated | Plasmid |
| 0 | 4.6 | 6.7 | 0.00 | 0.00 |
| 2 | 11.4 | 12.2 | 0.00 | 0.00 |
| 3 | 9.8 | 13.75 | 0.00 | 0.00 |
| 4 | 11 | 15 | 0.00 | 0.00 |
| 5.5 | 25 | 19 | 0.00 | 0.00 |
| 6.5 | 26 | 28.8 | 0.00 | 0.00 |
| 8.5 | 25.9 | 24 | 0.00 | 0.00 |
| 11.75 | 24 | 21 | 0.00 | 0.00 |
| 21.1 | 24 | | 6.40 | 21.40 |
| 23.25 | | | 7.57 | 22.47 |
| 25.5 | | | 9.48 | 28.83 |
| 28.3 | | | 9.28 | 25.08 |

### Example 8: Cloning and Characterization of Orthologs of Gox0313 Alcohol Dehydrogenase

While Examples 1 to 7 relate to strains expressing the genes Gox and AldA encoding the Gox0313 alcohol dehydrogenase and the AldA aldehyde dehydrogenase enzymes, respectively, Examples 8 and 9 relate to the cloning and characterization of orthologs thereof.

### 8.1 Materials and Methods

Enzyme expression and purification: The enzymes of interest were genetically engineered in the E. coli BL21 (DE3) strain on a high copy plasmid under control of the T7 promoter. The enzymes were tagged with a hexahistidine (His-tag) sequence to facilitate for purification. Post-expression, the His-tagged enzymes were selectively bound to a nickel-charged immobilized metal affinity chromatography (IMAC) resin, exploiting the high affinity between histidine residues and nickel ions. Upon washing away non-specifically bound proteins, the His-tagged enzymes were eluted with an imidazole-containing buffer and subsequently used for enzymatic assays.

Enzyme assays: Standard enzyme characterization protocols were performed to determine the activities of the enzymes. Dehydrogenase activity against the substrates was determined using a continuous assay following the increase in absorbance at 340 nm ε340 (NADH)=6.22 mM⁻¹. cm⁻¹, and ε340 (NADPH)=6.2 mM⁻¹·cm⁻¹]. Substrate screens were performed at 30 °C in a reaction mixture (200 μL) containing 50 mM of substrate in 100 mM Tris-HCl (pH 8.5), 1 mM NAD⁺, 1 mM substrate, and 10 μg of purified protein. Initial velocity patterns of the enzymatic reaction were obtained by varying the concentrations of ethylene glycol at fixed concentrations of enzyme concentration in the presence of the saturating NAD concentrations.

### 8.2 Expression and Purification of Candidate Alcohol Dehydrogenases Capable of Converting Ethylene Glycol to Glycolaldehyde

A first enzymatic step in the processes described herein relate to the conversion of an aliphatic polyhydric alcohol substrate (e.g., ethylene glycol) to its corresponding aldehyde (e.g., glycolaldehyde) via a zinc-dependent alcohol dehydrogenase or other oxygen-insensitive oxidoreductase (FIG. 7). While Zhang et al., 2015 reported the production of glycolaldehyde from oxidation of ethylene glycol using the zinc-dependent medium-chain alcohol dehydrogenase (Gox0313) from Gluconobacter oxydans, the authors reported enzyme characteristics that may not be favorable for industrial applications. Thus, we sought to benchmark the catalytic activity of Gox0313 for ethylene glycol against that of other zinc-dependent enzymes with potential alcohol dehydrogenase activity.

After identifying a plurality potential enzymes of interest through bioinformatic searches, several candidates were selected from different branches of a phylogenetic tree for cloning, expression and characterization. Candidate alcohol dehydrogenase enzymes, along with Gox0313 (herein referred to as “goxADH”) as a control, were cloned and expressed in E. coli as described in Example 1, and the yields of several candidates following purification are shown in Table 2. Two of the candidate enzymes, rjoADH and scoADH, were found to be insoluble following purification, precluding any activity measurements.

TABLE 2
Candidate alcohol dehydrogenase enzymes expressed and purified

| Name | Organism | SEQ ID NO: | Protein Yield (mg/L) |
|---|---|---|---|
| goxADH | Gluconobacter oxydans | 206 | 53 |
| gmoADH | Gluconobacter morbifer | 207 | 20 |
| gdiADH | Gluconobacter diazotrophicus | 208 | 35 |
| mtaADH | Moraxella sp. (strain TAE123) | 209 | 8 |
| ecoADHP | Escherichia coli | 210 | 56 |
| gstADH | Geobacillus stearothermophilus | 211 | 69 |
| | (Bacillus stearothermophilus) | | |
| scoADH | Streptomyces coelicolor | 212 | Insoluble |
| cpsADH | Colwellia psychrerythraea | 213 | 51 |
| | (Vibrio psychroerythus) | | |
| rjoADH | Rhodococcus jostii (strain RHA1) | 214 | Insoluble |
| mmaADH | Mycobacterium marinum | 215 | 13 |

### 8.3 Characterization of Alcohol Dehydrogenase Activity

The candidate alcohol dehydrogenase enzymes were next characterized for their enzymatic activity. Initial activity screening was performed in a reaction mixture consisting of 50 mM of ethylene glycol (EG) as substrate in 100 mM Tris-HCl buffer at pH 8.5. Reactions were initiated by the addition of the purified enzyme and enzymatic activity was measured by monitoring the formation of the product (glycolaldehyde) over time as described in Example 8.1. Robust EG oxidation activity was observed for goxADH, gmoADH, mtaADH, gdiADH, and mmaADH. While a detectable level of EG oxidation activity was observed for ecoADHP, no EG oxidation activity was detected for gstADH and cpsADH (data not shown).

Kinetic parameters for goxADH, gmoADH, mtaADH, gdiADH, and mmaADH were then determined by conducting enzyme activity assays at varying concentrations of EG (100, 50, 25, 15, 10, 5, 2, and 1 mM). The resultant activity data were fitted to the Michaelis-Menten equation to derive kinetic constants (Kₘ and Vₘₐₓ) for each enzyme. These constants provide insights into the enzyme's substrate affinity and maximum catalytic rate, contributing to a better understanding of the enzyme's operational efficiency. As seen in Table 3, mmaADH exhibited similar substrate affinity and maximum catalytic rate to goxADH. Interestingly, enzymes mtaADH, gmoADH, and gdiADH exhibited comparable substrate affinities to one another, albeit with maximum catalytic rates (Vₘₐₓ) significantly higher than that of goxADH and mmaADH. The maximum catalytic rates of gmoADH and gdiADH were particularly striking, being 2.5 and 3.8 fold higher than that of goxADH, respectively.

TABLE 3
Michaelis-Menten kinetic constants of candidate alcohol dehydrogenases

| | SEQ | Kₘ | Vₘₐₓ | |
|---|---|---|---|---|
| Enzyme | ID NO: | (μM EG) | (μmoles•min⁻¹•mg⁻¹) | Relative Vmax |
| goxADH | 206 | 53 ± 7 | 2.0 ± 0.1 | 1.0 |
| mmaADH | 215 | 77 ± 8 | 2.2 ± 0.1 | 1.1 |
| mtaADH | 209 | 210 ± 65 | 3.2 ± 0.7 | 1.6 |
| gmoADH | 207 | 200 ± 44 | 4.9 ± 0.8 | 2.5 |
| gdiADH | 208 | 230 ± 87 | 7.5 ± 2.1 | 3.8 |

8.4 Structural Comparisons Between goxADH, mtaADH, gmoADH, and gdiADH

Pairwise sequence alignment analyses between the full-length amino acid sequences of goxADH, mtaADH, gmoADH, and gdiADH were performed using the EMBOSS Needle online tool under default settings and the results are shown in Tables 4 and 5 for identity and similarity, respectively. Interestingly, both gmoADH and gdiADH share relatively high amino acid sequence identity with goxADH yet exhibited 2.5- and 3.8-fold higher EG turnover rate, respectively. This observation suggests one or more specific amino acid residue positions that may be engineered for increasing EG turnover rate, as illustrated in the bolded residues in the multiple sequence alignment shown in FIG. 8A.

TABLE 4
Sequence identity comparisons of goxADH, mtaADH,
gmoADH, and gdiADH (FIG. 8B)

| % Identity | goxADH | mtaADH | gmoADH | gdiADH |
|---|---|---|---|---|
| goxADH | 100% | 65.3% | 89.8% | 78.9% |
| mtaADH | 65.3% | 100% | 65.9% | 63% |
| gmoADH | 89.8% | 65.9% | 100% | 74.4% |
| gdiADH | 78.9% | 63% | 74.4% | 100% |

TABLE 5
Sequence similarity comparisons between goxADH,
mtaADH, gmoADH, and gdiADH

| % | | | | |
|---|---|---|---|---|
| Similarity | goxADH | mtaADH | gmoADH | gdiADH |
| goxADH | 100% | 78.7% | 95.6% | 86.5% |
| mtaADH | 78.7% | 100% | 79.6% | 77.1% |
| gmoADH | 95.6% | 79.6% | 100% | 85.1% |
| gdiADH | 86.5% | 77.1% | 85.1% | 100% |

8.5 Identification of gmoADH Orthologs

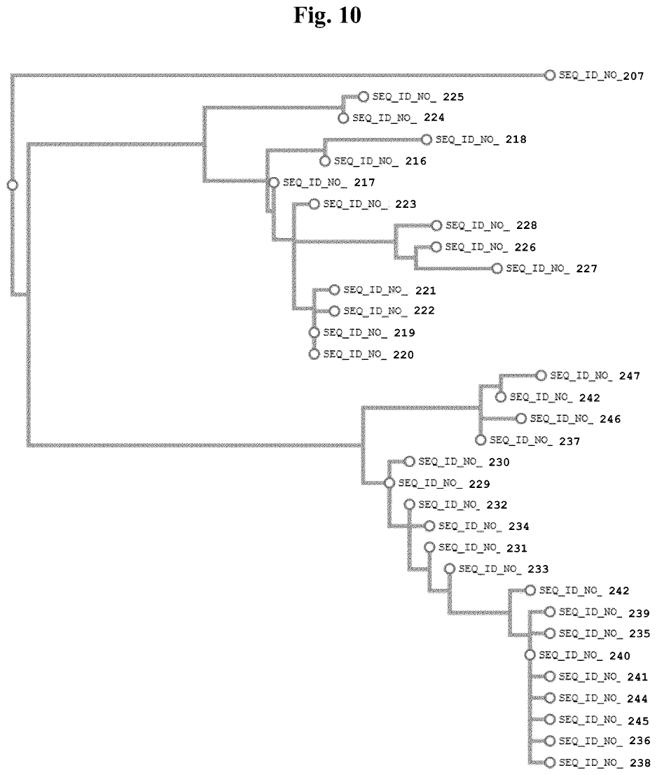

Orthologs of gmoADH were identified using sequence identity searches based on a cut-off of at least 85% overall sequence identity with respect to its full-length sequence (SEQ ID NO: 207). The hits are shown in Table 6, with corresponding multiple sequence alignments and phylogenetic trees shown in FIG. 9 and FIG. 10, respectively.

TABLE 6
Alcohol dehydrogenases at least 85% identical to gmoADH

| | | % Identity | | |
|---|---|---|---|---|
| SEQ ID NO: | Organism | to gmoADH | Length | Accession |
| 216 | Gluconobacter aidae | 90.67 | 343 | WP_153429267.1 |
| 217 | Gluconobacter oxydans | 90.38 | 343 | WP_062502113.1 |
| 218 | Gluconobacter roseus | 90.09 | 343 | WP_062509570.1 |
| 219 | Gluconobacter | 90.09 | 343 | WP_172491767.1 |
| 220 | Gluconobacter oxydans NBRC 3293 | 89.97 | 339 | GEM15596.1 |
| 221 | Gluconobacter oxydans | 89.8 | 343 | WP_011251899.1 |
| 222 | Gluconobacter oxydans | 89.8 | 343 | WP_062457674.1 |
| 223 | Gluconobacter oxydans | 89.8 | 343 | WP_041110782.1 |
| 224 | Gluconobacter | 89.21 | 343 | WP_194263585.1 |
| 225 | Gluconobacter potus | 88.92 | 343 | WP_062494900.1 |
| 226 | Gluconobacter sp. PIC6 b | 88.92 | 343 | WP_215745757.1 |
| 227 | Gluconobacter sp. Dm-62 | 88.92 | 343 | WP_211558619.1 |
| 228 | Gluconobacter oxydans | 88.92 | 343 | WP_225198465.1 |
| 229 | Gluconobacter thailandicus | 88.34 | 342 | WP_007282790.1 |
| 230 | Gluconobacter thailandicus | 88.05 | 342 | WP_148619246.1 |
| 231 | Gluconobacter frateurii | 88.05 | 342 | WP_099182847.1 |
| 232 | Gluconobacter japonicus | 88.05 | 342 | OAG72183.1 |
| 233 | Gluconobacter cerinus | 87.76 | 342 | WP_211516100.1 |
| 234 | Gluconobacter thailandicus | 87.76 | 342 | WP_061510882.1 |
| 235 | Gluconobacter | 87.46 | 342 | WP_211496647.1 |
| 236 | Gluconobacter cerinus | 87.46 | 342 | WP_211508717.1 |
| 237 | Gluconobacter japonicus | 87.46 | 342 | WP_010502028.1 |
| 238 | Gluconobacter cerinus | 87.17 | 342 | WP_211513430.1 |
| 239 | Gluconobacter | 87.17 | 342 | WP_215750737.1 |
| 240 | Gluconobacter | 87.17 | 342 | WP_046901231.1 |
| 241 | Gluconobacter cerinus | 87.17 | 342 | WP_203547627.1 |
| 242 | Gluconobacter japonicus | 87.17 | 342 | WP_062017061.1 |
| 243 | Gluconobacter cerinus | 86.88 | 342 | WP_099213207.1 |
| 244 | Gluconobacter cerinus | 86.88 | 342 | WP_211510153.1 |
| 245 | Gluconobacter cerinus | 86.88 | 342 | WP_211498472.1 |
| 246 | Gluconobacter japonicus | 86.88 | 342 | WP_062023663.1 |
| 247 | Gluconobacter japonicus | 86.59 | 342 | WP_282858877.1 |

8.6 Identification of gdiADH Orthologs

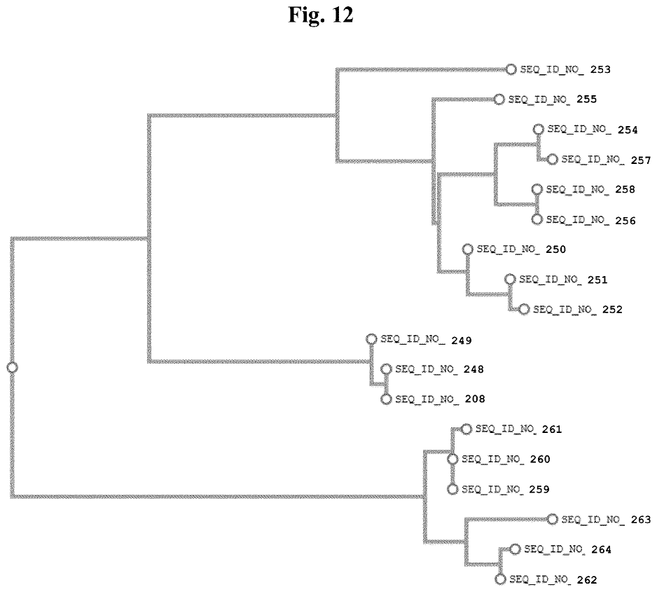

Orthologs of gdiADH were identified using sequence identity searches based on a cut-off of at least 85% overall sequence identity with respect to its full-length sequence (SEQ ID NO: 208). The hits are shown in Table 7, with corresponding multiple sequence alignments and phylogenetic trees shown in FIG. 11 and FIG. 12, respectively.

TABLE 7
Alcohol dehydrogenases at least 85% identical to gdiADH

| SEQ ID NO: | Organism | % Identity | Length | Accession |
|---|---|---|---|---|
| 248 | Gluconacetobacter diazotrophicus | 100 | 342 | WP_012553308.1 |
| 249 | Gluconacetobacter diazotrophicus | 99.71 | 343 | WP_144880052.1 |
| 250 | Gluconacetobacter asukensis | 91.5 | 342 | WP_182978775.1 |
| 251 | Gluconacetobacter tumulicola | 91.2 | 342 | WP_182964119.1 |
| 252 | Gluconacetobacter aggeris | 90.91 | 342 | WP_182986332.1 |
| 253 | Gluconacetobacter sacchari | 90.62 | 342 | WP_182998182.1 |
| 254 | Gluconacetobacter dulcium | 90.32 | 342 | WP_182973472.1 |
| 255 | Gluconacetobacter takamatsuzukensis | 90.32 | 342 | WP_182949773.1 |
| 256 | Gluconacetobacter liquefaciens | 90.03 | 342 | WP_114727250.1 |
| 257 | Gluconacetobacter dulcium | 90.03 | 342 | WP_183009106.1 |
| 258 | Gluconacetobacter liquefaciens NRIC | 89.91 | 338 | GBR10380.1 |
| | 0522 | | | |
| 259 | Gluconacetobacter azotocaptans DSM | 87.87 | 338 | GBQ33549.1 |
| | 13594 | | | |
| 260 | Gluconacetobacter azotocaptans | 87.43 | 342 | WP_183120793.1 |
| 261 | Gluconacetobacter azotocaptans | 87.13 | 342 | WP_204739834.1 |
| 262 | Gluconacetobacter johannae | 86.84 | 342 | WP_182943291.1 |
| 263 | Gluconacetobacter tumulisoli | 86.84 | 342 | WP_182961433.1 |
| 264 | Gluconacetobacter johannae | 86.55 | 346 | WP_220789739.1 |

### Example 9: Cloning and Characterization of Orthologs of AldA Alcohol Dehydrogenase

### 9.1 Expression and Purification of Candidate Alcohol Dehydrogenases Capable of Converting Ethylene Glycol to Glycolaldehyde

A second enzymatic step in the processes described herein relate to the conversion of an aliphatic polyhydric aldehyde substrate (e.g., glycolaldehyde) to a corresponding alpha-hydroxy acid (e.g., glycolic acid). E. coli aldehyde dehydrogenase aldA (ecoALDH) has been reported to catalyze the intracellular enzymatic conversion of glycolaldehyde (GA) to glycolic acid (e.g., Wada et al. 2005; Morishige et al., 2007; Pandit et al., 2019; Pandit et al., 2021). In order to identify potentially higher-performing enzymes for industrial use capable of converting glycolaldehyde to glycolic acid, we evaluated a plurality of candidates based on either close, medium, or distant phylogeny to ecoALDH (SEQ ID NO: 265). A subset of candidate enzymes were cloned, expressed, and purified, along with ecoALDH as a control, as described in Example 8.1, and their in vitro enzymatic activities were measured in a reaction mixture consisting of 50 mM glycolaldehyde in 100 mM Tris-HCl buffer (pH 8.5). The reaction was initiated by the addition of the purified enzyme, and activity was measured by monitoring the formation of glycolic acid over time. The resultant activity data were then used to calculate enzyme performance metrics for each enzyme, such as Kₘ (substrate affinity) and k_{cat} (enzyme turnover), as well as the inhibitor constant K_{I}, to assess whether the enzymes suffer from substrate inhibition (Kokkonen et al., 2021) with respect to glycolaldehyde.

The activity results for ecoALDH and two candidate enzymes of interest, aviALDH (from Azotobacter vinelandii; SEQ ID NO: 266) and ppuALDH (Pseudomonas putida; SEQ ID NO: 267), are shown in Table 8. Interestingly, the catalytic turnover rates of aviALDH and ppuALDH were 5.3- and 5.8-fold higher than that of ecoALDH, suggesting that these enzymes may be advantageous for industrial use (where saturating or near-saturating concentrations of glycolaldehyde are ideally employed). Furthermore, while both aviALDH and ppuALDH exhibited greater resistance to glycolaldehyde substrate inhibition than ecoALDH, the resistance to substrate inhibition of aviALDH was particularly striking at 5.6-fold higher than that of ecoALDH (Table 8).

TABLE 8
Michaelis-Menten kinetic constants of ecoALDH

| | | | Relative | | Relative |
|---|---|---|---|---|---|
| Enzyme | Kₘ (μM GA) | k_{cat} (s⁻¹) | k_{cat} | K_{I} (μM GA) | K_{I} |
| ecoALDH | 0.62 ± 0.14 | 5.7 ± 0.7 | 1.0 | 5.0 ± 1.3 | 1.0 |
| aviALDH | 7.5 ± 1.3 | 30 ± 4 | 5.3 | 28 ± 8 | 5.6 |
| ppuALDH | 7.2 ± 1.0 | 33 ± 3 | 5.8 | 6.3 ± 0.1 | 1.3 |

9.2 Structural Comparisons Between ecoALDH, aviALDH, and ppuALDH

Pairwise sequence alignment analyses between the full-length amino acid sequences of ecoALDH (SEQ ID NO: 265), aviALDH (SEQ ID NO: 266), and ppuALDH (SEQ ID NO: 267) were performed using the EMBOSS Needle online tool under default settings and the results are shown in Tables 9 and 10 for identity and similarity, respectively.

TABLE 9
Sequence identity comparisons between
ecoALDH, aviALDH, and ppuALDH

| % Identity | ecoALDH | aviALDH | ppuALDH |
|---|---|---|---|
| ecoALDH | 100% | 61.3% | 35% |
| aviALDH | 61.3% | 100% | 36.4% |
| ppuALDH | 35% | 3 6.4% | 100% |

TABLE 10
Sequence similarity comparisons between
ecoALDH, aviALDH, and ppuALDH

| % Similarity | ecoALDH | aviALDH | ppuALDH |
|---|---|---|---|
| ecoALDH | 100% | 74.4% | 53.2% |
| aviALDH | 74.4% | 100% | 51.6% |
| ppuALDH | 53.2% | 51.6% | 100% |

9.3 Identification of aviALDH Orthologs

Orthologs of aviALDH were identified using sequence identity searches based on a cut-off of at least 80% overall sequence identity with respect to its full-length sequence (SEQ ID NO: 266). The hits are shown in Table 11, with corresponding multiple sequence alignments shown in FIG. 13.

TABLE 11
Aldehyde dehydrogenases at least 80% identical to aviALDH

| | | % | | |
|---|---|---|---|---|
| SEQ ID NO: | Organism | Identity | Length | Accession |
| 268 | Pseudomonas sp. M47T1 | 85.5 | 477 | WP_008368327.1 |
| 269 | Stutzerimonas azotifigens | 84.7 | 478 | WP_028239809.1 |
| 270 | Pseudomonas | 84.42 | 477 | WP_029291268.1 |
| 271 | Pseudomonas proteolytica | 84.42 | 477 | WP_169890178.1 |
| 272 | Pseudomonas proteolytica | 84.42 | 477 | WP_169876320.1 |
| 273 | Pseudomonas proteolytica | 84.24 | 477 | WP_186765138.1 |
| 274 | Pseudomonas fluorescens | 84.24 | 477 | VVN72866.1 |
| 275 | Pseudomonas fluorescens | 84.21 | 477 | WP_038441569.1 |
| 276 | Pseudomonas proteolytica | 84.21 | 477 | WP_133714689.1 |
| 277 | Pseudomonas | 84.21 | 477 | WP_070994239.1 |
| 278 | Pseudomonas proteolytica | 84.21 | 477 | WP_092237063.1 |
| 279 | Pseudomonas | 84.03 | 477 | WP_161871916.1 |
| 280 | Pseudomonas orientalis | 83.61 | 477 | WP_057722499.1 |
| 281 | Pseudomonas sp. KBW05 | 83.61 | 477 | WP_124529786.1 |
| 282 | Pseudomonas fluorescens | 83.61 | 477 | WP_134924440.1 |
| 283 | Pseudomonas sp. Marseille-Q1929 | 83.61 | 477 | WP_207042074.1 |
| 284 | Pseudomonas brenneri | 83.58 | 477 | WP_314384139.1 |
| 285 | Pseudomonas sp. TNT2022 ID1044 | 83.58 | 477 | WP_273917539.1 |
| 286 | Pseudomonas fluorescens | 83.58 | 477 | WP_150771087.1 |
| 287 | Pseudomonas sp. 1912-s | 83.37 | 477 | WP_275966211.1 |
| 288 | Pseudomonas sp. ANT H12B | 83.37 | 477 | WP_149417056.1 |
| 289 | Pseudomonas azerbaijanoccidentalis | 83.33 | 477 | WP_248691361.1 |
| 290 | Pseudomonas | 83.33 | 477 | WP_150705504.1 |
| 291 | Pseudomonas sp. NFACC45 | 83.19 | 477 | WP_092453286.1 |
| 292 | Pseudomonas | 83.16 | 477 | WP_169378861.1 |
| 293 | Pseudomonas sp. YuFO8 | 83.16 | 477 | WP_323606829.1 |
| 294 | Pseudomonas putida | 83.16 | 477 | WP__274075407.1 |
| 295 | Pseudomonas sp. YuFO20 | 83.16 | 477 | WP_323627241.1 |
| 296 | Pseudomonas fluorescens | 83.16 | 477 | WP_101220559.1 |
| 297 | Pseudomonas sp. GM78 | 83.16 | 477 | WP_008055281.1 |
| 298 | Pseudomonas sp. TH32 | 83.16 | 477 | WP_201124215.1 |
| 299 | Pseudomonas | 83.16 | 477 | WP_259198831.1 |
| 300 | Pseudomonas | 82.98 | 477 | WP_149414508.1 |
| 301 | Pseudomonas fluorescens ABAC62 | 82.98 | 477 | KTC63164.1 |
| 302 | Pseudomonas sp. 2822-17 | 82.98 | 477 | WP_099583145.1 |
| 303 | Pseudomonas marginalis pv. | 82.98 | 477 | RMT89302.1 |
| | marginalis | | | |
| 304 | Pseudomonas cyclaminis | 82.98 | 477 | WP_193901897.1 |
| 305 | Pseudomonas mandelii | 82.95 | 477 | WP_154501285.1 |
| 306 | Pseudomonas sp. GL-RE-29 | 82.95 | 477 | WP_223503094.1 |
| 307 | Pseudomonas sp. CFBP13528 | 82.95 | 477 | WP_137200076.1 |
| 308 | Pseudomonas yamanorum | 82.95 | 477 | WP_177048909.1 |
| 309 | Pseudomonas sp. | 82.95 | 477 | WP_294739477.1 |
| 310 | Pseudomonas sp. Z18(2022) | 82.95 | 477 | WP_330213806.1 |
| 311 | Pseudomonas umsongensis | 82.91 | 476 | WP_151142874.1 |
| 312 | Pseudomonas kairouanensis | 82.77 | 477 | WP_135291809.1 |
| 313 | Pseudomonas cedrina | 82.77 | 477 | WP_307392962.1 |
| 314 | Pseudomonas | 82.77 | 477 | WP_032891120.1 |
| 315 | Pseudomonas lurida | 82.77 | 477 | WP_069023581.1 |
| 316 | Pseudomonas sp. 18.1.10 | 82.77 | 477 | WP_257781986.1 |
| 317 | Pseudomonas | 82.77 | 477 | WP_112195440.1 |
| 318 | Pseudomonas sp. S35 | 82.77 | 477 | WP_159935186.1 |
| 319 | Pseudomonas fluorescens | 82.77 | 477 | VVN50837.1 |
| 320 | Pseudomonas sp. | 82.74 | 477 | WP_288109654.1 |
| 321 | Pseudomonas sp. PD9R | 82.74 | 477 | WP_218436259.1 |
| 322 | Pseudomonas putida | 82.74 | 477 | HEF4758223.1 |
| 323 | Pseudomonas fluorescens | 82.74 | 477 | WP_034150932.1 |
| 324 | Pseudomonas | 82.74 | 477 | WP_020795476.1 |
| 325 | Pseudomonas yamanorum | 82.74 | 477 | WVN20288.1 |
| 326 | Pseudomonas yamanorum | 82.74 | 477 | WP_177134928.1 |
| 327 | Pseudomonas sp. GM79 | 82.74 | 477 | WP_008067014.1 |
| 328 | Pseudomonas fluorescens | 82.74 | 477 | WP_063340186.1 |
| 329 | Pseudomonas reinekei | 82.7 | 477 | WP_075945882.1 |
| 330 | Pseudomonas umsongensis | 82.7 | 476 | WP_168758619.1 |
| 331 | Pseudomonas sp. B14(2022) | 82.56 | 477 | WP_167655201.1 |
| 332 | Pseudomonas cedrina | 82.56 | 477 | WP_076954055.1 |
| 333 | Pseudomonas karstica | 82.56 | 477 | WP_154741605.1 |
| 334 | Pseudomonas sp. 2995-1 | 82.56 | 477 | WP_099602829.1 |
| 335 | Pseudomonas fluorescens | 82.56 | 477 | VVM13252.1 |
| 336 | unclassified Pseudomonas | 82.56 | 477 | WP_158878310.1 |
| 337 | Pseudomonas sivasensis | 82.56 | 477 | WP_179179121.1 |
| 338 | Pseudomonas brassicacearum | 82.53 | 477 | WP_123416563.1 |
| 339 | Pseudomonas sp. BIGb0381 | 82.53 | 477 | WP_259242465.1 |
| 340 | Pseudomonas | 82.53 | 477 | WP_145338818.1 |
| 341 | Pseudomonas sp. Xaverov 83 | 82.53 | 477 | WP_219273507.1 |
| 342 | Pseudomonas yamanorum | 82.53 | 477 | WP_309255961.1 |
| 343 | Pseudomonas sp. MWU16-30323 | 82.53 | 477 | WP_226478200.1 |
| 344 | Pseudomonas migulae | 82.45 | 474 | WP_084317994.1 |
| 345 | Pseudomonas sp. SJZ080 | 82.45 | 474 | WP_145317173.1 |
| 346 | Pseudomonas fluorescens | 82.45 | 474 | WP_017337646.1 |
| 347 | unclassified Pseudomonas | 82.35 | 477 | WP_184742015.1 |
| 348 | Pseudomonas lurida | 82.35 | 477 | WP_034108865.1 |
| 349 | Pseudomonas lurida | 82.35 | 477 | WP_305435308.1 |
| 350 | Pseudomonas fluorescens | 82.35 | 477 | WP_214917963.1 |
| 351 | Pseudomonas | 82.32 | 477 | WP_046817924.1 |
| 352 | Pseudomonas putida | 82.32 | 477 | WP_077185508.1 |
| 353 | Pseudomonas sp. A-R-19 | 82.28 | 477 | WP_223592911.1 |
| 354 | Pseudomonas sp. PB101 | 82.28 | 477 | WP_160389397.1 |
| 355 | Pseudomonas fluorescens | 82.14 | 477 | WP_078742242.1 |
| 356 | Pseudomonas | 82.14 | 477 | WP_315871898.1 |
| 357 | Pseudomonas azotoformans | 82.14 | 477 | WP_141606253.1 |
| 358 | Pseudomonas lurida | 82.14 | 477 | WP_186606063.1 |
| 359 | Pseudomonas lurida | 82.14 | 477 | WP_122767875.1 |
| 360 | Pseudomonas lurida | 82.14 | 477 | WP_187516619.1 |
| 361 | Pseudomonas fluorescens | 82.11 | 477 | WP_150817521.1 |
| 362 | Pseudomonas sp. | 82.11 | 477 | MBA4359864.1 |
| 363 | Pseudomonas pisciculturae | 81.93 | 477 | WP_194938147.1 |
| 364 | Pseudomonas sp. Tri1 | 81.93 | 477 | WP_210638821.1 |
| 365 | Pseudomonas marginalis | 81.93 | 477 | WP_281113802.1 |
| 366 | Pseudomonas sp. NFACC42-2 | 81.93 | 477 | WP_092489221.1 |

9.4 Identification of ppuALDH Orthologs

Orthologs of ppuALDH were identified using sequence identity searches based on a cut-off of at least 80% overall sequence identity with respect to its full-length sequence (SEQ ID NO: 267). The hits are shown in Table 12, with corresponding multiple sequence alignments shown in FIG. 14.

TABLE 12
Aldehyde dehydrogenases at least 80% identical to ppuALDH

| | | % | | |
|---|---|---|---|---|
| SEQ ID NO: | Organism | Identity | Length | Accession |
| 367 | Pseudomonadaceae | 100 | 499 | WP_024765054.1 |
| 368 | Pseudomonas aeruginosa | 99.8 | 499 | WP_023464836.1 |
| 369 | Pseudomonas aeruginosa | 99.8 | 499 | HBO7167994.1 |
| 370 | Pseudomonas aeruginosa | 99.8 | 499 | HBP6021006.1 |
| 371 | Pseudomonas sp. KBS0802 | 99.8 | 499 | WP_138868019.1 |
| 372 | Pseudomonas aeruginosa | 99.8 | 499 | MCO2870892.1 |
| 373 | Pseudomonas aeruginosa | 99.8 | 499 | WP_100857918.1 |
| 374 | Stutzerimonas balearica | 99.8 | 499 | WP_200291578.1 |
| 375 | Pseudomonas aeruginosa | 99.79 | 488 | WP_134462347.1 |
| 376 | Pseudomonas aeruginosa | 99.6 | 499 | MCT0894001.1 |
| 377 | Gammaproteobacteria bacterium | 99.4 | 499 | MBU1807863.1 |
| 378 | Pseudomonas sp. GOM6 | 99.4 | 499 | WP_277692171.1 |
| 379 | Pseudomonas sp. S9 | 99 | 499 | WP_010487157.1 |
| 380 | Pseudomonas sp. | 99 | 499 | WP_324522135.1 |
| 381 | Pseudomonas sp. | 98.87 | 443 | HCL15291.1 |
| 382 | Pseudomonas sp. | 98.8 | 499 | WP_298188589.1 |
| 383 | Pseudomonas aeruginosa | 98.8 | 499 | WP_031689744.1 |
| 384 | Pseudomonadota | 98.6 | 499 | WP_003291466.1 |
| 385 | Pseudomonas juntendi | 98.6 | 499 | WHL27328.1 |
| 386 | Pseudomonas sp. ES3-33 | 98.59 | 425 | WP_045057436.1 |
| 387 | Pseudomonas aeruginosa | 98.4 | 499 | MBG4687818.1 |
| 388 | Stutzerimonas zhaodongensis | 98.4 | 499 | WP_255831920.1 |
| 389 | Pseudomonadaceae | 98.4 | 499 | WP_057380853.1 |
| 390 | Pseudomonas putida | 98.4 | 499 | WP_100773692.1 |
| 391 | Pseudomonas fluorescens | 98.4 | 499 | WP_191833599.1 |
| 392 | Stutzerimonas frequens | 98.18 | 517 | WP_231098893.1 |
| 393 | Stutzerimonas frequens | 98.18 | 503 | KZX51374.1 |
| 394 | Pseudomonas monteilii | 98 | 499 | MCT8188980.1 |
| 395 | Pseudomonas | 97.6 | 499 | WP_004574278.1 |
| 396 | Pseudomonas | 97.39 | 499 | WP_010562393.1 |
| 397 | Pseudomonas | 97.39 | 499 | WP_110675911.1 |
| 398 | Pseudomonas sp. NIBR-H-19 | 97.19 | 499 | WP_232392560.1 |
| 399 | Pseudomonas veronii | 97.19 | 499 | WP_169886438.1 |
| 400 | Pseudomonas putida | 97.19 | 499 | WP_284396500.1 |
| 401 | Pseudomonas putida | 97.19 | 499 | WP_197889161.1 |
| 402 | Pseudomonas carnis | 97.19 | 499 | WP_181124933.1 |
| 403 | Pseudomonas shahriarae | 96.99 | 499 | WP_282405268.1 |
| 404 | Pseudomonas | 96.99 | 499 | WP_047300487.1 |
| 405 | Pseudomonas putida | 96.99 | 499 | WP_071984478.1 |
| 406 | Pseudomonas | 96.99 | 499 | WP_070095680.1 |
| 407 | Pseudomonas shahriarae | 96.99 | 499 | WP_273878237.1 |
| 408 | Pseudomonas putida | 96.79 | 499 | WP_323600815.1 |
| 409 | Azotobacter vinelandii | 89.58 | 499 | WP_012700465.1 |
| 410 | Stutzerimonas azotifigens | 87.78 | 499 | WP_028240402.1 |
| 411 | Stutzerimonas stutzeri | 87.78 | 499 | WP_186415956.1 |
| 412 | Stutzerimonas nitrititolerans | 87.58 | 499 | WP_312299267.1 |
| 413 | Pseudomonas sp. Q1-7 | 87.58 | 499 | WP_271408144.1 |
| 414 | Pseudomonas | 87.58 | 499 | WP_057384354.1 |
| 415 | Stutzerimonas stutzeri | 87.37 | 499 | MBA1235494.1 |
| 416 | Stutzerimonas nitrititolerans | 87.37 | 499 | WP_312919408.1 |
| 417 | Pseudomonas aeruginosa | 87.37 | 499 | WP_328263002.1 |
| 418 | Stutzerimonas nitrititolerans | 87.37 | 499 | WP_253162466.1 |
| 419 | Pseudomonadaceae bacterium T75 | 87.37 | 499 | WAD27030.1 |
| 420 | Stutzerimonas nitrititolerans | 87.37 | 499 | WP_313037550.1 |
| 421 | Pseudomonas urethralis | 86.97 | 499 | WP_176508822.1 |
| 422 | Pseudomonas | 86.77 | 499 | WP_054571898.1 |
| 423 | Pseudomonas | 86.77 | 499 | WP_061304122.1 |
| 424 | Stutzerimonas nitrititolerans | 86.77 | 499 | WP_313109891.1 |
| 425 | Pseudomonas asiatica | 86.57 | 499 | WP_208210496.1 |
| 426 | Pseudomonas sp. ACN8 | 85.37 | 499 | WP_095944740.1 |
| 427 | Cupriavidus basilensis | 85.37 | 499 | WP_059413894.1 |
| 428 | Cupriavidus sp. WS | 84.97 | 499 | WP_020200682.1 |
| 429 | Cupriavidus sp. BIC8F | 84.77 | 499 | WP_316157680.1 |
| 430 | Rhodococcus qingshengii | 83.57 | 494 | WP_219469071.1 |
| 431 | Rhodococcus ruber | 83.16 | 494 | WP_302071486.1 |
| 432 | Stutzerimonas nitrititolerans | 82.43 | 831 | WP_312937621.1 |
| 433 | Marinobacterium profundum | 82.16 | 526 | WP_245635137.1 |
| 434 | Mycobacteriaceae | 80.04 | 481 | WP_207550887.1 |

### REFERENCES

Boronat et al., (1983). Experimental Evolution of a Metabolic Pathway for Ethylene Glycol Utilization by Escherichia coli. Journal of Bacteriology, 153 (1): 134-139.
Kataoka et al., (2001). Glycolic Acid Production Using Ethylene Glycol-Oxidizing Microorganisms. Biosci. Biotechnol. Biochem., 65 (10), 2265-2270.
Morishige et al., (2007). Process For Producing Hydroxycarboxylic Acid. European Patent No. 2025760 B1.
Pandit et al., (2019). Production of Glycolate from Ethylene Glycol and Related Microbial Engineering. International Application Publication No. WO/2019/046946.
Pandit et al., (2021). Engineering Escherichia coli for the utilization of ethylene glycol. Microbial Cell Factories, 20 (1): 22. doi: 10.1186/s12934-021-01509-2.
Wada et al., (2005). Process For Producing Hydroxycarboxylic Acid. European Patent No. 1748076 B1. Zhang et al., (2015). Oxidation of ethylene glycol to glycolaldehyde using a highly selective alcohol dehydrogenase from Gluconobacter oxydans. Journal of Molecular Catalysis B: Enzymatic. 112:69-75.

1-32. (canceled)

33. A one-pot fermentation process for producing a hydroxycarboxylic acid, as a product or intermediate, from a substrate that is an aliphatic polyhydric alcohol having a terminal hydroxyl group, the process comprising:
(a) providing a microorganism comprising a first polynucleotide encoding a first enzyme which is a zinc-dependent alcohol dehydrogenase or other oxygen-insensitive oxidoreductase, that catalyzes the conversion of the aliphatic polyhydric alcohol substrate to its corresponding aldehyde; and a second polynucleotide encoding a second enzyme which is an aldehyde dehydrogenase that catalyzes the conversion of the corresponding aldehyde to a corresponding alpha-hydroxy acid,
(b) in a growth phase, culturing the microorganism for biomass accumulation in a fermentation broth comprising a native carbon source and under conditions of excess oxygen; and
(c) in a production phase, further culturing the microorganism for hydroxycarboxylic acid production in the fermentation broth in the presence of the aliphatic polyhydric alcohol substrate and under conditions of excess oxygen,
wherein at least the first polynucleotide encoding the first enzyme is comprised in an inducible expression cassette that is uninduced at the start of the growth phase and that becomes induced to initiate transition from the growth phase to the production phase upon the microorganism reaching a target biomass concentration suitable for hydroxycarboxylic acid production.

34. The process of claim 33, wherein the microorganism's endogenous glycolate oxidase activity is impaired or inactivated.

35. The process of claim 33, wherein the inducible expression cassette comprises a nutrient-sensing promoter or control element that turns on transcription of the expression cassette upon depletion of the nutrient in the fermentation broth below an expression threshold, and wherein the initial concentration of said nutrient in the fermentation broth prior to the start of the growth phase is tuned to achieve the target biomass concentration at which transcription from the expression cassette is turned on, thereby initiating transition from the growth phase to the production phase.

36. The process of claim 35, wherein the nutrient is phosphorous or phosphate, sulfur or sulfate, nitrogen, oxygen, or magnesium.

37. The process of claim 36, wherein the nutrient is phosphorous or phosphate and the concentration of the nutrient in the fermentation broth at the start of fermentation is less than 50 mM.

38. The process of claim 33, wherein the one-pot fermentation process is carried out at an industrial scale in at least a 50-L fermenter.

39. The process of claim 33, wherein:
(a) the expression cassette is a polycistronic expression cassette comprising the first and second polynucleotides;
(b) at least one of the first and second polynucleotides is heterologous or exogenous with respect to the microorganism;
(c) the expression cassette is comprised in an expression vector; or
(d) any combination of (a) to (c).

40. The process of claim 33, wherein the first enzyme is medium-chain zinc-dependent alcohol dehydrogenase.

41. The process of claim 33, wherein the second enzyme is an aldehyde dehydrogenase.

42. The process of claim 33, wherein the microorganism is one in which an endogenous hydroxycarboxylic acid-consuming activity is inactivated or decreased.

43. The process of claim 33, wherein the microorganism is one in which an endogenous glycolate oxidase and/or endogenous pyruvate formate-lyase activity is inactivated or decreased.

44. The process of claim 33, wherein the microorganism is bacteria.

45. The process of claim 33, wherein the aliphatic polyhydric alcohol substrate is present in the fermentation broth at the start of the growth phase, and/or is added to the fermentation broth after the start of the growth phase and prior to the start of the production phase.

46. The process of claim 33, wherein the process achieves a titer of glycolic acid of at least 25 g/L within 48 hours of production phase.

47. The process of claim 33, wherein the process achieves a peak productivity of at least 4 g/L/h.

48. The process of claim 33, wherein the aliphatic polyhydric alcohol substrate is a substrate of the zinc-dependent alcohol dehydrogenase.

49. The process of claim 33, wherein the aliphatic polyhydric alcohol substrate is ethylene glycol, diethylene glycol, triethylene glycol, tetraethylene glycol, propylene glycol, glycerol, 1,3-propanediol, 1,2-butanediol, 1,3-butanediol, 1,4-butanediol, or 1,2,4-butanetriol.

50. The process of claim 33, wherein the substrate is ethylene glycol and the hydroxycarboxylic acid is glycolic acid.

51. A process for reducing the production of by-products of overflow metabolism in a one-pot fermentation process, the process comprising:
(i) providing a microorganism engineered to express a biosynthetic pathway comprising multiple exogenous enzymes to produce a product of interest from a non-native feedstock, wherein expression of each of the biosynthetic pathway is controlled by a nutrient-sensing control element that turns on transcription of the multiple enzymes upon depletion of the nutrient below an expression threshold;
(ii) in a growth phase, culturing the microorganism for biomass accumulation in a fermentation broth comprising a native carbon source and under conditions of excess oxygen; and
(iii) in a production phase, further culturing the microorganism for production of the product of interest in the fermentation broth in the presence of the non-native feedstock and under conditions of excess oxygen,
wherein the initial concentration of said nutrient in the fermentation broth prior to the start of the growth phase is set to achieve a target biomass concentration at which nutrient depletion below the expression threshold turns on transcription of the biosynthetic pathway, thereby producing a delay in the expression of the biosynthetic pathway that reduces metabolic burden of the microorganism during the growth phase and a reduction in the production of by-products of overflow metabolism in the production phase.

52. The method of claim 51, wherein:
(a) the multiple exogenous enzymes are encoded by a polycistronic polynucleotide whose expression is controlled by the nutrient-sensing control element;
(b) the nutrient-sensing control element comprises or consists of a nutrient-sensing promoter;
(c) at least one of the multiple exogenous enzymes is heterologous with respect to the microorganism;
(d) at least one of the multiple exogenous enzymes encoded by a polynucleotide comprised in an expression vector or is integrated into the genome of the microbe in one or more copies;
(e) the microorganism is bacteria, yeast, or fungus;
(f) the by-products of overflow metabolism comprise one or more organic acids; or
(g) any combination of (a) to (f).
